(19) United States
(12) Patent Application Publication
(10) Pub. No.: US 2024/0051946 A1
(43) Pub. Date: Feb. 15, 2024

(54) TARGETED PROTEIN DEGRADATION OF PARP14 FOR USE IN THERAPY

(71) Applicant: Ribon Therapeutics, Inc., Cambridge, MA (US)

(72) Inventors: Nicholas Robert Perl, Somerville, MA (US); Kevin Wayne Kuntz, Woburn, MA (US)

(21) Appl. No.: 18/227,643

(22) Filed: Jul. 28, 2023

Related U.S. Application Data
(60) Provisional application No. 63/393,541, filed on Jul. 29, 2022.

(51) Int. Cl. C07D 403/14; A61K 45/06; C07D 405/14

(52) U.S. Cl. CPC C07D 403/14; A61K 45/06; C07D 405/14

(57) ABSTRACT

The present invention relates to quinazolinones and related compounds which degrade PARP14 and are useful, for example, in the treatment of cancer and inflammatory diseases.

### FIELD OF THE INVENTION

The present invention relates to quinazolinones and related compounds which cause intracellular proteolysis of PARP14 and are useful in the treatment of cancer and inflammatory diseases.

### BACKGROUND OF THE INVENTION

Poly(ADP-ribose) polymerases (PARPs) are members of a family of seventeen enzymes that regulate fundamental cellular processes including gene expression, protein degradation, and multiple cellular stress responses (Vyas S, et al. Nat Rev Cancer. 2014 Jun. 5; 14(7):502-509). The ability of cancer cells to survive under stress is a fundamental cancer mechanism and an emerging approach for novel therapeutics. One member of the PARP family, PARP1, has already been shown to be an effective cancer target in connection to cellular stress induced by DNA damage, either induced by genetic mutation or with cytotoxic chemotherapy, with three approved drugs in the clinic and several others in late stage development (Ohmoto A, et al. OncoTargets and Therapy. 2017; Volume 10:5195).

The seventeen members of the PARP family were identified in the human genome based on the homology within their catalytic domains (Vyas S, et al. Nat Commun. 2013 Aug. 7; 4:2240). However, their catalytic activities fall into 3 different categories. The majority of PARP family members catalyze the transfer of mono-ADP-ribose units onto their substrates (monoARTs), while others (PARP1, PARP2, TNKS, TNKS2) catalyze the transfer of poly-ADP-ribose units onto substrates (polyARTs). Finally, PARP13 is thus far the only PARP for which catalytic activity could not be demonstrated either in vitro or in vivo.

PARP14 is a cytosolic as well as nuclear monoART. It was originally identified as BAL2 (B Aggressive Lymphoma 2), a gene associated with inferior outcome of diffuse large B cell lymphoma (DLBCL), together with two other monoARTs (PARP9 or BAL1 and PARP15 or BAL3) (Aguiar R C, et al. Blood. 2000 Dec. 9; 96(13):4328-4334 and Juszczynski P, et al. Mol Cell Biol. 2006 Jul. 1; 26(14):5348-5359). PARP14, PARP9 and PARP15 are also referred to as macro-PARPs due to the presence of macro-domains in their N-terminus. The genes for the three macroPARPs are located in the same genomic locus suggesting co-regulation. Indeed, the gene expression of PARP14 and PARP9 is highly correlated across normal tissues and cancer types. PARP14 is overexpressed in tumors compared to normal tissues, including established cancer cell lines in comparison to their normal counterparts. Literature examples of cancers with high PARP14 expression are DLBCL (Aguiar R C T, et al. J Biol Chem. 2005 Aug. 1; 280(40):33756-33765), multiple myeloma (MM) (Barbarulo A, et al. Oncogene. 2012 Oct. 8; 32(36):4231-4242) and hepatocellular carcinoma (HCC) (Iansante V, et al. Nat Commun. 2015 Aug. 10; 6:7882). In MM and HCC cell lines RNA interference (RNAi) mediated PARP14 knockdown inhibits cell proliferation and survival. Other studies show that the enzymatic activity of PARP14 is required for survival of prostate cancer cell lines in vitro (Bachmann S B, et al. Mol Cancer. 2014 May 27; 13:125).

PARP14 is an interferon stimulated gene with its mRNA increased by stimulation of various cell systems with all types of interferon (I, II and III; www.interferome.org). PARP14 has been identified as a downstream regulator of IFN-γ and IL-4 signaling, influencing transcription downstream of STAT1 (in the case of IFN-γ) (Iwata H, et al. Nat Commun. 2016 Oct. 31; 7:12849) or STAT6 (in the case of IL-4) (Goenka S, et al. Proc Natl Acad Sci USA. 2006 Mar. 6; 103(11):4210-4215; Goenka S, et al. J Biol Chem. 2007 May 3; 282(26):18732-18739; and Mehrotra P, et al. J Biol Chem. 2010 Nov. 16; 286(3):1767-1776). Parp14 −/− knockout (KO) mice have reduced marginal zone B cells, and the ability of IL-4 to confer B cell survival in vitro was reduced as well in the Parp14 KO setting (Cho S H, et al. Blood. 2009 Jan. 15; 113(11):2416-2425). This decreased survival signaling was linked mechanistically to decreased abilities of Parp14 KO B cells to sustain metabolic fitness and to increased Mcl-1 expression. Parp14 KO can extend survival in the Eμ-Myc lymphoma model, suggesting a role of PARP14 in Myc-driven lymphomagenesis (Cho S H, et al. Proc Natl Acad Sci USA. 2011 Sep. 12; 108(38):15972-15977). Gene expression data point towards roles of PARP14 in human B cell lymphoma as well. The BAL proteins, including PARP14, are highly expressed in host response (HR) DLBCLs, a genomically defined B cell lymphoma subtype characterized by a robust inflammatory infiltrate of T and dendritic cells and presence of an IFN-γ gene signature (Molecular profiling of diffuse large B-cell lymphoma identifies robust subtypes including one characterized by host inflammatory response. Monti S, et al. Blood. 2005; 105(5):1851).

Due to its role downstream of IL-4 and IFN-γ signaling pathways PARP14 has been implicated in T helper cell and macrophage differentiation. Genetic PARP14 inactivation in macrophages skews to a pro-inflammatory M1 phenotype associated with antitumor immunity while reducing a pro-tumor M2 phenotype. M1 gene expression, downstream of IFN-γ, was found to be increased while M2 gene expression, downstream of IL-4, was decreased with PARP14 knockout or knockdown in human and mouse macrophage models. Similarly, genetic PARP14 knockout has been shown to reduce a Th2 T helper cell phenotype in the setting of skin and airway inflammation, again pertaining to the regulatory role of PARP14 in IL-4 signal transduction (Mehrotra P, et al. J Allergy Clin Immunol. 2012 Jul. 25; 131(2):521 and Krishnamurthy P, et al. Immunology. 2017 Jul. 27; 152(3):451-461).

PARP14 promotes signaling by Type 2 helper T cells (TH2) and Type 17 helper T cells (TH17) cytokines by acting as a coactivator of STAT6- and STAT3-driven transcription (Goenka et al. 2006 PMID 16537510, Mehrotra et al. 2015 PMID 26222149). PARP14 is upregulated in tissues with inflammatory disease, such as the skin lesions in atopic dermatitis or psoriasis patients (He et al. 2021 PMID: 32709423) or in endobronchial biopsies from mild atopic asthma patients (Yick et al. 2013 PMID: 23314903). Either genetic deletion or catalytic inhibition of PARP14 has been shown to block IL 4/STAT6 signaling in macrophages in vitro (Iwata et al. 2016 PMID 27796300, Schenkel et al. 2021 PMID: 33705687) and to suppress pathogenic changes associated with allergic airway disease in mouse models (Cho et al. 2013 PMID: 23956424, Mehrotra et al. 2013 PMID: 22841009, Eddie et al. 2022 PMID: 35817532). Antibodies and small molecules suppressing TH2/TH17-cytokine signaling and alarmins are either approved or being investigated as treatments for multiple inflammatory diseases such as atopic dermatitis, asthma, chronic rhinosinusitis, and eosinophilic esophagitis (Sastre et al. 2018, PMID: 29939132, Lyly et al. 2020 PMID: 33322143, Ahn et al. 2021 PMID: 33911806, Ahn et al. 2021 PMID: 33935450). Given the upregulation of PARP14 in tissues with inflammatory diseases, the central role of PARP14 in TH2- and TH17-driven cytokine signaling, and the common underlying biology of many inflammatory diseases, small molecules targeting PARP14 could be promising therapeutics for a broad range of inflammatory diseases.

Most clinically used pharmaceutical agents are based upon small-molecule inhibition of protein function. However, alternative approaches that provide for protein degradation, rather than inhibition, also have the potential to provide clinical efficacy. Accordingly, targeted protein degradation through ubiquitination of protein targets has emerged as an effective strategy in drug discovery. Heterobifunctional small molecules, which simultaneously bind to target proteins and recruit an ubiquitin ligase (e.g., ubiquitin E3 ligase) have been shown to result in the target protein's ubiquitination and degradation (Bondeson, D. P., et al. Nat Chem Biol. 2015 11(8):611-617). Examples of these small molecules, which can bind to both PARP14 and ubiquitin E3 ligase, have been described in PCT Patent Publication WO 2020/257416.

There is a need for the development of new drugs, such as small molecules that can bind to both PARP14 and ubiquitin E3 ligase to cause PARP14 degradation, which are useful in the treatment of various diseases, including cancer and inflammatory diseases.

### SUMMARY OF THE INVENTION

The present invention is directed to a compound of Formula (I):

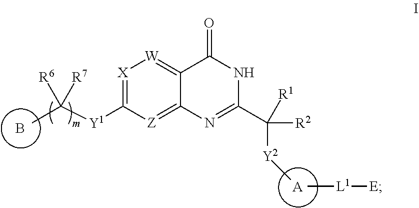

or a pharmaceutically acceptable salt thereof, wherein constituent members are defined below.

The present invention is further directed to a pharmaceutical composition comprising a compound of Formula (I), or a pharmaceutically acceptable salt thereof, and at least one pharmaceutically acceptable carrier.

The present invention is further directed to a method of degrading PARP14, comprising contacting a compound of Formula (I), or a pharmaceutically acceptable salt thereof, with PARP14.

The present invention is further directed to a method of treating a disease or disorder in a patient in need of treatment, where the disease or disorder is characterized by overexpression or increased activity of PARP14, comprising administering to the patient a therapeutically effective amount of a compound Formula (I), or a pharmaceutically acceptable salt thereof.

The present invention is further directed to a method of treating cancer in a patient in need thereof comprising administering to said patient a therapeutically effective amount of a compound of Formula (I), or a pharmaceutically acceptable salt thereof.

The present invention is further directed to a method of treating an inflammatory disease in a patient in need of treatment comprising administering to said patient a therapeutically effective amount of a compound of Formula (I), or a pharmaceutically acceptable salt thereof.

The present invention also provides uses of the compounds described herein in the manufacture of a medicament for use in therapy. The present disclosure also provides the compounds described herein for use in therapy.

### BRIEF DESCRIPTION OF THE DRAWINGS

FIG. 1 is a graph showing the dose-dependent reduction of Alternaria-induced cell infiltration in BALF following treatment with Example 17.

FIG. 2A is a graph demonstrating that Compound 17 significantly reduces eosinophil in BALF in a dose-dependent manner starting from 100 mg/kg.

FIG. 2B is a graph demonstrating that Compound 17 significantly reduces cytokine IL-33 in BALF in a dose-dependent manner starting from 100 mg/kg.

FIG. 2C is a graph demonstrating that Compound 17 significantly reduces cytokine IL-4 in BALF in a dose-dependent manner starting from 100 mg/kg.

FIG. 2D is a graph demonstrating that Compound 17 significantly reduces cytokine IL-5 in BALF in a dose-dependent manner starting from 100 mg/kg.

### DETAILED DESCRIPTION

The present disclosure provides, inter alia, a compound of Formula (I):

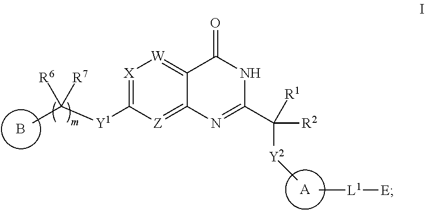

or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, —O—, and —(C₂₋₄ alkynyl)-;
Y² is selected from —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and (—NR³)CH₂—;
Ring A is selected from 6-10 membered aryl, 5-10 membered heteroaryl, C₃₋₁₄ cycloalkyl, and 3-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋₈ dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{d2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3} NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase;
L² is selected from the following:
   (i) bond, such that ring A is directly attached to moiety E;
   (ii) —(C₁₋₄ alkyl)-;
   (iii) —(C₂₋₄ alkenyl)-;
   (iv) —(C₂₋₄ alkynyl);
   (v) the following structure:

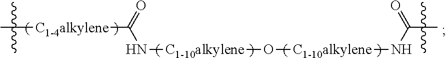

and
(vi) the following structure:

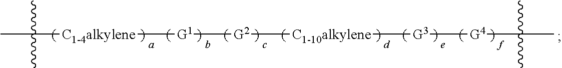

wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl;
G³ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G⁴ is 4-10 membered heterocycloalkyl;
each R^{G} is independently selected from H, methyl, and ethyl;
a is 0 or 1;
b is 0 or 1;
c is 0 or 1;
d is 0 or 1;
e is 0 or 1;
f is 0 or 1;
wherein at least one of b, c, e, and f is 1; and
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

In some embodiments, the compound is other than:
2-(4-(((7-(Cyclopentylamino)-5-fluoro-4-oxo-3,4-dihydroquinazolin-2-yl)methyl)thio)piperidin-1-yl)-N-(6-((2-(2,6-dioxopiperidin-3-yl)-1,3-dioxoisoindolin-4-yl)amino)hexyl)acetamide;
(2S,4R)-1-((S)-2-(7-(2-(4-(((7-(cyclopentylamino)-5-fluoro-4-oxo-3,4-dihydroquinazolin-2-yl)methyl)thio)piperidin-1-yl)acetamido)heptanamido)-3,3-dimethylbutanoyl)-4-hydroxy-N-(4-(4-methylthiazol-5-yl)benzyl)pyrrolidine-2-carboxamide;
8-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)octanamide; and
3-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)propanamide.

The present disclosure provides, inter alia, a compound of Formula (I):

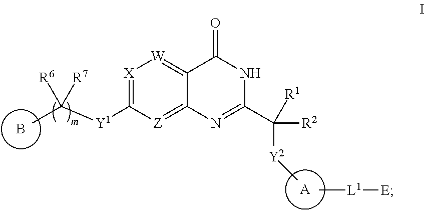

or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from NR³, CR⁴R⁵, and O;
Y² is selected from —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and (—NR³)CH₂—;
Ring A is selected from 6-10 membered aryl, 5-10 membered heteroaryl, C₃₋₁₄ cycloalkyl, and 3-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋₈ dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{b1}, R^{b2}, R^{c2}, R^{d2}, R^{a3}, R^{b3}, R³, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4}, R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase;
L¹ is selected from the following:
   (i) bond, such that ring A is directly attached to moiety E;
   (ii) —(C₁₋₄ alkyl)-;
   (iii) —(C₂₋₄ alkenyl)-;
   (iv) —(C₂₋₄ alkynyl);
   (v) the following structure:

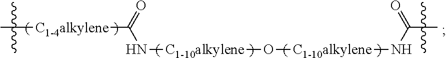

and
(vi) the following structure:

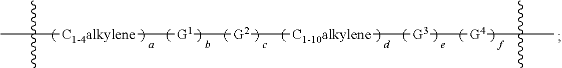

wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl;
G³ is selected from —C(O)—, —NR^{G}C(O)—, —NR^{G}—, —O—, —S—, —C(O)O—, —OC(O)NR^{G}_, —NR^{G}C(O)NR^{G}—, —S(O₂)—, and —S(O)NR^{G}_;
G⁴ is 4-10 membered heterocycloalkyl;
each R^{G} is independently selected from H, methyl, and ethyl;
a is 0 or 1;
b is 0 or 1;
c is 0 or 1;
d is 0 or 1;
e is 0 or 1;
f is 0 or 1;
wherein at least one of b, c, e, and f is 1; and
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

In some embodiments, the compound is other than:
2-(4-(((7-(Cyclopentylamino)-5-fluoro-4-oxo-3,4-dihydroquinazolin-2-yl)methyl)thio)piperidin-1-yl)-N-(6-((2-(2,6-dioxopiperidin-3-yl)-1,3-dioxoisoindolin-4-yl)amino)hexyl)acetamide;
(2S,4R)-1-((S)-2-(7-(2-(4-(((7-(cyclopentylamino)-5-fluoro-4-oxo-3,4-dihydroquinazolin-2-yl)methyl)thio)piperidin-1-yl)acetamido)heptanamido)-3,3-dimethylbutanoyl)-4-hydroxy-N-(4-(4-methylthiazol-5-yl)benzyl)pyrrolidine-2-carboxamide;
8-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)octanamide; and
3-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)propanamide.

In some embodiments, W is CR^{W}. In some embodiments, W is N.

In some embodiments, X is CR^{X}. In some embodiments, X is N.

In some embodiments, Z is CR^{Z}. In some embodiments, Z is N.

In some embodiments, Y¹ is —O—. In some embodiments, Y¹ is —CR⁴R⁵—. In some embodiments, Y¹ is —NR³—. In some embodiments, Y¹ is —O— or —NR³—.

In some embodiments, Y¹ is —(C₂₋₄ alkynyl)-. In some embodiments, Y¹ is —(C₂ alkynyl)-.

In some embodiments, Y¹ is —CR⁴R⁵— or —(C₂₋₄ alkynyl)-. In some embodiments, Y¹ is —CR⁴R⁵— or —(C₂ alkynyl)-.

In some embodiments, Y² is S. In some embodiments, Y² is —CH₂—. In some embodiments, Y² is —S— or —CH₂—. In some embodiments, Y² is selected from —S—, —S(O)—, —S(O)₂—, —CH₂—, —O—, and —N(R³)—. In some embodiments, Y² is selected from —SCH₂—, —S(O)CH₂—, —S(O)₂CH₂—, —CH₂CH₂—, —OCH₂—, and —(NR³)CH₂—. In some embodiments, Y² is —O—.

In some embodiments, Ring A is 4-18 membered heterocycloalkyl, wherein Ring A is optionally substituted by 1, 2, 3, or 4 R^{A}.

In some embodiments, Ring A is 4-7 membered heterocycloalkyl, wherein Ring A is optionally substituted by 1, 2, 3, or 4 R^{A}. In some embodiments, Ring A is 4-7 membered heterocycloalkyl, wherein Ring A is optionally substituted by 1 or 2 R^{A}. In some embodiments, Ring A is 4-7 membered heterocycloalkyl.

In some embodiments, Ring A is piperidinyl, optionally substituted by 1, 2, 3, or 4 R^{A}. In some embodiments, Ring A is piperidinyl, optionally substituted by R^{A}. In some embodiments, Ring A is piperidinyl. In some embodiments, Ring A is piperazinyl, optionally substituted by R^{A}.

In some embodiments, Ring A is 1-methylpiperidin-4-yl.

In some embodiments, Ring A is piperazinyl.

In some embodiments, Ring A is C₃₋₁₄ cycloalkyl, wherein Ring A is optionally substituted by 1, 2, 3, or 4 R^{A}. In some embodiments, Ring A is C₃₋₇ cycloalkyl, wherein Ring A is optionally substituted by 1, 2, 3, or 4 R^{A}. In some embodiments, Ring A is cyclohexyl.

In some embodiments, Ring B is C₃₋₇ cycloalkyl or 4-7 membered heterocycloalkyl, wherein Ring B is optionally substituted by 1, 2, 3, or 4 R^{B}. In some embodiments, Ring B is C₃₋₇ cycloalkyl wherein Ring B is optionally substituted by 1, 2, 3, or 4 R^{B}. In some embodiments, Ring B is 4-7 membered heterocycloalkyl, wherein Ring B is optionally substituted by 1, 2, 3, or 4 R^{B}.

In some embodiments, Ring B is C₃₋₇ cycloalkyl or 4-7 membered heterocycloalkyl, wherein Ring B is optionally substituted by 1 or 2 R^{B}. In some embodiments, Ring B is C₃₋₇ cycloalkyl, wherein Ring B is optionally substituted by 1 or 2 R^{B}. In some embodiments, Ring B is 4-7 membered heterocycloalkyl, wherein Ring B is optionally substituted by 1 or 2 R^{B}.

In some embodiments, Ring B is C₃₋₇ cycloalkyl. In some embodiments, Ring B is cyclopentyl or cyclopropyl. In some embodiments, Ring B is cyclopentyl. In some embodiments, Ring B is cyclopropyl.

In some embodiments, Ring B is piperidinyl or tetrahydro-2H-pyranyl optionally substituted by 1, 2, 3, or 4 R^{B}. In some embodiments, Ring B is piperidinyl or tetrahydro-2H-pyranyl optionally substituted by 1 or 2 R^{B}. In some embodiments, Ring B is piperidinyl or tetrahydro-2H-pyranyl, each optionally substituted by R^{B}.

In some embodiments, Ring B is piperidinyl optionally substituted by 1, 2, 3, or 4 R^{B}. In some embodiments, Ring B is piperidinyl optionally substituted by 1 or 2 R^{B}. In some embodiments, Ring B is piperidinyl substituted by R^{B}.

In some embodiments, Ring B is tetrahydro-2H-pyranyl optionally substituted by 1, 2, 3, or 4 R^{B}. In some embodiments, Ring B is tetrahydro-2H-pyranyl optionally substituted by 1 or 2 R^{B}. In some embodiments, Ring B is tetrahydro-2H-pyranyl.

In some embodiments, Ring B is tetrahydro-2H-pyran-4-yl or 1-acetylpiperidin-4-yl. In some embodiments, Ring B is 1-acetylpiperidin-4-yl.

In some embodiments, Ring B is piperidinyl, tetrahydro-2H-pyranyl, cyclopentyl, or cyclobutyl, wherein Ring B is optionally substituted by 1, 2, 3, or 4 R^{B}. In some embodiments, Ring B is piperidinyl, tetrahydro-2H-pyranyl, cyclopentyl, or cyclobutyl, wherein Ring B is optionally substituted by 1 or 2 R^{B}. In some embodiments, Ring B is piperidinyl, tetrahydro-2H-pyranyl, cyclopentyl, or cyclobutyl, wherein Ring B is optionally substituted by R^{B}. In some embodiments, Ring B is tetrahydro-2H-pyran-4-yl, 1-acetylpiperidin-4-yl, cyclobutyl, or cyclopentyl.

In some embodiments, R¹ and R² are each H. In some embodiments, R¹ is H. In some embodiments, R² is H.

In some embodiments, R³ is H.

In some embodiments, R⁴ and R⁵ are each H. In some embodiments, R⁴ is H. In some embodiments, R⁵ is H.

In some embodiments, R⁶ and R⁷ are each H. In some embodiments, R⁶ is H. In some embodiments, R⁷ is H.

In some embodiments, each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR_{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, and C₁₋₆ haloalkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, NR^{c1}R^{d1}, NR^{c1}C(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ alkyl, C₁₋₆ haloalkyl, CN, NO₂, or OR^{a1}. In some embodiments, each R^{A} is C₁₋₆ alkyl. In some embodiments, each R^{A} is C₁₋₆ alkyl or C₁₋₆ haloalkyl. In some embodiments, R^{A} is methyl.

In some embodiments, each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})R^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, and C₁₋₆ haloalkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, NR^{c2}R^{d2}, NR^{c2}C(O)R^{b2}, S(O)NR^{c2}R^{d2}, S(O)₂R^{b2}, and S(O)₂NR^{c2}R^{d2}.

In some embodiments, each R^{B} is independently selected from halo, C₁₋₆ alkyl, C₁₋₆ haloalkyl, CN, NO₂, C(O)R^{b2}, or OR^{a2}. In some embodiments, each R^{B} is independently selected from halo, C₁₋₆ alkyl, C₁₋₆ haloalkyl, and C(O)R^{b2}. In some embodiments, each R^{B} is independently selected from C(O)R^{b2}. In some embodiments, each R^{B} is independently selected from C(O)CH₃.

In some embodiments, R^{W}, R^{X}, and R^{Z} are each, independently, selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, and C₁₋₆ haloalkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR³C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{W} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{W} is selected from H, halo, C₁₋₆ alkyl, C₂₋₆ alkenyl, C₂₋₆ alkynyl, C₁₋₆ haloalkyl, CN, NO₂, and OR^{a3}. In some embodiments, R^{W} is selected from H, halo, and C₁₋₆ haloalkyl. In some embodiments, R^{W} is H or F. In some embodiments, R^{W} is F. In some embodiments, R^{W} is H.

In some embodiments, R^{X} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{X} is selected from C₆₋₁₀ aryl and 5-10 membered heteroaryl, wherein said C₆₋₁₀ aryl and 5-10 membered heteroaryl are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, OR^{a3}, and SR^{a3}.

In some embodiments, R^{X} is selected from H, halo, C₁₋₆ alkyl, C₂₋₆ alkenyl, C₂₋₆ alkynyl, C₁₋₆ haloalkyl, CN, OR^{a3}, and C₆₋₁₀ aryl. In some embodiments, R^{X} is H.

In some embodiments, R^{Z} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{Z} is selected from C₆₋₁₀ aryl and 5-10 membered heteroaryl, wherein said C₆₋₁₀ aryl and 5-10 membered heteroaryl are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, OR^{a3}, and SR^{a3}.

In some embodiments, R^{Z} is selected from H, halo, C₁₋₆ alkyl, C₂₋₆ alkenyl, C₂₋₆ alkynyl, C₁₋₆ haloalkyl, CN, OR^{a3}, and C₆₋₁₀ aryl. In some embodiments, R^{Z} is H.

In some embodiments, m is 1. In some embodiments, m is 0. In some embodiments, m is 2. In some embodiments, m is 0 or 1. In some embodiments, m is 1 or 2.

In some embodiments, L¹ is a bond, such that ring A is directly attached to moiety E.

In some embodiments, L¹ is —(C₁₋₄ alkyl)-.

In some embodiments, L¹ is —(C₂₋₄ alkenyl)-.

In some embodiments, L¹ is —(C₂₋₄ alkynyl)-.

In some embodiments, L¹ is —(C₂₋₄ alkynyl)-(G³)-.

In some embodiments, L¹ has the following structure:

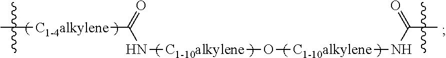

In some embodiments, L¹ has the following structure:

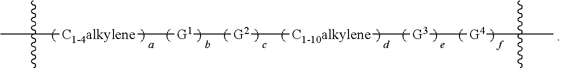

In some embodiments, L¹ is

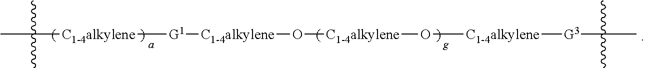

In some embodiments, G¹ is —NR^{G}C(O)— or —C(O)—. In some embodiments, G¹ is —NR^{G}C(O)—. In some embodiments, G¹ is —C(O)—. In some embodiments, G¹ is —NR^{G}C(O)—, —C(O)—, or —O—. In some embodiments, G1 is —O—.

In some embodiments, G² is 4-10 membered heterocycloalkyl. In some embodiments, G² is piperidinyl, piperazinyl, or azetidinyl. In some embodiments, G² is piperidinyl or piperazinyl. In some embodiments, G² is piperidinyl. In some embodiments, G² is piperazinyl. In some embodiments, G² is azetidinyl. In some embodiments, G² is pyrrolidinyl, piperidinyl, piperazinyl, or azetidinyl. In some embodiments, G² is pyrrolidinyl.

In some embodiments, G² is C₃₋₇ cycloalkyl. In some embodiments, G² is cyclobutyl.

In some embodiments, G³ is —NR^{G}C(O)—, —NR^{G}—, or —C(O)—. In some embodiments, G³ is —NR^{G}— or —O—. In some embodiments, G³ is —NR^{G}—. In some embodiments, G³ is —O—.

In some embodiments, G⁴ is piperidinyl or piperazinyl. In some embodiments, G⁴ is piperidinyl. In some embodiments, G⁴ is piperazinyl.

In some embodiments, a is 0. In some embodiments, a is 1.

In some embodiments, b is 0. In some embodiments, b is 1.

In some embodiments, c is 0. In some embodiments, c is 1.

In some embodiments, d is 0. In some embodiments, d is 1.

In some embodiments, e is 0. In some embodiments, e is 1.

In some embodiments, f is 0. In some embodiments, f is 1.

In some embodiments, g is 0. In some embodiments, g is 1.

In some embodiments, R^{G} is H.

Ubiquitin ligase binding moieties and linkers are known and well-described in the art, for example: Bondeson, D. P., et al. Nat Chem Biol. 2015 11(8):611-617; An S, et al. EBioMedicine 2018 36:553-562; Paiva S-L. et al, Curr. Op. in Chem. Bio. 2010, 50:111-119; and International Patent Application Publication No. WO 2017/197056, each of which is incorporated by reference in its entirety.

In some embodiments, E is a Von Hippel-Lindau (VHL) E3 ubiquitin ligase binding moiety, a MDM2 E3 ubiquitin ligase binding moiety, a cereblon E3 ubiquitin ligase binding moiety, or an inhibitor of apoptosis proteins (IAP) E3 ubiquitin ligase binding moiety, each of which has an IC₅₀ of less than about 10 μM as determined in a binding assay. For example, E is a cereblon E3 ubiquitin ligase binding moiety. E can be a Von Hippel-Lindau (VHL) E3 ubiquitin ligase binding moiety. E can be a MDM2 E3 ubiquitin ligase binding moiety. E can be an IAP E3 ubiquitin ligase binding moiety.

In some embodiments, E is an E3 ubiquitin ligase binding moiety that binds to cereblon.

In some embodiments, E comprises a chemical group derived from an imide, a thioimide, an amide, or a thioamide.

In some embodiments, E is thalidomide, lenalidomide, pomalidomide, analogs thereof, isosteres thereof, or derivatives thereof.

In some embodiments, E is selected from the following:

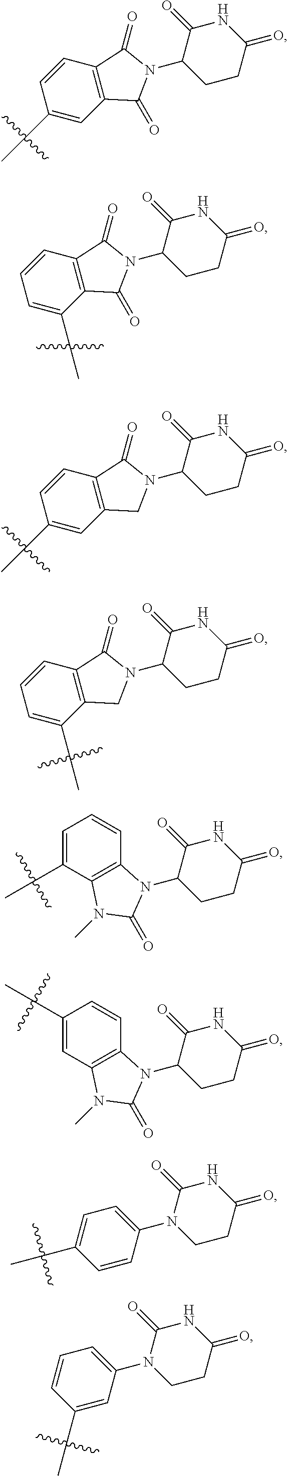

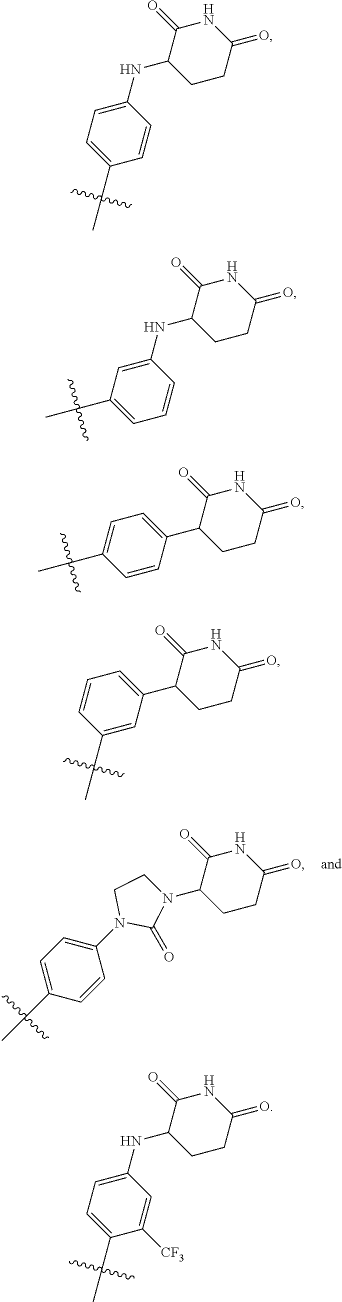 [SMILES: *c1ccc(NC2CCC(=O)NC2=O)cc1 ; *c1cccc(NC2CCC(=O)NC2=O)c1 ; *c1ccc(C2CCC(=O)NC2=O)cc1 ; *c1cccc(C2CCC(=O)NC2=O)c1 ; *c1ccc(N2CCN(C3CCC(=O)NC3=O)C2=O)cc1 ; *c1ccc(NC2CCC(=O)NC2=O)cc1C(F)(F)F]

wherein the wavy lines represent the point of attachment to group L¹.

In some embodiments, E is selected from:

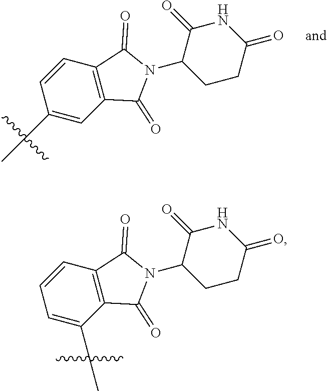 [SMILES: *c1ccc2c(c1)C(=O)N(C1CCC(=O)NC1=O)C2=O ; *c1cccc2c1C(=O)N(C1CCC(=O)NC1=O)C2=O]

wherein the wavy lines represent the point of attachment to group L¹.

In some embodiments, E is:

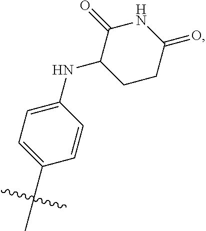 [SMILES: *c1ccc(NC2CCC(=O)NC2=O)cc1]

wherein the wavy line represents the point of attachment to group L¹.

In some embodiments, E is selected from the following:

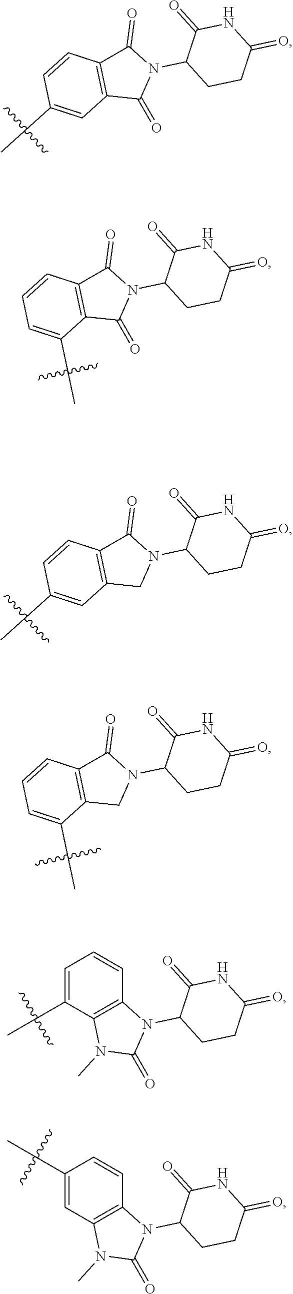 [SMILES: *c1ccc2c(c1)C(=O)N(C1CCC(=O)NC1=O)C2=O ; *c1cccc2c1C(=O)N(C1CCC(=O)NC1=O)C2=O ; *c1ccc2c(c1)CN(C1CCC(=O)NC1=O)C2=O ; *c1cccc2c1CN(C1CCC(=O)NC1=O)C2=O ; *c1cccc2c1n(C)c(=O)n2C1CCC(=O)NC1=O ; *c1ccc2c(c1)n(C)c(=O)n2C1CCC(=O)NC1=O]

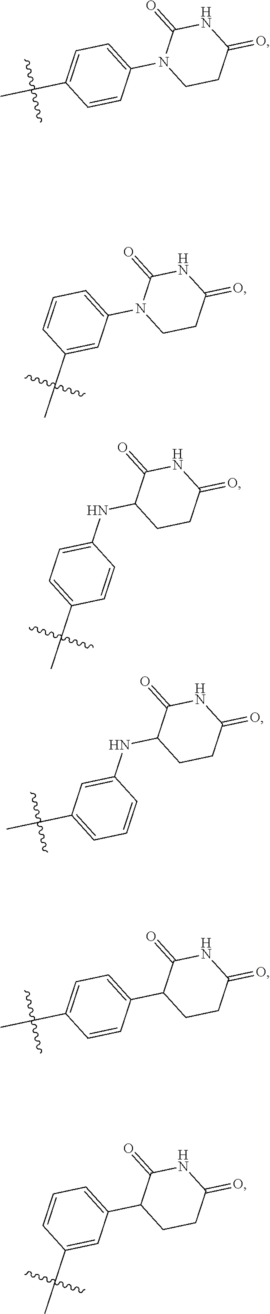 [SMILES: *c1ccc(N2CCC(=O)NC2=O)cc1 ; *c1cccc(N2CCC(=O)NC2=O)c1 ; *c1ccc(NC2CCC(=O)NC2=O)cc1 ; *c1cccc(NC2CCC(=O)NC2=O)c1 ; *c1ccc(C2CCC(=O)NC2=O)cc1 ; *c1cccc(C2CCC(=O)NC2=O)c1]

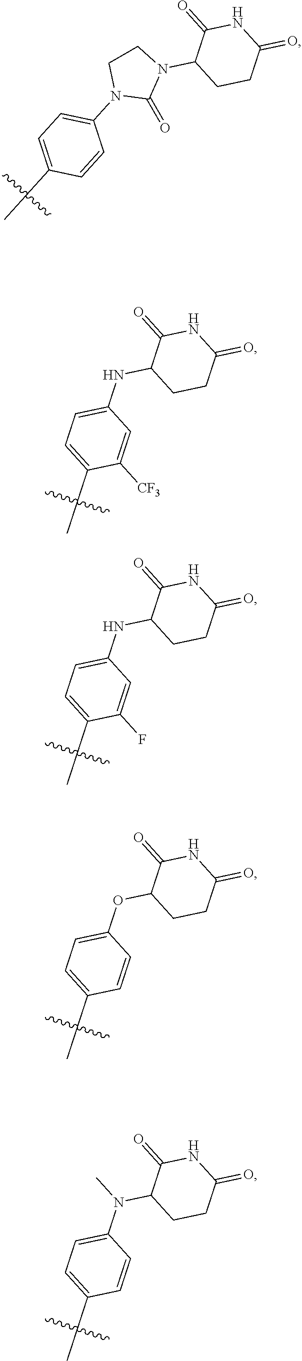 [SMILES: *c1ccc(N2CCN(C3CCC(=O)NC3=O)C2=O)cc1 ; *c1ccc(NC2CCC(=O)NC2=O)cc1C(F)(F)F ; *c1ccc(NC2CCC(=O)NC2=O)cc1F ; *c1ccc(OC2CCC(=O)NC2=O)cc1 ; *c1ccc(N(C)C2CCC(=O)NC2=O)cc1]

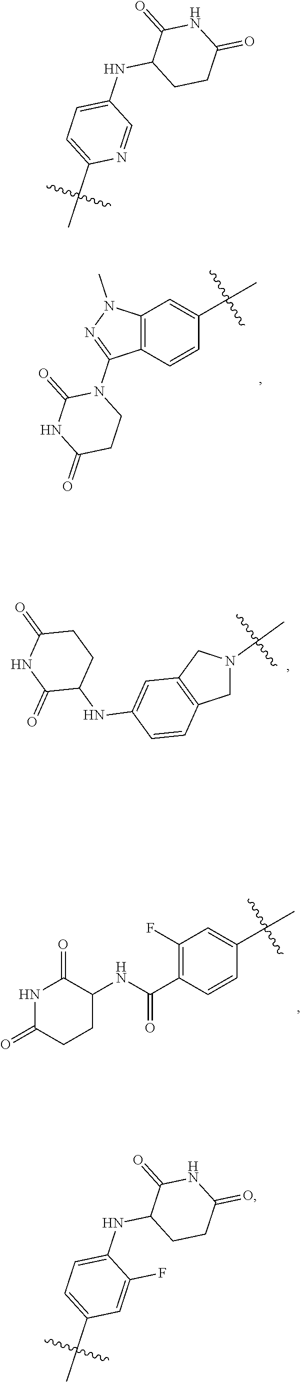 [SMILES: *c1ccc(NC2CCC(=O)NC2=O)cn1 ; *c1ccc2c(N3CCC(=O)NC3=O)nn(C)c2c1 ; *N1Cc2ccc(NC3CCC(=O)NC3=O)cc2C1 ; *c1ccc(C(=O)NC2CCC(=O)NC2=O)c(F)c1 ; *c1ccc(NC2CCC(=O)NC2=O)c(F)c1]

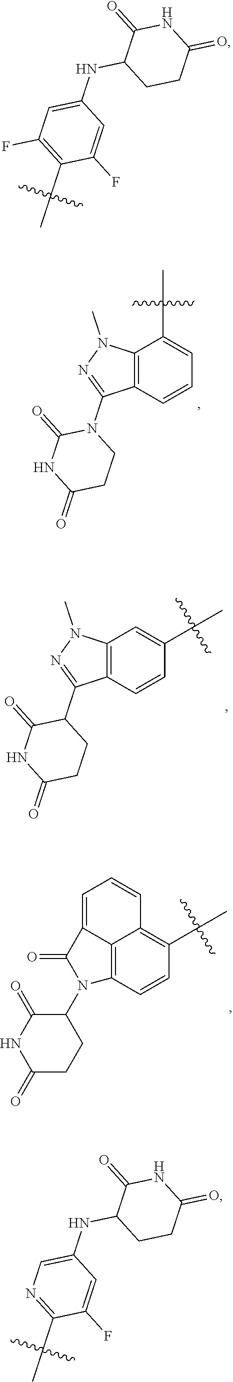 [SMILES: *c1c(F)cc(NC2CCC(=O)NC2=O)cc1F ; *c1cccc2c(N3CCC(=O)NC3=O)nn(C)c12 ; *c1ccc2c(C3CCC(=O)NC3=O)nn(C)c2c1 ; *c1ccc2c3c(cccc13)C(=O)N2C1CCC(=O)NC1=O ; *c1ncc(NC2CCC(=O)NC2=O)cc1F]

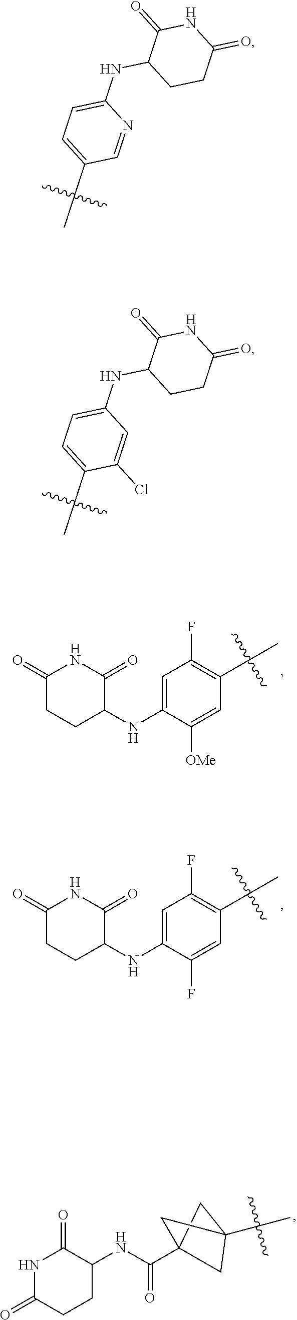 [SMILES: *c1ccc(NC2CCC(=O)NC2=O)nc1 ; *c1ccc(NC2CCC(=O)NC2=O)cc1Cl ; *c1cc(OC)c(NC2CCC(=O)NC2=O)cc1F ; *c1cc(F)c(NC2CCC(=O)NC2=O)cc1F ; *C12CC(C(=O)NC3CCC(=O)NC3=O)(C1)C2]

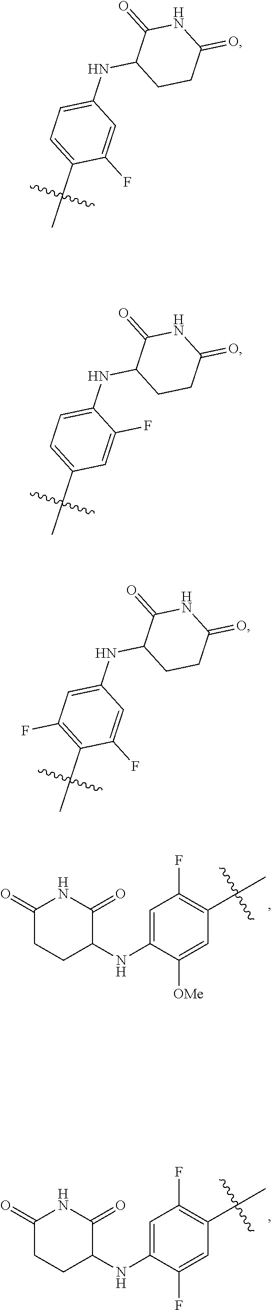 [SMILES: *c1ccc(NC2CCC(=O)NC2=O)cc1F ; *c1ccc(NC2CCC(=O)NC2=O)c(F)c1 ; *c1c(F)cc(NC2CCC(=O)NC2=O)cc1F ; *c1cc(OC)c(NC2CCC(=O)NC2=O)cc1F ; *c1cc(F)c(NC2CCC(=O)NC2=O)cc1F]

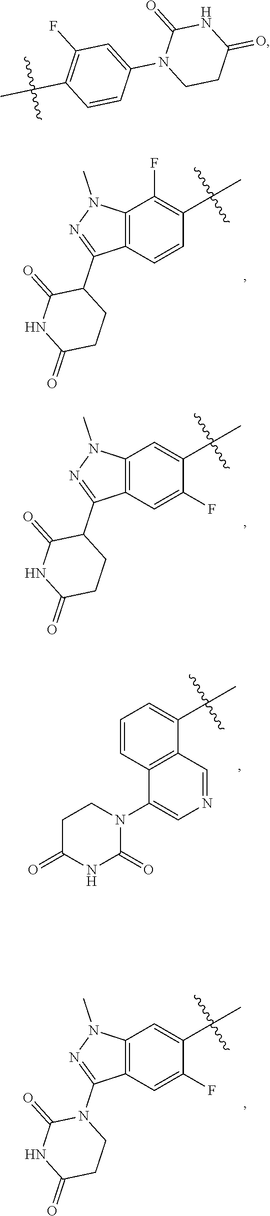

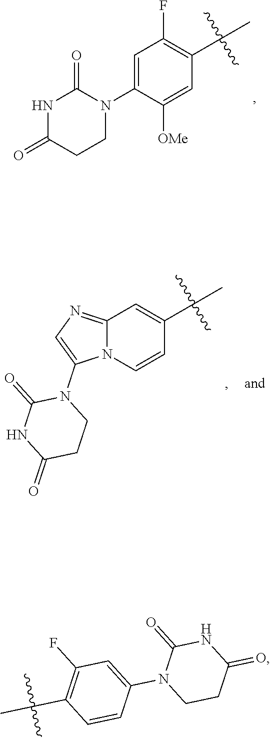 [SMILES: *c1cc(OC)c(N2CCC(=O)NC2=O)cc1F ; *c1ccn2c(N3CCC(=O)NC3=O)cnc2c1 ; *c1ccc(N2CCC(=O)NC2=O)cc1F]

wherein the wavy line represents the point of attachment to group L¹.

In some embodiments, the compound has Formula II:

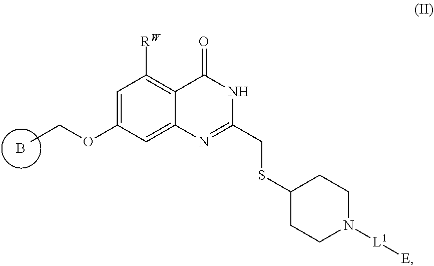

or a pharmaceutically acceptable salt thereof.

In some embodiments, the compound has Formula IIIa:

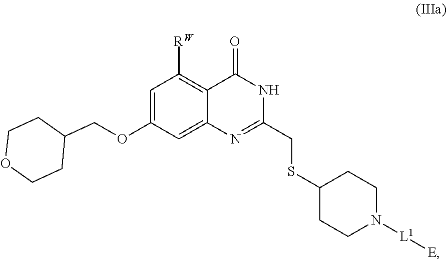

or a pharmaceutically acceptable salt thereof.

In some embodiments, the compound has Formula IIIb:

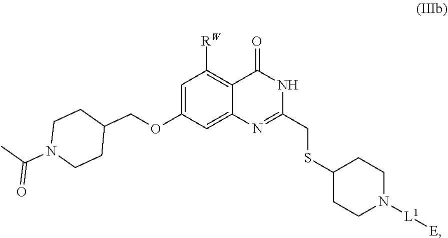

or a pharmaceutically acceptable salt thereof.

In some embodiments, the compound has Formula IIc:

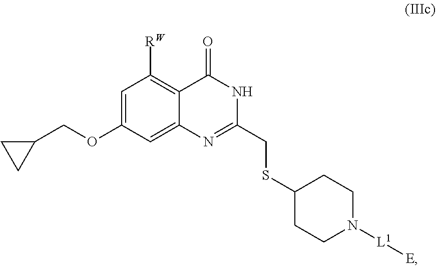

or a pharmaceutically acceptable salt thereof.

In some embodiments, the compound is selected from the following:
4-(4-((1-(2-(4-(((7-(cyclopentylamino)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
2-(2,6-dioxopiperidin-3-yl)-4-(4-((1-(2-(4-(((5-fluoro-4-oxo-7-((tetrahydro-2H-pyran-4-yl)methoxy)-3,4-dihydroquinazolin-2-yl)methyl)thio)piperidin-1-yl)acetyl)piperidin-4-yl)methyl)piperazin-1-yl)isoindoline-1,3-dione;
3-(2,4-dioxotetrahydropyrimidin-1(2H)-yl)-N-(6-(2-(4-(((5-fluoro-4-oxo-7-((tetrahydro-2H-pyran-4-yl)methoxy)-3,4-dihydroquinazolin-2-yl)methyl)thio)piperidin-1-yl)acetamido)hexyl)benzamide;
3-(2,4-dioxotetrahydropyrimidin-1(2H)-yl)-N-(4-(2-(4-(((5-fluoro-4-oxo-7-((tetrahydro-2H-pyran-4-yl)methoxy)-3,4-dihydroquinazolin-2-yl)methyl)thio)piperidin-1-yl)acetamido)butyl)benzamide;
N-(6-(4-(4-((2,6-dioxopiperidin-3-yl)amino)phenyl)piperidin-1-yl)hexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(6-(4-(4-((2,6-dioxopiperidin-3-yl)amino)phenyl)piperidin-1-yl)-6-oxohexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(4-((2,6-dioxopiperidin-3-yl)amino)phenyl)piperidin-1-yl)butyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(4-((2,6-dioxopiperidin-3-yl)amino)phenyl)piperidin-1-yl)-4-oxobutyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(4-((2,6-dioxopiperidin-3-yl)amino)phenyl)piperidin-1-yl)ethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(4-((2,6-dioxopiperidin-3-yl)amino)phenyl)piperidin-1-yl)-2-oxoethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(3-((2,6-dioxopiperidin-3-yl)amino)phenyl)piperidin-1-yl)-2-oxoethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(6-(4-(3-((2,6-dioxopiperidin-3-yl)amino)phenyl)piperidin-1-yl)-6-oxohexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(3-((2,6-dioxopiperidin-3-yl)amino)phenyl)piperidin-1-yl)butyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(3-((2,6-dioxopiperidin-3-yl)amino)phenyl)piperidin-1-yl)-4-oxobutyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(3-((2,6-dioxopiperidin-3-yl)amino)phenyl)piperidin-1-yl)ethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(6-(4-(3-((2,6-dioxopiperidin-3-yl)amino)phenyl)piperidin-1-yl)hexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
3-((4-(1-((1-(2-(4-(((5-fluoro-4-oxo-7-((tetrahydro-2H-pyran-4-yl)methoxy)-3,4-dihydroquinazolin-2-yl)methyl)thio)piperidin-1-yl)acetyl)piperidin-4-yl)methyl)piperidin-4-yl)phenyl)amino)piperidine-2,6-dione;
3-(3-(4-(1-(2-(4-(((7-((1-acetylpiperidin-4-yl)methoxy)-5-fluoro-4-oxo-3,4-dihydroquinazolin-2-yl)methyl)thio)piperidin-1-yl)acetyl)piperidin-4-yl)phenyl)-2-oxoimidazolidin-1-yl)piperidine-2,6-dione;
4-(4-((1-(2-(4-(((7-((1-acetylpiperidin-4-yl)methoxy)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
1-(4-(1-((1-(2-(4-(((5-fluoro-4-oxo-7-((tetrahydro-2H-pyran-4-yl)methoxy)-3,4-dihydroquinazolin-2-yl)methyl)thio)piperidin-1-yl)acetyl)piperidin-4-yl)methyl)piperidin-4-yl)phenyl)dihydropyrimidine-2,4(1H,3H)-dione;
4-((2-(4-(((7-((1-acetylpiperidin-4-yl)methoxy)-5-fluoro-4-oxo-3,4-dihydroquinazolin-2-yl)methyl)thio)piperidin-1-yl)-2-oxoethyl)amino)-2-(2,6-dioxopiperidin-3-yl)isoindoline-1,3-dione;
4-(4-((4-(4-(((7-((1-acetylpiperidin-4-yl)methoxy)-5-fluoro-4-oxo-3,4-dihydroquinazolin-2-yl)methyl)thio)piperidine-1-carbonyl)piperidin-1-yl)methyl)piperidin-1-yl)-2-(2,6-dioxopiperidin-3-yl)isoindoline-1,3-dione;
4-(4-((4-(4-(((7-(cyclopentylamino)-5-fluoro-4-oxo-3,4-dihydroquinazolin-2-yl)methyl)thio)piperidine-1-carbonyl)piperidin-1-yl)methyl)piperidin-1-yl)-2-(2,6-dioxopiperidin-3-yl)isoindoline-1,3-dione;
5-((2-(4-(((7-((1-acetylpiperidin-4-yl)methoxy)-5-fluoro-4-oxo-3,4-dihydroquinazolin-2-yl)methyl)thio)piperidin-1-yl)-2-oxoethyl)amino)-2-(2,6-dioxopiperidin-3-yl)isoindoline-1,3-dione;
3-((4-(4-(((7-((1-acetylpiperidin-4-yl)methoxy)-5-fluoro-4-oxo-3,4-dihydroquinazolin-2-yl)methyl)thio)piperidine-1-carbonyl)phenyl)amino)piperidine-2,6-dione;
4-(4-((4-(4-(2-(7-((1-acetylpiperidin-4-yl)methoxy)-5-fluoro-4-oxo-3,4-dihydroquinazolin-2-yl)ethyl)piperidine-1-carbonyl)piperidin-1-yl)methyl)piperidin-1-yl)-2-(2,6-dioxopiperidin-3-yl)isoindoline-1,3-dione;
4-(4-((1-(2-(4-(2-(7-((1-acetylpiperidin-4-yl)methoxy)-5-fluoro-4-oxo-3,4-dihydroquinazolin-2-yl)ethyl)piperidin-1-yl)acetyl)piperidin-4-yl)methyl)piperazin-1-yl)-2-(2,6-dioxopiperidin-3-yl)isoindoline-1,3-dione;
5-(4-((1-(2-(4-(((7-((1-acetylpiperidin-4-yl)methoxy)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
5-(4-((1-(2-(4-(((7-(cyclopentylamino)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
5-(4-(((7-((1-acetylpiperidin-4-yl)methoxy)-5-fluoro-4-oxo-3,4-dihydroquinazolin-2-yl)methyl)thio)piperidin-1-yl)-2-(2,6-dioxopiperidin-3-yl)isoindoline-1,3-dione;
2-(4-(((7-((1-acetylpiperidin-4-yl)methoxy)-5-fluoro-4-oxo-3,4-dihydroquinazolin-2-yl)methyl)thio)piperidin-1-yl)-N-(2-(2,6-dioxopiperidin-3-yl)-1,3-dioxoisoindolin-4-yl)acetamide;
2-(4-(((7-((1-acetylpiperidin-4-yl)methoxy)-5-fluoro-4-oxo-3,4-dihydroquinazolin-2-yl)methyl)thio)piperidin-1-yl)-N-(2-(2,6-dioxopiperidin-3-yl)-1,3-dioxoisoindolin-5-yl)acetamide;
5-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione;
5-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-2-(2,6-dioxopiperidin-3-yl)isoindoline-1,3-dione;
3-(4-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-3-methyl-2-oxo-2,3-dihydro-1H-benzo[d]imidazol-1-yl)piperidine-2,6-dione;
3-(5-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-3-methyl-2-oxo-2,3-dihydro-1H-benzo[d]imidazol-1-yl)piperidine-2,6-dione;
1-(4-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)phenyl)dihydropyrimidine-2,4(1H,3H)-dione;
1-(3-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)phenyl)dihydropyrimidine-2,4(1H,3H)-dione;
4-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione;
3-(4-(3-((4-(((7-((1-acetylpiperidin-4-yl)methoxy)-5-fluoro-4-oxo-3,4-dihydroquinazolin-2-yl)methyl)thio)piperidin-1-yl)methyl)azetidin-1-yl)-1-oxoisoindolin-2-yl)piperidine-2,6-dione; and
5-(4-((4-(((7-(cyclopropylmethoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione;
   or a pharmaceutically acceptable salt of any of the aforementioned.

In some embodiments, the compound is selected from:
3-((4-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)phenyl)amino)piperidine-2,6-dione;
3-((4-(4-(2-(4-(((5-fluoro-4-oxo-7-((tetrahydro-2H-pyran-4-yl)methoxy)-3,4-dihydroquinazolin-2-yl)methyl)thio)piperidin-1-yl)ethyl)piperazin-1-yl)phenyl)amino)piperidine-2,6-dione;
3-((3-fluoro-4-(4-(2-(4-((5-fluoro-4-oxo-7-((tetrahydro-2H-pyran-4-yl)methoxy)-3,4-dihydroquinazolin-2-yl)methoxy)piperidin-1-yl)ethyl)piperazin-1-yl)phenyl)amino)piperidine-2,6-dione;
3-((4-(4-(2-(4-((7-(cyclopropylmethoxy)-5-fluoro-4-oxo-3,4-dihydroquinazolin-2-yl)methoxy)piperidin-1-yl)ethyl)piperazin-1-yl)-3-fluorophenyl)amino)piperidine-2,6-dione;
3-((3-fluoro-4-(4-(2-(4-(((5-fluoro-4-oxo-7-((tetrahydro-2H-pyran-4-yl)methoxy)-3,4-dihydroquinazolin-2-yl)methyl)thio)piperidin-1-yl)ethyl)piperazin-1-yl)phenyl)amino)piperidine-2,6-dione;
3-((4-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)-3-fluorophenyl)amino)piperidine-2,6-dione;
3-((4-(4-(3-(4-(((7-(cyclopropylmethoxy)-5-fluoro-4-oxo-3,4-dihydroquinazolin-2-yl)methyl)thio)piperidin-1-yl)azetidin-1-yl)piperidin-1-yl)-3-fluorophenyl)amino)piperidine-2,6-dione;
3-(6-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)-2-oxobenzo[cd]indol-1(2H)-yl)piperidine-2,6-dione;
3-(5-(4-((4-(((7-(cyclopropylmethoxy)-5-fluoro-4-oxo-3,4-dihydroquinazolin-2-yl)methyl)thio)piperidin-1-yl)methyl)piperidin-1-yl)-1-oxoisoindolin-2-yl)piperidine-2,6-dione;
3-((3-fluoro-4-(4-(3-(4-(((5-fluoro-4-oxo-7-((tetrahydro-2H-pyran-4-yl)methoxy)-3,4-dihydroquinazolin-2-yl)methyl)thio)piperidin-1-yl)azetidin-1-yl)piperidin-1-yl)phenyl)amino)piperidine-2,6-dione;
3-((3-fluoro-4-(4-(((5-fluoro-4-oxo-7-((tetrahydro-2H-pyran-4-yl)methoxy)-3,4-dihydroquinazolin-2-yl)methyl)thio)-[1,4′-bipiperidin]-1′-yl)phenyl)amino)piperidine-2,6-dione;
3-((4-(4-((7-(cyclopropylmethoxy)-5-fluoro-4-oxo-3,4-dihydroquinazolin-2-yl)methoxy)-[1,4′-bipiperidin]-1′-yl)-3-fluorophenyl)amino)piperidine-2,6-dione;
3-((4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)amino) piperidine-2,6-dione;
3-((4-(4-(2-(((1r,4r)-4-(((7-(cyclopropylmethoxy)-5-fluoro-4-oxo-3,4-dihydroquinazolin-2-yl)methyl)thio)cyclohexyl)oxy)ethyl)piperazin-1-yl)-3-fluorophenyl)amino)piperidine-2,6-dione;
3-((4-(4-((4-(((7-(cyclopropylmethoxy)-5-fluoro-4-oxo-3,4-dihydroquinazolin-2-yl)methyl)thio)piperidin-1-yl)methyl)piperidin-1-yl)-3-fluorophenyl)amino)piperidine-2,6-dione;
3-((4-(4-(((7-(cyclopropylmethoxy)-5-fluoro-4-oxo-3,4-dihydroquinazolin-2-yl)methyl)thio)-[1,4′-bipiperidin]-1′-yl)-3-(trifluoromethyl)phenyl)amino)piperidine-2,6-dione;
3-((4-(4-(((7-(cyclopropylmethoxy)-5-fluoro-4-oxo-3,4-dihydroquinazolin-2-yl)methyl)thio)-[1,4′-bipiperidin]-1′-yl)-2,5-difluorophenyl)amino)piperidine-2,6-dione;
3-((4-(3-(4-(((7-(cyclopropylmethoxy)-5-fluoro-4-oxo-3,4-dihydroquinazolin-2-yl)methyl)thio)piperidin-1-yl)pyrrolidin-1-yl)-3-fluorophenyl)amino)piperidine-2,6-dione;
1-(4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)dihydropyrimidine-2,4(1H,3H)-dione;
1-(6-(4-(((7-(cyclopropylmethoxy)-5-fluoro-4-oxo-3,4-dihydroquinazolin-2-yl)methyl)thio)-[1,4′-bipiperidin]-1′-yl)-1-methyl-1H-indazol-3-yl)dihydropyrimidine-2,4(1H,3H)-dione;
1-(8-(4-(((7-(cyclopropylmethoxy)-5-fluoro-4-oxo-3,4-dihydroquinazolin-2-yl)methyl)thio)-[1,4′-bipiperidin]-1′-yl)isoquinolin-4-yl)dihydropyrimidine-2,4(1H,3H)-dione
3-((4-(4-(4-(2-(7-(cyclopropylmethoxy)-5-fluoro-4-oxo-3,4-dihydroquinazolin-2-yl)ethyl)piperazin-1-yl)piperidin-1-yl)-3-fluorophenyl)amino)piperidine-2,6-dione
3-((4-(1′-(2-(7-(cyclopropylmethoxy)-5-fluoro-4-oxo-3,4-dihydroquinazolin-2-yl)ethyl)-[4,4′-bipiperidin]-1-yl)-3-fluorophenyl)amino)piperidine-2,6-dione
3-((4-(4-(2-(7-(cyclopropylmethoxy)-5-fluoro-4-oxo-3,4-dihydroquinazolin-2-yl)ethyl)-[1,4′-bipiperidin]-1′-yl)-3-fluorophenyl)amino)piperidine-2,6-dione
3-((4-(3-(4-(((7-(cyclopropylmethoxy)-5-fluoro-4-oxo-3,4-dihydroquinazolin-2-yl)methyl)thio)piperidin-1-yl)azetidin-1-yl)-3-fluorophenyl)amino)piperidine-2,6-dione
3-((4-(4-(((7-(cyclopropylethynyl)-5-fluoro-4-oxo-3,4-dihydroquinazolin-2-yl)methyl)thio)-[1,4′-bipiperidin]-1′-yl)-3-fluorophenyl)amino)piperidine-2,6-dione

or a pharmaceutically acceptable salt of any of the aforementioned.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “pyridinyl,” “pyridyl,” or “a pyridine ring” may refer to a pyridin-2-yl, pyridin-3-yl, or pyridin-4-yl ring.

The term “n-membered,” where “n” is an integer, typically describes the number of ring-forming atoms in a moiety where the number of ring-forming atoms is “n”. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —C(O)NR^{G}— includes both —C(O)NR^{G}— and —NR^{G}C(O)— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. It is to be understood that substitution at a given atom is limited by valency.

As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “halo” or “halogen”, employed alone or in combination with other terms, includes fluoro, chloro, bromo, and iodo. In some embodiments, halo is F or Cl.

As used herein, the term “haloalkyl,” employed alone or in combination with other terms, refers to an alkyl group having up to the full valency of halogen atom substituents, which may either be the same or different. In some embodiments, the halogen atoms are fluoro atoms. In some embodiments, the alkyl group has 1 to 6 or 1 to 4 carbon atoms. Example haloalkyl groups include CF₃, C₂F₅, CHF₂, CCl₃, CHCl₂, C₂Cl₅, and the like.

As used herein, the term “alkoxy,” employed alone or in combination with other terms, refers to a group of formula —O-alkyl. Example alkoxy groups include methoxy, ethoxy, propoxy (e.g., n-propoxy and isopropoxy), t-butoxy, and the like. In some embodiments, the alkyl group has 1 to 6 or 1 to 4 carbon atoms.

As used herein, “haloalkoxy,” employed alone or in combination with other terms, refers to a group of formula —O-(haloalkyl). In some embodiments, the alkyl group has 1 to 6 or 1 to 4 carbon atoms. An example haloalkoxy group is —OCF₃.

As used herein, “amino,” employed alone or in combination with other terms, refers to NH₂.

As used herein, the term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

As used herein, the term “dialkylamino,” employed alone or in combination with other terms, refers to a group of formula —N(alkyl)₂. Example dialkylamino groups include dimethylamino, diethylamino, dipropylamino (e.g., di(n-propyl)amino and di(isopropyl)amino), and the like. In some embodiments, each alkyl group independently has 1 to 6 or 1 to 4 carbon atoms.

As used herein, the term “cycloalkyl,” employed alone or in combination with other terms, refers to a non-aromatic cyclic hydrocarbon including cyclized alkyl and alkenyl groups. Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to 10 ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, tetrahydropyran ring, tetrahyropyridine, azetidine ring, or tetrahydrofuran ring.

As used herein, the term “heterocycloalkylalkyl,” employed alone or in combination with other terms, refers to a group of formula heterocycloalkyl-alkyl-. In some embodiments, the alkyl portion has 1 to 4, 1 to 3, 1 to 2, or 1 carbon atom(s). In some embodiments, the alkyl portion is methylene. In some embodiments, the heterocycloalkyl portion has 3 to 10 ring members, 4 to 10 ring members, or 3 to 7 ring members. In some embodiments, the heterocycloalkyl group is monocyclic or bicyclic. In some embodiments, the heterocycloalkyl portion is monocyclic. In some embodiments, the heterocycloalkyl portion is a C₂₋₇ monocyclic heterocycloalkyl group.

As used herein, the term “aryl,” employed alone or in combination with other terms, refers to a monocyclic or polycyclic (e.g., a fused ring system) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl.

As used herein, the term “arylalkyl,” employed alone or in combination with other terms, refers to a group of formula aryl-alkyl-. In some embodiments, the alkyl portion has 1 to 4, 1 to 3, 1 to 2, or 1 carbon atom(s). In some embodiments, the alkyl portion is methylene. In some embodiments, the aryl portion is phenyl. In some embodiments, the aryl group is a monocyclic or bicyclic group. In some embodiments, the arylalkyl group is benzyl.

As used herein, the term “heteroaryl,” employed alone or in combination with other terms, refers to a monocyclic or polycyclic (e.g., a fused ring system) aromatic hydrocarbon moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has from 3 to 10 carbon atoms, from 3 to 8 carbon atoms, from 3 to 5 carbon atoms, from 1 to 5 carbon atoms, or from 5 to 10 carbon atoms. In some embodiments, the heteroaryl group contains 3 to 14, 4 to 12, 4 to 8, 9 to 10, or 5 to 6 ring-forming atoms. In some embodiments, the heteroaryl group has 1 to 4, 1 to 3, or 1 to 2 heteroatoms.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to 10 carbon atoms.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention also include all isotopes of atoms occurring in the intermediates or final compounds. Isotopes include those atoms having the same atomic number but different mass numbers. For example, isotopes of hydrogen include tritium and deuterium. In some embodiments, the compounds of the invention include at least one deuterium atom.

The term “compound,” as used herein, is meant to include all stereoisomers, geometric isomers, tautomers, and isotopes of the structures depicted, unless otherwise specified.

All compounds, and pharmaceutically acceptable salts thereof, can be found together with other substances such as water and solvents (e.g., in the form of hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

The term “small molecule PARP14 targeting moiety” refers to a chemical group that binds to PARP14. The small molecule PARP14 targeting moiety can be a group derived from a compound that inhibits the activity of PARP14. In some embodiments, the small molecule PARP14 targeting moiety inhibits the activity of PARP14 with an DC₅₀ of less than 1 μM in an enzymatic assay (see, e.g., Example A).

The term “Ubiquitin Ligase” refers to a family of proteins that facilitate the transfer of ubiquitin to a specific substrate protein, targeting the substrate protein for degradation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid

Compounds of the invention can be prepared according to numerous preparatory routes known in the literature. Example synthetic methods for preparing compounds of the invention are provided in the Schemes below.

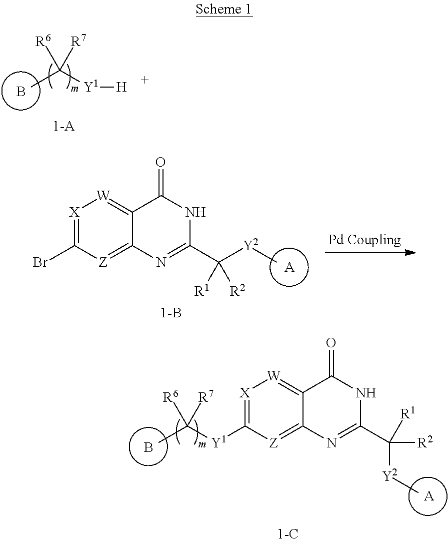

Scheme 1 shows a general synthesis of quinazolinone compounds of the disclosure. Compounds of formula (1-A), many of which are commercially available or can be made via routes known to one skilled in the art, can be coupled with compounds of formula (1-B), many of which are known in the art and described in, for example, U.S. Pat. No. 10,562,891. The coupling can be performed under Pd coupling conditions (e.g., in the presence of a Pd reagent such as [Pd(allyl)Cl]₂) and provides compounds of formula (1-C).

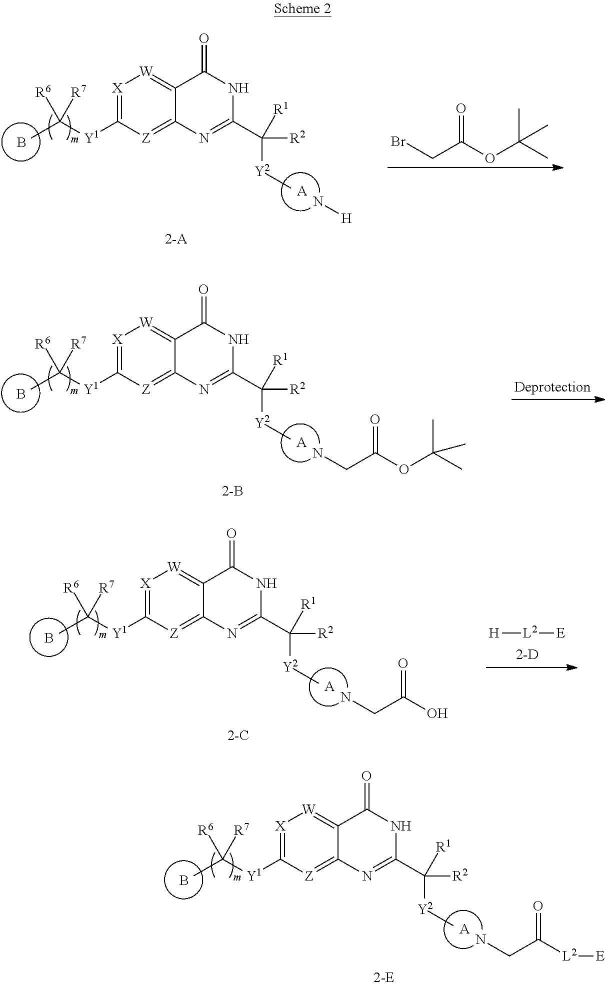

Scheme 2 shows a general synthesis of certain compounds of the invention. Compounds of formula (2-A) can be prepared according to the route provided in Scheme 1 or according to the processes disclosed in U.S. Pat. No. 10,562,891. An N-atom of the A-ring of a compound of formula (2-A) can be treated with tert-butyl 2-bromoacetate to provide a compound of formula (2-B). Compounds of formula (2-B) can be treated with acid (e.g., trifluoroacetic acid) to provide compounds of formula (2-C). Compounds of formula (2-C) can be coupled with compounds of formula (2-D), wherein group L² refers to the internal portion of a linker moiety, L¹, as defined herein. Compounds of formula (2-D) are commercially available and are also known in the art. The coupling of compounds of formula (2-C) and formula (2-D) can be performed under peptide coupling conditions (e.g., EDCI, HOBt, and DIPEA; or HATU, DIPEA) to provide a compound of formula (2-E). The “—CH₂—C(O)-L²-” group of the compound of formula (2-E) is equivalent to an L¹ group as defined herein.

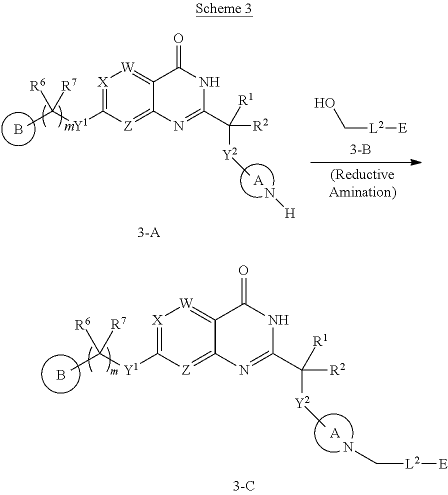

Scheme 3 shows a general synthesis of certain compounds of the invention. Compounds of formula (3-A) can be prepared according to the route provided in Scheme 1. Compounds of formula (3-B), wherein group L² refers to the internal portion of a linker moiety, L¹, as defined herein, can be oxidized (e.g., with Dess-Martin periodinane) to provide an aldehyde intermediate in situ (not shown). Treatment of the resulting reaction mixture with compounds of formula (3-A) followed by a hydride reducing agent (e.g., NaBH(OAc)₃) can provide compounds of formula (3-C). The “—CH₂-L²-” group of the compound of formula (3-C) is equivalent to an L¹ group as defined herein.

### Methods of Use

Compounds of the present disclosure can bind to both PARP14 and ubiquitin E3 ligase to cause PARP14 degradation, which is useful in the treatment of various diseases including cancer. In some embodiments, the compounds provided herein can degrade PARP14 in a cell, which comprises contacting the cell with the compound or a pharmaceutically acceptable salt or a stereoisomer thereof. In some embodiments, provided herein is a method for degrading PARP14 in a patient, where the method comprises administering to the patient an effective amount of a compound described herein or a pharmaceutically acceptable salt or a stereoisomer thereof. By “degrading PARP14,” it is meant rendering the PARP14 inactive by, for example, altering its structure or breaking down PARP14 into multiple peptide or amino acid fragments.

The compounds of the invention are useful in the treatment of various diseases associated with abnormal expression or activity of PARP14. For example, the compounds of the invention are useful in the treatment of cancer. In some embodiments, the cancers treatable according to the present invention include hematopoietic malignancies such as leukemia and lymphoma. Example lymphomas include Hodgkin's or non-Hodgkin's lymphoma, multiple myeloma, B-cell lymphoma (e.g., diffuse large B-cell lymphoma (DLBCL)), chronic lymphocytic lymphoma (CLL), T-cell lymphoma, hairy cell lymphoma, and Burkett's lymphoma. Example leukemias include acute lymphocytic leukemia (ALL), acute myelogenous leukemia (AML), chronic lymphocytic leukemia (CLL), and chronic myelogenous leukemia (CML).

Other cancers treatable by the administration of the compounds of the invention include liver cancer (e.g., hepatocellular carcinoma), bladder cancer, bone cancer, glioma, breast cancer, cervical cancer, colon cancer, endometrial cancer, epithelial cancer, esophageal cancer, Ewing's sarcoma, pancreatic cancer, gallbladder cancer, gastric cancer, gastrointestinal tumors, head and neck cancer, intestinal cancers, Kaposi's sarcoma, kidney cancer, laryngeal cancer, liver cancer (e.g., hepatocellular carcinoma), lung cancer, prostate cancer, rectal cancer, skin cancer, stomach cancer, testicular cancer, thyroid cancer, and uterine cancer.

In some embodiments, the cancer treatable by administration of the compounds of the invention is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

The compounds of the invention may also have therapeutic utility in PARP14-related disorders in disease areas such as cardiology, virology, neurodegeneration, inflammation, and pain, particularly where the diseases are characterized by overexpression or increased activity of PARP14.

In some embodiments, the compounds of the invention are useful in the treatment of an inflammatory disease. It was found that genetic inactivation of Poly(ADP-Ribose) Polymerase Family Member 14 (PARP14), also referred to as ADP-Ribosyltransferase Diphtheria Toxin-Like 8 (ARTD8) or B Aggressive Lymphoma Protein (BAL2), protected mice against allergen-induced airway disease (Mehrothra et al., J Allergy Clin Immunol, Jul. 25, 2012, 131(2):521-531; and Cho et al., Proc Natl Acad Sci USA, Sep. 20, 2011, 108(38):15972-15977), suppressed the infiltration of immune cells such as eosinophils and neutrophils into the lung, and reduced the release of inflammatory Th2 cytokines. In addition, treatment with a PARP14 inhibitor protected mice in a severe asthma model induced by a sensitization and recall challenge with inhaled Alternaria alternata extract (Eddie et al PMID 35817532). PARP14 inhibitor-treated animals showed a reduced level of airway mucus, blood serum IgE, infiltration of immune cells (eosinophils, neutrophils, and lymphocytes), Th2 cytokines (IL-4, IL-5, and IL13) and alarmins (IL-33 and TSLP) (Eddie et al PMID 35817532 and Ribon internal data).

While not being bound by theory, PARP14 has been shown to affect STAT6 signaling and STAT3 signaling, signaling induced by Th2 cytokines and Th17 cytokines, M1/M2 macrophage polarization, and signaling by lymphocytes. PARP14 has also been shown to be a regulator of Th2/Th17/THF T cell development, involved in B cell development, and involved in eosinophils/neutrophils recruitment/activation. It is believed that the lymphocytes are likely the ILCs (e.g., ILC2 and ILC3) that get activated by the alarmins (e.g., TSLP and IL-33) and are the main producers of the downstream cytokines (e.g., IL-4, IL-5, and IL-13).

It is suggested that PARP14 inhibition affects the asthma phenotype not only at the level of the second order cytokines (e.g., IL-4, IL-5, and IL-13) and the signaling to the myeloid cells, but that PARP14 inhibition also suppresses the alarmins TSLP and IL-33, which are the key upstream drivers of asthma that get released in response to the allergens.

The present invention is directed, inter alia, to a method of treating or preventing an inflammatory disease in a patient comprising administering to the patient a therapeutically effective amount of a compound of the disclosure, or a pharmaceutically acceptable salt thereof. Exemplary inflammatory diseases that are treatable by the disclosed methods include, e.g., asthma, atopic dermatitis, psoriasis, rhinitis, systemic sclerosis, keloids, eosinophilic disorders, pulmonary fibrosis, and other type 2 cytokine pathologies. In some embodiments, the pulmonary fibrosis is idiopathic pulmonary fibrosis.

Additional exemplary inflammatory diseases that are treatable by the disclosed methods include inflammatory bowel diseases (“IBD”), which include ulcerative colitis (“UC” or “colitis”) and Crohn's disease. In some embodiments, the inflammatory disease is inflammatory bowel disease. In some embodiments, the inflammatory disease is ulcerative colitis. In some embodiments, the inflammatory disease is Crohn's disease.

In some embodiments, the inflammatory disease is irritable bowel syndrome.

Eosinophilic disorders that are treatable by the disclosed methods include, e.g., eosinophilic esophagitis (esophagus—EoE), eosinophilic gastritis (stomach—EG), eosinophilic gastroenteritis (stomach and small intestine—EGE), eosinophilic enteritis (small intestine—EE), eosinophilic colitis (large intestine—EC), and eosinophilic chronic rhinosinusitis.

The present invention is further directed, inter alia, to a method of treating or preventing asthma in a patient comprising administering to the patient a therapeutically effective amount of a compound described herein, or a pharmaceutically acceptable salt thereof.

In some embodiments, the asthma is steroid-insensitive asthma, steroid-refractory asthma, steroid-resistant asthma, atopic asthma, nonatopic asthma, persistent asthma, severe asthma, or steroid-refractory severe asthma. In some embodiments, the severe asthma is T2 high endotype, T2 low endotype, or non-T2 endotype. In some embodiments, the severe asthma is T2 high endotype. In some embodiments, the severe asthma is T2 low endotype or non-T2 endotype. In some embodiments, the severe asthma is T2 low endotype. In some embodiments, the severe asthma is non-T2 endotype.

The present invention is further directed, inter alia, to a method of treating or preventing fibrotic diseases such as, but not limited to, pulmonary fibrosis, renal fibrosis, hepatic fibrosis (e.g., NASH and NAFLD), systemic fibrosis, and idiopathic pulmonary fibrosis (IPF). In some embodiments, the fibrotic disease is systemic fibrosis.

The present invention is further directed, inter alia, to a method of treating or preventing chronic obstructive pulmonary disease (COPD), emphysema, and chronic bronchitis.

The present invention is further directed, inter alia, to a method of treating or preventing a skin inflammatory disease such as atopic dermatitis or rosacea.

The present invention further provides a method of:
(a) reducing the level of airway mucus in lung tissue,
(b) reducing blood serum IgE,
(c) reducing immune cell infiltration and activation in bronchoalveolar fluid,
(d) reducing the level of one or more inflammatory cytokines in bronchoalveolar fluid or in lung tissue, or
(e) reducing the level of one or more alarmins in bronchoalveolar fluid or lung tissue,
in a patient, where the method comprises administering to the patient a therapeutically effective amount of a compound disclosed herein, or a pharmaceutically acceptable salt thereof.

In some embodiments, the present invention provides a method of reducing the level of airway mucus in lung tissue in a patient.

In some embodiments, the present invention provides a method of reducing immune cell infiltration and activation in bronchoalveolar fluid in a patient. In some embodiments, the immune cells are eosinophils, neutrophils, or lymphocytes.

In some embodiments, the present invention provides a method of reducing one or more inflammatory cytokines in bronchoalveolar fluid or in lung tissue in a patient. In some embodiments, the inflammatory cytokine is a Th2 cytokine or Th17 cytokine. In some embodiments, the inflammatory cytokine is a Th2 cytokine. In some embodiments, the inflammatory cytokine is IL-4, IL-5, IL13, or IL-17A. In some embodiments, the inflammatory cytokine is IL-4, IL-5, or IL 13.

In some embodiments, the present invention provides a method of reducing an alarmin in bronchoalveolar fluid or in lung tissue in a patient. In some embodiments, the alarmin is IL-25, IL-33 or TSLP.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” PARP14 or “contacting” a cell with a compound of the invention includes the administration of a compound of the present invention to an individual or patient, such as a human, having PARP14, as well as, for example, introducing a compound of the invention into a sample containing a cellular or purified preparation containing PARP14.

As used herein, the term “individual” or “patient,” used interchangeably, refers to mammals, and particularly humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease.

As used herein, the term “reducing” is with respect to the level in the patient prior to administration. More specifically, when a biomarker or symptom is reduced in a patient, the reduction is with respect to the level of or severity of the biomarker or symptom in the patient prior to administration of the compound of Formula (I), or a pharmaceutically acceptable salt thereof.

### Combination Therapy

One or more additional pharmaceutical agents or treatment methods such as, for example, chemotherapeutics or other anti-cancer agents, immune enhancers, immunosuppressants, immunotherapies, radiation, anti-tumor and anti-viral vaccines, cytokine therapy (e.g., IL2, GM-CSF, etc.), and/or kinase (tyrosine or serine/threonine), epigenetic or signal transduction inhibitors can be used in combination with the compounds of the present invention. The agents can be combined with the present compounds in a single dosage form, or the agents can be administered simultaneously or sequentially as separate dosage forms.

Suitable agents for use in combination with the compounds of the present invention for the treatment of cancer include chemotherapeutic agents, targeted cancer therapies, immunotherapies or radiation therapy. Compounds of this invention may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other anti-cancer agent(s) include antibody therapeutics to costimulatory molecules such as CTLA-4, 4-1BB, PD-1, and PD-L1, or antibodies to cytokines (IL-10, TGF-β, etc.). Exemplary cancer immunotherapy antibodies include alemtuzumab, ipilimumab, nivolumab, ofatumumab and rituximab.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

Suitable agents for use in combination with the compounds of the present invention for the treatment of inflammatory diseases include but are not limited to corticosteroids (e.g., prednisone, prednisolone, methylprednisolone, and hydrocortisone); disease-modifying antihreumatic drugs (“DMARDs”, e g, immunosuppressive or anti-inflammatory agents); anti-malarial agents (e.g. hydroxychloroquine and chloroquine); immunosuppressive agents (e.g., cyclophosphamide, azathioprine, mycophenolate mofetil, methotrexate); anti-inflammatory agents (e.g., aspirin, NSAIDs (e.g., ibuprofen, naproxen, indomethacin, nabumetone, celecoxib)); anti-hypertensive agents (e.g., calcium channel blockers (e.g., amlodipine, nifedipine) and diuretics (e.g., furosemide)); statins (e.g., atorvastatin, fluvastatin, lovastatin, pitavastatin, pravastatin, rosuvastatin and simvastatin); anti-B-cell agents (e.g., anti-CD20 (e.g., rituximab), anti-CD22); anti-B-lymphocyte stimulator agents (“anti-BLyS”, e.g., belimumab, blisibimod); type-1 interferon receptor antagonist (e.g., anifrolumab); T-cell modulators (e.g., rigerimod); abatacept; anticoagulants (e.g., heparin, warfarin); and vitamin D supplements.

Additional suitable agents for use in combination of the present invention for the treatment of inflammatory diseases include but not are not limited to sulfonylureas, meglitinides, biguanides, alpha-glucosidase inhibitors, peroxisome proliferators-activated receptor-gamma (i.e., PPAR-gamma) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), anti-platelet agents (for example, aspirin and adenosine diphosphate receptor antagonists that include: clopidogrel, ticlopidine and the like), angiotensin-converting enzyme inhibitors, angiotensin II receptor antagonists and adiponectin.

Suitable agents for use in combination with the compounds of the present invention for the treatment of asthma include but are not limited to beclomethasone (Qvar™), budesonide (Pulmicort Flexhaler™), budesonide/formoterol (Symbicort™), ciclesonide (Alvesco™), flunisolide (Aerospan™), fluticasone (Flovent Diskus™, flovent HFA™, Arnuity Ellipta™), fluticasone/salmeterol (Advair™), mometasone (Asmanex™), mometasone/formoterol (Dulera™), albuterol sulfate (VoSpireER™), formoterol fumarate (Aerolizer™), salmeterol xinafoate (Serevent™), arformoterol tartrate (Brovana™), olodaterol (Striverdi™), fluticasone furoate/vilanterol (Breo Ellipta™), fluticasone furoate/umeclidinium/vilanterol (Trelegy Ellipta™), fluticasone propionate/salmeterol (AirDuo™), glycopyrrolate/formoterol fumarate (Bevespi Aerosphere™), indacaterol/glycopyrrolate (Utibron Neohaler™), tiotropium/olodaterol (Stiolto Respimat™), umeclidinium/vilanterol (Anoro Ellipta™), omalizumab (Xolair™), mepolizumab (NUCALA™), benralizumab (Fasenra™), reslizumab (Cinqair™), dupilumab, tralokinumab, lebrikizumab, etanercept, golimumab brodalumab, and tezepelumab.

### Pharmaceutical Formulations and Dosage Forms

When employed as pharmaceuticals, the compounds of the invention can be administered in the form of pharmaceutical compositions. A pharmaceutical composition refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, or parenteral.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, anti-cancer agents, vaccines, antibodies, immune enhancers, immune suppressants, anti-inflammatory agents and the like.

### EXAMPLES

Equipment: ¹H NMR Spectra were recorded at 300 or 400 MHz using a Bruker AVANCE 300 MHz/400 MHz spectrometer. NMR interpretation was performed using Bruker Topspin software to assign chemical shift and multiplicity. In cases where two adjacent peaks of equal or unequal height were observed, these two peaks may be labeled as either a multiplet or as a doublet. In the case of a doublet, a coupling constant using this software may be assigned. In any given example, one or more protons may not be observed due to obscurity by water and/or solvent peaks. LCMS equipment and conditions are as follows:

1. LC (Basic condition): Shimadzu LC-20ADXR, Binary Pump, Diode Array Detector. Column: Shim-pack scepter C18 33*3.0 mm, 3.0 μm. Mobile phase: A: Water/6.5 mM (NH₄)HCO₃; B: Acetonitrile. Flow Rate: 1.5 mL/min at 40° C. Detector: 190-400 nm. Gradient stop time 2.0 min. Timetable:

| T (min) | A(%) | B(%) |
|---|---|---|
| 0.01 | 90 | 10 |
| 1.20 | 5 | 95 |
| 1.80 | 5 | 95 |
| 1.82 | 90 | 10 |

2. LC (Basic condition): Shimadzu LC-20ADXR, Binary Pump, Diode Array Detector. Column: Shim-pack scepter C18 33*3.0 mm, 3.0 μm. Mobile phase: A: Water/5 mM (NH₄)HCO₃; B: Acetonitrile. Flow Rate: 1.5 mL/min at 40° C. Detector: 190-400 nm. Gradient stop time 2.0 min. Timetable:

| T(min) | A(%) | B(%) |
|---|---|---|
| 0.01 | 90 | 10 |
| 1.20 | 5 | 95 |
| 1.80 | 5 | 95 |
| 1.82 | 90 | 10 |

3. LC (acidic condition): Shimadzu LC-20ADXR, Binary Pump, Diode Array Detector. Column: Halo C18, 30*3.0 mm, 2.0 μm. Mobile phase: A: Water/0.05% TFA, B: Acetonitrile/0.05% TFA. Flow Rate: 1.5 mL/min at 40° C. Detector: 190-400 nm. Gradient stop time, 2.0 min. Timetable:

| T (min) | A(%) | B(%) |
|---|---|---|
| 0.01 | 90 | 5 |
| 1.20 | 5 | 100 |
| 1.80 | 5 | 100 |
| 1.82 | 90 | 5 |

4. LC (Acidic condition): Shimadzu LC-20AD, Binary Pump, Diode Array Detector. Column: Halo C18, 30*3.0 mm, 2.0 μm. Mobile Phase A: Water/0.1% FA; B: Acetonitrile/0.1% FA. Flow Rate: 1.5 mL/min at 40° C. Detector: 190-400 nm. Gradient stop time 3.0 min. Timetable:

| T (min) | A(%) | B(%) |
|---|---|---|
| 0.01 | 90 | 5 |
| 1.20 | 5 | 100 |
| 1.80 | 5 | 100 |
| 1.82 | 90 | 5 |

5. The MS detector is configured with electrospray ionization as ionizable source. Acquisition mode: Scan; Nebulizing Gas Flow: 1.5 L/min; Drying Gas Flow: 15 L/min; Detector Voltage: 0.95-1.25 kv; DL Temperature: 250° C.; Heat Block Temperature: 250° C.; Scan Range: 90.00-900.00 m/z.

6. Sample preparation: samples were dissolved in ACN or methanol at 1˜10 mg/mL, then filtered through a 0.22 m filter membrane. Injection volume: 1-3 μL.

### Definitions

ACN (acetonitrile); AcOH (acetic acid); B₂(OH)₄ (tetrahydroxydiboron); Boc₂O (di-tert-butyl decarbonate); t-BuOK (potassium tert-butoxide); t-BuONa (sodium tert-butoxide); Cs₂CO₃ (cesium carbonate); CH₃CN (acetonitrile); CuI (copper(I) iodide); DCM (dichloromethane); DIEA (N,N-diisopropylethylamine); DMA (N,N-dimethylacetamide); DMF (N,N-dimethylformamide); DMAP (4-dimethyl aminopyridine); DMP (Dess-Martin periodinane); DMSO (dimethylsulfoxide); DMSO-d₆ (deuterated dimethylsulfoxide); EDCI (1-ethyl-3-(3-dimethylaminopropyl)carbodiimide); equiv (equivalent); ESI (electrospray ionization); EtOAc (ethyl acetate); Et₂O (diethyl ether); EtOH (ethanol); FA (formic acid); Fe (iron); FeCl₃ (iron(III) chloride); FeCl₃·6H₂O (iron(III) chloride hexahydrate); g (gram); h (hour); HATU (1-[Bis(dimethylamino)methylene]-1H-1,2,3-triazolo[4,5-b]pyridinium 3-oxid hexafluorophosphate); ¹H NMR (proton nuclear magnetic resonance); HCl (hydrochloric acid); HOBT (1-Hydroxybenzotriazole); Hz (hertz); K₂CO₃ (potassium carbonate); KI (potassium iodide); KOH (potassium hydroxide); K₃PO₄ (potassium phosphate tribasic); L (liter); LCMS (liquid chromatography-mass spectrometry); M (molar); MeCN (acetonitrile); MeOH (methanol); mg (milligrams); MHz (megahertz); min (minutes); mL (milliliters), mmol (millimoles); NaBH₃CN (Sodium cyanoborohydride); NaBH(OAc)₃ (sodium triacetoxyborohydride); Na₂CO₃ (sodium carbonate); NaH (sodium hydride); NH₄Cl (ammoinium chloride); NaHCO₃ (sodium bicarbonate); NaOH (sodium hydroxide); Na₂SO₄ (sodium sulfate); (NH₄)HCO₃ (ammonium bicarbonate); nm (namometers), NMP (N-methylpyrrolidone); PBS (phosphate buffered saline); Pd/C (palladium on carbon); Pd₂(dba)₃ (tris(dibenzylideneacetone)dipalladium(0)); Pd-PEPPSI-IPentCl 2-methylpyridine (o-picoline) ((SP-4-1)-[1,3-bis[2,6-bis(1-ethylpropyl)phenyl]-4,5-dichloro-1,3-dihydro-2H-imidazol-2-ylidene]dichloro(2-methylpyridine)palladium); Pd(PPh₃)₂Cl₂ (trans-dichlorobis(triphenylphosphine)palladium(II)); PE (petroleum ether); prep-HPLC (preparative high-performance liquid chromatography); ppm (parts per million); STAB (sodium triacetoxyborohydride); TBAB (tetrabutylammonium bromide); TBAF (tetrabutylammonium fluoride); TBHP (tert-butyl hydroperoxide); TEA (triethylamine); TFA (trifluoroacetic acid); TfOH (trifluoromethanesulfonic acid); THF (tetrahydrofuran); RT (retention time); UV (ultraviolet); Xphos (2-dicyclohexylphosphino-2′,4′,6′-triisopropylbiphenyl).

### Intermediate A1: Synthesis of 2-(2,6-dioxopiperidin-3-yl)-4-(4-(piperidin-4-ylmethyl)piperazin-1-yl)isoindoline-1,3-dione

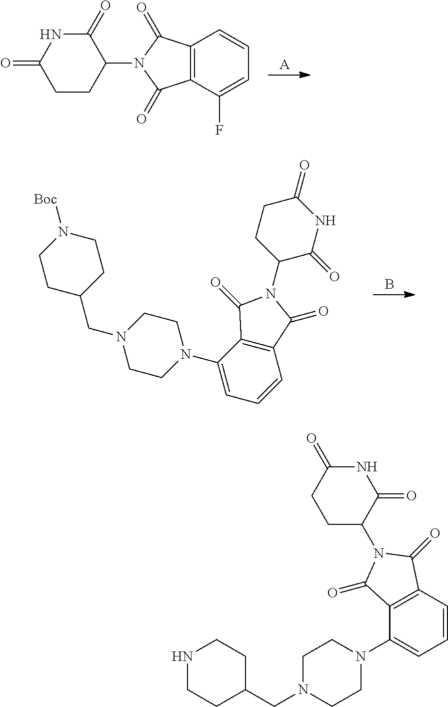

### Step A

A solution of tert-butyl 4-(piperazin-1-ylmethyl)piperidine-1-carboxylate (8.90 g, 31.4 mmol, 1.2 equiv) and 2-(2,6-dioxopiperidin-3-yl)-4-fluoroisoindoline-1,3-dione (7.23 g, 26.2 mmol, 1.0 equiv) and TEA (7.95 g, 78.6 mmol, 3.0 equiv) in NMP (70 mL) was stirred for 3 h at 70° C. The resulting mixture was diluted with brine (200 mL) and EtOAc (300 mL). The precipitated solids were collected by filtration and washed with EtOAc (30 mL) to afford tert-butyl 4-((4-(2-(2,6-dioxopiperidin-3-yl)-1,3-dioxoisoindolin-4-yl)piperazin-1-yl)methyl)piperidine-1-carboxylate (4.2 g, 30% yield) as a yellow solid. LCMS (ESI, m/z): 540.05 [M+H]⁺.

### Step B

A solution of tert-butyl 4-((4-(2-(2,6-dioxopiperidin-3-yl)-1,3-dioxoisoindolin-4-yl)piperazin-1-yl)methyl)piperidine-1-carboxylate (4.2 g, 7.8 mmol, 1.0 equiv) in TFA (30 mL) and DCM (90 mL) was stirred for 2 hours at room temperature. The resulting mixture was concentrated under vacuum and then diluted with DCM (60 mL) and water (50 ml). The mixture was neutralized to pH 7 with saturated aqueous Na₂CO₃. The resulting mixture was concentrated under vacuum to remove DCM. The precipitated solids were collected by filtration to afford 2-(2,6-dioxopiperidin-3-yl)-4-(4-(piperidin-4-ylmethyl)piperazin-1-yl)isoindoline-1,3-dione (3.9 g) as a yellow crude solid. The product was used without further purification. LCMS (ESI, m/z): 440.10 [M+H]⁺.

Intermediates A1-a-A1-e were synthesized according to the procedure described for the synthesis of 2-(2,6-dioxopiperidin-3-yl)-4-(4-(piperidin-4-ylmethyl)piperazin-1-yl)isoindoline-1,3-dione using appropriate building blocks and modified reaction conditions (such as reagents, reagent ratio, temperature, and reaction time) and purification conditions as needed.

| Intermediate | | |
|---|---|---|
| No. | Structure | Analytical Data |
| A1-a | 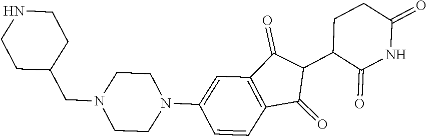 [SMILES: O=C1CCC(C2C(=O)c3ccc(N4CCN(CC5CCNCC5)CC4)cc3C2=O)C(=O)N1] | LCMS (ESI, m/z): 440.10 [M + H]+ |
| A1-b | 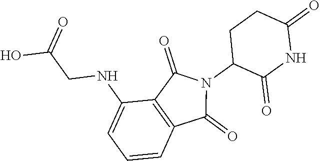 [SMILES: O=C(O)CNc1cccc2c1C(=O)N(C1CCC(=O)NC1=O)C2=O] | LCMS (ESI, m/z): 332.05 [M + H]+. |
| A1-c | 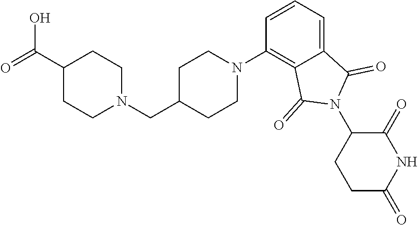 [SMILES: O=C1CCC(N2C(=O)c3cccc(N4CCC(CN5CCC(C(=O)O)CC5)CC4)c3C2=O)C(=O)N1] | LCMS (ESI, m/z): 483.22 [M + H]⁺ |
| | 1-((1-(2-(2,6-dioxopiperidin-3-yl)- | |
| | 1,3-dioxoisoindolin-4-yl)piperidin-4- | |
| | yl)methyl)piperidine-4-carboxylic | |
| | acid | |
| A1-d | 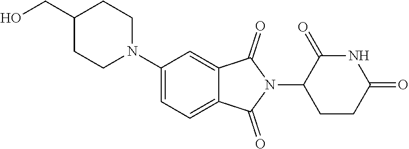 [SMILES: O=C1CCC(N2C(=O)c3ccc(N4CCC(CO)CC4)cc3C2=O)C(=O)N1] | LCMS (ESI, m/z): 372.15 [M + H]⁺. |
| A1-e | 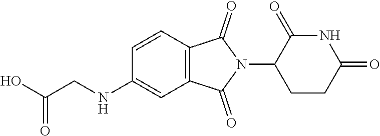 [SMILES: O=C(O)CNc1ccc2c(c1)C(=O)N(C1CCC(=O)NC1=O)C2=O] | LCMS: 332.05 [M + H]⁺. |

### Intermediate A2: Synthesis of N-(6-aminohexyl)-3-(2,4-dioxo-1,3-diazinan-1-yl)benzamide HCl salt

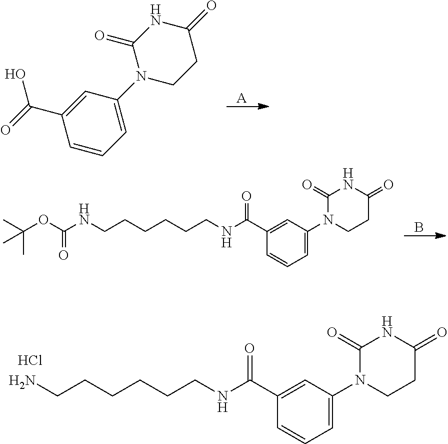

### Step A

To a solution of 3-(2,4-dioxo-1,3-diazinan-1-yl)benzoic acid (170 mg, 0.72 mmol, 1.0 equiv) and tert-butyl N-(6-aminohexyl)carbamate (157 mg, 0.72 mmol, 1.0 equiv) in DMF (2 mL) was added DIEA (281 mg, 2.18 mmol, 3.0 equiv) and HATU (414 mg, 1.09 mmol, 1.5 equiv). The resulting mixture was stirred for 1 hour. The residue was purified by reverse flash chromatography with the following conditions: column: C18 silica gel; mobile phase: MeCN in water (0.1% FA), 0% to 40% gradient in 20 min; detector: UV 254 nm to afford tert-butyl N-(6-{[3-(2,4-dioxo-1,3-diazinan-1-yl)phenyl]formamido}hexyl)carbamate (256 mg, 82% yield) as a white solid. LCMS (ESI, m/z): 433.24[M+H]⁺.

### Step B

A solution of tert-butyl N-(6-{[3-(2,4-dioxo-1,3-diazinan-1-yl)phenyl]formamido}hexyl)carbamate (100 mg, 0.23 mmol, 1.0 equiv) and HCl in 1,4-dioxane (1 mL, 4 M) in 1,4-dioxane (1 mL) was stirred for 2 hours. The resulting mixture was concentrated under vacuum to afford N-(6-aminohexyl)-3-(2,4-dioxo-1,3-diazinan-1-yl)benzamide (71 mg, 92% yield) as a white solid. LCMS (ESI, m/z): 333.18 [M+H]⁺.

Intermediate A2-a was synthesized according to the procedure described for the synthesis of N-(6-aminohexyl)-3-(2,4-dioxo-1,3-diazinan-1-yl)benzamide using appropriate building blocks and modified reaction conditions (such as reagents, reagent ratio, temperature, and reaction time) and purification conditions as needed.

| Intermediate | | |
|---|---|---|
| No. | Structure | Analytical Data |
| A2-a | 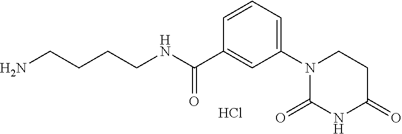 [SMILES: NCCCCNC(=O)c1cccc(N2CCC(=O)NC2=O)c1 ; Cl] | LCMS (ESI, m/z): 304.45 [M + H]⁺ |
| | N-(4-aminobutyl)-3-(2,4- | |
| | dioxotetrahydropyrimidin-1(2H)- | |
| | yl)benzamide hydrochloride | |

### Intermediate A3: Synthesis of 3-((4-(1-(6-aminohexyl)piperidin-4-yl)phenyl)amino)piperidine-2,6-dione hydrochloride

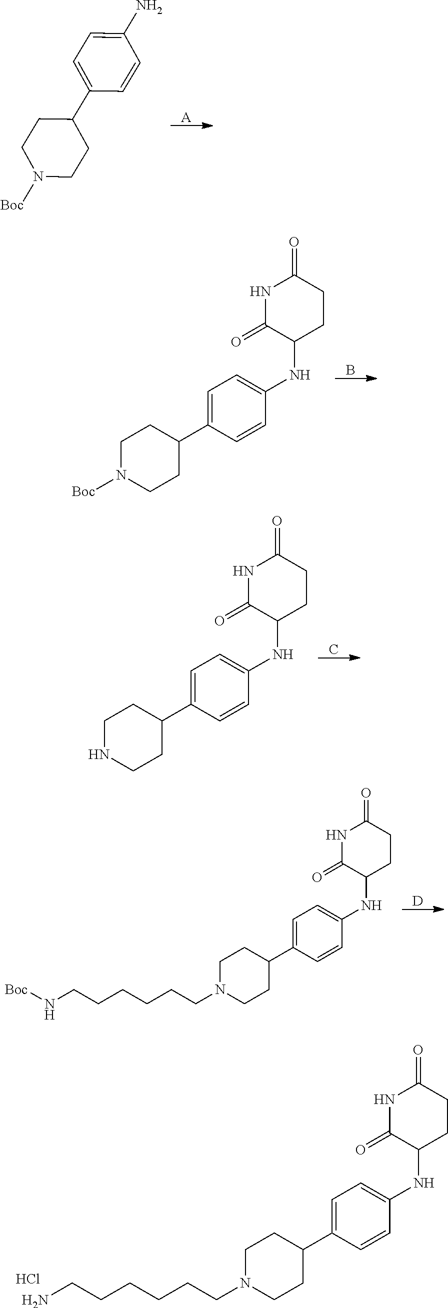

### Step A

A solution of tert-butyl 4-(4-aminophenyl)piperidine-1-carboxylate (1.0 g, 3.6 mmol, 1.0 equiv), 3-bromopiperidine-2,6-dione (1.4 g, 7.2 mmol, 2.0 equiv) and DIEA (0.94 g, 7.2 mmol, 2.0 equiv) in 1,4-dioxane was stirred for 24 hours at 80° C. The resulting mixture was concentrated under reduced pressure and dissolved in DMSO (10 mL). The residue was purified by reverse flash chromatography with the following conditions: column: C18 silica gel; mobile phase: MeCN in water (0.1% FA), 0% to 6 0% gradient in 20 min; UV 254 nm to afford tert-butyl 4-(4-((2,6-dioxopiperidin-3-yl)amino)phenyl)piperidine-1-carboxylate (1.2 g, 86% yield) as a white solid. LCMS (ESI, m/z): 388.22 [M+H]⁺.

### Step B

A solution of tert-butyl 4-(4-((2,6-dioxopiperidin-3-yl)amino)phenyl)piperidine-1-carboxylate (1.2 g, 3.1 mmol, 1.0 equiv) and HCl in 1,4-dioxane (10 mL, 4M) was stirred for 2 hours. The resulting mixture was concentrated under reduced pressure and the residue purified by trituration with Et₂O (50 mL) to afford 3-((4-(piperidin-4-yl)phenyl)amino)piperidine-2,6-dione (998 mg) as a white crude solid that was used without further purification. LCMS (ESI, m/z): 288.16 [M+H]⁺.

### Step C

A solution of 3-{[4-(4-aminobutan-2-yl)phenyl]amino}piperidine-2,6-dione (350 mg, 1.27 mmol, 1.0 equiv), tert-butyl N-(6-bromohexyl)carbamate (410 mg, 1.46 mmol, 1.2 equiv) and K₂CO₃ (337 mg, 2.43 mmol, 2.0 equiv) in DMF (5 mL) was stirred for 4 hours at 60° C. The mixture was acidified to pH 5 with citric acid. The mixture was purified directly by reverse flash chromatography with the following conditions: column: C18 silica gel; mobile phase: MeCN in water (0.1% FA), 0% to 50% gradient in 20 min; detector: UV 254 nm to afford tert-butyl (6-(4-(4-((2,6-dioxopiperidin-3-yl)amino)phenyl)piperidin-1-yl)hexyl)carbamate (254 mg, 43% yield) as a green solid. LCMS (ESI, m/z): 487.32[M+H]⁺.

### Step D

A solution of tert-butyl N-[6-(4-{4-[(2,6-dioxopiperidin-3-yl)amino]phenyl}piperidin-1-yl)hexyl]carbamate (150 mg, 0.31 mmol, 1.0 equiv) and HCl in 1,4-dioxane (1.5 mL, 4M) was stirred for 6 hours. The resulting mixture was concentrated under reduced pressure to afford 3-((4-(1-(6-aminohexyl)piperidin-4-yl)phenyl)amino)piperidine-2,6-dione hydrochloride (256 mg) as a green crude solid that was used in the next step without further purification. LCMS (ESI, m/z): 387.25 [M+H]⁺.

Intermediates A3-a-A3-d were synthesized according to the procedure described for the synthesis of 3-((4-(1-(6-aminohexyl)piperidin-4-yl)phenyl)amino)piperidine-2,6-dione hydrochloride using appropriate building blocks and modified reaction conditions (such as reagents, reagent ratio, temperature, and reaction time) and purification conditions as needed.

| Intermediate | | |
|---|---|---|
| No. | Structure | Analytical Data |
| A3-a | 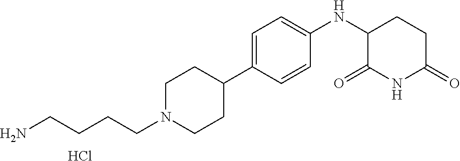 [SMILES: NCCCCN1CCC(c2ccc(NC3CCC(=O)NC3=O)cc2)CC1 ; Cl] | LCMS (ESI, m/z): 359.24 [M + H]⁺ |
| | 3-((4-(1-(4-aminobutyl)piperidin-4- | |
| | yl)phenyl)amino)piperidine-2,6-dione | |
| | hydrochloride | |
| A3-b | 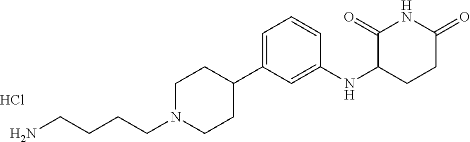 [SMILES: Cl ; NCCCCN1CCC(c2cccc(NC3CCC(=O)NC3=O)c2)CC1] | LCMS (ESI, m/z): 359.24 [M + H]⁺ |
| | 3-((3-(1-(4-aminobutyl)piperidin-4- | |
| | yl)phenyl)amino)piperidine-2,6-dione | |
| | hydrochloride | |
| A3-c | 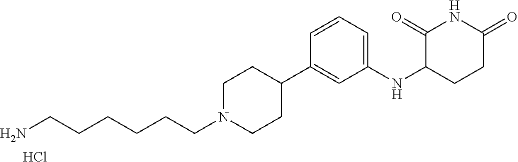 [SMILES: NCCCCCCN1CCC(c2cccc(NC3CCC(=O)NC3=O)c2)CC1 ; Cl] | LCMS (ESI, m/z): 388.10 [M + H]⁺ |
| | 3-((3-(1-(6-aminohexyl)piperidine-4- | |
| | yl)phenyl)amino)piperidine-2,6-dione | |
| | hydrochloride | |
| A3-d | 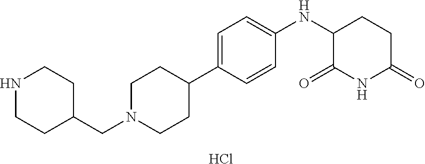 [SMILES: O=C1CCC(Nc2ccc(C3CCN(CC4CCNCC4)CC3)cc2)C(=O)N1 ; Cl] | LCMS (ESI, m/z): 385.35 [M + H]⁺ |
| | 3-((4-(1-(piperidin-4-ylmethyl)piperidin-4- | |
| | yl)phenyl)amino)piperidine-2,6-dione | |
| | hydrochloride | |

### Intermediate A4: Synthesis of 3-((4-(1-(6-aminohexanoyl)piperidin-4-yl)phenyl)amino)piperidine-2,6-dione hydrochloride

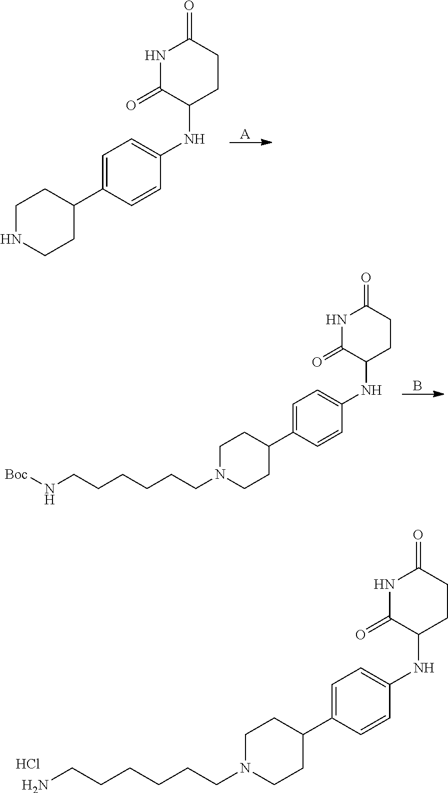

### Step A

To a stirred solution of 3-{[4-(piperidin-4-yl)phenyl]amino}piperidine-2,6-dione (300 mg, 1.04 mmol, 1.5 equiv) and 6-[(tert-butoxycarbonyl)amino]hexanoic acid (161 mg, 0.70 mmol, 1.0 equiv) in DMF (5 mL) was added HATU (317 mg, 0.835 mmol, 1.2 equiv) and DIEA (270 mg, 2.09 mmol, 3.0 equiv). The resulting mixture was stirred for 4 hours. The mixture was purified by reverse flash chromatography with the following conditions: column: C18 silica gel; mobile phase: MeCN in water (0.1% FA), 0% to 46% gradient in 20 min; detector: UV 254 nm to afford tert-butyl (6-(4-(4-((2,6-dioxopiperidin-3-yl)amino)phenyl)piperidin-1-yl)-6-oxohexyl)carbamate (219 mg, 63% yield) as a white solid. LCMS (ESI, m/z): 501.30 [M+H]⁺.

### Step B

A solution of tert-butyl (6-(4-(4-((2,6-dioxopiperidin-3-yl)amino)phenyl)piperidin-1-yl)-6-oxohexyl)carbamate (169 mg, 0.338 mmol, 1.0 equiv) in HCl in 1,4-dioxane (2.5 mL, 4M) was stirred for 6 hours. The resulting mixture was concentrated under reduced pressure to afford 3-((4-(1-(6-aminohexanoyl)piperidin-4-yl)phenyl)amino)piperidine-2,6-dione (238 mg) as a green crude solid. LCMS (ESI, m/z): 401.25 [M+H]⁺.

Intermediates A4-a-A4-e were synthesized according to the procedure described for the synthesis of 3-((4-(1-(6-aminohexanoyl)piperidin-4-yl)phenyl)amino)piperidine-2,6-dione hydrochloride using appropriate building blocks and modified reaction conditions (such as reagents, reagent ratio, temperature, and reaction time) and purification conditions as needed.

| Intermediate | | |
|---|---|---|
| No. | Structure | Analytical Data |
| A4-a | 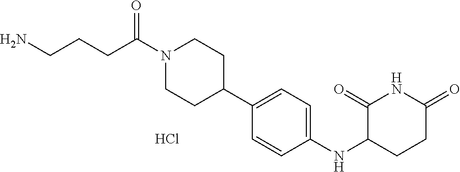 [SMILES: NCCCC(=O)N1CCC(c2ccc(NC3CCC(=O)NC3=O)cc2)CC1 ; Cl] | LCMS (ESI, m/z): 373.22 [M + H]⁺ |
| | 3-((4-(1-(4-aminobutanoyl)piperidin- | |
| | 4-yl)phenyl)amino)piperidine-2,6- | |
| | dione hydrochloride | |
| A4-b | 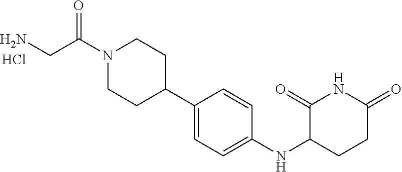 [SMILES: Cl ; NCC(=O)N1CCC(c2ccc(NC3CCC(=O)NC3=O)cc2)CC1] | LCMS (ESI, m/z): 345.18 [M + H]⁺ |
| | 3-((4-(1-glycylpiperidin-4- | |
| | yl)phenyl)amino)piperidine-2,6-dione | |
| | hydrochloride | |
| A4-c | 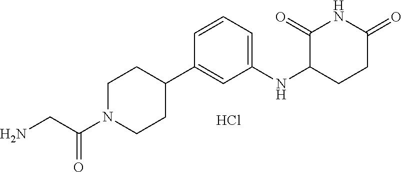 [SMILES: NCC(=O)N1CCC(c2cccc(NC3CCC(=O)NC3=O)c2)CC1 ; Cl] | LCMS (ESI, m/z): 345.10 [M + H]⁺ |
| | 3-((3-(1-glycylpiperidin-4- | |
| | yl)phenyl)amino)piperidine-2,6-dione | |
| A4-d | 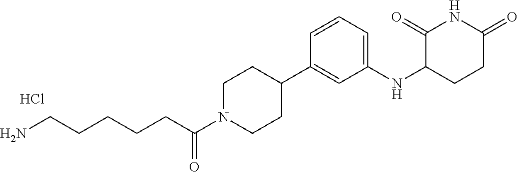 [SMILES: NCCCCCC(=O)N1CCC(c2cccc(NC3CCC(=O)NC3=O)c2)CC1 ; Cl] | LCMS (ESI, m/z): 401.30 [M + H]⁺ |
| | 3-((3-(1-(6-aminohexanoyl)piperidin- | |
| | 4-yl)phenyl)amino)piperidine-2,6- | |
| | dione hydrochloride | |
| A4-e | 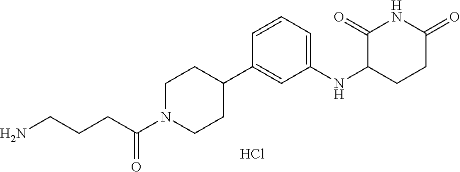 [SMILES: NCCCC(=O)N1CCC(c2cccc(NC3CCC(=O)NC3=O)c2)CC1 ; Cl] | LCMS (ESI, m/z): 373.10 [M + H]⁺ |
| | 3-((3-(1-(4-aminobutanoyl)piperidin- | |
| | 4-yl)phenyl)amino)piperidine-2,6- | |
| | dione hydrochloride | |

### Intermediate A5: Synthesis of 3-((4-(1-(2-aminoethyl)piperidin-4-yl)phenyl)amino)piperidine-2,6-dione hydrochloride

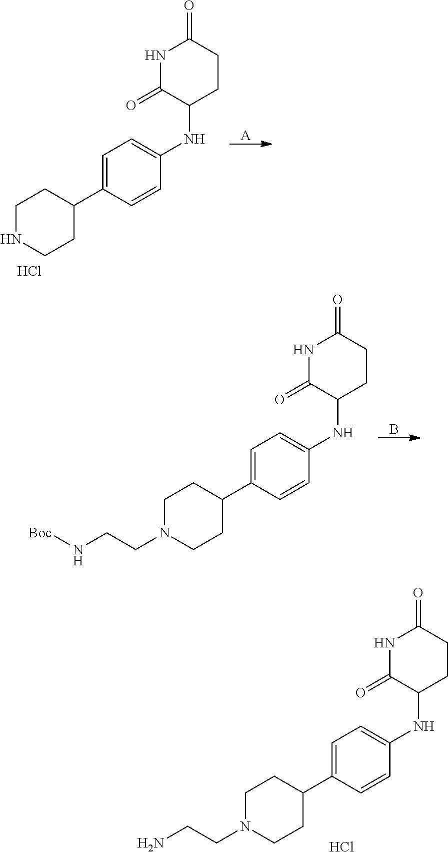

### Step A

A solution of 3-((4-(piperidin-4-yl)phenyl)amino)piperidine-2,6-dione hydrochloride (250 mg, 0.77 mmol, 1.0 equiv), tert-butyl (2-oxoethyl)carbamate (184 mg, 1.16 mmol, 1.5 equiv) and NaBH₃CN (97 mg, 1.5 mmol, 2.0 equiv) in MeOH (10 ml) was stirred for 2 hours. The resulting mixture was concentrated under reduced pressure. The residue was purified by silica gel column chromatography, eluting with DCM/MeOH (9:1) to afford tert-butyl (2-(4-(4-((2,6-dioxopiperidin-3-yl)amino)phenyl)piperidin-1-yl)ethyl)carbamate (200 mg, 48% yield) as a white solid. LCMS (ESI, m/z): 431.25 [M+H]⁺.

### Step B

A solution of tert-butyl (2-(4-(4-((2,6-dioxopiperidin-3-yl)amino)phenyl)piperidin-1-yl)ethyl)carbamate (200 mg, 0.465 mmol, 1.0 equiv) in HCl in 1,4-dioxane (11 mL, 4M) was stirred for 1 hour. The resulting mixture was concentrated under reduced pressure to afford 3-((4-(1-(2-aminoethyl)piperidin-4-yl)phenyl)amino)piperidine-2,6-dione hydrochloride (240 mg) as a crude white solid. The crude product was used without further purification. LCMS (ESI, m/z): 331.25 [M+H]⁺.

Intermediate A5-a was synthesized according to the procedure described for the synthesis of 3-((4-(1-(2-aminoethyl)piperidin-4-yl)phenyl)amino)piperidine-2,6-dione hydrochloride using appropriate building blocks and modified reaction conditions (such as reagents, reagent ratio, temperature, and reaction time) and purification conditions as needed.

| Intermediate | | |
|---|---|---|
| No. | Structure | Analytical Data |
| A5-a | 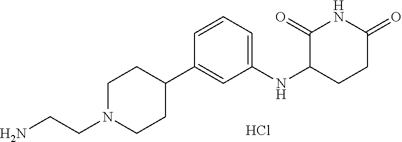 [SMILES: NCCN1CCC(c2cccc(NC3CCC(=O)NC3=O)c2)CC1 ; Cl] | LCMS (ESI, m/z): 331.35 [M + H]⁺ |
| | 3-((3-(1-(2-aminoethyl)piperidin-4- | |
| | yl)phenyl)amino)piperidine-2,6-dione | |
| | hydrochloride | |

### Intermediate A6: Synthesis of 2-bromo-N-(2-(2,6-dioxopiperidin-3-yl)-1,3-dioxoisoindolin-4-yl)acetamide

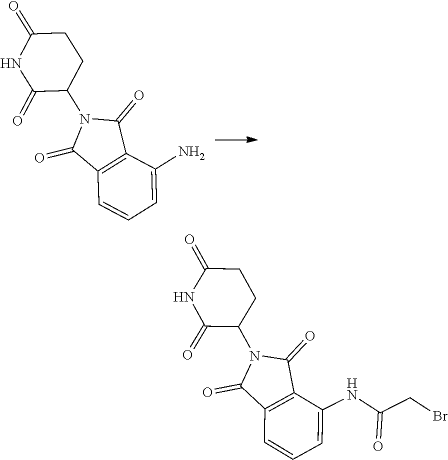 [SMILES: Nc1cccc2c1C(=O)N(C1CCC(=O)NC1=O)C2=O ; O=C1CCC(N2C(=O)c3cccc(NC(=O)CBr)c3C2=O)C(=O)N1]

Bromoacetyl chloride (1.44 g, 9.15 mmol, 2.5 equiv) was added into a solution of pomalidomide (1.0 g, 3.7 mmol, 1.0 equiv) in THF (20 mL) at 0° C. The resulting mixture was stirred for 2 hours at 70° C. The resulting mixture was concentrated under vacuum. The crude product was dissolved in diethyl ether and then stirred for 20 min. The precipitated solids were collected by filtration and washed with diethyl ether (3×20 mL) to afford 2-bromo-N-(2-(2,6-dioxopiperidin-3-yl)-1,3-dioxoisoindolin-4-yl)acetamide (1.2 g, 83%) as a yellow solid. LCMS (ESI, m/z): 412.95 [M+H]⁺.

Intermediate A6-a was synthesized according to the procedure described for the synthesis of 2-bromo-N-(2-(2,6-dioxopiperidin-3-yl)-1,3-dioxoisoindolin-4-yl)acetamide using appropriate building blocks and modified reaction conditions (such as reagents, reagent ratio, temperature, and reaction time) and purification conditions as needed.

| Intermediate | | |
|---|---|---|
| No. | Structure | Analytical Data |
| A6-a | 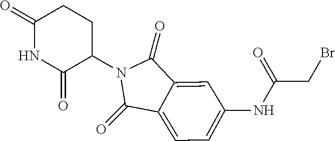 [SMILES: O=C1CCC(N2C(=O)c3ccc(NC(=O)CBr)cc3C2=O)C(=O)N1] | LCMS (ESI, m/z): 394.35 [M + H]⁺ |
| | 2-bromo-N-(2-(2,6-dioxopiperidin-3-yl)- | |
| | 1,3-dioxoisoindolin-5-yl)acetamide | |

### Intermediate A7: Synthesis of 1-(4-(1-(piperidin-4-ylmethyl)piperidin-4-yl)phenyl)dihydropyrimidine-2,4(1H,3H)-dione

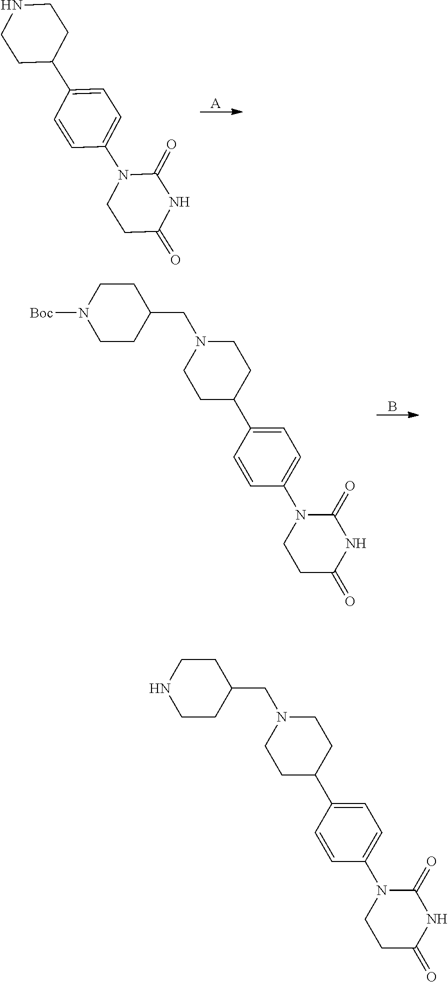

### Step A

A solution of 1-[4-(piperidin-4-yl)phenyl]-1,3-diazinane-2,4-dione (85 mg, 0.31 mmol, 1.0 equiv), tert-butyl 4-(bromomethyl)piperidine-1-carboxylate (130 mg, 0.47 mmol, 1.5 equiv) and DIEA (120 mg, 0.93 mmol, 3.0 equiv) in NMP (5 mL) was stirred for 2 hours at 120° C. The resulting mixture was diluted with water (20 mL) and extracted with EtOAc (3×35 mL). The combined organic layers were washed with brine (3×50 mL), dried over anhydrous Na₂SO₄, filtered, and the filtrate was concentrated under reduced pressure. The residue was purified by reverse-phase flash chromatography with the following conditions: column: C18 silica gel; mobile phase: MeCN in water (10 mmol/L NH₄HCO₃), 5% to 95% gradient in 35 min; detector: UV 254 nm to afford tert-butyl 4-((4-(4-(2,4-dioxotetrahydropyrimidin-1(2H)-yl)phenyl)piperidin-1-yl)methyl)piperidine-1-carboxylate (35 mg, 24% yield) as a yellow solid. LCMS (ESI, m/z): 471.35 [M+H]⁺.

### Step B

A solution of tert-butyl 4-((4-(4-(2,4-dioxotetrahydropyrimidin-1(2H)-yl)phenyl)piperidin-1-yl)methyl)piperidine-1-carboxylate (35 mg, 0.074 mmol, 1.0 equiv) and TFA (1 mL) in DCM (1 mL) was stirred for 50 min. The mixture was concentrated under vacuum to afford 1-(4-(1-(piperidin-4-ylmethyl)piperidin-4-yl)phenyl)dihydropyrimidine-2,4(1H,3H)-dione (28 mg) as a yellow crude solid that was used in the next step directly without further purification. LCMS (ESI, m/z): 371.10 [M+H]⁺.

### Intermediate A8: Synthesis of 3-(2-oxo-3-(4-(piperidin-4-yl)phenyl)imidazolidin-1-yl)piperidine-2,6-dione hydrochloride

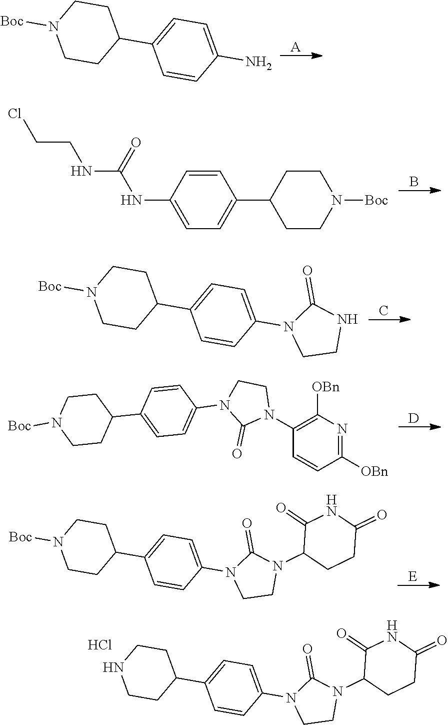

### Step A

To a solution of tert-butyl 4-(4-aminophenyl)piperidine-1-carboxylate (2.0 g, 7.2 mmol, 1.0 equiv) in DCM (10 mL) was added 1-chloro-2-isocyanatoethane (916 mg, 8.68 mmol, 1.2 equiv) portion-wise at 0° C. The solution was stirred for 1 hour at 0° C. and then concentrated under vacuum to afford tert-butyl 4-(4-(3-(2-chloroethyl)ureido)phenyl)piperidine-1-carboxylate (2.9 g, 99% yield) as a white solid. The product was used in the next step without further purification. LCMS (ESI, m/z): 326.25 [M+H-t-Bu]⁺.

### Step B

To a solution of tert-butyl 4-(4-(3-(2-chloroethyl)ureido)phenyl)piperidine-1-carboxylate (2.9 grams, 7.6 mmol, 1.0 equiv) in THF (10 mL) was added NaH (220 mg, 9.19 mmol, 1.2 equiv, 60% dispersion in oil) portion-wise at 0° C. The resulting mixture was stirred for 1 hour at 0° C. After completion and then water was added and extracted with ethyl acetate (3×100 mL). The combined organic layers were concentrated under vacuum to afford tert-butyl 4-(4-(2-oxoimidazolidin-1-yl)phenyl)piperidine-1-carboxylate (2.74 g, 93% yield) as a white solid. LCMS (ESI, m/z): 346.10 [M+H]⁺.

### Step C

A solution of tert-butyl 4-(4-(2-oxoimidazolidin-1-yl)phenyl)piperidine-1-carboxylate (500 mg, 1.5 mmol, 1.0 equiv), 2,6-bis(benzyloxy)-3-bromopyridine (536 mg, 1.45 mmol, 1.0 equiv), (1R,2S)-N1,N2-dimethylcyclohexane-1,2-diamine (206 mg, 1.45 mmol, 1.0 equiv), K₃PO₄ (922 mg, 4.34 mmol, 3.0 equiv) and CuI (28 mg, 0.14 mmol, 0.10 equiv) in toluene (10 mL) was stirred for 4 hours at 120° C. The solution was concentrated under vacuum and applied onto a silica gel column eluting with ethyl acetate/petroleum ether (20:80) to afford tert-butyl 4-(4-(3-(2,6-bis(benzyloxy)pyridin-3-yl)-2-oxoimidazolidin-1-yl)phenyl)piperidine-1-carboxylate (720 mg, 63%) as a white solid. LCMS (ESI, m/z): 635.25 [M+H]^{m}.

### Step D

A solution of tert-butyl 4-(4-(3-(2,6-bis(benzyloxy)pyridin-3-yl)-2-oxoimidazolidin-1-yl)phenyl)piperidine-1-carboxylate (600 mg, 0.95 mmol, 1.0 equiv) and Pd/C (503 mg, 4.72 mmol, 5.0 equiv) in ethyl acetate (10 mL) was stirred for 15 min under an atmosphere of hydrogen. The resulting mixture was filtered, and the filter cake was washed with DCM (3×200 mL). The filtrate was concentrated under vacuum to afford tert-butyl 4-(4-(3-(2,6-dioxopiperidin-3-yl)-2-oxoimidazolidin-1-yl)phenyl)piperidine-1-carboxylate (360 mg, 75%) as a brown solid. LCMS (ESI, m/z): 457.10 [M+H]⁺.

### Step E

A solution of tert-butyl 4-(4-(3-(2,6-dioxopiperidin-3-yl)-2-oxoimidazolidin-1-yl)phenyl)piperidine-1-carboxylate (340 mg, 0.745 mmol, 1.0 equiv) in HCl in 1,4-dioxane (5 mL, 4 M) was stirred for 30 min. The resulting mixture was concentrated under vacuum to afford 3-(2-oxo-3-(4-(piperidin-4-yl)phenyl)imidazolidin-1-yl)piperidine-2,6-dione hydrochloride (380 mg) as a brown crude solid that was used without further purification. LCMS (ESI, m/z): 357.30 [M+H]⁺.

### Intermediate A9: Synthesis of 3-(4-bromo-3-methyl-2-oxo-2,3-dihydro-1H-benzo[d]imidazol-1-yl)piperidine-2,6-dione

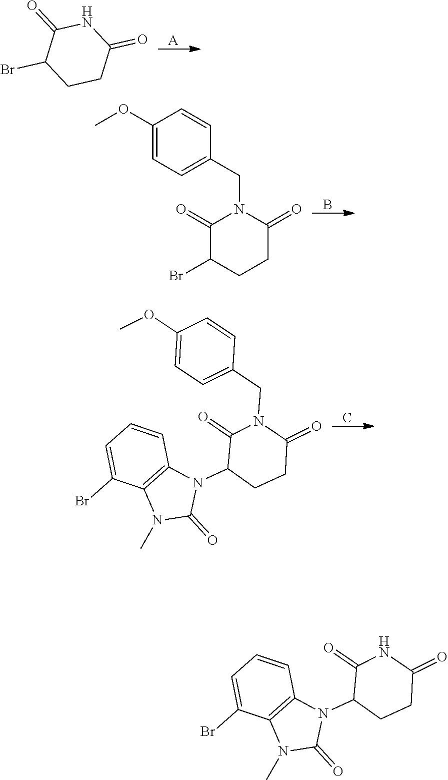

### Step A

To a stirred solution of 3-bromopiperidine-2,6-dione (5.0 g, 26.0 mmol, 1.0 equiv), PPh₃ (10.3 g, 39.1 mmol, 1.5 equiv) and (4-methoxyphenyl)methanol (5.40 g, 39.1 mmol, 1.5 equiv) in THF (50 mL) was added DEAD (6.80 g, 39.1 mmol, 1.5 equiv) at 0° C. The resulting solution was stirred for 4 hours at room temperature. After concentration, the residue was purified by a silica gel column eluting with PE/EA(1:1) to afford 3-bromo-1-(4-methoxybenzyl)piperidine-2,6-dione (7.3 g, 90% yield) as a yellow solid.

### Step B

A solution of 7-bromo-1-methyl-3H-1,3-benzodiazol-2-one (1.0 g, 4.4 mmol, 1 equiv) in ACN (8 mL) was treated with Cs₂CO₃ (4.30 g, 13.2 mmol, 3.0 equiv) for 5 min at 0° C. followed by the addition of 3-bromo-1-(4-methoxybenzyl)piperidine-2,6-dione (1.65 g, 5.29 mmol, 1.2 equiv) dropwise at 0° C. The resulting solution was stirred at room temperature for 15 hours. After concentration, the residue was purified by a silica gel column eluting with PE/EA (1:1) to afford 3-(4-bromo-3-methyl-2-oxo-2,3-dihydro-1H-benzo[d]imidazol-1-yl)-1-(4-methoxybenzyl)piperidine-2,6-dione (900 mg, 45% yield) as a white solid. LCMS (ESI, m/z): 458.15 [M+H]⁺.

### Step C

A solution of 3-(4-bromo-3-methyl-2-oxo-2,3-dihydro-1H-benzo[d]imidazol-1-yl)-1-(4-methoxybenzyl)piperidine-2,6-dione (1.16 g, 2.53 mmol, 1.0 equiv) in methane sulfonic acid (4 mL) and toluene (8 mL) was stirred for 2 hours at 120° C. under a nitrogen atmosphere. The resulting mixture was concentrated under reduced pressure. The reaction was quenched by the addition of ice water (20 mL). The precipitated solids were collected by filtration and washed with water. The crude product was purified by a silica gel chromatography eluting with PE/EA (1:3) to afford 3-(4-bromo-3-methyl-2-oxo-2,3-dihydro-1H-benzo[d]imidazol-1-yl)piperidine-2,6-dione (800 mg, 93% yield) as a grey solid. LCMS (ESI, m/z): 337.80 [M+H]⁺.

Intermediate A9-a was synthesized according to the procedure described for the synthesis of 3-(4-bromo-3-methyl-2-oxo-2,3-dihydro-1H-benzo[d]imidazol-1-yl)piperidine-2,6-dione using appropriate building blocks and modified reaction conditions (such as reagents, reagent ratio, temperature, and reaction time) and purification conditions as needed.

| Example | | |
|---|---|---|
| No. | Structure | Analytical Data |
| A9-a | 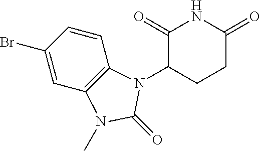 [SMILES: Cn1c(=O)n(C2CCC(=O)NC2=O)c2ccc(Br)cc21] | LCMS (ESI, m/z): 338.00 [M + H]+ |
| | 3-(5-bromo-3-methyl-2-oxo-2,3-dihydro- | |
| | 1H-benzo[d]imidazol-1-yl)piperidine-2,6- | |
| | dione | |

### Intermediate A10: Synthesis of 3-(5-(4-(hydroxymethyl)piperidin-1-yl)-1-oxoisoindolin-2-yl)piperidine-2,6-dione

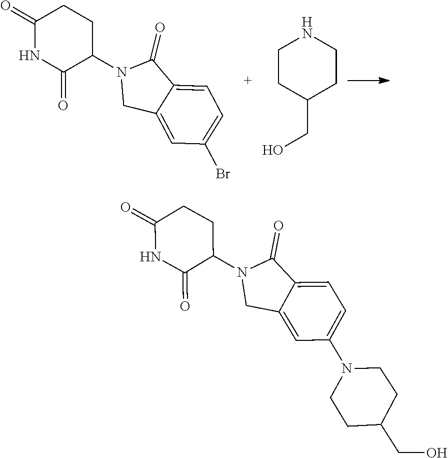 [SMILES: O=C1CCC(N2Cc3cc(Br)ccc3C2=O)C(=O)N1 ; OCC1CCNCC1 ; O=C1CCC(N2Cc3cc(N4CCC(CO)CC4)ccc3C2=O)C(=O)N1]

A solution of 3-(5-bromo-1-oxoisoindolin-2-yl)piperidine-2,6-dione (500 mg, 1.55 mmol, 1 equiv), piperidin-4-ylmethanol (178 mg, 1.55 mmol, 1 equiv), Pd-PEPPSI-IPentCl 2-methylpyridine (o-picoline) (67 mg, 0.077 mmol, 0.05 equiv) and Cs₂CO₃ (1008 mg, 3.09 mmol, 2 equiv) in dioxane (5 mL) was stirred for 1 hour at 100° C. under nitrogen atmosphere. The mixture was concentrated and the residue purified by silica gel column chromatography, eluting with PE/EtOAc (1:1) to afford 3-(5-(4-(hydroxymethyl)piperidin-1-yl)-1-oxoisoindolin-2-yl)piperidine-2,6-dione (240 mg, 43%) as a yellow solid. LCMS (ESI, m/z): 358.25 [M+H]⁺.

### Intermediate A11: Synthesis of 1-(2-fluoro-4-nitrophenyl)piperidin-4-one

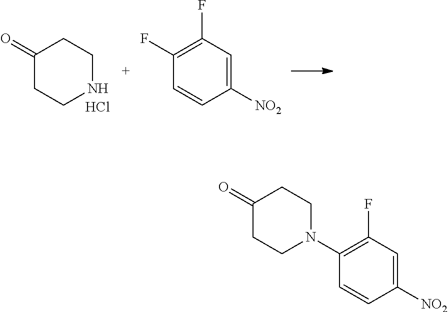 [SMILES: O=C1CCNCC1 ; O=[N+]([O-])c1ccc(F)c(F)c1 ; Cl ; O=C1CCN(c2ccc([N+](=O)[O-])cc2F)CC1]

A solution of piperidin-4-one hydrochloride (6.0 g, 44 mmol, 1 equiv), 1,2-difluoro-4-nitrobenzene (7.04 g, 44.3 mmol, 1 equiv) and TEA (13.4 g, 133 mmol, 3 equiv) in DMF (40 mL) was stirred overnight at 80° C. The product was precipitated by the addition of water. The precipitated solids were collected by filtration and washed with water (3×80 mL) to give 1-(2-fluoro-4-nitrophenyl)piperidin-4-one (9 g, 85%) as a yellow solid. LCMS (ESI, m/z): 239.10 [M+H]⁺.

### Intermediate A12: Synthesis of 3-(2-oxo-6-(piperazin-1-yl)benzo[cd]indol-1(2H)-yl)piperidine-2,6-dione hydrochloride

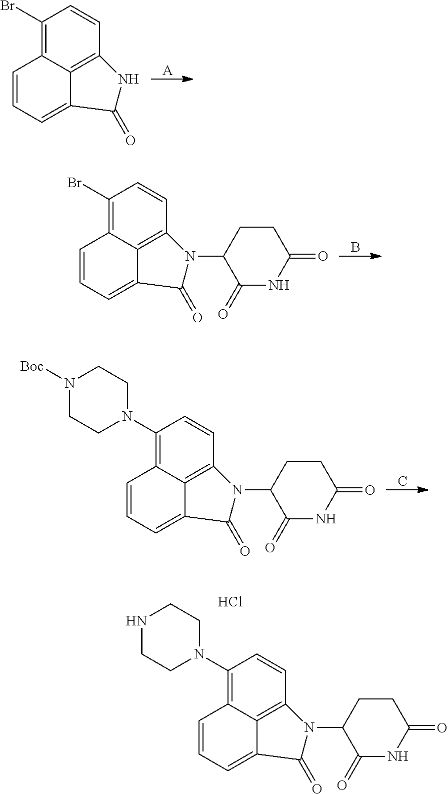

### Step A

To a stirred solution of 6-bromobenzo[cd]indol-2(1H)-one (2.0 g, 8.1 mmol, 1 equiv) and THF (150 mL) was added NaH (1.61 g, 40.3 mmol, 5 equiv, 60% dispersion in oil) portion-wise at 0° C. The mixture was stirred for 1 hour at room temperature. A solution of 3-bromopiperidine-2,6-dione (3.87 g, 20.2 mmol, 2.5 equiv) in THF (10 mL) was added dropwise at 0° C. The mixture was stirred overnight at 60° C. The reaction was quenched with the slow addition of saturated aqueous NH₄Cl (80 mL) at 0° C. The mixture was extracted with EtOAc (2×100 mL). The combined organic layers were washed with brine (80 mL) and dried over anhydrous Na₂SO₄. After filtration, the filtrate was concentrated under reduced pressure. The residue was purified by trituration with DCM (10 mL) to afford 3-(6-bromo-2-oxobenzo[cd]indol-1(2H)-yl)piperidine-2,6-dione (940 mg, 33%) as a yellow-green solid. LCMS (ESI, m/z): 359.18 [M+H]⁺.

### Step B

A solution of 3-(6-bromo-2-oxobenzo[cd]indol-1(2H)-yl)piperidine-2,6-dione (400 mg, 1.11 mmol, 1 equiv), tert-butyl piperazine-1-carboxylate (311 mg, 1.67 mmol, 1.5 equiv), Pd-PEPPSI-IPentCl 2-methylpyridine (o-picoline) (94 mg, 0.111 mmol, 0.1 equiv) and Cs₂CO₃ (544 mg, 1.67 mmol, 1.5 equiv) in dioxane (10 mL) was stirred overnight at 100° C. under a nitrogen atmosphere. The mixture was concentrated and the residue was purified by silica gel column chromatography, eluting with PE/EtOAc (1:1) to afford tert-butyl 4-(1-(2,6-dioxopiperidin-3-yl)-2-oxo-1,2-dihydrobenzo[cd]indol-6-yl)piperazine-1-carboxylate (361 mg, 70%) as a yellow solid. LCMS (ESI, m/z): 465.52 [M+H]⁺.

### Step C

A solution of tert-butyl 4-(1-(2,6-dioxopiperidin-3-yl)-2-oxo-1,2-dihydrobenzo[cd]indol-6-yl)piperazine-1-carboxylate (341 mg, 0.73 mmol, 1 equiv) and HCl in 1,4-dioxane (10 mL, 4 M) was stirred for 30 min. The mixture was concentrated to afford 3-(2-oxo-6-(piperazin-1-yl)benzo[cd]indol-1(2H)-yl)piperidine-2,6-dione hydrochloride (335 mg) as a yellow solid. The crude product was used in the next step directly without further purification. LCMS (ESI, m/z): 365.41 [M+H]⁺.

### Intermediate A13: Synthesis of 1-(3-fluoro-4-(4-oxopiperidin-1-yl)phenyl)dihydropyrimidine-2,4(1H,3H)-dione

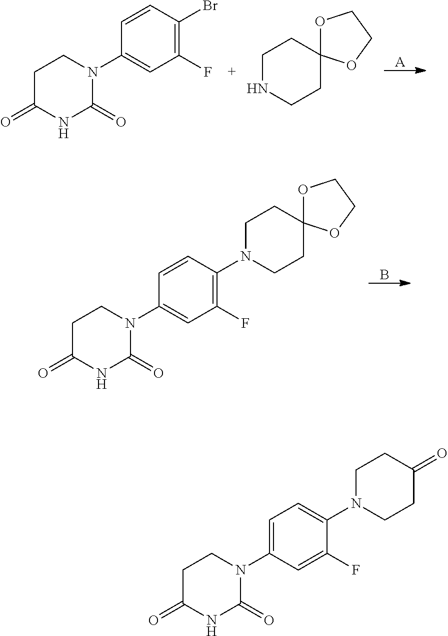

### Step A

A solution of 1-(4-bromo-3-fluorophenyl)dihydropyrimidine-2,4(1H,3H)-dione (500 mg, 1.74 mmol, 1 equiv), 1,4-dioxa-8-azaspiro[4.5]decane (374 mg, 2.61 mmol, 1.5 equiv), Cs₂CO₃ (1135 mg, 3.48 mmol, 2 equiv) and Pd-PEPPSI-IPentCl 2-methylpyridine o-picoline (147 mg, 0.174 mmol, 0.1 equiv) in dioxane (8 mL) was stirred for 1 hour at 85° C. under nitrogen atmosphere. The mixture was concentrated and the residue purified by silica gel column chromatography, eluting with PE/EtOAc (1:3) to afford 1-(3-fluoro-4-(1,4-dioxa-8-azaspiro[4.5]decan-8-yl)phenyl)dihydropyrimidine-2,4(1H,3H)-dione (520 mg, 85%) as a white solid. LCMS (ESI, m/z): 350.35 [M+H]⁺.

### Step B

A solution of 1-(3-fluoro-4-(1,4-dioxa-8-azaspiro[4.5]decan-8-yl)phenyl)dihydropyrimidine-2,4(1H,3H)-dione (510 mg, 1.46 mmol, 1 equiv) and HCl (10 mL, 6 M) in THF (10 mL) was stirred overnight. The mixture was neutralized to pH 7 with saturated aqueous NaHCO₃. The mixture was extracted with DCM (3×50 mL). The combined organic layers were dried over anhydrous Na₂SO₄. After filtration, the filtrate was concentrated under reduced pressure to afford 1-(3-fluoro-4-(4-oxopiperidin-1-yl)phenyl)dihydropyrimidine-2,4(1H,3H)-dione (460 mg) as a white solid. The crude product was used in the next step directly without further purification LCMS (ESI, m/z): 306.15 [M+H]⁺.

### Intermediate A14: Synthesis of 3-((3-fluoro-4-(4-(piperazin-1-yl)piperidin-1-yl)phenyl)amino)piperidine-2,6-dione

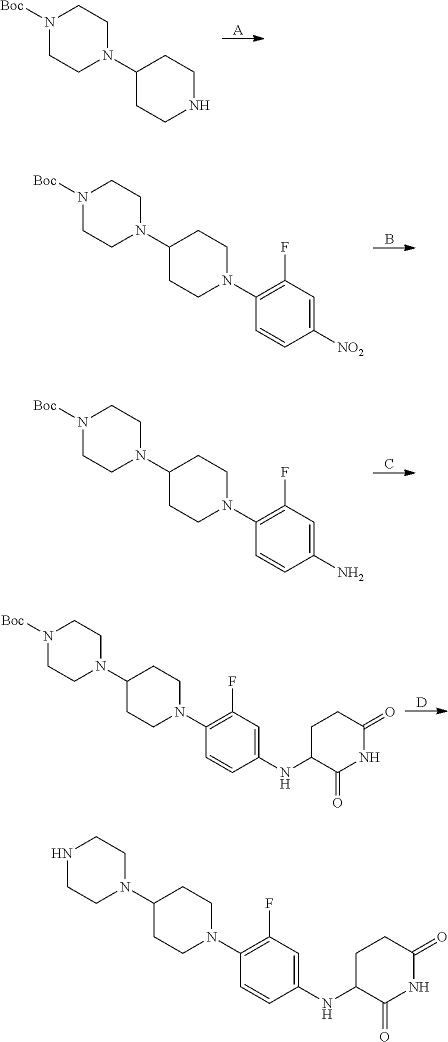

### Step A

A solution of tert-butyl 4-(piperidin-4-yl)piperazine-1-carboxylate (2.0 g, 7.4 mmol, 1 equiv), 1,2-difluoro-4-nitrobenzene (1.18 g, 7.42 mmol, 1 equiv) and TEA (2.25 g, 22.3 mmol, 3 equiv) in ACN (50 mL) was stirred for 1 hour at 80° C. The solution was concentrated and the residue was purified by silica gel column chromatography, eluting with PE/EtOAc (5:1) to afford tert-butyl 4-(1-(2-fluoro-4-nitrophenyl)piperidin-4-yl)piperazine-1-carboxylate (2.2 g, 72%) as a brown solid. LCMS (ESI, m/z): 409.30 [M+H]⁺.

### Step B

A solution of tert-butyl 4-(1-(2-fluoro-4-nitrophenyl)piperidin-4-yl)piperazine-1-carboxylate (2.2 g, 5.39 mmol, 1 equiv), NH₄Cl (0.86 g, 16 mmol, 3 equiv) and Fe (1.50 g, 26.9 mmol, 5 equiv) in EtOH (50 mL) and water (10 mL) was stirred for 1 hour at 80° C. The mixture was concentrated and purified by silica gel column chromatography, eluting with PE/EtOAc (1:4) to afford tert-butyl 4-(1-(4-amino-2-fluorophenyl)piperidin-4-yl)piperazine-1-carboxylate (1.9 g, 99%) as a brown solid. LCMS (ESI, m/z): 379.30 [M+H]⁺.

### Step C

A solution of tert-butyl 4-(1-(4-amino-2-fluorophenyl)piperidin-4-yl)piperazine-1-carboxylate (1.9 g, 5.02 mmol, 1 equiv), 3-bromopiperidine-2,6-dione (4.82 g, 25.1 mmol, 5 equiv) and NaHCO₃ (2.11 g, 25.1 mmol, 5 equiv) in ACN (50 mL) was stirred overnight at 90° C. The mixture was concentrated and the residue purified by silica gel column chromatography, eluting with DCM/MeOH (2:3) to afford tert-butyl 4-(1-(4-((2,6-dioxopiperidin-3-yl)amino)-2-fluorophenyl)piperidin-4-yl)piperazine-1-carboxylate (1.7 g, 69%) as a brown solid. LCMS (ESI, m/z): 490.25 [M+H]⁺.

### Step D

A solution of tert-butyl 4-(1-(4-((2,6-dioxopiperidin-3-yl)amino)-2-fluorophenyl)piperidin-4-yl)piperazine-1-carboxylate (1.7 g, 3.5 mmol, 1 equiv) in HCl in 1,4-dioxane (30 mL, 4 M) was stirred for 1 hour. The mixture was concentrated to afford 3-((3-fluoro-4-(4-(piperazin-1-yl)piperidin-1-yl)phenyl)amino)piperidine-2,6-dione (2.2 g) as a brown solid. The crude product was used in the next step directly without further purification. LCMS (ESI, m/z): 390.20 [M+H]⁺.

### Intermediate A15: Synthesis of 1-(1-methyl-6-(4-oxopiperidin-1-yl)-1H-indazol-3-yl)dihydropyrimidine-2,4(1H,3H)-dione

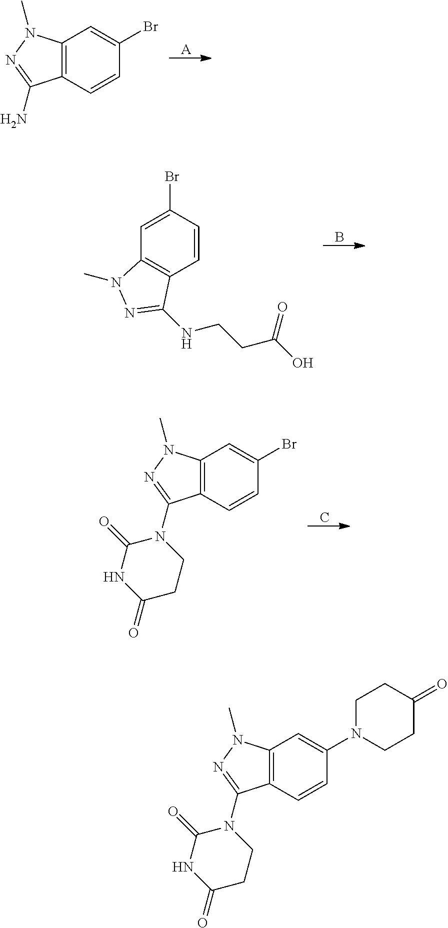

### Step A

A solution of 6-bromo-1-methyl-1H-indazol-3-amine (30 g, 132 mmol, 1 equiv), acrylic acid (14.3 g, 199 mmol, 1.5 equiv) and TBAB (4.28 g, 13.3 mmol, 0.1 equiv) in 2 M HCl (1.2 L) was stirred overnight at 100° C. The mixture was neutralized to pH 7 with aqueous NaOH (4 M). The precipitated solids were collected by filtration and washed with water (3×100 mL). The resulting solid was dried under infrared light to give 3-((6-bromo-1-methyl-1H-indazol-3-yl)amino)propanoic acid (35.9 g, 77%) as a gray solid. LCMS (ESI, m/z): 300.10 [M+H]⁺.

### Step B

A solution of 3-((6-bromo-1-methyl-1H-indazol-3-yl)amino)propanoic acid (10 g, 34 mmol, 1 equiv) in AcOH (150 mL) was treated with sodium cyanate (6.54 g, 101 mmol, 3 equiv) for 12 hours at 80° C. followed by the addition of HCl (150 mL, 4 M) dropwise at room temperature. Then the solution was stirred overnight at 80° C. The precipitated solids were collected by filtration and washed with water (3×30 mL). The resulting solid was dried under infrared light to give 1-(6-bromo-1-methyl-1H-indazol-3-yl)dihydropyrimidine-2,4(1H,3H)-dione (4.28 g, 40%) as a white solid. LCMS (ESI, m/z): 325.00 [M+H]⁺.

### Step C

A solution of 1-(6-bromo-1-methyl-1H-indazol-3-yl)dihydropyrimidine-2,4(1H,3H)-dione (1.2 g, 3.7 mmol, 1 equiv), piperidin-4-one (0.55 g, 5.6 mmol, 1.5 equiv), Cs₂CO₃ (3.63 g, 11.1 mmol, 3 equiv) and Pd-PEPPSI-IPentCl 2-methylpyridine (o-picoline) (0.31 g, 0.37 mmol, 0.1 equiv) in dioxane (8 mL) was stirred overnight at 85° C. After concentration, the residue was purified by silica gel column chromatography, eluting with DCM/MeOH (9:1) to afford 1-(1-methyl-6-(4-oxopiperidin-1-yl)-1H-indazol-3-yl)dihydropyrimidine-2,4(1H,3H)-dione (480 mg, 36%) as a yellow solid. LCMS (ESI, m/z): 342.10 [M+H]⁺.

### Intermediate A16: Synthesis of 3-((4-([4,4′-bipiperidin]-1-yl)-3-fluorophenyl)amino)piperidine-2,6-dione hydrochloride

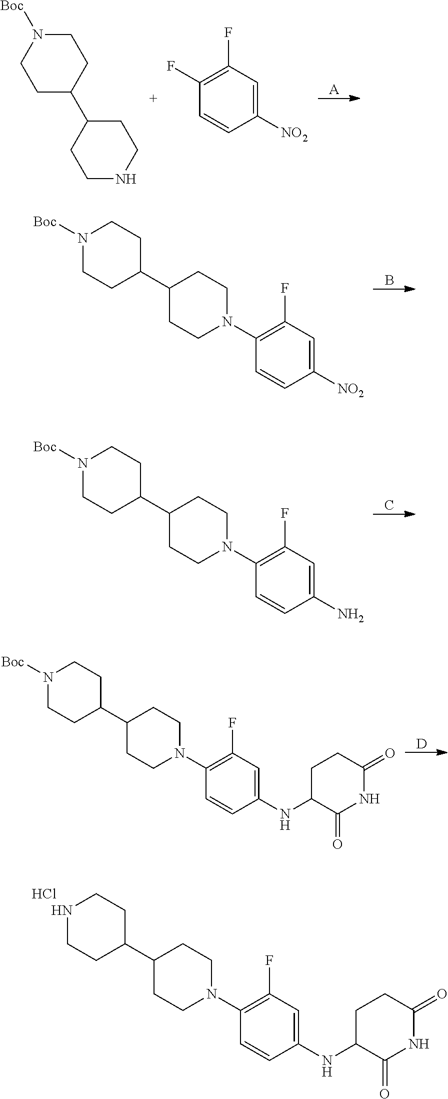

### Step A

A mixture of tert-butyl [4,4′-bipiperidine]-1-carboxylate (2.0 g, 7.5 mmol, 1 equiv), 1,2-difluoro-4-nitrobenzene (1.78 g, 11.2 mmol, 1.5 equiv), and NaHCO₃ (2.50 g, 29.8 mmol, 4 equiv) in ACN (5 mL) was stirred for 4 hours at 90° C. After concentration, the residue was purified by silica gel column chromatography, eluting with PE/EtOAc (4:1) to afford tert-butyl 1′-(2-fluoro-4-nitrophenyl)-[4,4′-bipiperidine]-1-carboxylate (2.7 g, 89%) as a yellow solid. LCMS (ESI, m/z): 408.35 [M+H]⁺.

### Step B

A mixture of tert-butyl 1′-(2-fluoro-4-nitrophenyl)-[4,4′-bipiperidine]-1-carboxylate (2.7 g, 6.6 mmol, 1 equiv), Fe (1.85 g, 33.1 mmol, 5 equiv) and NH₄Cl (0.71 g, 13 mmol, 2 equiv) in EtOH (4 mL) and water (1 mL) was stirred for 4 hours at 80° C. After concentration, the residue was purified by silica gel column chromatography, eluting with PE/EtOAc (3:7) to afford tert-butyl 1′-(4-amino-2-fluorophenyl)-[4,4′-bipiperidine]-1-carboxylate (2.34 g, 94%) as a yellow solid. LCMS (ESI, m/z): 378.15 [M+H]⁺.

### Step C

A mixture of tert-butyl 1′-(4-amino-2-fluorophenyl)-[4,4′-bipiperidine]-1-carboxylate (2.3 g, 6.09 mmol, 1 equiv), 3-bromopiperidine-2,6-dione (3.51 g, 18.3 mmol, 3 equiv), and NaHCO₃ (2.56 g, 30.5 mmol, 5 equiv) in ACN (6 mL) was stirred overnight at 90° C. After concentration the residue was purified by silica gel column chromatography, eluting with PE/EtOAc (1:1) to afford tert-butyl 1′-(4-((2,6-dioxopiperidin-3-yl)amino)-2-fluorophenyl)-[4,4′-bipiperidine]-1-carboxylate (2.6 g, 87%) as a green solid. LCMS (ESI, m/z): 487.25 [M−H]⁻.

### Step D

A mixture of tert-butyl 1′-(4-((2,6-dioxopiperidin-3-yl)amino)-2-fluorophenyl)-[4,4′-bipiperidine]-1-carboxylate (2.6 g, 5.3 mmol, 1 equiv) in HCl in 1,4-dioxane (8 mL, 4 M) was stirred for 3 hours. The mixture was concentrated to give 3-((4-([4,4′-bipiperidin]-1-yl)-3-fluorophenyl)amino)piperidine-2,6-dione hydrochloride (2.8 g) as a white solid. The product was used in the next step without further purification. LCMS (ESI, m/z): 389.20 [M+H]⁺.

### Intermediate A17: Synthesis of 1-(8-(piperazin-1-yl)isoquinolin-4-yl)dihydropyrimidine-2,4(1H,3H)-dione hydrochloride

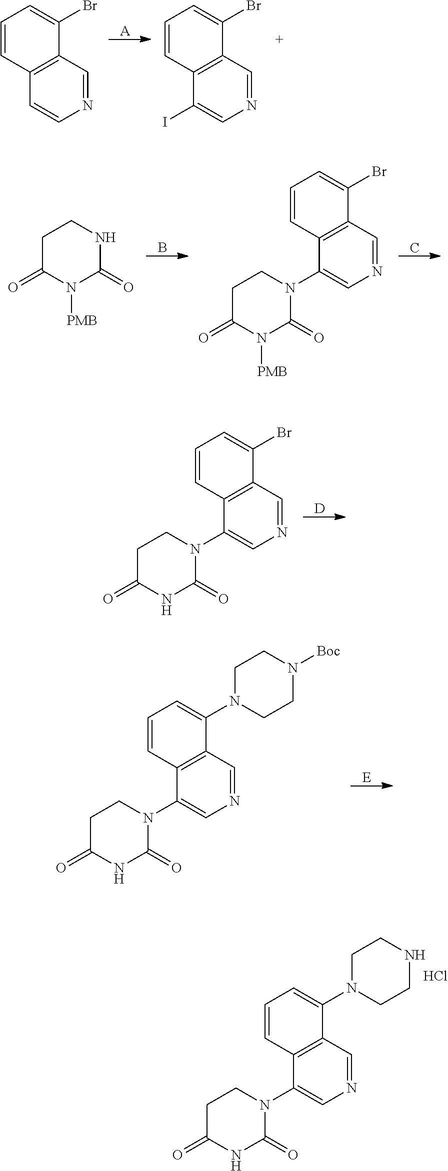

### Step A

A solution of 8-bromoisoquinoline (5 g, 24.0 mmol, 1 equiv), I₂ (12.2 g, 48.1 mmol, 2 equiv) and TBHP (6.50 g, 72.1 mmol, 3 equiv, 70% aqueous) in DCE (50 mL) was stirred overnight at 120° C. The reaction was quenched with aqueous sodium sulfite at room temperature. The aqueous layer was extracted with DCM (3×20 mL). The resulting mixture was concentrated under reduced pressure affording 8-bromo-4-iodoisoquinoline (5.3 g, 66%) as a red solid. LCMS (ESI, m/z): 333.80 [M+H]⁺.

### Step B

A solution of 8-bromo-4-iodoisoquinoline (1 g, 3.0 mmol, 1 equiv), 3-(4-methoxybenzyl)dihydropyrimidine-2,4(1H,3H)-dione (0.91 g, 3.89 mmol, 1.3 equiv), (1R,2R)-N1,N2-dimethylcyclohexane-1,2-diamine (0.21 g, 1.50 mmol, 0.5 equiv), Cs₂CO₃ (1.95 g, 6.0 mmol, 2 equiv) and CuI (0.23 g, 1.20 mmol, 0.4 equiv) in dioxane (6 mL) was stirred overnight at 65° C. under nitrogen atmosphere. The resulting mixture was concentrated and the residue was purified by silica gel column chromatography, eluting with PE/EtOAc (45:55) to afford 1-(8-bromoisoquinolin-4-yl)-3-(4-methoxybenzyl)dihydropyrimidine-2,4(1H,3H)-dione (730 mg, 55%) as a yellow solid. LCMS (ESI, m/z): 440.05 [M+H]⁺.

### Step C

A solution of 1-(8-bromoisoquinolin-4-yl)-3-(4-methoxybenzyl)dihydropyrimidine-2,4(1H,3H)-dione (1.6 g, 3.63 mmol, 1 equiv) in TFA (5 mL) and TfOH (1 mL) was stirred for 4 hours. The residue was diluted with EtOAc (4 mL) then basified to pH 8 with TEA. The precipitated solids were collected by filtration and washed with water (3×5 mL). This resulted in 1-(8-bromoisoquinolin-4-yl)dihydropyrimidine-2,4(1H,3H)-dione (1.5 g) as a yellow solid. The crude product was used in the next step directly without further purification LCMS (ESI, m/z): 320.00 [M+H]⁺.

### Step D

A solution of 1-(8-bromoisoquinolin-4-yl)dihydropyrimidine-2,4(1H,3H)-dione (500 mg, 1.56 mmol, 1 equiv), tert-butyl piperazine-1-carboxylate (436 mg, 2.34 mmol, 1.5 equiv), Pd-PEPPSI-IPentCl 2-methylpyridine (o-picoline) (131 mg, 0.16 mmol, 0.1 equiv) and Cs₂CO₃ (1018 mg, 3.12 mmol, 2 equiv) in dioxane (4 mL) was stirred for 3 hours at 85° C. under nitrogen atmosphere. The resulting mixture was concentrated and the residue was purified by silica gel column chromatography, eluting with DCM/EtOH (92:8) to afford tert-butyl 4-(4-(2,4-dioxotetrahydropyrimidin-1(2H)-yl)isoquinolin-8-yl)piperazine-1-carboxylate (377 mg, 57%) as a yellow solid. LCMS (ESI, m/z): 426.21 [M+H]⁺.

### Step E

A solution of tert-butyl 4-(4-(2,4-dioxotetrahydropyrimidin-1(2H)-yl)isoquinolin-8-yl)piperazine-1-carboxylate (367 mg, 0.86 mmol, 1 equiv) in HCl in 1,4-dioxane (5 mL, 4 M) was stirred for 1 hour. The mixture was concentrated to dryness to afford 1-(8-(piperazin-1-yl)isoquinolin-4-yl)dihydropyrimidine-2,4(1H,3H)-dione hydrochloride (400 mg) as a yellow solid. The crude product was used in the next step directly without further purification. LCMS (ESI, m/z): 326.15 [M+H]⁺.

### Intermediate B1: Synthesis of 2-(4-(((7-(cyclopentylamino)-5-fluoro-4-oxo-3,4-dihydroquinazolin-2-yl)methyl)thio)piperidin-1-yl)acetic acid

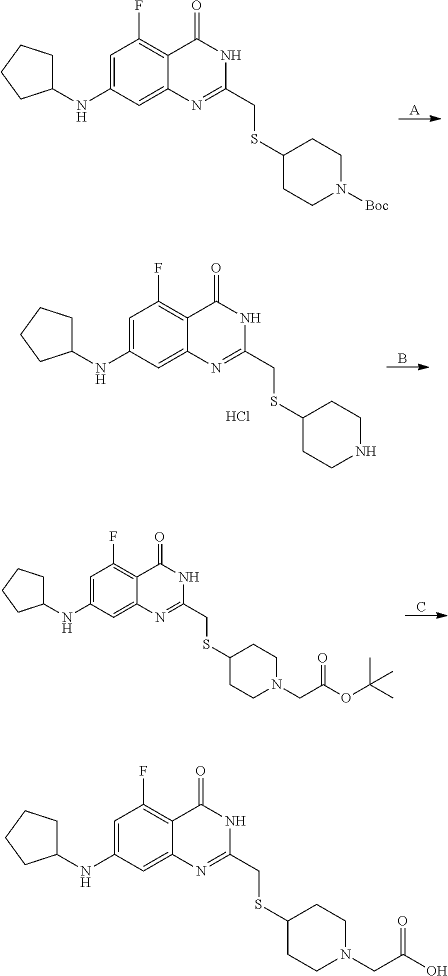

### Step A

A solution of tert-butyl 4-(((7-(cyclopentylamino)-5-fluoro-4-oxo-3,4-dihydroquinazolin-2-yl)methyl)thio)piperidine-1-carboxylate (5.0 g, 10.5 mmol, 1.0 equiv) in HCl in 1,4-dioxane (60 mL, 4 M) was stirred for 2 hours at 40° C. The resulting mixture was concentrated under vacuum. The mixture was diluted with ethyl acetate (60 mL) and the precipitated solids were collected by filtration to afford the HCl salt of 7-(cyclopentylamino)-5-fluoro-2-((piperidin-4-ylthio)methyl)quinazolin-4(3H)-one (5.0 g) as a grey solid. The crude product mixture was used directly without further purification. LCMS (ESI, m/z): 377.15 [M+H]⁺.

### Step B

A solution of 7-(cyclopentylamino)-5-fluoro-2-((piperidin-4-ylthio)methyl)quinazolin-4(3H)-one hydrogen chloride salt (4.28 g, 11.4 mmol, 1.0 equiv) and tert-butyl 2-bromoacetate (1.69 g, 8.65 mmol, 0.76 equiv) and K₂CO₃ (6.28 g, 45.5 mmol, 4.0 equiv) in DMF (45 mL) was stirred for 3 hours at 70° C. The resulting mixture was diluted with brine (30 mL) and extracted with EtOAc (3×80 mL). The combined organic layers were washed with brine (3×20 mL), dried over anhydrous Na₂SO₄, filtered and concentrated under reduced pressure. The crude product was purified by trituration with EtOAc (20 mL) to afford tert-butyl 2-(4-(((7-(cyclopentylamino)-5-fluoro-4-oxo-3,4-dihydroquinazolin-2-yl)methyl)thio)piperidin-1-yl)acetate (2.6 g, 47%) as an off-white solid. LCMS (ESI, m/z): 491.15 [M+H]⁺.

### Step C

A solution of tert-butyl 2-(4-(((7-(cyclopentylamino)-5-fluoro-4-oxo-3,4-dihydroquinazolin-2-yl)methyl)thio)piperidin-1-yl)acetate (2.6 g, 5.3 mmol, 1.0 equiv) in trifluoroacetic acid (20 mL) and DCM (40 mL) was stirred for 16 hours. The resulting mixture was concentrated under vacuum and then diluted with DCM (20 mL) and water (20 ml). The mixture was neutralized to pH 6.0 with saturated aqueous Na₂CO₃. The resulting mixture was concentrated under vacuum to remove DCM. The precipitated solids were collected by filtration to afford 2-(4-(((7-(cyclopentylamino)-5-fluoro-4-oxo-3,4-dihydroquinazolin-2-yl)methyl)thio)piperidin-1-yl)acetic acid (1.4 g, 61%) as a white solid. LCMS (ESI, m/z): 435.10 [M+H]⁺.

### Intermediate B2: Synthesis of 2-(4-(((5-fluoro-4-oxo-7-((tetrahydro-2H-pyran-4-yl)methoxy)-3,4-dihydroquinazolin-2-yl)methyl)thio)piperidin-1-yl)acetic acid

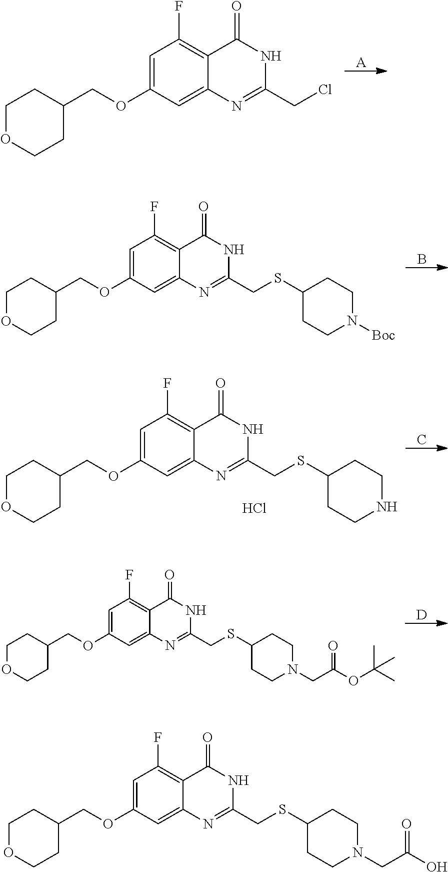

### Step A

To a solution of NaOH (0.37 g, 9.2 mmol, 3.0 equiv) in water (10 mL) was added into 2-(chloromethyl)-5-fluoro-7-((tetrahydro-2H-pyran-4-yl)methoxy)quinazolin-4(3H)-one (1.0 g, 3.0 mmol, 1.0 equiv) and tert-butyl 4-mercaptopiperidine-1-carboxylate (0.80 g, 3.7 mmol, 1.2 equiv) at room temperature. The solution was stirred for 4 hours and then acidified to pH 6 with aqueous HCl. The precipitated solids were collected by filtration and washed with water (3×10 mL) to afford tert-butyl 4-(((5-fluoro-4-oxo-7-((tetrahydro-2H-pyran-4-yl)methoxy)-3,4-dihydroquinazolin-2-yl)methyl)thio)piperidine-1-carboxylate (1.47 g, 95% yield) as a white solid. LCMS (ESI, m/z): 508.30 [M+H]⁺.

### Step B

A solution of tert-butyl 4-(((5-fluoro-4-oxo-7-((tetrahydro-2H-pyran-4-yl)methoxy)-3,4-dihydroquinazolin-2-yl)methyl)thio)piperidine-1-carboxylate (1.46 g, 2.88 mmol, 1.0 equiv) in HCl in 1,4-dioxane (10 mL, 4 M) was stirred for 1 hour at room temperature. The resulting mixture was concentrated under vacuum to afford the HCl salt of 5-fluoro-2-((piperidin-4-ylthio)methyl)-7-((tetrahydro-2H-pyran-4-yl)methoxy)quinazolin-4(3H)-one (1.52 g) as a white crude solid. The crude product was used in the next step without further purification. LCMS (ESI, m/z): 408.19 [M+H]⁺.

### Step C

A solution of 5-fluoro-2-((piperidin-4-ylthio)methyl)-7-((tetrahydro-2H-pyran-4-yl)methoxy)quinazolin-4(3H)-one hydrochloride (1.51 g, 3.40 mmol, 1.0 equiv), K₂CO₃ (0.94 g, 6.8 mmol, 2.0 equiv) and tert-butyl 2-bromoacetate (0.66 g, 3.4 mmol, 1.0 equiv) in ACN (20 mL) was stirred for 16 hours at 70° C. The organics were removed under vacuum. The precipitated solids were collected by filtration and washed with water (3×10 mL). The resulting solids were dried in an oven to afford tert-butyl 2-(4-(((5-fluoro-4-oxo-7-((tetrahydro-2H-pyran-4-yl)methoxy)-3,4-dihydroquinazolin-2-yl)methyl)thio)piperidin-1-yl)acetate (1.1 g, 62% yield) as a white solid. LCMS (ESI, m/z): 522.25 [M+H]⁺.

### Step D

A solution of tert-butyl 2-(4-(((5-fluoro-4-oxo-7-((tetrahydro-2H-pyran-4-yl)methoxy)-3,4-dihydroquinazolin-2-yl)methyl)thio)piperidin-1-yl)acetate (1.15 g, 2.20 mmol, 1.0 equiv) and TFA (1 mL) in DCM (5 mL) was stirred for 16 hours at room temperature. The resulting mixture was concentrated under vacuum to afford 2-(4-(((5-fluoro-4-oxo-7-((tetrahydro-2H-pyran-4-yl)methoxy)-3,4-dihydroquinazolin-2-yl)methyl)thio)piperidin-1-yl)acetic acid (15.8 g) as a brown crude oil. The crude product was used in the next step without further purification. LCMS (ESI, m/z): 466.20 [M+H]⁺.

Intermediate B3 was synthesized according to the procedure described for the synthesis of 2-(4-(((5-fluoro-4-oxo-7-((tetrahydro-2H-pyran-4-yl)methoxy)-3,4-dihydroquinazolin-2-yl)methyl)thio)piperidin-1-yl)acetic acid using appropriate building blocks and modified reaction conditions (such as reagents, reagent ratio, temperature, and reaction time) and purification conditions as needed.

| Intermediate | | |
|---|---|---|
| No | Structure | Analytical Data |
| B3 | 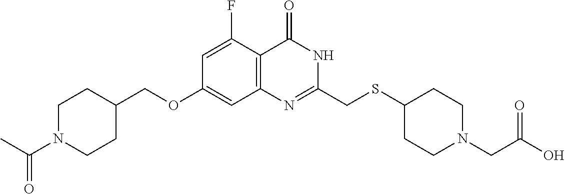 [SMILES: CC(=O)N1CCC(COc2cc(F)c3c(=O)[nH]c(CSC4CCN(CC(=O)O)CC4)nc3c2)CC1] | LCMS (ESI, m/z): 507.15 [M + H]+ |
| | 2-(4-(((7-((1-acetylpiperidin-4-yl)methoxy)-5- | |
| | fluoro-4-oxo-3,4-dihydroquinazolin-2- | |
| | yl)methyl)thio)piperidin-1-yl)acetic acid | |

### Intermediate B4: Synthesis of 2-(4-(2-(7-((1-acetylpiperidin-4-yl)methoxy)-5-fluoro-4-oxo-3,4-dihydroquinazolin-2-yl)ethyl)piperidin-1-yl)acetic acid

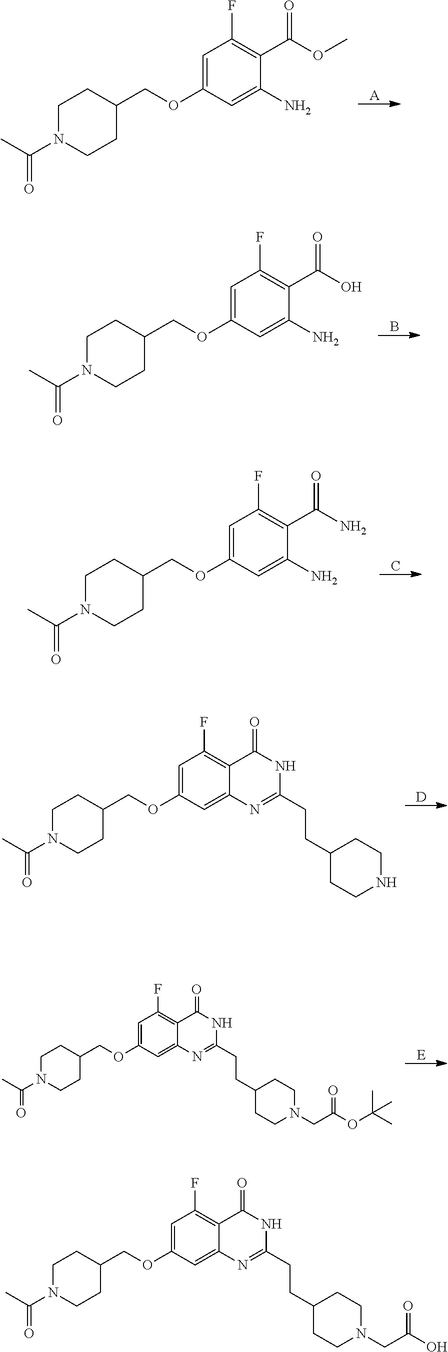

### Step A

To a solution of methyl 4-((1-acetylpiperidin-4-yl)methoxy)-2-amino-6-fluorobenzoate (2.0 g, 6.2 mmol, 1.0 equiv) in MeOH (18 mL) was added a solution of KOH (3.46 g, 61.7 mmol, 10.0 equiv) in water (6 mL). The resulting mixture was stirred for overnight at 40° C. and then concentrated under reduced pressure. The residue was purified by silica gel column chromatography eluting with MeOH/DCM (1:9) to afford 4-((1-acetylpiperidin-4-yl)methoxy)-2-amino-6-fluorobenzoic acid (1.12 g, 59% yield) as a yellow solid. LCMS (ESI, m/z): 311.15[M+H]⁺.

### Step B

To a solution of 4-((1-acetylpiperidin-4-yl)methoxy)-2-amino-6-fluorobenzoic acid (580 mg, 1.87 mmol, 1.0 equiv) and NH₄Cl (200 mg, 3.74 mmol, 2.0 equiv) in DMF (4 mL) was added DIEA (725 mg, 5.61 mmol, 3.0 equiv) and HATU (1066 mg, 2.804 mmol, 1.5 equiv) portion-wise at room temperature. The resulting mixture was stirred for 1 hour and then purified directly by reversed-phase flash chromatography with the following conditions: column: C18 silica gel; mobile phase: MeCN in water (10 mmol/L NH₄HCO₃), 10% to 50% gradient in 10 min; detector: UV 254 nm) to afford 4-((1-acetylpiperidin-4-yl)methoxy)-2-amino-6-fluorobenzamide (428 mg, 72% yield) as a yellow solid. LCMS (ESI, m/z): 310.15 [M+H]⁺.

### Step C

To a stirred solution of 4-((1-acetylpiperidin-4-yl)methoxy)-2-amino-6-fluorobenzamide (569 mg, 1.84 mmol, 1.0 equiv) and tert-butyl 4-(3-oxopropyl)piperidine-1-carboxylate (882 mg, 3.66 mmol, 2.0 equiv) in water (10 mL) was added FeCl₃ (594 mg, 3.65 mmol, 2.0 equiv) portion-wise at room temperature. The resulting mixture was stirred for 1 hour at 100° C. and then the solution was cooled to room temperature and the pH was adjusted pH 7 with saturated aqueous NaHCO₃. The resulting mixture was filtered and the filter cake was washed with MeOH (2×10 mL). The filtrate was concentrated under reduced pressure and the residue was purified by silica gel column chromatography, eluting with DCM/MeOH (4:1) to afford impure 7-((1-acetylpiperidin-4-yl)methoxy)-5-fluoro-2-(2-(piperidin-4-yl)ethyl)quinazolin-4(3H)-one (529 mg) as a brown yellow oil. LCMS (ESI, m/z): 431.25 [M+H]⁺.

### Step D

A solution of 7-((1-acetylpiperidin-4-yl)methoxy)-5-fluoro-2-(2-(piperidin-4-yl)ethyl)quinazolin-4(3H)-one (339 mg, 0.787 mmol, 1.0 equiv), DIEA (305 mg, 2.36 mmol, 3.0 equiv) and tert-butyl 2-bromoacetate (461 mg, 2.36 mmol, 3.0 equiv) in NMP (3 mL) was stirred for 2 hours at room temperature. The solution was purified directly by reverse-phase flash chromatography with the following conditions: column: C18 silica gel; mobile phase, MeCN in water (10 mmol/L NH₄HCO₃), 10% to 50% gradient in 10 min; detector: UV 254 nm) to afford tert-butyl 2-(4-(2-(7-((1-acetylpiperidin-4-yl)methoxy)-5-fluoro-4-oxo-3,4-dihydroquinazolin-2-yl)ethyl)piperidin-1-yl)acetate (240 mg, 51% yield) as a colorless oil. LCMS (ESI, m/z): 545.30 [M+H]⁺.

### Step E

A solution of tert-butyl 2-(4-(2-(7-((1-acetylpiperidin-4-yl)methoxy)-5-fluoro-4-oxo-3,4-dihydroquinazolin-2-yl)ethyl)piperidin-1-yl)acetate (224 mg, 0.411 mmol, 1.0 equiv) and TFA (2 mL) in DCM (10 mL) was stirred for 16 hours at room temperature. The resulting mixture was concentrated under reduced pressure to afford 2-(4-(2-(7-((1-acetylpiperidin-4-yl)methoxy)-5-fluoro-4-oxo-3,4-dihydroquinazolin-2-yl)ethyl)piperidin-1-yl)acetic acid a colorless oil. The crude product was used in without further purification. LCMS (ESI, m/z): 489.30[M+H]⁺.

### Intermediate B5: Synthesis of 7-((1-acetylpiperidin-4-yl)methoxy)-5-fluoro-2-(((1-(prop-2-yn-1-yl)piperidin-4-yl)thio)methyl)quinazolin-4(3H)-one

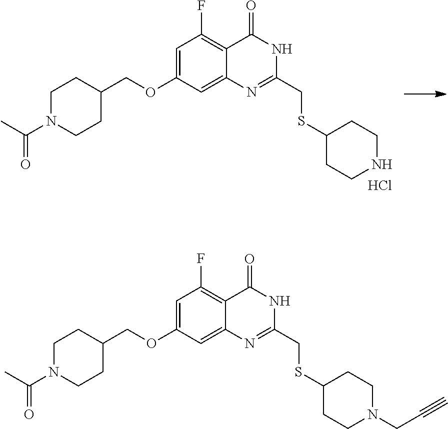 [SMILES: CC(=O)N1CCC(COc2cc(F)c3c(=O)[nH]c(CSC4CCNCC4)nc3c2)CC1 ; Cl ; C#CCN1CCC(SCc2nc3cc(OCC4CCN(C(C)=O)CC4)cc(F)c3c(=O)[nH]2)CC1]

A solution of 7-((1-acetylpiperidin-4-yl)methoxy)-5-fluoro-2-((piperidin-4-ylthio)methyl)quinazolin-4(3H)-one hydrochloride (500 mg, 1.03 mmol, 1.0 equiv), DIEA (400 mg, 3.09 mmol, 3.0 equiv) and 3-bromoprop-1-yne (172 mg, 1.44 mmol, 1.4 equiv) in DMSO (2 mL) was stirred for 1 hour at room temperature. The residue was purified by reverse phase flash chromatography with the following conditions: (column, C18 silica gel; mobile phase, MeCN in water (0.1% NH₄HCO₃), 0% to 35% gradient in 20 min; detector, UV 254 nm) to afford of 7-((1-acetylpiperidin-4-yl)methoxy)-5-fluoro-2-(((1-(prop-2-yn-1-yl)piperidin-4-yl)thio)methyl)quinazolin-4(3H)-one (200 mg, 40% yield) as a white solid. LCMS (ESI, m/z): 487.20 [M+H]⁺.

### Intermediate B6: Synthesis of 7-(cyclopropylmethoxy)-5-fluoro-2-((piperidin-4-ylthio)methyl)quinazolin-4(3H)-one hydrochloride

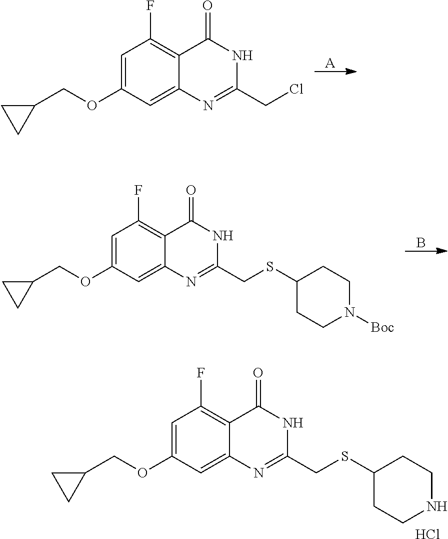

### Step A

A solution of 2-(chloromethyl)-7-(cyclopropylmethoxy)-5-fluoroquinazolin-4(3H)-one (3 g, 10.6 mmol, 1.0 equiv), tert-butyl 4-(acetylthio)piperidine-1-carboxylate (2.78 g, 10.7 mmol, 1.01 equiv) and NaOH (1.27 g, 31.8 mmol, 3.0 equiv) in H₂O (30 mL) was stirred for 1.5 hours at room temperature. The mixture was acidified to pH 3 with conc. HCl. The precipitated solids were collected by filtration and washed with water (3×5 mL). The solid was dried under infrared light to afford title compound (4.8 g, 97% yield) as a white solid. LCMS (ESI, m/z): 464.19 [M+H]⁺.

### Step B

A solution of tert-butyl 4-(((7-(cyclopropylmethoxy)-5-fluoro-4-oxo-3,4-dihydroquinazolin-2-yl)methyl)thio)piperidine-1-carboxylate (2.5 g, 5.4 mmol, 1.0 equiv) and HCl in 1,4-dioxane (10 mL, 4M) was stirred for 1 hour at room temperature. The resulting mixture was concentrated under reduced pressure to afford title compound (2.09 g, 97% yield) as a white solid. LCMS (ESI, m/z): 364.14 [M+H]⁺.

### Intermediate B7: Synthesis of 2-(((1-(2-chloroethyl)piperidin-4-yl)thio)methyl)-7-(cyclopropylmethoxy)-5-fluoroquinazolin-4(3H)-one

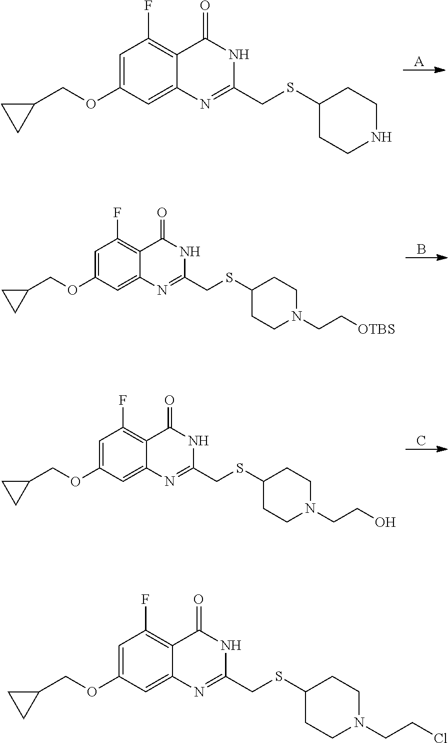

### Step A

A solution of 7-(cyclopropylmethoxy)-5-fluoro-2-((piperidin-4-ylthio)methyl)quinazolin-4(3H)-one (230 mg, 0.63 mmol, 1 equiv) in NMP (5 mL) was treated with (2-bromoethoxy)(tert-butyl)dimethylsilane (167 mg, 0.70 mmol, 1.1 equiv) and DIEA (245 mg, 1.90 mmol, 3 equiv) and then stirred overnight at 80° C. Water was added and the resulting mixture was extracted with EtOAc. The combined organic layers were washed with brine, dried over anhydrous Na₂SO₄ and filtered. The filtrate was concentrated, and the residue purified by reverse-phase flash chromatography with the following conditions: column, C18 silica gel; mobile phase, MeCN in water (10 mmol/L NH₄HCO₃), 10% to 72% gradient in 20 min; detector, UV 254 nm to give 2-(((1-(2-((tert-butyldimethylsilyl)oxy)ethyl)piperidin-4-yl)thio)methyl)-7-(cyclopropylmethoxy)-5-fluoroquinazolin-4(3H)-one (97 mg, 29%) as a brown solid. LCMS (ESI, m/z): 522.25 [M+H]⁺.

### Step B

A solution of 2-(((1-(2-((tert-butyldimethylsilyl)oxy)ethyl)piperidin-4-yl)thio)methyl)-7-(cyclopropylmethoxy)-5-fluoroquinazolin-4(3H)-one (97 mg, 0.19 mmol, 1 equiv) was treated with HCl in 1,4-dioxane (15 mL, 4 M) for 3 hours. The resulting mixture was concentrated to dryness to afford 7-(cyclopropylmethoxy)-5-fluoro-2-(((1-(2-hydroxyethyl)piperidin-4-yl)thio)methyl)quinazolin-4(3H)-one (118 mg) as a brown solid that was used in the next step directly without further purification. LCMS (ESI, m/z): 408.15 [M+H]⁺.

### Step C

A solution of 7-(cyclopropylmethoxy)-5-fluoro-2-(((1-(2-hydroxyethyl)piperidin-4-yl)thio)methyl)quinazolin-4(3H)-one (100 mg, 0.25 mmol, 1 equiv) in DCM (2 mL) was treated with 3-nitrobenzenesulfonyl chloride (54 mg, 0.25 mmol, 1 equiv) and TEA (75 mg, 0.74 mmol, 3 equiv) and stirred for 2 hours. The resulting mixture was concentrated and the residue was purified by silica gel column chromatography, eluting with DCM/MeOH (1/9) to afford 2-(((1-(2-chloroethyl)piperidin-4-yl)thio)methyl)-7-(cyclopropylmethoxy)-5-fluoroquinazolin-4(3H)-one (30 mg, 29%) as a yellow solid. LCMS (ESI, m/z): 426.15 [M+H]⁺.

### Intermediate B8: Synthesis of 2-(((1-(2-chloroethyl)piperidin-4-yl)thio)methyl)-5-fluoro-7-((tetrahydro-2H-pyran-4-yl)methoxy)quinazolin-4(3H)-one

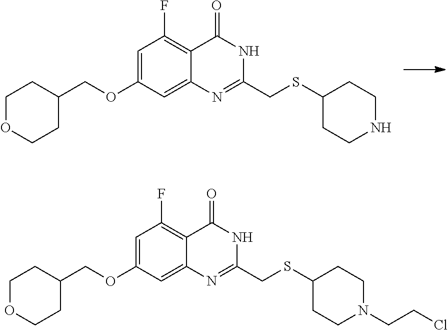 [SMILES: O=c1[nH]c(CSC2CCNCC2)nc2cc(OCC3CCOCC3)cc(F)c12 ; O=c1[nH]c(CSC2CCN(CCCl)CC2)nc2cc(OCC3CCOCC3)cc(F)c12]

A solution of 5-fluoro-7-(oxan-4-ylmethoxy)-2-[(piperidin-4-ylsulfanyl)methyl]-3H-quinazolin-4-one (500 mg, 1.23 mmol, 1 equiv), chloroacetaldehyde in water (239 mg, 1.23 mmol, 1 equiv, 40 wt. %) and STAB (520 mg, 2.45 mmol, 2 equiv) in DCM (5 mL) was stirred for 1 hour. The solution was concentrated and the residue was purified by silica gel column chromatography, eluting with PE/EtOAc (1:1) to afford 2-(((1-(2-chloroethyl)piperidin-4-yl)thio)methyl)-5-fluoro-7-((tetrahydro-2H-pyran-4-yl)methoxy)quinazolin-4(3H)-one (200 mg, 35%) as a white solid. LCMS (ESI, m/z): 470.20 [M+H]⁺.

### Intermediate B9: Synthesis of 2-(((1-(azetidin-3-yl)piperidin-4-yl)thio)methyl)-5-fluoro-7-((tetrahydro-2H-pyran-4-yl)methoxy)quinazolin-4(3H)-one hydrochloride

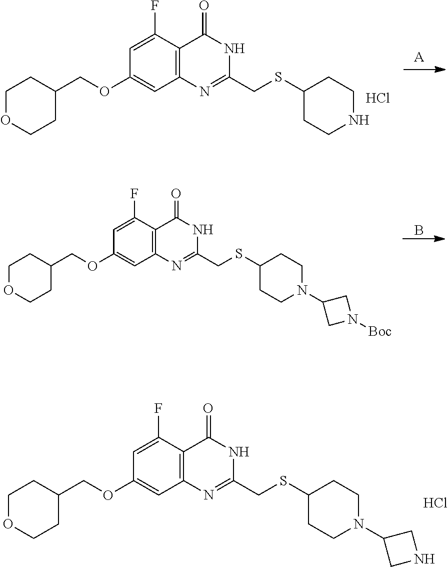

### Step A

A solution of 5-fluoro-2-((piperidin-4-ylthio)methyl)-7-((tetrahydro-2H-pyran-4-yl)methoxy)quinazolin-4(3H)-one hydrochloride (2.0 g, 4.9 mmol, 1 equiv), tert-butyl 3-oxoazetidine-1-carboxylate (1.54 g, 9.01 mmol, 2 equiv) and STAB (1.91 g, 9.01 mmol, 2 equiv) in DCM (7 mL) was stirred for 3 hours. The mixture was concentrated and the residue was purified by silica gel column chromatography, eluting with DCM/MeOH (97:3) to afford tert-butyl 3-(4-(((5-fluoro-4-oxo-7-((tetrahydro-2H-pyran-4-yl)methoxy)-3,4-dihydroquinazolin-2-yl)methyl)thio)piperidin-1-yl)azetidine-1-carboxylate (1.17 g, 46%) as a yellow solid. LCMS (ESI, m/z): 563.26 [M+H].

### Step B

A solution of tert-butyl 3-(4-(((5-fluoro-4-oxo-7-((tetrahydro-2H-pyran-4-yl)methoxy)-3,4-dihydroquinazolin-2-yl)methyl)thio)piperidin-1-yl)azetidine-1-carboxylate (1.17 g, 2.08 mmol, 1 equiv) in HCl in 1,4-dioxane (7 mL, 4 M) was stirred for 30 min. The mixture was concentrated to dryness to afford 2-(((1-(azetidin-3-yl)piperidin-4-yl)thio)methyl)-5-fluoro-7-((tetrahydro-2H-pyran-4-yl)methoxy)quinazolin-4(3H)-one hydrochloride (1.06 g) as a yellow solid. The crude product was used in the next step directly without further purification. LCMS (ESI, m/z): 463.21 [M+H]⁺.

Intermediates B9-a to B9-c were synthesized according to the procedure described for the synthesis of 2-(((1-(azetidin-3-yl)piperidin-4-yl)thio)methyl)-5-fluoro-7-((tetrahydro-2H-pyran-4-yl)methoxy)quinazolin-4(3H)-one hydrochloride using appropriate building blocks and modified reaction conditions (such as reagents, reagent ratio, temperature, and reaction time) and purification conditions as needed.

| Intermediate | | |
|---|---|---|
| No | Structure | Analytical Data |
| B9-a | 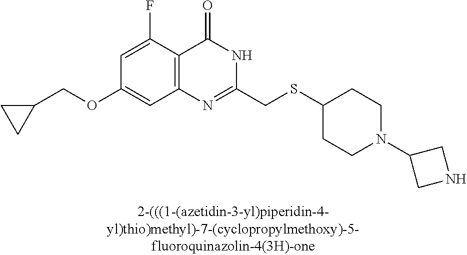 [SMILES: O=c1[nH]c(CSC2CCN(C3CNC3)CC2)nc2cc(OCC3CC3)cc(F)c12] | LCMS (ESI, m/z): 419.10 [M + H]⁺ |
| B9-b | 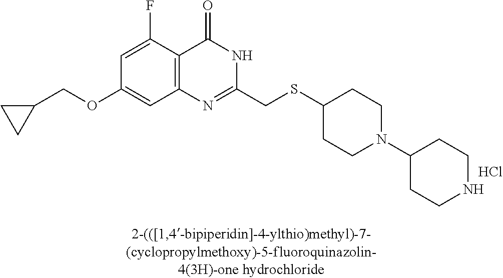 [SMILES: O=c1[nH]c(CSC2CCN(C3CCNCC3)CC2)nc2cc(OCC3CC3)cc(F)c12 ; Cl] | LCMS (ESI, m/z): 447.40 [M + H]⁺ |
| B9-c | 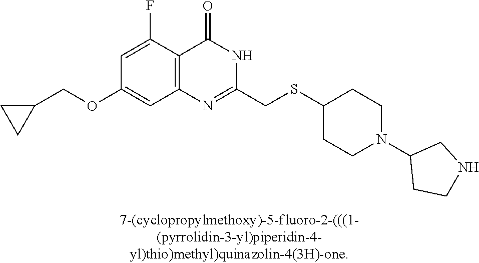 [SMILES: O=c1[nH]c(CSC2CCN(C3CCNC3)CC2)nc2cc(OCC3CC3)cc(F)c12] | LCMS (ESI, m/z): 433.15 [M + H]⁺ |

### Intermediate B10: Synthesis of 2-(((1-(2-chloroethyl)piperidin-4-yl)oxy)methyl)-5-fluoro-7-((tetrahydro-2H-pyran-4-yl)methoxy)quinazolin-4(3H)-one

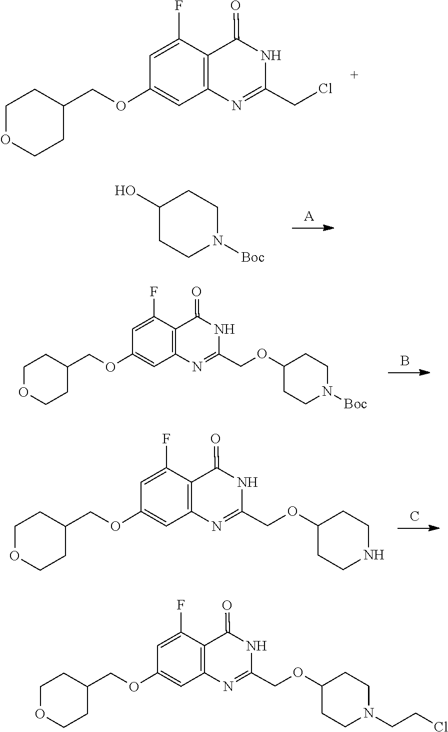

### Step A

A solution of 2-(chloromethyl)-5-fluoro-7-((tetrahydro-2H-pyran-4-yl)methoxy)quinazolin-4(3H)-one (2.0 g, 6.1 mmol, 1 equiv), tert-butyl 4-hydroxypiperidine-1-carboxylate (1.97 g, 9.79 mmol, 1.6 equiv) and t-BuOK (2.06 g, 18.4 mmol, 3 equiv) in DMA (10 mL) was stirred for 5 hours at 40° C. The residue was purified by reverse-phase flash chromatography with the following conditions: column, C18 silica gel; mobile phase, MeCN in water (10 mmol/L NH₄HCO₃), 10% to 50% gradient in 25 min; detector, UV 254 nm to give tert-butyl 4-((5-fluoro-4-oxo-7-((tetrahydro-2H-pyran-4-yl)methoxy)-3,4-dihydroquinazolin-2-yl)methoxy)piperidine-1-carboxylate (2 g, 59%) as a light pink solid. LCMS (ESI, m/z): 492.10 [M+H]⁺.

### Step B

A solution of tert-butyl 4-((5-fluoro-4-oxo-7-((tetrahydro-2H-pyran-4-yl)methoxy)-3,4-dihydroquinazolin-2-yl)methoxy)piperidine-1-carboxylate (1.99 g, 4.05 mmol, 1 equiv) in HCl in 1,4-dioxane (20 mL, 4 M) was stirred for 1 hour. The mixture was concentrated under vacuum to give 5-fluoro-2-((piperidin-4-yloxy)methyl)-7-((tetrahydro-2H-pyran-4-yl)methoxy)quinazolin-4(3H)-one (2 g) as a light brown solid that was used in the next step directly without further purification. LCMS (ESI, m/z): 392.15 [M+H]⁺.

### Step C

A solution of 5-fluoro-2-((piperidin-4-yloxy)methyl)-7-((tetrahydro-2H-pyran-4-yl)methoxy)quinazolin-4(3H)-one (500 mg, 1.28 mmol, 1 equiv), 2-chloroacetaldehyde (501 mg, 2.55 mmol, 2.0 equiv, 40 wt. %), and STAB (541 mg, 2.55 mmol, 2 equiv) in DCE (2 mL) was stirred for 1 hour. The mixture was purified by silica gel column chromatography, eluting with DCM/MeOH (12:1) to afford 2-(((1-(2-chloroethyl)piperidin-4-yl)oxy)methyl)-5-fluoro-7-((tetrahydro-2H-pyran-4-yl)methoxy)quinazolin-4(3H)-one (180 mg, 31%) as a white solid. LCMS (ESI, m/z): 454.25 [M+H]⁺.

### Intermediate B11: Synthesis of 2-(2-chloroethyl)-7-(cyclopropylmethoxy)-5-fluoroquinazolin-4(3H)-one

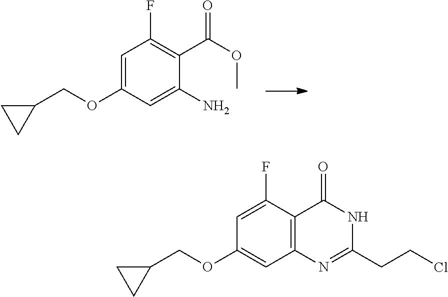 [SMILES: COC(=O)c1c(N)cc(OCC2CC2)cc1F ; O=c1[nH]c(CCCl)nc2cc(OCC3CC3)cc(F)c12]

A solution of methyl 2-amino-4-(cyclopropylmethoxy)-6-fluorobenzoate (300 mg, 1.2 mmol, 1 equiv) and acrylonitrile (332 mg, 6.27 mmol, 5 equiv) in HCl in 1,4-dioxane (10 mL, 4 M) was stirred for 1 hour. The mixture was concentrated and the residue was purified by silica gel column chromatography, eluting with PE/EtOAc (3:2) to afford 2-(2-chloroethyl)-7-(cyclopropylmethoxy)-5-fluoroquinazolin-4(3H)-one (250 mg, 67%) as a brown oil. LCMS (ESI, m/z): 297.10 [M+H]⁺.

### Intermediate B12: Synthesis of 2-(2-([1,4′-bipiperidin]-4-yl)ethyl)-7-(cyclopropylmethoxy)-5-fluoroquinazolin-4(3H)-one hydrochloride

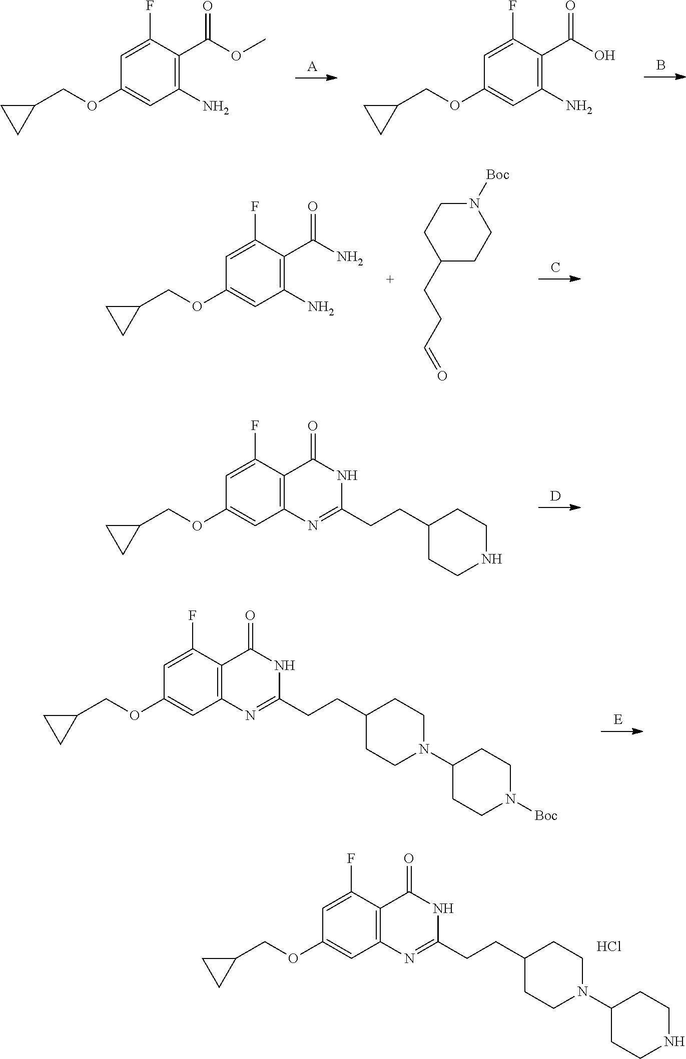

### Step A

A solution of methyl 2-amino-4-(cyclopropylmethoxy)-6-fluorobenzoate (2 g, 8.36 mmol, 1 equiv) and KOH (3.75 g, 66.9 mmol, 8 equiv) in MeOH (40 mL) and water (10 mL) was stirred overnight at 60° C. The mixture was concentrated and then acidified to pH 4 with concentrated HCl. The precipitated solids were collected by filtration and washed with water (3×100 mL) to afford 2-amino-4-(cyclopropylmethoxy)-6-fluorobenzoic acid (1.8 g, 96%) as a light yellow solid. LCMS (ESI, m/z): 226.0 [M+H]⁺.

### Step B

A solution of 2-amino-4-(cyclopropylmethoxy)-6-fluorobenzoic acid (1.8 g, 7.99 mmol, 1 equiv), NH₄Cl (0.43 g, 7.99 mmol, 1 equiv), DIEA (4.13 g, 32.0 mmol, 4 equiv) and HATU (4.56 g, 12.0 mmol, 1.5 equiv) in DMF (10 mL) was stirred for 1 hour. The mixture was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. This afforded 2-amino-4-(cyclopropylmethoxy)-6-fluorobenzamide (1.5 g, 84%) as a white solid. LCMS (ESI, m/z): 225.01 [M+H]⁺.

### Step C

A solution of 2-amino-4-(cyclopropylmethoxy)-6-fluorobenzamide (0.50 g, 2.23 mmol, 1 equiv), tert-butyl 4-(3-oxopropyl)piperidine-1-carboxylate (1.08 g, 4.46 mmol, 2 equiv) and FeCl₃·6H₂O (1.21 g, 4.46 mmol, 2 equiv) in water (7 mL) was stirred for 1 hour at 100° C. The residue was purified by silica gel column chromatography, eluting with DCM/MeOH (9:1) to afford crude compound (300 mg) as a yellow solid. The crude product was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. This afforded 7-(cyclopropylmethoxy)-5-fluoro-2-(2-(piperidin-4-yl)ethyl)quinazolin-4(3H)-one (155 mg, 20%) as a white solid. LCMS (ESI, m/z): 346.01 [M+H]⁺.

### Step D

A solution of 7-(cyclopropylmethoxy)-5-fluoro-2-(2-(piperidin-4-yl)ethyl)quinazolin-4(3H)-one (135 mg, 0.391 mmol, 1 equiv), tert-butyl 4-oxopiperidine-1-carboxylate (156 mg, 0.782 mmol, 2 equiv) and STAB (166 mg, 0.782 mmol, 2 equiv) in DCE (13.5 mL) was stirred overnight. The mixture was concentrated and the residue purified by silica gel column chromatography, eluting with DCM/MeOH (10:1) to afford tert-butyl 4-(2-(7-(cyclopropylmethoxy)-5-fluoro-4-oxo-3,4-dihydroquinazolin-2-yl)ethyl)-[1,4′-bipiperidine]-1′-carboxylate (80 mg, 36%) as a yellow solid. LCMS (ESI, m/z): 529.3 [M+H]⁺.

### Step E

A solution of tert-butyl 4-(2-(7-(cyclopropylmethoxy)-5-fluoro-4-oxo-3,4-dihydroquinazolin-2-yl)ethyl)-[1,4′-bipiperidine]-1′-carboxylate (80 mg, 0.151 mmol, 1 equiv) and HCl in 1,4-dioxane (10 mL, 4 M) was stirred for 1 hour. The mixture was concentrated to dryness to afford 2-(2-([1,4′-bipiperidin]-4-yl)ethyl)-7-(cyclopropylmethoxy)-5-fluoroquinazolin-4(3H)-one hydrochloride (100 mg) as a white solid. The crude product was used in next step without further purification. LCMS (ESI, m/z): 429.16 [M+H]⁺.

### Example 1: Synthesis of 4-(4-((1-(2-(4-(((7-(cyclopentylamino)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione

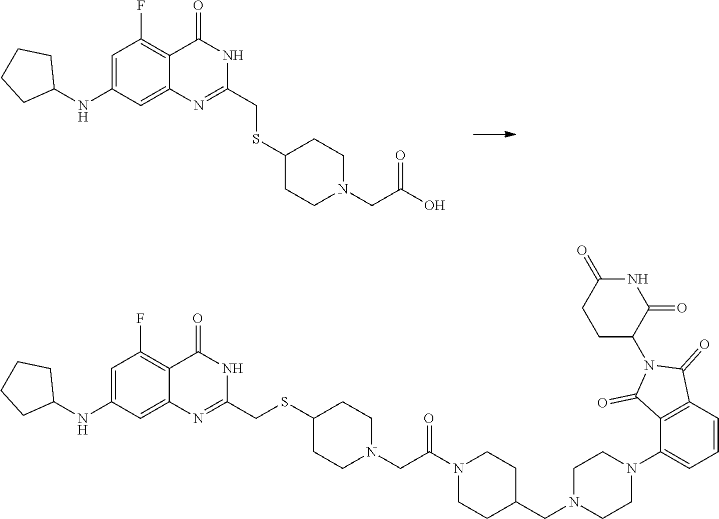 [SMILES: O=C(O)CN1CCC(SCc2nc3cc(NC4CCCC4)cc(F)c3c(=O)[nH]2)CC1 ; O=C1CCC(N2C(=O)c3cccc(N4CCN(CC5CCN(C(=O)CN6CCC(SCc7nc8cc(NC9CCCC9)cc(F)c8c(=O)[nH]7)CC6)CC5)CC4)c3C2=O)C(=O)N1]

A solution of 2-(2,6-dioxopiperidin-3-yl)-4-(4-(piperidin-4-ylmethyl)piperazin-1-yl)isoindoline-1,3-dione (1.31 g, 3.00 mmol, 1.0 equiv), 2-(4-(((7-(cyclopentylamino)-5-fluoro-4-oxo-3,4-dihydroquinazolin-2-yl)methyl)thio)piperidin-1-yl)acetic acid (1.37 g, 3.15 mmol, 1.05 equiv), HOBT (608 mg, 4.50 mmol, 1.5 equiv), EDCI (863 mg, 4.50 mmol, 1.5 equiv) and DIEA (1.55 g, 12.0 mmol, 4.0 equiv) in DMF (12 mL) and DCM (9 mL) was stirred for 5 hours at 45° C. The resulting mixture was diluted with brine (100 mL) and then extracted with EtOAc (3×100 mL). The combined organic layers were washed with brine (100 mL), dried over anhydrous Na₂SO₄, filtered, and concentrated under reduced pressure. The crude product was purified by trituration with ethyl acetate (20 mL×3), ethanol (20 mL×3) and MeCN (20 mL×3) to afford 4-(4-((1-(2-(4-(((7-(cyclopentylamino)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione (1.28 g, 50% yield) as a yellow solid. LCMS (ESI, m/z): 856.45 [M+H]⁺. 1H NMR (300 MHz, DMSO-d6) δ 11.66 (s, 1H), 11.10 (s, 1H), 7.71 (t, J=9.0 1H), 7.35 (t, J=9.0 Hz, 2H), 6.84 (d, J=6.5 Hz, 1H), 6.42 (dd, J=13.9, 2.1 Hz, 1H), 6.36 (d, J=2.1 Hz, 1H), 5.10 (dd, J=12.7, 5.4 Hz, 1H), 4.31 (d, J=12.8 Hz, 1H), 4.03 (d, J=12.9 Hz, 1H), 3.85-3.74 (m, 1H), 3.53 (s, 2H), 3.38-3.30 (m, 4H), 3.24-3.15 (m, 1H), 3.03-2.69 (m, 6H), 2.67-2.53 (m, 5H), 2.25-2.16 (m, 2H), 2.14-2.00 (m, 3H), 1.99-1.85 (m, 4H), 1.84-1.30 (m, 12H), 1.19-0.81 (m, 3H).

Examples 2-29 were synthesized according to the procedure described for the synthesis of 4-(4-((1-(2-(4-(((7-(cyclopentylamino)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione using appropriate building blocks and modified reaction conditions (such as reagents, reagent ratio, temperature, and reaction time) and purification conditions as needed.

| Example | Structure | Analytical Data |
|---|---|---|
| 2 | 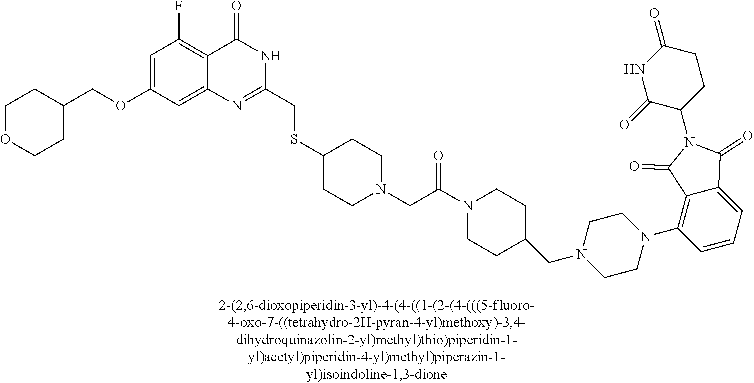 [SMILES: O=C1CCC(N2C(=O)c3cccc(N4CCN(CC5CCN(C(=O)CN6CCC(SCc7nc8cc(OCC9CCOCC9)cc(F)c8c(=O)[nH]7)CC6)CC5)CC4)c3C2=O)C(=O)N1] | LCMS (ESI, m/z): 887.50 [M + H]⁺; ¹H NMR (300 MHz, DMSO-d₆) δ 12.14 (s, 1H), 11.08 (s, 1H), 7.69 (dd, J = 7.2, 8.4 Hz, 1H), 7.33 (t, J = 9.9 Hz, 2H), 6.93-6.82 (m, 2H), 5.08 (dd, J = 12.6, 5.4 Hz, 1H), 4.29 (d, J = 12.9 Hz, 1H), 3.97 (d, J = 6.3 Hz, 2H), 3.86 (dd, J = 11.4, 3.9 Hz, 2H), 3.58 (s, 2H), 3.38-3.32 (m, 3H), 3.29-3.24 (m, 6H), 3.17-2.89 (m, 6H), 2.73-2.56 (m, 6H), 2.21- 2.12 (m, 2H), 2.22-1.82 (m, 6H), 1.81-1.55 (m, 5H), 1.45-1.22 (m, 4H), 1.12-0.81 (m, 2H) |
| 3 | 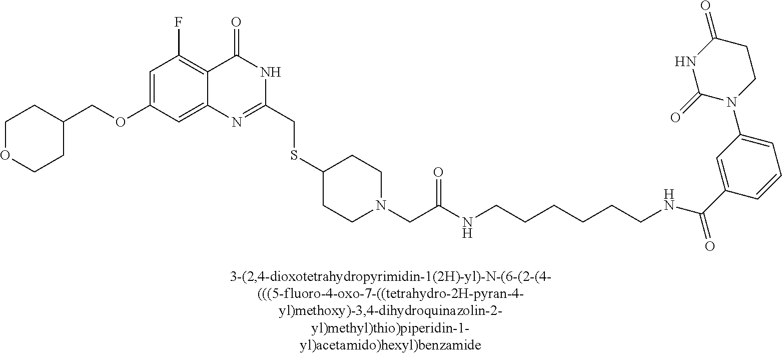 [SMILES: O=C(CN1CCC(SCc2nc3cc(OCC4CCOCC4)cc(F)c3c(=O)[nH]2)CC1)NCCCCCCNC(=O)c1cccc(N2CCC(=O)NC2=O)c1] | LCMS (ESI, m/z): 780.40 [M + H]⁺; ¹H NMR (400 MHz,DMSO-d₆) δ 12.10 (s, 1H), 10.42 (s, 1H), 8.47 (t, J = 5.6 Hz, 1H), 7.77 (d, J = 2.1 Hz, 1H), 7.73- 7.63 (m, 2H), 7.52-7.41 (m, 2H), 6.87 (d, J = 10.3 Hz, 2H), 3.98 (d, J = 6.4 Hz, 2H), 3.91-3.79 (m, 4H), 3.59 (s, 2H), 3.38-3.24 (m, 1H), 3.27-3.19 (m, 2H), 3.11-3.01 (m, 2H), 2.85 (s, 2H), 2.83- 2.67 (m, 5H), 2.11 (t, J = 10.6 Hz, 2H), 2.01 (dd, J = 9.9, 5.2 Hz, 1H), 1.91 (d, J = 12.4 Hz, 2H), 1.71-1.63 (m, 2H), 1.59- 1.46 (m, 4H), 1.44-1.36 (m, 1H), 1.40- 1.21 (m, 8H). |
| 4 | 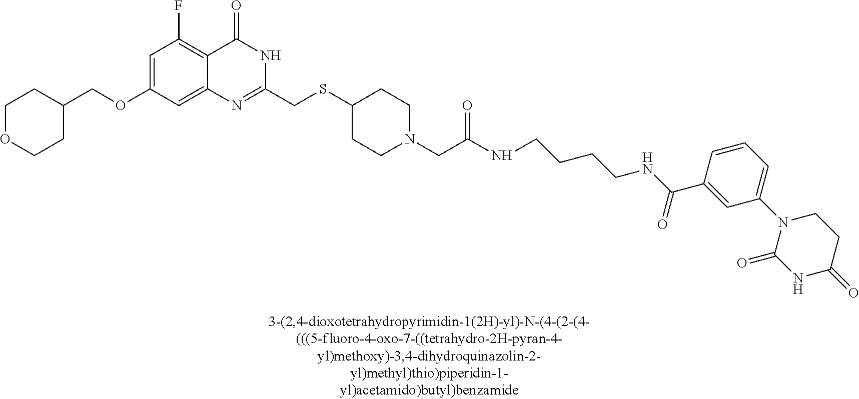 [SMILES: O=C(CN1CCC(SCc2nc3cc(OCC4CCOCC4)cc(F)c3c(=O)[nH]2)CC1)NCCCCNC(=O)c1cccc(N2CCC(=O)NC2=O)c1] | LCMS (ESI, m/z): 752.35 [M + H]⁺; ¹H NMR (400 MHz, DMSO-d₆) δ 12.11 (s, 1H), 10.41 (s, 1H), 8.49 (t, J = 5.7 Hz, 1H), 7.77 (d, J = 2.1 Hz, 1H), 7.73- 7.64 (m, 2H), 7.51-7.41 (m, 2H), 6.87 (d, J = 10.3 Hz, 2H), 3.98 (d, J = 6.4 Hz, 2H), 3.91-3.79 (m, 4H), 3.59 (s, 2H), 3.29-3.19 (m, 4H), 3.15-3.08 (m, 2H), 2.85 (s, 2H), 2.83-2.76 (m, 1H), 2.75- 2.70 (m, 4H), 2.19-2.05 (m, 2H), 2.04- 1.96 (m, 1H), 1.94-1.85 (m, 2H), 1.73- 1.33 (m, 10H). |
| 5 | 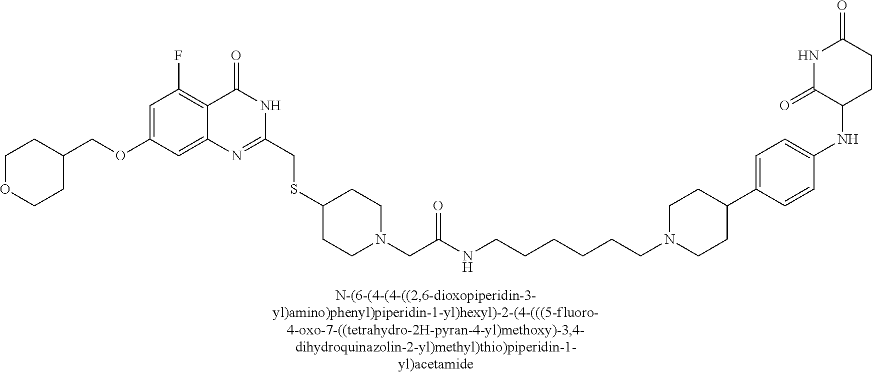 [SMILES: O=C(CN1CCC(SCc2nc3cc(OCC4CCOCC4)cc(F)c3c(=O)[nH]2)CC1)NCCCCCCN1CCC(c2ccc(NC3CCC(=O)NC3=O)cc2)CC1] | LCMS (ES, m/z): 834.60 [M + H]⁺, ¹H NMR (300 MHz, DMSO-d₆) δ 12.12 (s, 1H), 10.77 (s, 1H), 7.66 (t, J = 5.9 Hz, 1H), 7.03-6.80 (m, 4H), 6.60 (d, J = 8.5 Hz, 2H), 5.63 (d, J = 7.4 Hz, 1H), 4.32-4.19 (m, 1H), 4.02-3.96 (m, 2H), 3.93-3.82 (m, 2H), 3.60 (s, 2H), 3.39- 3.36 (m, 1H), 3.30-3.29 (m, 2H), 3.10- 2.99 (m, 2H), 2.92-2.80 (m, 5H), 2.73- 2.69 (m, 2H), 2.60-2.55 (m, 1H), 2.30- 2.19 (m, 3H), 2.17-1.98 (m, 4H), 1.95- 1.84 (m, 5H), 1.72-1.59 (m, 5H), 1.58- 1.49 (m, 3H), 1.45-1.30 (m, 6H), 1.28- 1.20 (m, 4H) |
| 6 | 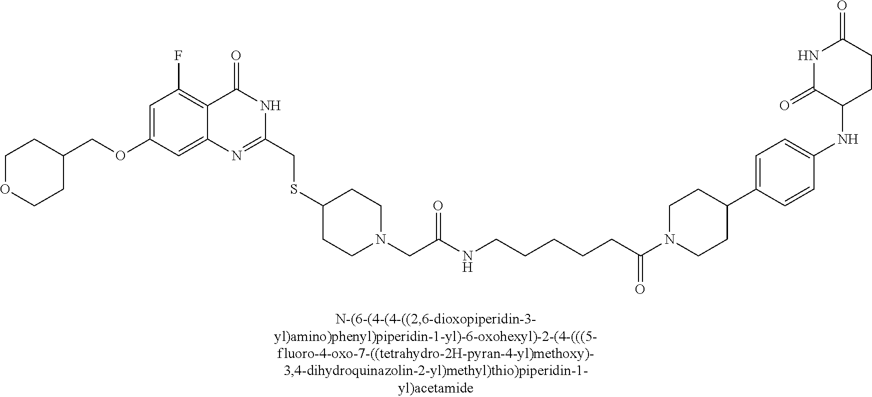 [SMILES: O=C(CN1CCC(SCc2nc3cc(OCC4CCOCC4)cc(F)c3c(=O)[nH]2)CC1)NCCCCCC(=O)N1CCC(c2ccc(NC3CCC(=O)NC3=O)cc2)CC1] | LCMS (ESI, m/z): 848.25 [M + H]⁺; ¹H NMR (400 MHz, DMSO-d₆) δ 12.14 (s, 1H), 10.76 (s, 1H), 7.66 (t, J = 6.0 Hz, 1H), 7.09-6.92 (m, 2H), 6.87 (dd, J = 10.8, 2.2 Hz, 2H), 6.74-6.53 (m, 2H), 5.66 (d, J = 7.5 Hz, 1H), 4.52 (d, J = 12.8 Hz, 1H), 4.26-4.21 (m , 1H), 3.9.- 3.77 (d, J = 6.4 Hz, 2-H), 3.88-376 (m, 3H), 3.60 (s, 2H), 3.35-3.30 (m, 1H), 3.20-3.07 (m, 3H), 2.85 (s, 2H), 2.84-2.79 (m, 1H), 2.74-2.66 (m, 3H), 2.64-2.57 (m, 3H), 2.31-2.14 (m, 2H), 2.13-1.99 (m, 3H), 1.99-1.78 (m, 3H), 1.77-1.63 (m, 4H), 1.63-1.45 (m, 5H), 1.44-1.37 (m, 3H), 1.37-1.30 (m, 2H), 1.30-1.18 (m, 3H). |
| 7 | 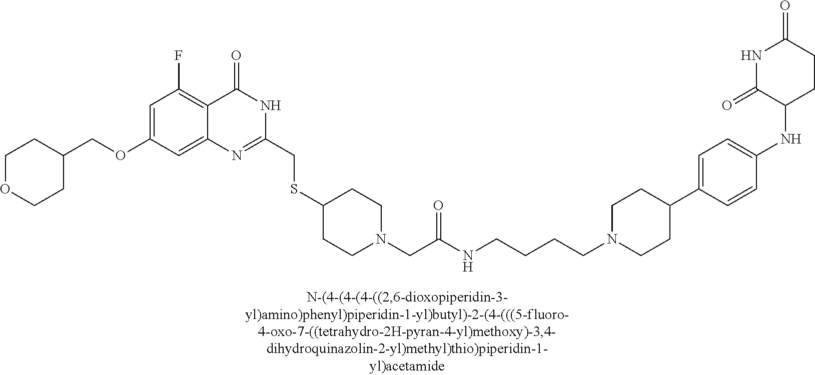 [SMILES: O=C(CN1CCC(SCc2nc3cc(OCC4CCOCC4)cc(F)c3c(=O)[nH]2)CC1)NCCCCN1CCC(c2ccc(NC3CCC(=O)NC3=O)cc2)CC1] | LCMS (ESI, m/z): 806.45 [M + H]⁺, ¹H NMR (400 MHz, DMSO-d₆) δ 12.13 (s, 1H), 10.77 (s, 1H), 7.70 (t, J = 6.1 Hz, 1H), 6.98-6.91 (m, 2H), 6.93-6.84 (m, 2H), 6.00 (d, J = 8.0 Hz, 2H), 5.65 (d, J = 7.4 Hz, 1H), 4.29-4.21 (m, 1H), 3.98 (d, J = 6.4 Hz, 2H), 3.92-3.83 (m, 2H), 3.60 (s, 2H), 3.37-3.26 (m, 2H), 3.13-3.05 (m, 2H), 3.00-2.87 (m, 2H), 2.86 (s, 2H), 2.83-2.80 (m, 1H), 2.79- 2.67 (m, 3H), 2.63-2.52 (m, 1H), 2.17- 2.07 (m, 3H), 2.16-2.03 (m, 4H), 1.96- 1.92 (m, 2H), 1.92-1.79 (m, 2H), 1.75- 1.63 (m, 5H), 1.62-1.53 (m, 4H), 1.42- 1.25 (m, 6H) |
| 8 | 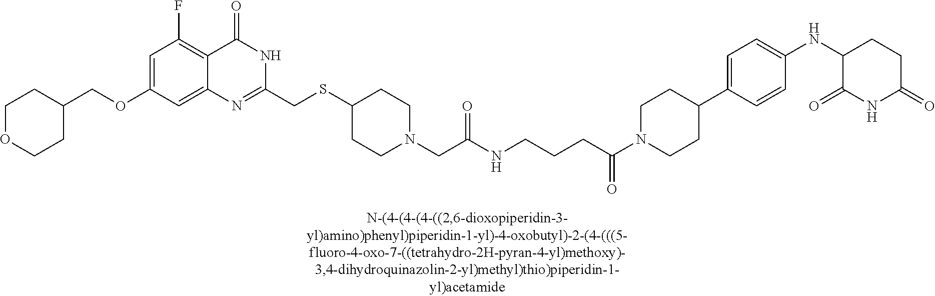 [SMILES: O=C(CN1CCC(SCc2nc3cc(OCC4CCOCC4)cc(F)c3c(=O)[nH]2)CC1)NCCCC(=O)N1CCC(c2ccc(NC3CCC(=O)NC3=O)cc2)CC1] | LCMS (ESI, m/z): 820.50 [M + H]⁺; ¹H NMR (300 MHz, Methanol-d₄) δ 7.12- 7.02 (m, 2H), 6.93 (s, 1H), 6.89-6.74 (m, 3H), 4.94-4.90 (m, 1H), 4.65 (d, J = 13.1 Hz, 1H), 4.31 (dd, J = 12.0, 4.9 Hz, 1H), 4.11-3.93 (m, 5H), 3.90 (s, 2H), 3.72-3.37 (m, 6H), 3.29-2.95 (m, 4H), 2.88-2.61 (m, 4H), 2.55-2.37 (m, 2H), 2.36-2.22 (m, 3H), 2.09-1.70 (m, 10H), 1.70-1.33 (m, 5H) |
| 9 | 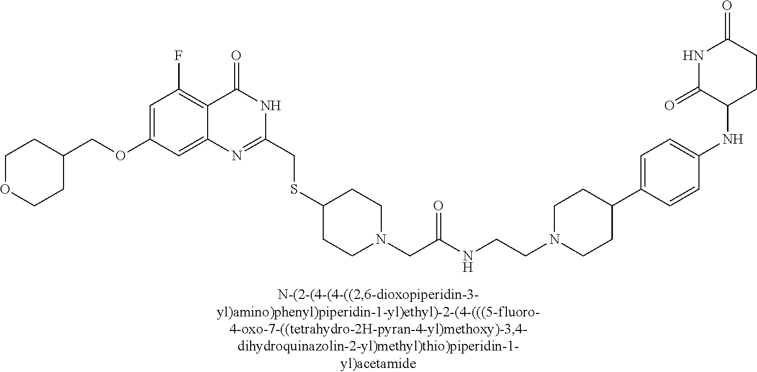 [SMILES: O=C(CN1CCC(SCc2nc3cc(OCC4CCOCC4)cc(F)c3c(=O)[nH]2)CC1)NCCN1CCC(c2ccc(NC3CCC(=O)NC3=O)cc2)CC1] | LCMS (ESI, m/z): 776.45 [M + H]⁺; ¹H NMR (400 MHz, DMSO-d₆) δ 12.12 (s, 1H), 10.76 (s, 1H), 7.68 (t, J = 5.5 Hz, 1H), 6.95 (d, J = 8.3 Hz, 2H), 6.92- 6.77 (m, 2H), 6.60 (d, J = 8.3 Hz, 2H), 5.62 (d, J = 7.4 Hz, 1H), 4.30-4.18 (m, 1H), 3.96 (d, J = 6.4 Hz, 2H), 3.95-3.87 (m, 2H), 3.62 (s, 2H), 3.45-3.36 (m, 1H) 3.29-3.14 (m, 3H), 2.93-2.58 (m, 9H), 2.42-2.24 (m, 3H), 2.15-1.89 (m, 8H), 1.83-1.76 (m, 1H), 1.75-1.61 (m, 4H), 1.60-1.51 (m, 4H), 1.40-1.21 (m, 2H) |
| 10 | 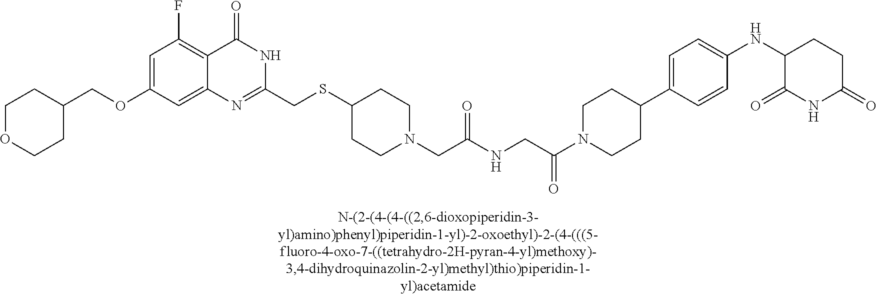 [SMILES: O=C(CN1CCC(SCc2nc3cc(OCC4CCOCC4)cc(F)c3c(=O)[nH]2)CC1)NCC(=O)N1CCC(c2ccc(NC3CCC(=O)NC3=O)cc2)CC1] | LCMS (ESI, m/z): 792.45 [M + H]⁺. ¹H NMR (300 MHz, Methanol-d₄) δ 7.12- 7.04 (m, 2H), 6.96 (d, J = 2.4 Hz, 1H), 6.88-6.76 (m, 3H), 4.67-4.45 (m, 1H), 4.36-4.24 (m, 1H), 4.24-4.07 (m, 2H), 4.07-3.89 (m, 7H), 3.78-3.37 (m, 5H), 3.25-3.14 (m, 2H), 3.10-2.95 (m, 1H), 2.88-2.68 (m, 4H), 2.66-2.64 (m, 1H), 2.40-2.20 (m, 3H), 2.19-2.02 (m, 1H), 2.00-1.70 (m, 7H), 1.68-1.37 (m, 5H). |
| 11 | 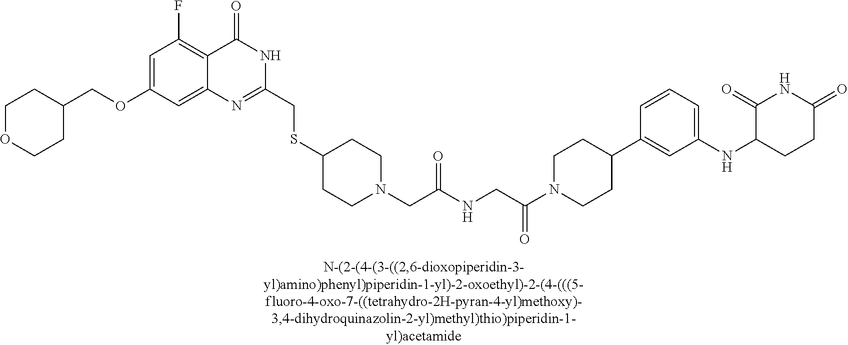 [SMILES: O=C(CN1CCC(SCc2nc3cc(OCC4CCOCC4)cc(F)c3c(=O)[nH]2)CC1)NCC(=O)N1CCC(c2cccc(NC3CCC(=O)NC3=O)c2)CC1] | LCMS (ESI, m/z): 792.40 [M + H]⁺. ¹HNMR (300 MHz, DMSO-d₆) δ 12.15 (s, 1H), 10.75 (s, 1H), 7.86 (t, J = 5.1 Hz, 1H), 6.98 (t, J = 7.7 Hz, 1H), 6.87 (d, J = 9.9 Hz, 2H), 6.56-6.39 (m, 3H), 5.73 (d, J = 7.6 Hz, 1H), 4.46 (d, J = 12.9 Hz, 1H), 4.29 (d, J = 7.1 Hz, 1H), 4.05-3.97 (m, 3H), 3.86 (dd, J = 11.1, 4.3 Hz, 3H), 3.60 (s, 2H), 3.59- 3.37 (m, 2H), 3.15-3.10 (m, 2H), 2.91 (s, 2H), 2.85-2.75 (m, 4H), 2.66-2.50 (m, 4H), 2.21-1.88 (m, 7H), 1.86-1.22 (m, 9H). |
| 12 | 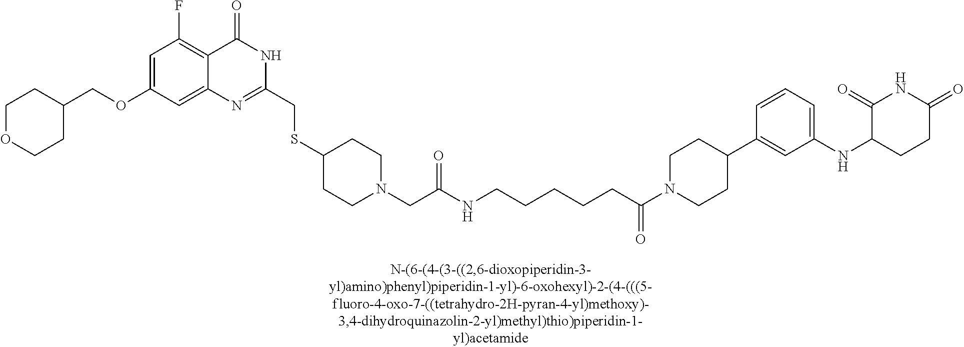 [SMILES: O=C(CN1CCC(SCc2nc3cc(OCC4CCOCC4)cc(F)c3c(=O)[nH]2)CC1)NCCCCCC(=O)N1CCC(c2cccc(NC3CCC(=O)NC3=O)c2)CC1] | LCMS (ESI, m/z): 424.95 [M + H]⁺. ¹H NMR (300 MHz, DMSO-d₆) δ 12.09 (s, 1H), 10.75 (s, 1H), 7.66 (t, J = 3.0 Hz, 1H), 6.97 (t, J = 9.0 Hz, 1H), 6.91- 6.82 (m, 2H), 6.55-6.39 (m, 3H), 5.73 (d, J = 6.0 Hz, 1H), 4.51 (d, J = 12.0 Hz, 1H), 4.37-4.25 (m, 1H), 3.97 (d, J = 6.0 Hz 2H), 3.90-3.84 (m, 3H), 3.58 (s, 2H), 3.08-3.02 (m, 3H), 2.85-2.79 (m, 3H), 2.73-2.68 (m, 3H), 2.58-2.51 (m, 1H), 2.32-2.25 (m, 3H), 2.14-2.05 (m, 4H), 1.94-1.85 (m, 4H), 1.77-1.64 (m, 5H), 1.50-1.25 (m, 13H). |
| 13 | 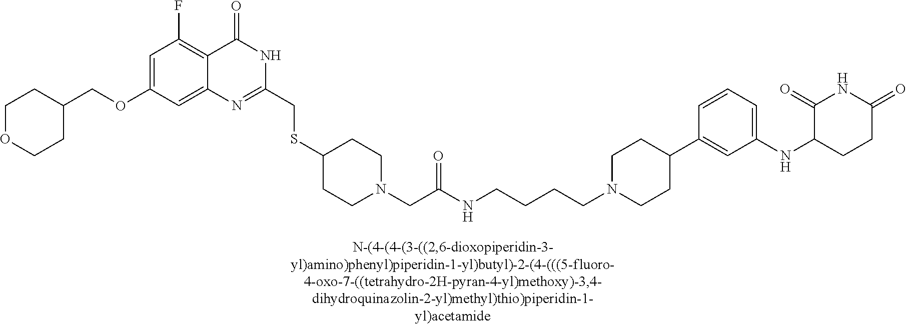 [SMILES: O=C(CN1CCC(SCc2nc3cc(OCC4CCOCC4)cc(F)c3c(=O)[nH]2)CC1)NCCCCN1CCC(c2cccc(NC3CCC(=O)NC3=O)c2)CC1] | LCMS (ESI, m/z): 806.50 [M + H]⁺ . ¹H NMR (300 MHz, DMSO-d₆) δ 12.11 (s, 1H), 10.76 (s, 1H), 7.69 (t, J = 5.9 Hz, 1H), 6.98 (t, J = 7.8 Hz, 1H), 6.93- 6.84 (m, 2H), 6.55 (s, H), 6.52-6.38 (m, 2H), 5.71 (d, J = 7.5 Hz, 1H), 4.39- 4.22 (m, 1H), 3.99 (d, J = 6.4 Hz, 2H), 3.95-3.79 (m, 2H), 3.60 (s, 2H), 3.39- 3.36 (m, 1H), 3.29-3.27 (m, 1H), 3.12- 3.03 (m, 2H), 2.96-2.89 (m, 2H), 2.86 (s, 2H), 2.82-2.66 (m, 4H), 2.62-2.55 (m, 1H), 2.31-2.21 (m, 3H), 2.17-2.07 (m, 3H), 2.02-1.78 (m, 5H), 1.79-1.59 (m, 5H), 1.61-1.46 (m, 4H), 1.45-1.39 (m, 4H), 1.37-1.21 (m, 2H). |
| 14 | 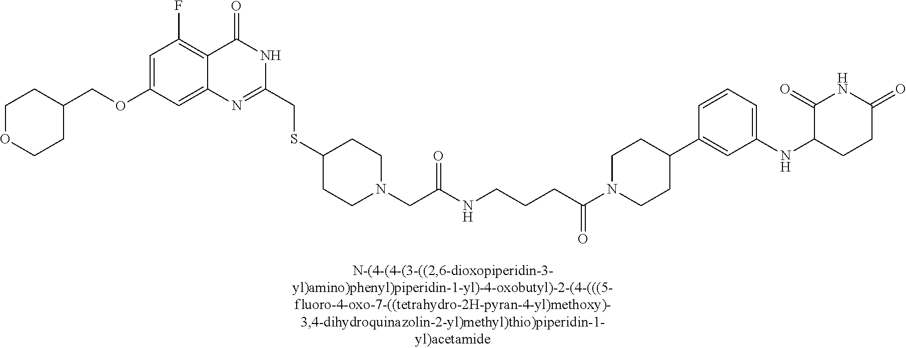 [SMILES: O=C(CN1CCC(SCc2nc3cc(OCC4CCOCC4)cc(F)c3c(=O)[nH]2)CC1)NCCCC(=O)N1CCC(c2cccc(NC3CCC(=O)NC3=O)c2)CC1] | LCMS (ESI, m/z): 820.35 [M + H]⁺. ¹H NMR (300 MHz, Methanol-d₄) δ 7.10- 7.09 (m, 1H), 6.97 (d, J = 2.5 Hz, 1H), 6.85 (dd, J = 12.4, 2.3 Hz, 1H), 6.69- 6.56 (m, 3H), 4.69 (d, J = 13.1 Hz, 1H), 4.53-4.32 (m, 1H), 4.24-4.12 (m, 3H), 4.08-3.95 (m, , 3H), 3.94-3.90 (m, 1H), 3.88-3.67 (m, 2H), 3.51-3.43 (m, 3H), 3.31-3.26 (m, 2H), 3.12-3.04 (m, 2H), 2.68-2.57 (m, 4H), 2.50-2.39 (m, 2H), 2.39-2.31 (m, 1H), 2.30-2.05 (m, 3H), 2.01-1.83 (m, 9H), 1.60-1.37 (m, 5H), 1.35-1.29 (m, 2H). |
| 15 | 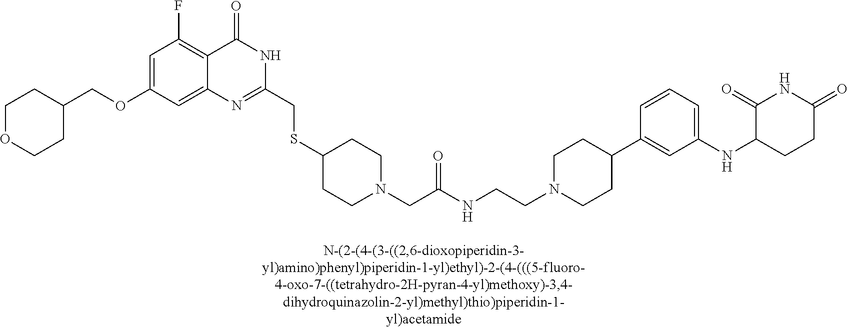 [SMILES: O=C(CN1CCC(SCc2nc3cc(OCC4CCOCC4)cc(F)c3c(=O)[nH]2)CC1)NCCN1CCC(c2cccc(NC3CCC(=O)NC3=O)c2)CC1] | LCMS (ESI, m/z): 778.40 [M + H]⁺. ¹H NMR (400 MHz, DMSO-d₆) δ 12.09 (s, 1H), 10.76 (s, 1H), 7.67 (t, J = 5.5 Hz, 1H), 6.98 (t, J = 7.8 Hz, 1H), 6.90- 6.85 (m, 2H), 6.54 (t, J = 2.0 Hz, 1H), 6.47 (t, J = 8.0 Hz, 2H), 5.70 (d, J = 7.5 Hz, 1H), 4.40-4.25 (m, 1H), 3.96 (d, J = 6.4 Hz, 2H), 3.91-3.82 (m, 2H), 3.61 (s, 2H), 3.52-3.40 (m, 1H), 3.23-3.13 (m, 2H), 2.96-291 (m, 2H), 2.87 (s, 2H), 2.84-2.66 (m, 4H), 2.59-2.52 (m, 1H), 2.43-2.27 (m, 3H), 2.18-2.06 (m, 3H), 2.05-1.92 (m, 5H), 1.85-1.45 (m, 9H), 1.39-1.22 (m, 2H). |
| 16 | 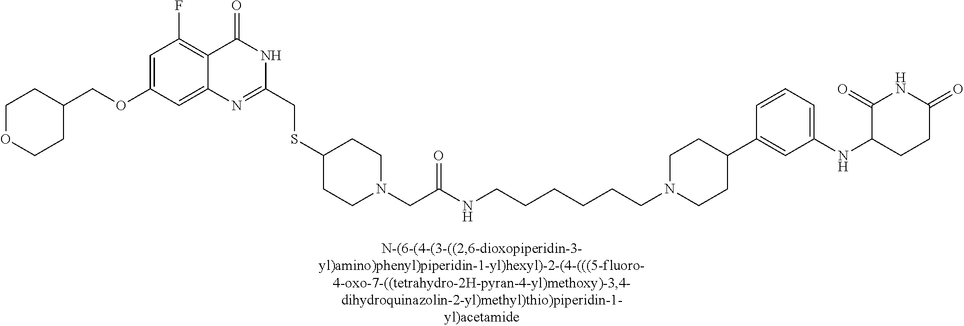 [SMILES: O=C(CN1CCC(SCc2nc3cc(OCC4CCOCC4)cc(F)c3c(=O)[nH]2)CC1)NCCCCCCN1CCC(c2cccc(NC3CCC(=O)NC3=O)c2)CC1] | LCMS (ESI, m/z): 834.35 [M + H]⁺. ¹H NMR (300 MHz, Methanol-d₄) δ 8.50 (s, 1H), 7.19-7.13 (m, 1H), 6.98-6.91 (m, 1H), 6.85 (dd, J = 12.6, 2.4 Hz, 1H), 6.69-6.57 (m, 3H), 4.31 (dd, J = 11.7, 4.8 Hz, 1H), 4.07-3.96 (m, 4H), 3.66 (s, 2H), 3.65-3.50 (m, 2H), 3.49-3.30 (m, 2H), 3.25 (t, J = 6.9 Hz, 2H), 3.18- 3.04 (m, 4H), 3.01 (s, 2H), 2.91-2.67 (m, 6H), 2.30-2.24 (m, 3H), 2.13-1.85 (m, 8H), 1.83-1.35 (m, 14H). |
| 17 | 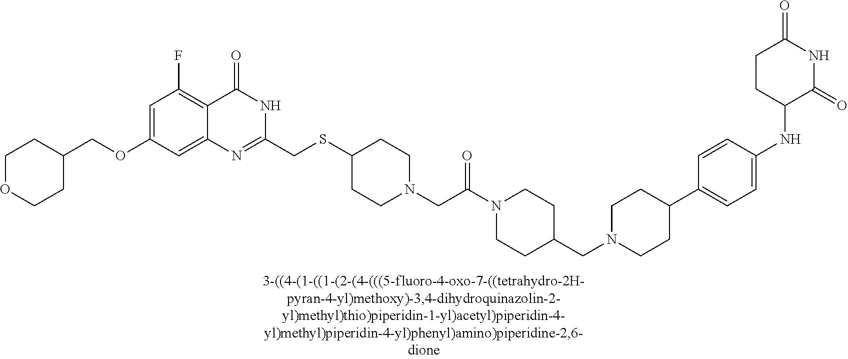 [SMILES: O=C1CCC(Nc2ccc(C3CCN(CC4CCN(C(=O)CN5CCC(SCc6nc7cc(OCC8CCOCC8)cc(F)c7c(=O)[nH]6)CC5)CC4)CC3)cc2)C(=O)N1] | LCMS (ESI, m/z): 832.50 [M + H]⁺. ¹H NMR (300 MHz, DMSO-d₆) δ 12.15 (s, 1H), 10.77 (s, 1H), 6.94 (d, J = 8.4 Hz, 2H), 6.90-6.87 (m, 2H), 6.60 (d, J = 9.2 Hz, 2H), 5.63 (d, J = 7.8, Hz, 1H), 4.33-4.24 (m, 2H), 4.03-3.98 (m, 3H), 3.90-3.85 (m, 2H), 3.60 (s, 2H), 3.22 (s, 1H), 3.18-3.11 (m, 1H), 2.96-2.88 (m, 4H), 2.76-2.69 (m, 4H), 2.62-2.58 (m, 1H), 2.35-2.27 (m, 1H), 2.12-2.07 (m, 6H), 1.95-1.89 (m, 5H), 1.73-1.65 (m, 7H), 1.57-1.48 (m, 3H), 1.41-1.34 (m, 3H), 1.08-1..00 (m, 1H), 0.90-0.83 (m, 1H). |
| 18 | 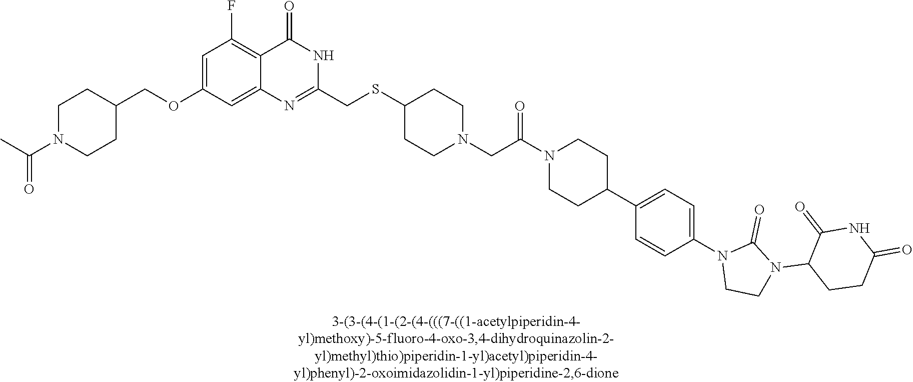 [SMILES: CC(=O)N1CCC(COc2cc(F)c3c(=O)[nH]c(CSC4CCN(CC(=O)N5CCC(c6ccc(N7CCN(C8CCC(=O)NC8=O)C7=O)cc6)CC5)CC4)nc3c2)CC1] | LCMS (ESI, m/z): 845.35 [M + H]⁺. ¹H NMR (400 MHz, DMSO-d₆) δ 12.15 (s, 1H), 10.90 (s, 1H), 8.20 (s, 1H, HCOOH), 7.49 (d, J = 8.0 Hz, 2H), 7.18 (d, J = 8.5 Hz, 2H), 6.88 (d, J = 8.0 Hz, 2H), 4.68 (dd, J = 13.3, 5.1 Hz, 1H), 4.47 (d, J = 12.7 Hz, 1H), 4.39 (d, J = 12.9 Hz, 1H), 4.14 (d, J = 12.9 Hz, 1H), 3.99 (d, J = 6.3 Hz, 2H), 3.91-.74 (m, 3H), 3.59 (s, 2H), 3.51-3.41 (m, 2H), 3.37-3.21 (m, 3H), 3.09-2.96 (m, 3H), 2.92-2.65 (m, 5H), 2.64-2.51 (m, 2H), 2.33-2.18 (m, 1H), 2.16-2.05 (m, 2H), 1.99 (s, 3H), 1.96-1.86 (m, 3H), 1.80- 1.70 (m, 4H), 1.61-1.32 (m, 4H), 1.31- 1.18 (m, 1H), 1.17-1.04 (m, 1H). |
| 19 | 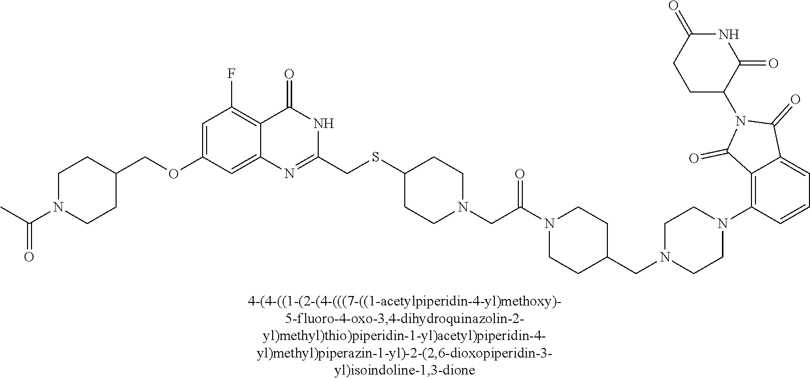 [SMILES: CC(=O)N1CCC(COc2cc(F)c3c(=O)[nH]c(CSC4CCN(CC(=O)N5CCC(CN6CCN(c7cccc8c7C(=O)N(C7CCC(=O)NC7=O)C8=O)CC6)CC5)CC4)nc3c2)CC1] | LCMS (ESI, m/z): 928.50 [M + H]⁺. ¹H NMR (300 MHz, DMSO-d₆) δ 12.13 (s, 1H), 11.09 (s, 1H), 7.71 (dd, J = 8.4, 7.1 Hz, 1H), 7.35 (t, J = 7.7 Hz, 2H), 6.99-6.71 (m, 2H), 5.10 (dd, J = 12.8, 5.4 Hz, 1H), 4.45-4.26 (m, 2H), 4.10-3.99 (m 3H), 3.85 (d, J = 13.6 Hz, 1H), 3.60 (s, 2H), 3.25-3.18 (m, 1H), 3.15-2.60 (m, 9H), 2.55 (s, 6H), 2.19- 1.58 (m, 16H), 1.45-0.75 (m, 8H). |
| 20 | 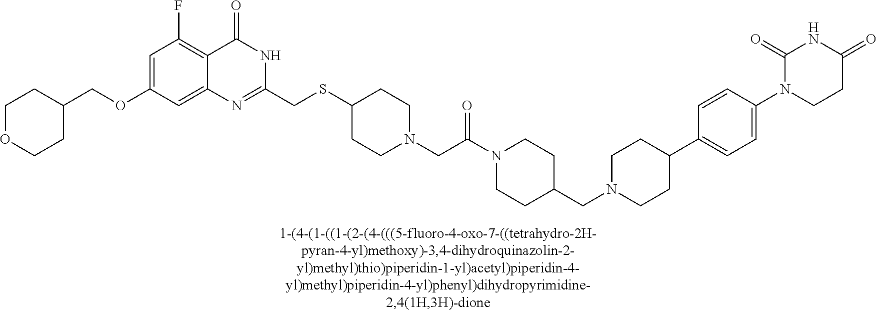 [SMILES: O=C1CCN(c2ccc(C3CCN(CC4CCN(C(=O)CN5CCC(SCc6nc7cc(OCC8CCOCC8)cc(F)c7c(=O)[nH]6)CC5)CC4)CC3)cc2)C(=O)N1] | LCMS (ESI, m/z): 818.35 [M + H]⁺. ¹H NMR (300 MHz, DMSO-d₆) δ 8.24 (s, 1H), 7.25 (s, 3H), 6.94-6.82 (m, 2H), 4.29 (d, J = 11.4 Hz, 1H), 4.05-3.92 (m, 2H), 3.91-3.81 (m, 2H), 3.80-3.75 (m, 6H), 3.73-3.70 (m, 7H), 3.61-3.55 (m, 1H), 3.39-3.22 (m, 2H), 3.21-3.10 (m, 1H), 3.08-2.87 (m, 2H), 2.85-2.64 (m, 4H), 2.63-2.61 (m, 3H), 2.45-2.40 (m, 3H), 2.20-2.05 (m, 2H), 2.04-1.96 (m, 1H), 1.95-1.40 (m, 9H), 1.39-1.24 (m, 2H) |
| 21 | 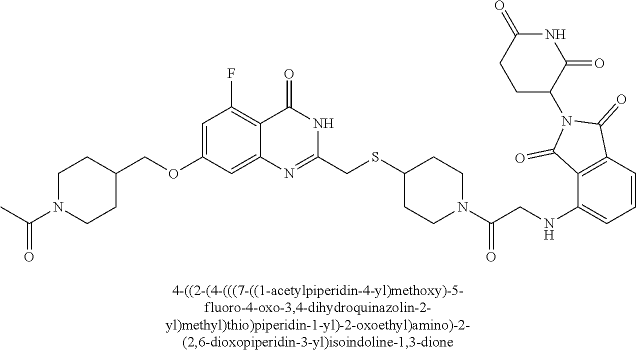 [SMILES: CC(=O)N1CCC(COc2cc(F)c3c(=O)[nH]c(CSC4CCN(C(=O)CNc5cccc6c5C(=O)N(C5CCC(=O)NC5=O)C6=O)CC4)nc3c2)CC1] | LCMS (ESI, m/z): 381.80 [M + H]⁺. ¹H NMR (300 MHz, DMSO-d6) δ 12.06 (s, 1H), 11.10 (s, 1H), 7.64-7.53 (m, 1H), 7.14-7.01 (m, 3H), 6.87 (m, 2H), 5.05 (dd, J = 12.6, 5.3 Hz, 1H), 4.38 (d, J = 12.9 Hz, 1H), 4.15 (s, 3H), 3.98 (d, J = 6.4 Hz, 2H), 3.82 (d, J = 8.4 Hz, 2H), 3.63 (s, 2H), 3.19-3.02 (m, 3H), 2.95-2.81 (m, 2H), 2.63-2.54 (m, 3H), 1.98 (s, 6H), 1.93 (s, 1H), 1.77 (t, J = 12.1 Hz, 2H), 1.27-1.19 (m, 2H), 1.19-1.05 (m, 3H). |
| 22 | 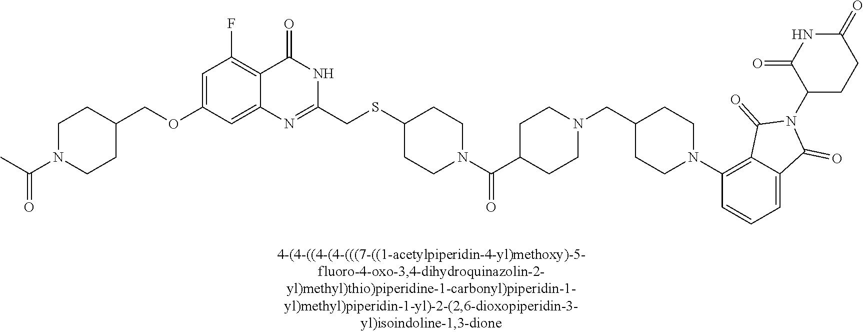 [SMILES: CC(=O)N1CCC(COc2cc(F)c3c(=O)[nH]c(CSC4CCN(C(=O)C5CCN(CC6CCN(c7cccc8c7C(=O)N(C7CCC(=O)NC7=O)C8=O)CC6)CC5)CC4)nc3c2)CC1] | LCMS (ESI, m/z): 913.60 [M + H]⁺. ¹H NMR (300 MHz, DMSO-d₆) δ 12.17 (s, 1H), 11.09 (s, 1H), 7.67 (t, J = 7.7 Hz, 1H), 7.43-7.18 (m, 2H), 6.97-6.80 (m, 2H), 5.16-4.99 (m, 1H), 4.40 (d, J = 12.9 Hz, 1H), 4.16 (d, J = 12.5 Hz, 1H), 4.00 (d, J = 6.3 Hz, 2H), 3.84 (d, J = 13.5 Hz, 2H), 3.69-3.63 (m, 4H), 3.15- 3.02 (m, 3H), 2.95-2.73 (m, 6H), 2.68- 2.51 (m, 3H), 2.15 (d, J = 6.6 Hz, 2H), 2.08 1.43 (m, 19H), 1.41-1.01 (m, 7H). |
| 23 | 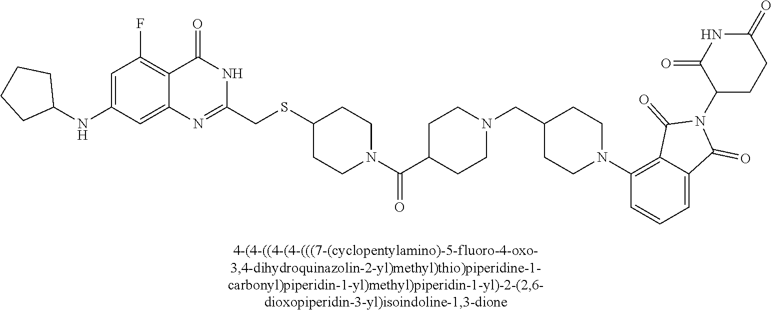 [SMILES: O=C1CCC(N2C(=O)c3cccc(N4CCC(CN5CCC(C(=O)N6CCC(SCc7nc8cc(NC9CCCC9)cc(F)c8c(=O)[nH]7)CC6)CC5)CC4)c3C2=O)C(=O)N1] | LCMS (ESI, m/z): 421.50 [M + H]⁺. ¹H NMR (300 MHz, DMSO-d₆) δ 11.65 (s, 1H), 11.07 (s, 1H), 7.66 (dd, J = 8.5, 7.1 Hz, 1H), 7.31 (dd, J = 7.8, 5.7 Hz, 2H), 6.82 (d, J = 6.5 Hz, 1H), 6.40 (dd, J = 13.8, 2.1 Hz, 1H), 6.34 (d, J = 2.2 Hz, 1H), 5.07 (dd, J = 12.7, 5.4 Hz, 1H), 4.14 (d, J = 12.9 Hz, 1H), 3.88-3.72 (m, 2H), 3.67 (d, J = 11.6 Hz, 2H), 3.55 (s, 2H), 3.15-3.03 (m, 2H), 2.92-2.77 (m, 6H), 2.60-2.54 (m, 2H), 2.14 (d, J = 6.9 Hz, 2H), 2.09- 1.86 (m, 8H), 1.85-1.24 (m, 17H). |
| 24 | 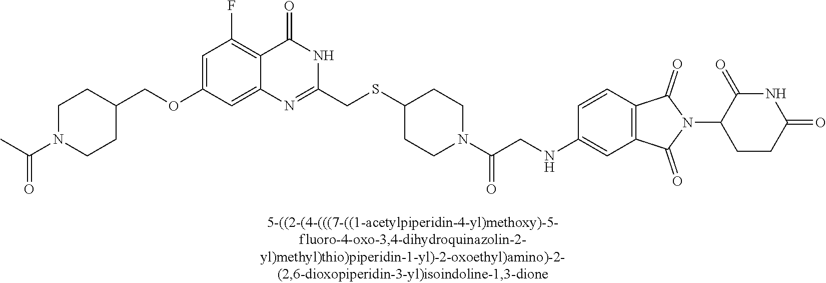 [SMILES: CC(=O)N1CCC(COc2cc(F)c3c(=O)[nH]c(CSC4CCN(C(=O)CNc5ccc6c(c5)C(=O)N(C5CCC(=O)NC5=O)C6=O)CC4)nc3c2)CC1] | LCMS (ESI, m/z): 762.40 [M + H]⁺. ¹H NMR (300 MHz, DMSO-d₆) δ 12.00 (s, 1H), 11.20-10.70 (m, 1H), 7.55 (d, J = 8.4 Hz, 1H), 7.19-6.95 (m, 2H), 6.98- 6.84 (m, 2H), 6.80-6.45 (m, 1H), 5.95- 5.85 (m, 1H), 5.08-4.97 (m, 1H), 4.63- 4.34 (m, 1H), 4.21-4.05 (m, 1H), 4.00 (d, J = 6.4 Hz, 2H), 3.85 (d, J = 13.1 Hz, 2H), 3.65 (s, 2H), 3.18-2.97 (m, 4H), 2.97-2.80 (m, 2H), 2.64-2.54 (m, 2H), 2.07-1.89 (m, 7H), 1.85-1.64 (m, 2H), 1.58-0.98 (m, 6H). |
| 25 | 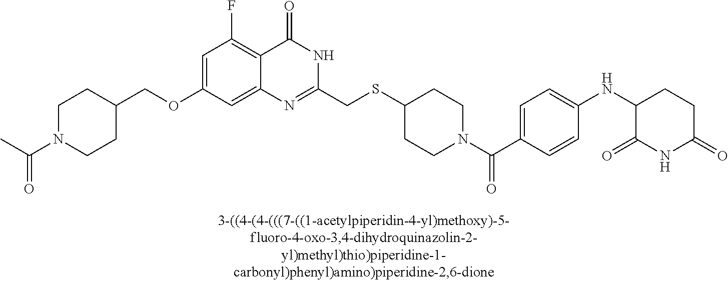 [SMILES: CC(=O)N1CCC(COc2cc(F)c3c(=O)[nH]c(CSC4CCN(C(=O)c5ccc(NC6CCC(=O)NC6=O)cc5)CC4)nc3c2)CC1] | LCMS (ES, m/z): 679.30 [M + H]⁺. ¹H NMR (300 MHz, DMSO-d₆) δ 12.12 (s, 1H), 10.82 (s, 1H), 7.18 (d, J = 8.7 Hz, 2H), 6.88 (d, J = 10.5 Hz, 2H), 6.73 (d, J = 8.7 Hz, 2H), 6.28 (d, J = 7.8 Hz, 1H), 4.52-4.28 (m, 2H), 4.10- 3.80 (m, 5H), 3.63 (s, 2H), 3.15-2.98 (m, 4H), 2.82-2.70 (m, 1H), 2.66-2.52 (m, 3H), 2.15-1.84 (m, 7H), 1.83-1.70 (m, 2H), 1.52-1.39 (m, 2H), 1.29-1.07 (m, 2H). |
| 26 | 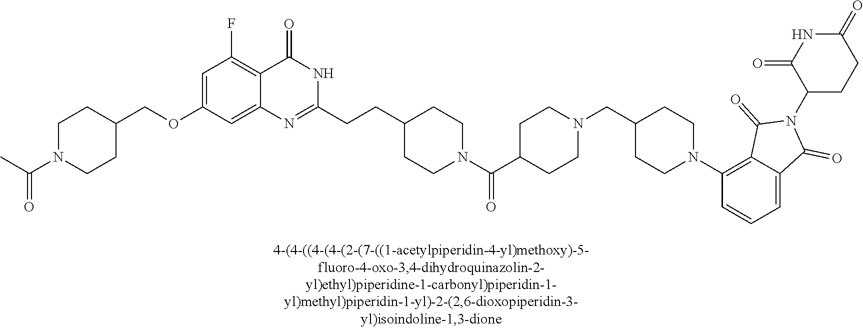 [SMILES: CC(=O)N1CCC(COc2cc(F)c3c(=O)[nH]c(CCC4CCN(C(=O)C5CCN(CC6CCN(c7cccc8c7C(=O)N(C7CCC(=O)NC7=O)C8=O)CC6)CC5)CC4)nc3c2)CC1] | LCMS (ESI, m/z): 895.50 [M + H]⁺. ¹H NMR (300 MHz, DMSO-d₆) δ 12.00 (s, 1H), 11.07 (s, 1H), 7.66 (dd, J = 8.5, 7.1 Hz, 1H), 7.40-7.18 (m, 2H), 6.95-6.70 (m, 2H), 5.07 (dd, J = 12.7, 5.4 Hz, 1H), 4.38 (d, J = 13.0 Hz, 2H), 3.98 (d, J = 6.3 Hz, 2H), 3.91-3.76 (m, 2H), 3.67 (d, J = 11.7 Hz, 2H), 3.10- 2.80 (m, 5H), 2.86-2.58 (m, 5H), 2.16 (d, J = 6.3 Hz, 2H),2.14-1.83 (m, 7H), 1.76-1.55 (m, 14H),1.30-0.88 (m, 8H). |
| 27 | 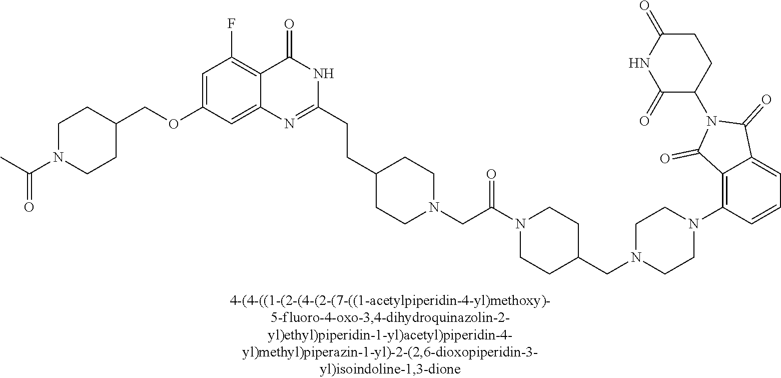 [SMILES: CC(=O)N1CCC(COc2cc(F)c3c(=O)[nH]c(CCC4CCN(CC(=O)N5CCC(CN6CCN(c7cccc8c7C(=O)N(C7CCC(=O)NC7=O)C8=O)CC6)CC5)CC4)nc3c2)CC1] | LCMS (ESI, m/z): 910.45. [M + H]⁺. ¹H NMR (300 MHz, DMSO-d₆) δ 11.93 (s, 1H), 11.10 (s, 1H), 7.69 (dd, J = 8.4, 7.2 Hz, 1H), 7.33 (t, J = 7.3 Hz, 2H), 6.97-6.74 (m, 2H), 5.08 (dd, J = 12.8, 5.4 Hz, 1H), 4.50-4.15 (m, 2H), 4.05 (d, J = 12.9 Hz, 1H), 3.97 (d, J = 6.4 Hz, 2H), 3.82 (d, J = 13.5 Hz, 1H), 3.48-3.37 (m, 4H), 3.33-3.28 (m, 2H), 3.21-3.16 (m, 1H), 3.15-2.93 (m, 3H), 2.94-2.73 (m, 3H), 2.55-2.50 (m, 4H), 2.15-2.08 (m, 2H), 2.00-1.50 (m, 18H), 1.47-0.52 (m, 9H). |
| 28 | 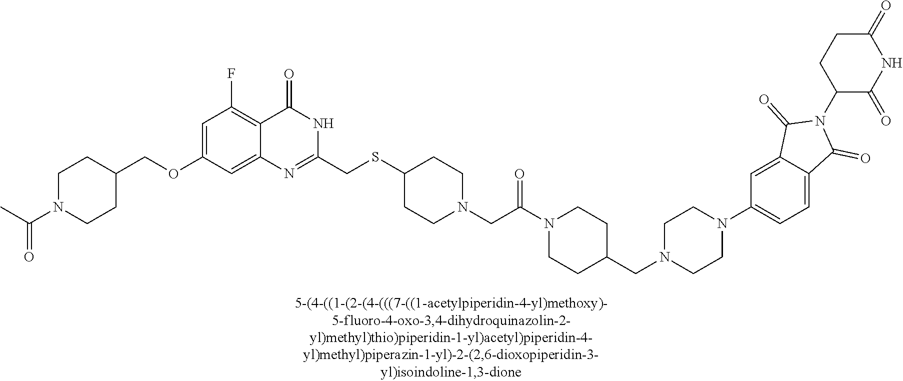 [SMILES: CC(=O)N1CCC(COc2cc(F)c3c(=O)[nH]c(CSC4CCN(CC(=O)N5CCC(CN6CCN(c7ccc8c(c7)C(=O)N(C7CCC(=O)NC7=O)C8=O)CC6)CC5)CC4)nc3c2)CC1] | LCMS (ESI, m/z): 926.45 [M + H]⁺. ¹H NMR (400 MHz, DMSO-d₆) δ 12.18 (s, 1H), 11.09 (s, 1H), 8.23 (s, 1H), 7.68 (d, J = 8.6 Hz, 1H), 7.34 (s, 1H), 7.29-7.22 (m, 1H), 6.94-6.85 (m, 2H), 5.08 (dd, J = 12.9, 5.4 Hz, 1H), 4.40 (d, J = 13.2 Hz, 2H), 4.31 (d, J = 12.6 Hz, 3H), 4.00 (d, J = 6.3 Hz, 1H), 3.84 (d, J = 13.5 Hz, 2H), 3.64 (s, 2H), 3.62-3.20 (m, 6H), 3.08-2.73 (m, 6H), 2, 58-2.48 (m, 3H), 2.17 (d, J = 7.0 Hz, 2H), 2.11- 2.02 (m, 2H), 2.00-1.91 (m, 7H), 1.84- 1.81 (m, 2H), 1.81-1.72 (m, 5H), 1.51- 1.40 (m, 2H), 1.25-1.18 (m, 1H), 1.17- 1.01 (m, 3H), 0.93-0.84 (m, 1H). |
| 29 | 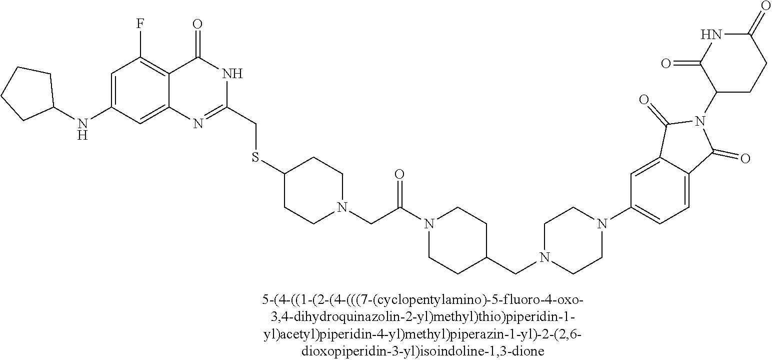 [SMILES: O=C1CCC(N2C(=O)c3ccc(N4CCN(CC5CCN(C(=O)CN6CCC(SCc7nc8cc(NC9CCCC9)cc(F)c8c(=O)[nH]7)CC6)CC5)CC4)cc3C2=O)C(=O)N1] | LCMS (ESI, m/z): 856.40 [M + H]⁺. ¹H NMR (400 MHz, DMSO-d₆) δ 11.66 (s, 1H), 11.08 (s, 1H), 7.68 (d, J = 8.5 Hz, 1H), 7.34 (d, J = 2.2 Hz, 1H), 7.25 (dd, J = 8.7, 2.3 Hz, 1H), 6.84 (d, J = 6.5 Hz, 1H), 6.42 (dd, J = 13.9, 2.2 Hz, 1H), 6.36 (d, J = 2.1 Hz, 1H), 5.08 (dd, J = 12.9, 5.4 Hz, 1H), 4.17 (dd, J = 114.5, 12.7 Hz, 2H), 3.85-3.75 (m, 1H), 3.53 (s, 3H), 3.49-3.32 (m, 5H) 3.24-2.94 (m, 4H), 2.89-2.53 (m, 6H), 2.47 (s, 2H), 2.17 (d, J = 6.9 Hz, 2H), 2.10-1.83 (m, 7H), 1.81-1.61 (m, 5H), 1.60- 1.51 (m, 2H), 1.50-1.31 (m, 4H), 1.11- 0.83 (m, 2H). |

### Example 30: Synthesis of 5-(4-(((7-((1-acetylpiperidin-4-yl)methoxy)-5-fluoro-4-oxo-3,4-dihydroquinazolin-2-yl)methyl)thio)piperidin-1-yl)-2-(2,6-dioxopiperidin-3-yl)isoindoline-1,3-dione

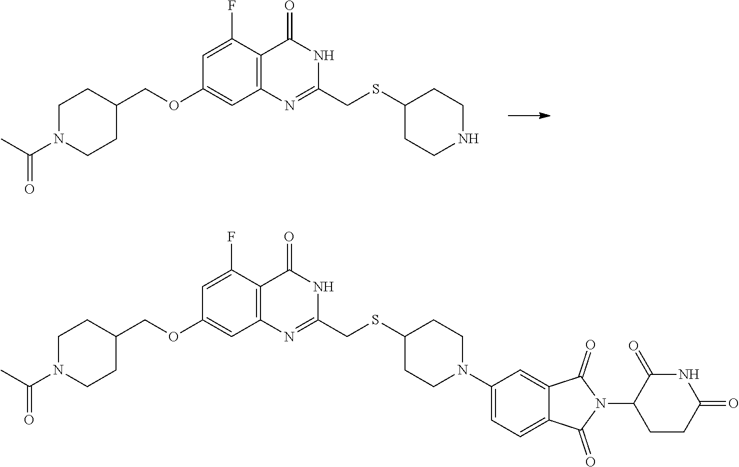 [SMILES: CC(=O)N1CCC(COc2cc(F)c3c(=O)[nH]c(CSC4CCNCC4)nc3c2)CC1 ; CC(=O)N1CCC(COc2cc(F)c3c(=O)[nH]c(CSC4CCN(c5ccc6c(c5)C(=O)N(C5CCC(=O)NC5=O)C6=O)CC4)nc3c2)CC1]

A solution of 7-((1-acetylpiperidin-4-yl)methoxy)-5-fluoro-2-((piperidin-4-ylthio)methyl)quinazolin-4(3H)-one (200 mg, 0.45 mmol, 1.0 equiv), 2-(2,6-dioxopiperidin-3-yl)-5-fluoroisoindoline-1,3-dione (123 mg, 0.45 mmol, 1.0 equiv) and DIEA (230 mg, 1.78 mmol, 4.0 equiv) in NMP (5 mL) was stirred for 12 hours at 120° C. The residue was purified directly by reverse flash chromatography with the following conditions: (column, C18 silica gel; mobile phase, MeCN in water (10 mmol/L NH₄HCO₃), 10% to 70% gradient in 15 min; detector, UV 254 nm) to afford 5-(4-(((7-((1-acetylpiperidin-4-yl)methoxy)-5-fluoro-4-oxo-3,4-dihydroquinazolin-2-yl)methyl)thio)piperidin-1-yl)-2-(2,6-dioxopiperidin-3-yl)isoindoline-1,3-dione (47 mg, 15%) as a green solid. LCMS (ES, m/z): 705.35 [M+H]⁺. ¹H NMR (300 MHz, Methanol-d₄) δ 7.66 (d, J=8.5 Hz, 1H), 7.33 (d, J=2.3 Hz, 1H), 7.20 (dd, J=8.6, 2.4 Hz, 1H), 6.96-6.90 (m, 1H), 6.84 (dd, J=12.5, 2.4 Hz, 1H), 5.08 (dd, J=12.3, 5.4 Hz, 1H), 4.59 (d, J=13.2 Hz, 1H), 4.04-3.88 (m, 5H), 3.72 (d, J=4.8 Hz, 1H), 3.26-3.04 (m, 4H), 2.97-2.63 (m, 4H), 2.21-2.01 (m, 7H), 2.00-1.81 (m, 2H), 1.77-1.56 (m, 2H), 1.47-1.20 (m, 3H).

### Example 31: Synthesis of 2-(4-(((7-((1-acetylpiperidin-4-yl)methoxy)-5-fluoro-4-oxo-3,4-dihydroquinazolin-2-yl)methyl)thio)piperidin-1-yl)-N-(2-(2,6-dioxopiperidin-3-yl)-1,3-dioxoisoindolin-4-yl)acetamide

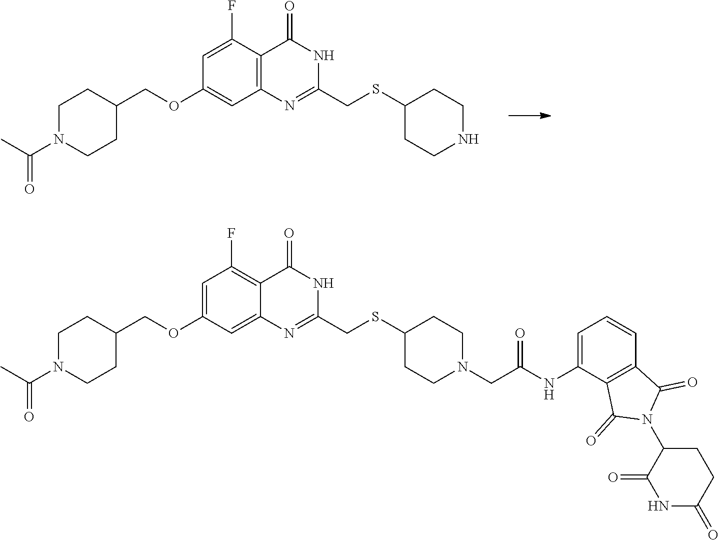 [SMILES: CC(=O)N1CCC(COc2cc(F)c3c(=O)[nH]c(CSC4CCNCC4)nc3c2)CC1 ; CC(=O)N1CCC(COc2cc(F)c3c(=O)[nH]c(CSC4CCN(CC(=O)Nc5cccc6c5C(=O)N(C5CCC(=O)NC5=O)C6=O)CC4)nc3c2)CC1]

A solution of 7-[(1-acetylpiperidin-4-yl)methoxy]-5-fluoro-2-[(piperidin-4-ylsulfanyl)methyl]-3H-quinazolin-4-one (100 mg, 0.223 mmol, 1.0 equiv), 2-bromo-N-[2-(2,6-dioxopiperidin-3-yl)-1,3-dioxoisoindol-4-yl]acetamide (83 mg, 0.21 mmol, 0.95 equiv) and K₂CO₃ (62 mg, 0.45 mmol, 2.0 equiv) in DMF (2 mL) was stirred for 2 hours at 70° C. The residue was purified directly by reverse-phase flash chromatography with the following conditions: (column, C18 silica gel; mobile phase, MeCN in water (10 mmol/L NH₄HCO₃), 5% to 95% gradient in 25 min; detector, UV 254 nm) and further purified by Prep-HPLC with the following condition: (Column: YMC-Actus Triart C18 ExRS, 30*150 mm, 5 m; Mobile Phase A: water(10 mmol/L NH₄HCO₃), Mobile Phase B: ACN; Flow rate: 60 mL/min; Gradient: 17% B to 42% B in 9 min, 42% B; Wave Length: 254/220 nm; RT (min): 8.9) to afford 2-(4-(((7-((1-acetylpiperidin-4-yl)methoxy)-5-fluoro-4-oxo-3,4-dihydroquinazolin-2-yl)methyl)thio)piperidin-1-yl)-N-(2-(2,6-dioxopiperidin-3-yl)-1,3-dioxoisoindolin-4-yl)acetamide (12 mg, 7%) as a white solid. LCMS (ESI, m/z): 762.25 [M+H]*; ¹H NMR (300 MHz, DMSO-d₆) δ 11.12 (s, 1H), 11.01 (s, 1H), 8.79 (d, J=9.0 Hz, 1H), 8.22 (s, 1H), 7.85 (t, J=9.0 Hz, 1H), 7.59 (d, J=6.0 Hz, 1H), 6.95-6.82 (m, 2H), 5.22-5.10 (m, 1H), 4.39 (d, J=12.0 Hz, 1H), 4.00 (d, J=6.0 Hz, 2H), 3.91-3.71 (m, 2H), 3.62 (s, 2H), 3.21 (d, J=9.0 Hz, 2H), 3.11-2.99 (m, 1H), 2.98-2.81 (m, 4H), 2.68-2.52 (m, 2H), 2.39-2.28 (m, 2H), 2.12-1.85 (m, 7H), 1.82-1.61 (m, 4H), 1.32-1.05 (m, 2H).

Example 32 was synthesized according to the procedure described for the synthesis of 2-(4-(((7-((1-acetylpiperidin-4-yl)methoxy)-5-fluoro-4-oxo-3,4-dihydroquinazolin-2-yl)methyl)thio)piperidin-1-yl)-N-(2-(2,6-dioxopiperidin-3-yl)-1,3-dioxoisoindolin-4-yl)acetamide using appropriate building blocks and modified reaction conditions (such as reagents, reagent ratio, temperature, and reaction time) and purification conditions as needed.

| Ex- | | |
|---|---|---|
| ample | Structure | Analytical Data |
| 32 | 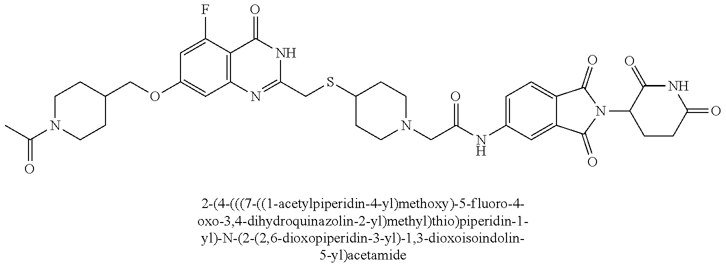 [SMILES: CC(=O)N1CCC(COc2cc(F)c3c(=O)[nH]c(CSC4CCN(CC(=O)Nc5ccc6c(c5)C(=O)N(C5CCC(=O)NC5=O)C6=O)CC4)nc3c2)CC1] | LCMS (ESI, m/z): 762.30 [M + H]⁺. ¹H NMR (300 MHz, DMSO-d₆) δ 12.10 (s, 1H), 11.10 (s, 1H), 10.30 (s, 1H), 8.29 (d, J = 1.8 Hz, 1H), 8.04 (dd, J = 8.1, 1.8 Hz, 1H), 7.86 (d, J = 8.1 Hz, 1H), 6.91-6.83 (m, 2H), 5.11 (dd, J = 12.8, 5.4 Hz, 1H), 4.38 (d, J = 12.9 Hz, 1H), 3.98 (d, J = 6.4 Hz, 2H), 3.82 (d, J = 13.1 Hz, 1H), 3.60 (s, 2H), 3.18 (s, 2H), |
| | | 3.03 (t, J = 12.3 Hz, |
| | | 1H), 2.95-2.74 (m, |
| | | 4H), 2.71-2.52 (m, |
| | | 3H), 2.82-2.19 (m, |
| | | 2H), 2.08-1.82 (m, |
| | | 6H), 1.80-1.71 (m, |
| | | 2H), 1.68-1.51 (m, |
| | | 2H), 1.32-1.01 (m, |
| | | 3H). |

### Example 33: Synthesis of 5-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione

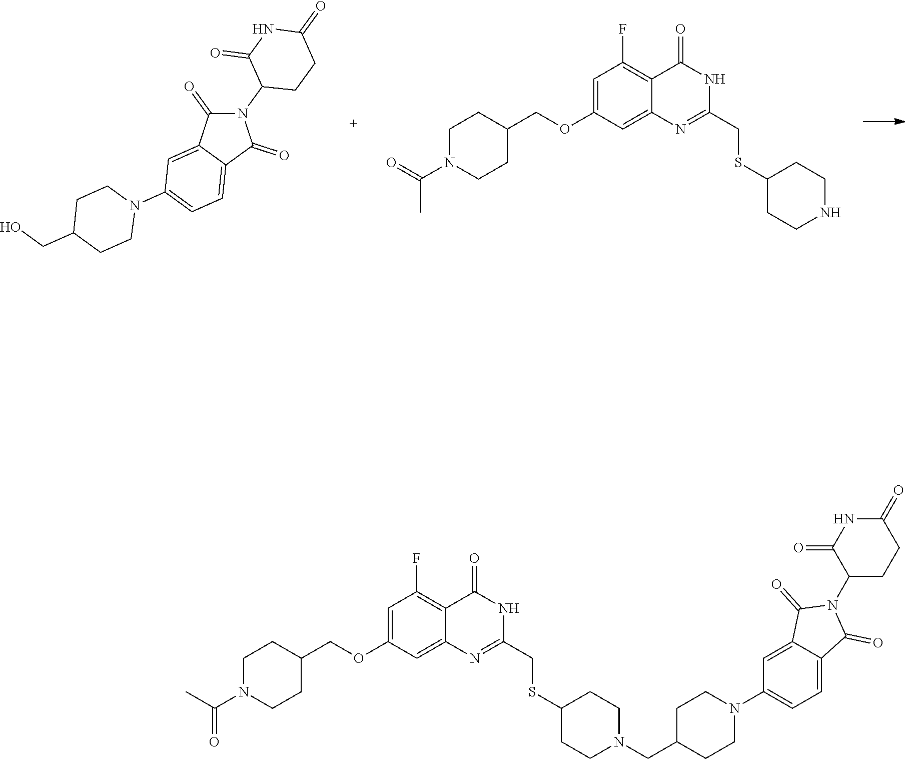 [SMILES: O=C1CCC(N2C(=O)c3ccc(N4CCC(CO)CC4)cc3C2=O)C(=O)N1 ; CC(=O)N1CCC(COc2cc(F)c3c(=O)[nH]c(CSC4CCNCC4)nc3c2)CC1 ; CC(=O)N1CCC(COc2cc(F)c3c(=O)[nH]c(CSC4CCN(CC5CCN(c6ccc7c(c6)C(=O)N(C6CCC(=O)NC6=O)C7=O)CC5)CC4)nc3c2)CC1]

A solution of 2-(2,6-dioxopiperidin-3-yl)-5-[4-(hydroxymethyl)piperidin-1-yl]isoindole-1,3-dione (200 mg, 0.54 mmol, 1.0 equiv) and Dess-Martin periodinane (251 mg, 0.59 mmol, 1.1 equiv) in DCM (3 mL) was stirred for 2 hours. To the mixture was added 7-[(1-acetylpiperidin-4-yl)methoxy]-5-fluoro-2-[(piperidin-4-ylsulfanyl)methyl]-3H-quinazolin-4-one (241 mg, 0.539 mmol, 1 equiv) dropwise. The resulting mixture was stirred for 2 hours and then NaBH(OAc)₃ (5716 mg, 2.69 mmol, 5.0 equiv) was added then stirred for 16 hours. The mixture was concentrated under vacuum and the residue was purified by reverse flash chromatography with the following conditions: (column, C18 silica gel; mobile phase, MeCN in water (10 mmol/L NH₄HCO₃), 0% to 43% gradient in 25 min; detector, UV 254 nm). The crude product was further purified by prep-HPLC with the following conditions: (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 15% B to 45% B in 7 min, 45% B; Wave Length: 254/220 nm; RT (min): 5.5) to afford 5-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione (4.2 mg, 1%) as a yellow solid. LCMS (ES, m/z): 802.33 [M+H]⁺; ¹H NMR (300 MHz, DMSO-d₆) δ 12.14 (s, 1H), 11.06 (s, 1H), 7.63 (d, J=8.5 Hz, 1H), 7.28 (d, J=2.1 Hz, 1H), 7.21 (d, J=8.6 Hz, 1H), 6.93-6.82 (m, 2H), 5.11-5.05 (m, 1H), 4.38 (d, J=12.7 Hz, 1H), 4.08-3.95 (m, 4H), 3.83 (d, J=13.8 Hz, 1H), 3.58 (s, 2H), 3.05-2.74 (m, 6H), 2.59-2.51 (m, 1H), 2.09-1.69 (m, 17H), 1.51-1.48 (m, 2H), 1.29-1.05 (m, 5H).

### Example 34: Synthesis of 5-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-2-(2,6-dioxopiperidin-3-yl)isoindoline-1,3-dione

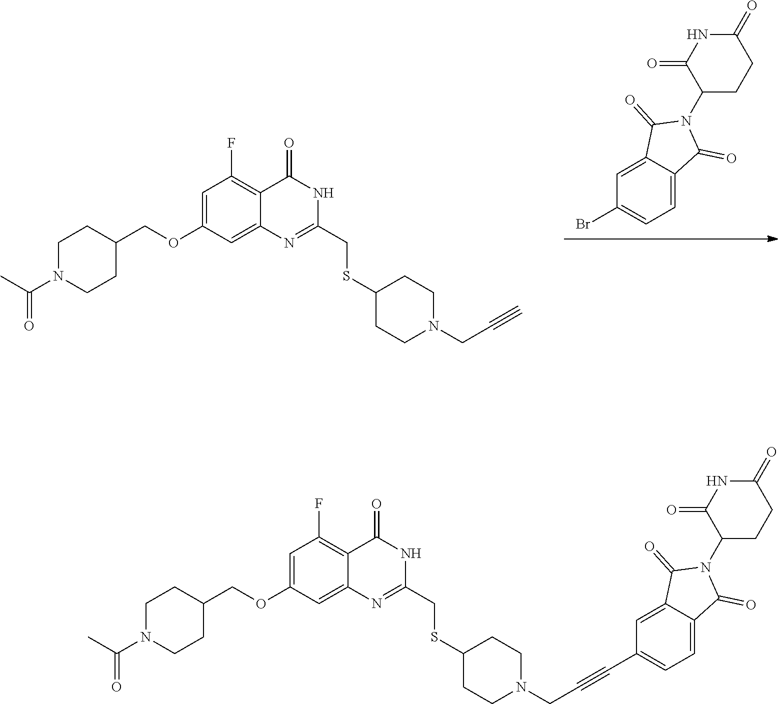

A solution of 7-((1-acetylpiperidin-4-yl)methoxy)-5-fluoro-2-(((1-(prop-2-yn-1-yl)piperidin-4-yl)thio)methyl)quinazolin-4(3H)-one (200 mg, 0.41 mmol, 1.0 equiv), 5-bromo-2-(2,6-dioxopiperidin-3-yl)isoindoline-1,3-dione (139 mg, 0.41 mmol, 1.0 equiv), TEA (83 mg, 0.82 mmol, 2.0 equiv) and Pd(PPh₃)₂Cl₂ (29 mg, 0.041 mmol, 0.10 equiv) in DMSO (2 mL) was stirred for 2 hours at 80° C. After concentration, the residue was purified by C18 reverse phase chromatography eluting with water/CH₃CN (56:44) and further purified by prep-HPLC with the following condition: (Column: XSelect CSH Fluoro Phenyl, 30*150 mm, 5 m; Mobile Phase A: water (10 mmol/L NH₄HCO₃), Mobile Phase B: ACN; Flow rate: 60 mL/min; Gradient: 28% B to 49% B in 9.5 min; detector: 254/220 nm; RT (min): 8.55) to afford 5-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-2-(2,6-dioxopiperidin-3-yl)isoindoline-1,3-dione (24 mg, 8% yield) as a white solid. LCMS (ESI, m/z): 743.30 [M+H]*; 1H NMR (400 MHz, DMSO-d6) δ 12.16 (s, 1H), 11.15 (s, 1H), 7.89-7.75 (m, 2H), 7.88 (s, 1H), 6.89 (s, 1H), 6.87 (s, 1H), 5.17 (dd, J=12.9, 5.6 Hz, 1H), 4.38 (d, J=13.0 Hz, 1H), 3.97 (d, J=6.5 Hz, 2H), 3.80-3.70 (m, 1H), 3.60 (d, J=16.0 Hz, 4H), 3.01-2.89 (m, 1H), 2.84-2.68 (m, 4H), 2.61-2.54 (m, 2H), 2.28-2.19 (m, 2H), 2.01-1.75 (m, 8H), 1.76-1.59 (m, 2H), 1.54-1.30 (m, 2H), 1.22-1.10 (m, 2H).

Examples 35-38 were synthesized according to the procedure described for the synthesis of 5-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-2-(2,6-dioxopiperidin-3-yl)isoindoline-1,3-dione as using appropriate building blocks and modified reaction conditions (such as reagents, reagent ratio, temperature, and reaction time) and purification conditions as needed.

| Ex- | | |
|---|---|---|
| ample | Structure | Analytical Data |
| 35 | 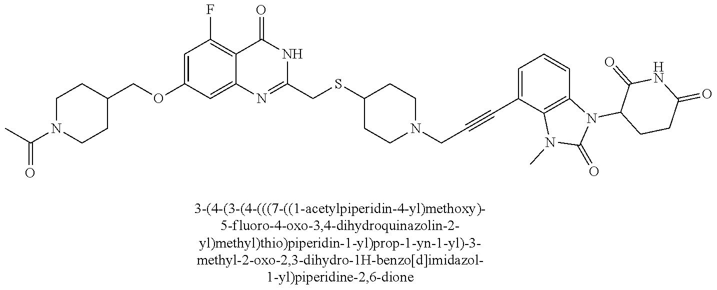 [SMILES: CC(=O)N1CCC(COc2cc(F)c3c(=O)[nH]c(CSC4CCN(CC#Cc5cccc6c5n(C)c(=O)n6C5CCC(=O)NC5=O)CC4)nc3c2)CC1] | LCMS (ESI, m/z): 744.30 [M + H]⁺; ¹H NMR (400 MHz, DMSO-d₆) δ 12.08 (s, 1H), 1.11 (s, 1H), 7.15 (d, J = 7.8 Hz, 1H), 7.10 (d, J = 7.6 Hz, 1H), 7.01 (t, J = 7.8 Hz, 1H), 6.88 (d, J = 12.3 Hz, 2H), 5.40 (dd, J = 12.7, 5.4 Hz, 1H), 4.39 (d, J = 12.9 Hz, 1H), 3.98 (d, J = 6.3 Hz, 2H), 3.83 (d, J = 13.6 Hz, 1H), 3.63 (s, 3H), 3.61 (d, J = 8.7 Hz, 2H), 3.04 (t, J = 12.5 Hz, 1H), 2.94-2.75 (m, 4H), 2.74-2.57 (m, 3H), |
| | | 2.41-2.29 (m, 2H), 2.10- |
| | | 1.95 (m, 7H), 1.79-1.65 |
| | | (m, 2H), 1.55-1.47 (m, |
| | | 2H), 1.27-1.05 (m, 2H). |
| 36 | 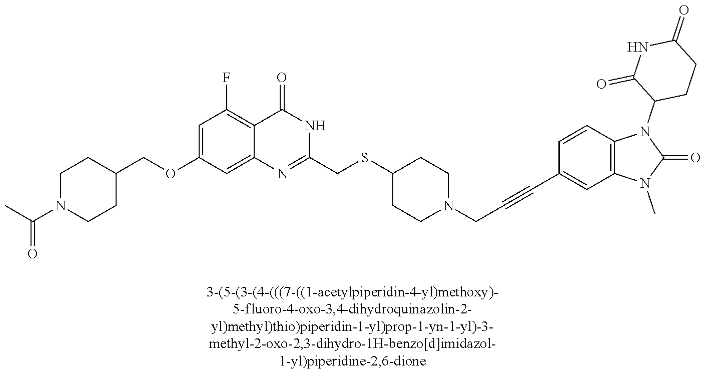 [SMILES: CC(=O)N1CCC(COc2cc(F)c3c(=O)[nH]c(CSC4CCN(CC#Cc5ccc6c(c5)n(C)c(=O)n6C5CCC(=O)NC5=O)CC4)nc3c2)CC1] | LCMS (ESI, m/z): 744.35 [M + H]⁺; ¹H NMR (400 MHz, DMSO-d₆) δ 12.17 (s, 1H), 11.12 (s, 1H), 8.35 (s, 2H, HCOOH), 7.28 (s, 1H), 7.12 (s, 2H), 6.90 (d, J = 12.0 Hz, 2H), 5.39 (dd, J = 12.8, 5.4 Hz, 1H), 4.39 (d, J = 13.1 Hz, 1H), 3.98 (d, J = 6.4 Hz, 2H), 3.83 (d, J = 13.5 Hz, 2H), 3.62 (s, 2H), 3.48 (s, 2H), 3.03 (t, J = 12.4 Hz, 2H), 2.90-2.79 (m, 5H), 2.78- 2.55 (m, 3H), 2.26 (t, J = 10.3 Hz, 2H), 2.07-1.93 (m, 7H), 1.81-1.69 (m, 2H), 1.60-1.51 (m, 2H), 1.29-1.08 (m, 1H). |
| 37 | 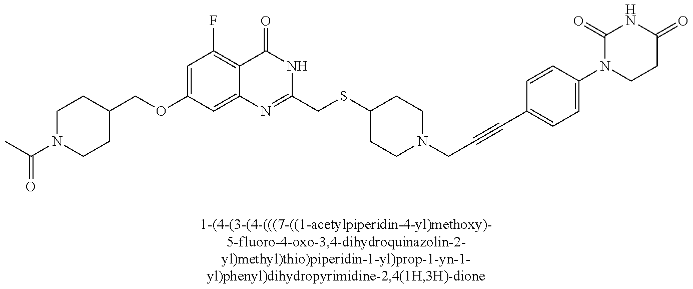 [SMILES: CC(=O)N1CCC(COc2cc(F)c3c(=O)[nH]c(CSC4CCN(CC#Cc5ccc(N6CCC(=O)NC6=O)cc5)CC4)nc3c2)CC1] | LCMS (ESI, m/z): 675.45 [M + H]⁺; ¹H NMR (400 MHz, DMSO-d₆) δ 11.92 (s, 1H), 10.42 (s, 1H), 7.43 (d, J = 8.6 Hz, 2H), 7.33 (d, J = 8.5 Hz, 2H), 6.88 (d, J = 11.0 Hz, 2H), 4.39 (d, J = 12.9 Hz, 1H), 3.98 (d, J = 6.4 Hz, 2H), 3.87-3.77 (m, 3H), 3.61 (s, 2H), 3.49 (s, 2H), 3.04 (t, J = 12.2 Hz, 1H), 2.85-2.79 (m, 3H), 2.72 (t, J = 6.6 Hz, 2H), 2.60-2.52 (m, 1H), 2.26 (t, J = 10.4 Hz, 2H), 2.05-1.89 (m, 6H), 1.80- |
| | | 1.70 (m, 2H), 1.57-1.44 |
| | | (m, 2H), 1.30-1.19 (m, |
| | | 1H), 1.17-1.04 (m, 1H). |
| 38 | 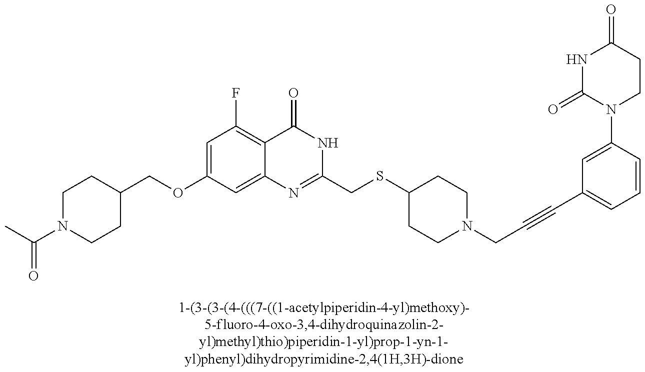 [SMILES: CC(=O)N1CCC(COc2cc(F)c3c(=O)[nH]c(CSC4CCN(CC#Cc5cccc(N6CCC(=O)NC6=O)c5)CC4)nc3c2)CC1] | LCMS (ESI, m/z): 675.25 [M + H]⁺; ¹H NMR (400 MHz, DMSO-d₆) δ 12.13 (s, 1H), 10.39 (s, 1H), 7.43- 7.24 (m, 4H), 6.89 (s, 1H), 6.88 (s, 1H), 4.39 (d, J = 12.9 Hz, 1H), 3.98 (d, J = 6.4 Hz, 2H), 3.87-3.76 (m, 3H), 3.61 (s, 2H), 3.50 (s, 2H), 3.10-2.98 (m, 1H), 2.90-2.78 (m, 3H), 2.79- 2.70 (m, 2H), 2.61-2.52 (m, 1H), 2.31-2.21 (m, 2H), 2.02-1.92 (m, 6H), 1.84-1.76 (m, 2H), 1.57- 1.44 (m, 2H), 1.30-1.03 (m, 2H). |

### Example 39: Synthesis of 4-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione

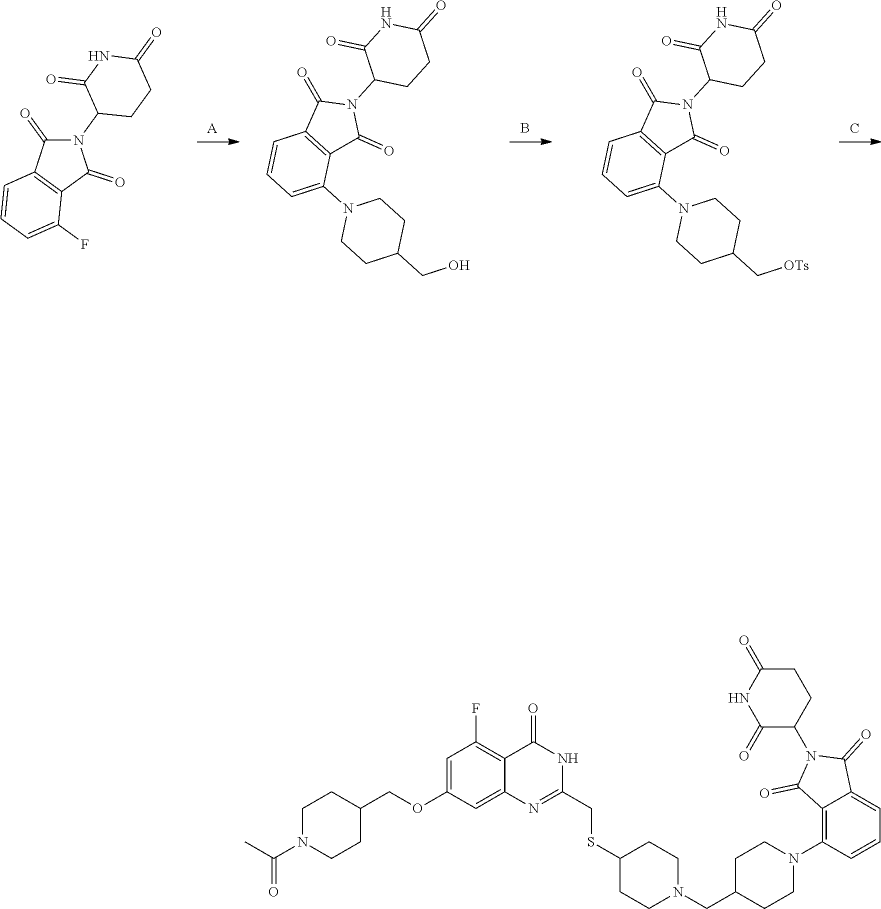

### Step A

A solution of 2-(2,6-dioxopiperidin-3-yl)-4-fluoroisoindole-1,3-dione (5.0 g, 18 mmol, 1.0 equiv) piperidin-4-ylmethanol (2.50 g, 21.7 mmol, 1.2 equiv) and DIEA (7.02 g, 54.3 mmol, 3.0 equiv) in NMP (100 mL) was stirred for 4 hours at 120° C. The resulting mixture was diluted with water and extracted with EtOAc (3×150 mL). The combined organic layers were washed with brine (3×200 mL), dried over anhydrous Na₂SO₄, filtered, and the filtrate was concentrated under reduced pressure. The residue was purified by silica gel column chromatography, eluting with PE/EtOAc (2:3) to afford 2-(2,6-dioxopiperidin-3-yl)-4-(4-(hydroxymethyl)piperidin-1-yl)isoindoline-1,3-dione (6.7 g, 99%) as a yellow oil. LCMS (ESI, m/z): 372.15 [M+H]⁺.

### Step B

A solution of 2-(2,6-dioxopiperidin-3-yl)-4-(4-(hydroxymethyl)piperidin-1-yl)isoindole-1,3-dione (500 mg, 1.35 mmol, 1.0 equiv), p-toluenesulfonyl chloride (308 mg, 1.25 mmol, 1.2 equiv), TEA (409 mg, 4.04 mmol, 3.0 equiv) and DMAP (82 mg, 0.67 mmol, 0.5 equiv) in DCM (40 mL) was stirred for 16 hours. The resulting mixture was concentrated under vacuum and the residue was purified by silica gel column chromatography, eluting with PE/EtOAc (3:1) to afford (1-(2-(2,6-dioxopiperidin-3-yl)-1,3-dioxoisoindolin-4-yl)piperidin-4-yl)methyl 4-methylbenzenesulfonate (620 mg, 88%) as a yellow solid. LCMS (ESI, m/z): 526.15 [M+H]⁺.

### Step C

A solution of (1-(2-(2,6-dioxopiperidin-3-yl)-1,3-dioxoisoindolin-4-yl)piperidin-4-yl)methyl 4-methylbenzenesulfonate (100 mg, 0.19 mmol, 1.0 equiv), 7-[(1-acetylpiperidin-4-yl)methoxy]-5-fluoro-2-[(piperidin-4-ylsulfanyl)methyl]-3H-quinazolin-4-one (85 mg, 0.19 mmol, 1.0 equiv), KI (16 mg, 0.095 mmol, 0.5 equiv) and DIEA (74 mg, 0.57 mmol, 3.0 equiv) in ACN (5 mL) was stirred for 1 hour at 60° C. The resulting mixture was concentrated under vacuum and the residue was purified by reverse-phase flash chromatography with the following conditions: (column, C18 silica gel; mobile phase, MeCN in water (10 mmol/L NH₄HCO₃), 5% to 95% gradient in 20 min; detector, UV 254 nm) and further purified by prep-HPLC with the following condition: Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 20% B to 45% B in 9.5 min; detector: 254/220 nm; RT (min): 9.78) to afford 4-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione (17 mg, 11%) as a yellow solid. LCMS (ESI, m/z): 802.35 [M+H]⁺; ¹H NMR (300 MHz, DMSO-d₆) δ 12.15 (s, 1H), 11.05 (s, 1H), 7.56 (dd, J=8.6, 6.9 Hz, 1H), 7.16-7.04 (m, 2H), 6.95-6.84 (m, 2H), 5.06 (dd, J=12.6, 5.6 Hz, 1H), 4.40 (d, J=13.0 Hz, 1H), 4.00 (d, J=6.3 Hz, 2H), 3.84 (d, J=13.4 Hz, 1H), 3.74-3.54 (m, 4H) 3.53-3.42 (m, 1H), 3.11-3.00 (m, 1H) 2.94-2.52 (m, 8H), 2.39-2.34 (m, 2H), 2.34-2.12 (m, 1H), 2.12-1.68 (m, 11H), 1.63-1.20 (m, 7H), 1.01-0.89 (m, 1H).

Example 40 was synthesized according to the procedure described for the synthesis of 4-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione using appropriate building blocks and modified reaction conditions (such as reagents, reagent ratio, temperature, and reaction time) and purification conditions as needed.

| Ex- | | |
|---|---|---|
| ample | Structure | Analytical Data |
| 40 | 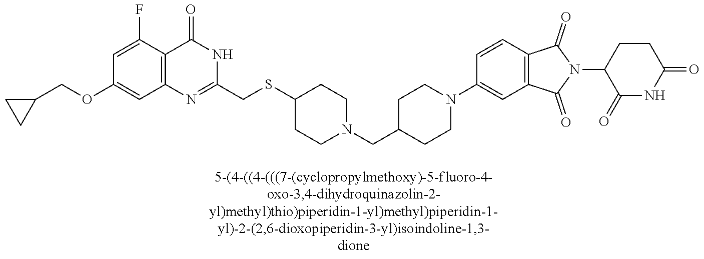 [SMILES: O=C1CCC(N2C(=O)c3ccc(N4CCC(CN5CCC(SCc6nc7cc(OCC8CC8)cc(F)c7c(=O)[nH]6)CC5)CC4)cc3C2=O)C(=O)N1] | LCMS (ESI, m/z): 717.35 [M + H]⁺. ¹H NMR (400 MHz, DMSO-d₆) δ 12.03 (s, 1H), 11.07 (s, 1H), 7.64 (dd, J = 8.4, 3.6 Hz, 1H), 7.30-.29 (m, 1H), 6.90- 6.85 (m, 3H), 5.08-5.05 (m, 1H), 4.01-3.96 (m, 3H), 3.60 (s, 2H), 3.50 (t, J = 8.0 Hz, 1H), 3.39- 3.32 (m, 1H), 2.95-2.89 (m, 2H), 2.86-2.73 (m, 3H), 2.55-2.50 (m, 2H), 2.33-2.32 (m, 1H), 2.12- 2.10 (m, 2H), 2.03-1.90 |
| | | (m, 5H), 1.84-1.53 (m, |
| | | 3H), 1.51-1.44 (m, 2H), |
| | | 1.30-1.21 (m, 1H), 1.12- |
| | | 1.01 (m, 1H), 0.61-0.59 |
| | | (m, 2H), 0.38-0.31 (m, |
| | | 2H). |

### Example 41: Synthesis of 3-(4-(3-((4-(((7-((1-acetylpiperidin-4-yl)methoxy)-5-fluoro-4-oxo-3,4-dihydroquinazolin-2-yl)methyl)thio)piperidin-1-yl)methyl)azetidin-1-yl)-1-oxoisoindolin-2-yl)piperidine-2,6-dione

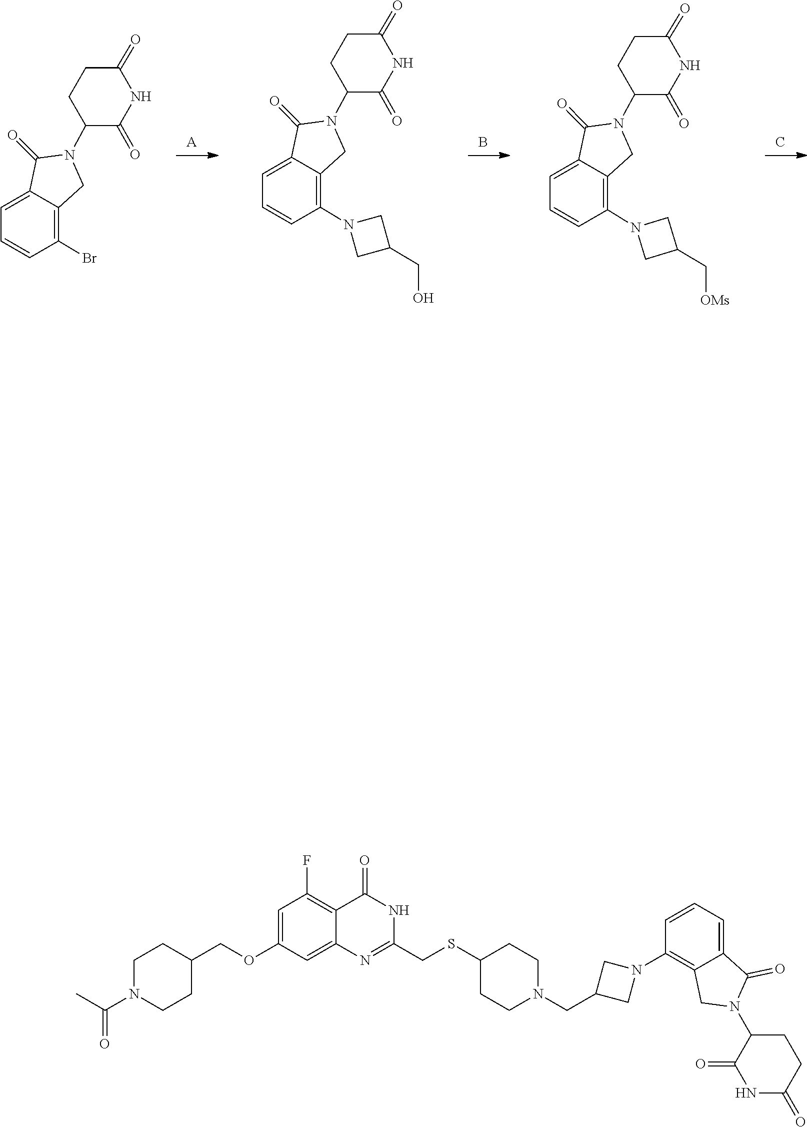

### Step A

A solution of 3-(4-bromo-1-oxoisoindolin-2-yl)piperidine-2,6-dione (160 mg, 0.49 mmol, 1.0 equiv), azetidin-3-yl methanol hydrochloride (73 mg, 0.59 mmol, 1.2 equiv), Pd-PEPPSI-IPentCl2-methylpyridine (o-picoline) (42 mg, 0.050 mmol, 0.1 equiv) and Cs₂CO₃ (323 mg, 0.99 mmol, 2.0 equiv) in dioxane (10 mL) was stirred overnight at 100° C. under nitrogen atmosphere. The resulting mixture was filtered, the filter cake was washed with DCM (3×30 mL). The filtrate was concentrated under reduced pressure and the residue purified by reversed-phase flash chromatography with the following conditions: (column, C18 silica gel; mobile phase, MeCN in water, 5% to 20% gradient in 10 min; detector, UV 254 nm) to afford 3-(4-(3-(hydroxymethyl)azetidin-1-yl)-1-oxoisoindolin-2-yl)piperidine-2,6-dione (95 mg, 58%) as a white solid. LCMS (ESI, m/z): 330.15 [M+H]⁺.

### Step B

To a stirred solution of 3-(4-(3-(hydroxymethyl)azetidin-1-yl)-1-oxoisoindolin-2-yl)piperidine-2,6-dione (90 mg, 0.27 mmol, 1.0 equiv), TEA (138 mg, 1.37 mmol, 5.0 equiv) and DMAP (17 mg, 0.14 mmol, 0.5 equiv) in DCM (25 mL) was added methanesulfonic anhydride (143 mg, 0.82 mmol, 3.0 equiv) portion-wise at 0° C. then stirred for overnight at room temperature. The resulting mixture was concentrated under reduced pressure and the residue purified by reversed-phase flash chromatography with the following conditions: (column, C18 silica gel; mobile phase, MeCN in water, 5% to 30% gradient in 10 min; detector, UV 254 nm) to afford (1-(2-(2,6-dioxopiperidin-3-yl)-1-oxoisoindolin-4-yl)azetidin-3-yl)methyl methanesulfonate (78.2 mg, 70%) as a white solid. LCMS (ESI, m/z): 408.15 [M+H]⁺.

### Step C

A mixture of (1-(2-(2,6-dioxopiperidin-3-yl)-1-oxoisoindolin-4-yl)azetidin-3-yl)methyl methanesulfonate (61 mg, 0.15 mmol, 1.0 equiv), 7-((1-acetylpiperidin-4-yl)methoxy)-5-fluoro-2-((piperidin-4-ylthio)methyl)quinazolin-4(3H)-one (67 mg, 0.15 mmol, 1.0 equiv), DIEA (96 mg, 0.75 mmol, 5.0 equiv) and KI (50 mg, 0.29 mmol, 2.0 equiv) in ACN (30 mL) was stirred overnight at 80° C. The resulting mixture was concentrated under reduced pressure. The residue was purified by C18 reverse phase chromatography eluting with water/CH₃CN (67:33) and further purified by prep-HPLC with the following condition (Column: XSelect CSH Prep C18 OBD Column, 19*250 mm, 5 m; Mobile Phase A: Water (10 mmol/L NH₄HCO₃), Mobile Phase B: ACN; Flow rate: 20 mL/min; Gradient: 35% B to 40% B in 8 min, 40% B; Wave Length: 254 nm; RT (min): 8) to afford 3-(4-(3-((4-(((7-((1-acetylpiperidin-4-yl)methoxy)-5-fluoro-4-oxo-3,4-dihydroquinazolin-2-yl)methyl)thio)piperidin-1-yl)methyl)azetidin-1-yl)-1-oxoisoindolin-2-yl)piperidine-2,6-dione (7 mg, 6%) as a white solid. LCMS (ESI, m/z): 760.30 [M+H]⁺. ¹H NMR (300 MHz, DMSO-d₆) δ 12.11 (s, 1H), 10.95 (s, 1H), 7.31 (t, J=7.7 Hz, 1H), 7.04 (d, J=7.3 Hz, 1H), 6.97-6.80 (m, 2H), 6.54 (d, J=7.9 Hz, 1H), 5.08 (dd, J=13.1, 5.2 Hz, 1H), 4.48-4.23 (m, 3H), 4.21-3.94 (m, 4H), 3.91-3.80 (m, 1H), 3.79-3.46 (m, 5H), 2.92-2.72 (m, 4H), 2.67-2.59 (m, 1H), 2.58-2.54 (m, 2H), 2.10-1.82 (m, 8H), 1.82-1.71 (m, 2H), 1.47-1.36 (m, 2H), 1.32-1.09 (m, 5H), 0.90-0.80 (m, 1H).

### Example 42: Synthesis of 3-((4-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)phenyl)amino)piperidine-2,6-dione

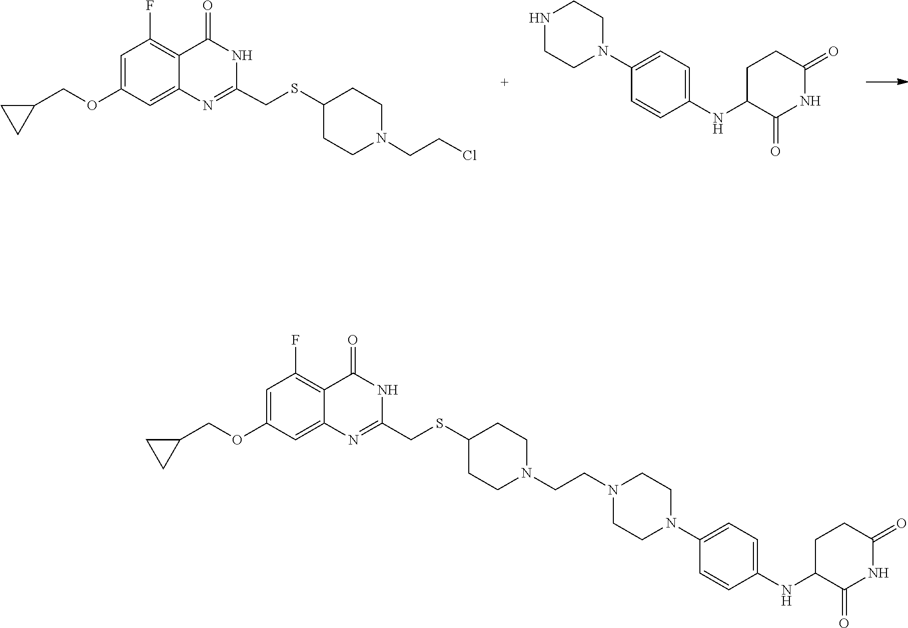 [SMILES: O=c1[nH]c(CSC2CCN(CCCl)CC2)nc2cc(OCC3CC3)cc(F)c12 ; O=C1CCC(Nc2ccc(N3CCNCC3)cc2)C(=O)N1 ; O=C1CCC(Nc2ccc(N3CCN(CCN4CCC(SCc5nc6cc(OCC7CC7)cc(F)c6c(=O)[nH]5)CC4)CC3)cc2)C(=O)N1]

A solution of 2-(((1-(2-chloroethyl)piperidin-4-yl)thio)methyl)-7-(cyclopropylmethoxy)-5-fluoroquinazolin-4(3H)-one (173 mg, 0.406 mmol, 1 equiv) in DMSO (5 mL) was treated with 3-((4-(piperazin-1-yl)phenyl)amino)piperidine-2,6-dione (117 mg, 0.406 mmol, 1 equiv) and DIEA (210 mg, 1.62 mmol, 4 equiv) for 3 hours at 80° C. The mixture was concentrated and the residue purified by reverse-phase flash chromatography with the following conditions: column, C18 silica gel; mobile phase, MeCN in water (10 mmol/L NH₄HCO₃), 10% to 41% gradient in 20 min; detector, UV 254 nm. This afforded the product (85 mg) as a brown solid. The product was purified by Prep-HPLC with the following conditions (Column: XBridge Prep OBD C18 Column, 30*150 mm, 5 m; Mobile Phase A: water (10 mmol/L NH₄HCO₃), Mobile Phase B: ACN; Flow rate: 60 mL/min; Gradient: 24% B to 34% B in 10 min, 34% B; Wavelength: 220/254 nm; RT(min): 11.80) to afford 3-((4-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)phenyl)amino)piperidine-2,6-dione (49.2 mg, 18%) as a brown solid. LCMS (ESI, m/z): 678.20 [M+H]⁺. 1H NMR (300 MHz, DMSO-d6) δ 12.02 (br, 1H), 10.77 (s, 1H), 6.90 (s, 1H), 6.85 (s, 1H), 6.74 (d, J=8.5 Hz, 2H), 6.60 (d, J=8.5 Hz, 2H), 5.38 (d, J=7.2 Hz, 1H), 4.25-4.15 (m, 1H), 3.96 (d, J=7.1 Hz, 2H), 3.70-3.59 (m, 5H), 2.91 (s, 3H), 2.85-2.65 (m, 3H), 2.62-2.55 (m, 2H), 2.41 (s, 4H), 2.05-1.84 (m, 5H), 1.51-1.35 (m, 2H), 1.30-1.15 (m, 2H), 0.63-0.52 (m, 2H), 0.41-0.31 (m, 2H).

Examples 43-49 were synthesized according to the procedure described for the synthesis 3-((4-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)phenyl)amino)piperidine-2,6-dione Example 42 using appropriate building blocks and modified reaction conditions (such as reagents, reagent ratio, temperature, and reaction time) and purification conditions as needed.

| Ex- | | |
|---|---|---|
| am- | | |
| ple | Structure | Analytical Data |
| 43 | 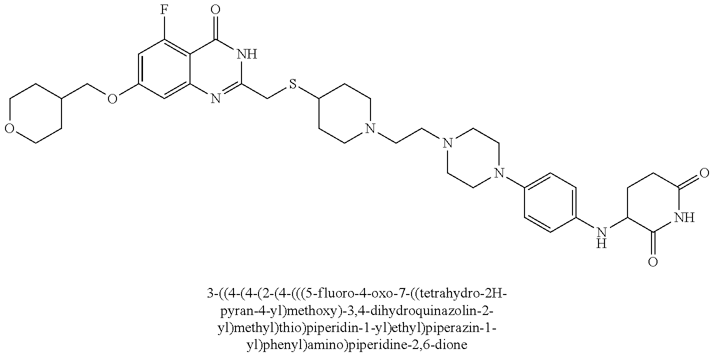 [SMILES: O=C1CCC(Nc2ccc(N3CCN(CCN4CCC(SCc5nc6cc(OCC7CCOCC7)cc(F)c6c(=O)[nH]5)CC4)CC3)cc2)C(=O)N1] | LCMS (ESI, m/z): 722.35 [M + H]⁺. 1H NMR (400 MHz, DMSO-d6) δ 10.74 (s, 1H), 6.88 (d, J = 10.1 Hz, 2H), 6.74 (d, J = 8.5 Hz, 2H), 6.61 (d, J = 8.5 Hz, 2H), 5.36 (d, J = 16 Hz, 1H), 4.23-4.14 (m, 2H), 3.99 (d, J = 6.4 Hz, 3H), 3.90-3.85 (m, 4H), 3.70-3.50 (m, 4H), 3.30-3.16 (m, 2H), 2.94-2.86 (m, 4H), 2.82-2.69 (m, 3H), 2.41 (s, 3H), 2.15-1.99 (m, 5H), 1.90-1.76 (m, 2H), 1.86-1.81 (m, 1H), 1.70-1.61 (m, 2H), 1.48-1.36 (m, 3H), 1.36-1.32 (m, 2H). |
| 44 | 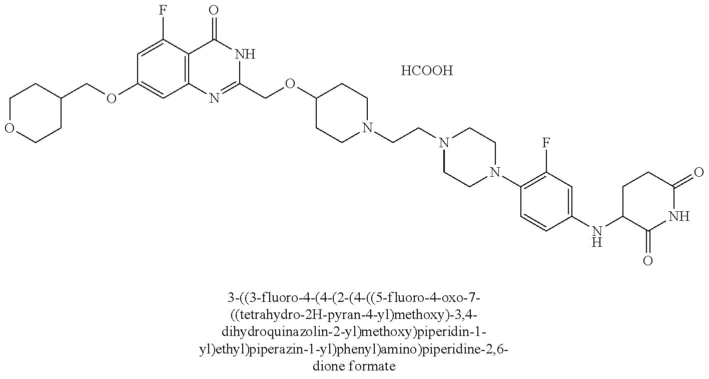 [SMILES: O=C1CCC(Nc2ccc(N3CCN(CCN4CCC(OCc5nc6cc(OCC7CCOCC7)cc(F)c6c(=O)[nH]5)CC4)CC3)c(F)c2)C(=O)N1 ; "HCOOH"] | LCMS (ESI, m/z): 724.40 [M + H]+. 1H NMR (400 MHz, DMSO-d6) δ 12.00 (br, 1H), 10.77 (s, 1H), 8.20 (s, 1H), 6.97-6.87 (m, 2H), 6.82 (t, J = 9.3 Hz, 1H), 6.51 (dd, J = 15.1, 2.5 Hz, 1H), 6.42 (dd, J = 8.7, 2.6 Hz, 1H), 5.80 (d, J = 7.6 Hz, 1H), 4.35 (s, 2H), 4.30-4.20 (m, 1H), 3.99 (d, J = 6.4 Hz, 2H), 3.96-3.84 (m, 2H), 3.55-3.45 (m, 1H), 3.42-3.31 (m, 2H), 2.90-2.67 (m, 7H), 2.62-2.52 (m, 5H), 2.50-2.40 (m, 4H), 2.20 (d, J = 11.3 Hz, 2H), 2.20-2.00 (m, 4H), 1.99-1.79 (m, 3H), 1.71-1.65 (m, |
| | | 2H), 1.60-1.40 (m, |
| | | 2H), 1.38-1.30 (m, |
| | | 2H). |
| 45 | 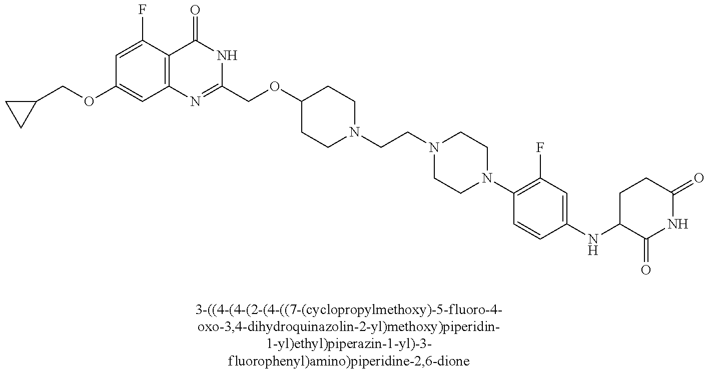 [SMILES: O=C1CCC(Nc2ccc(N3CCN(CCN4CCC(OCc5nc6cc(OCC7CC7)cc(F)c6c(=O)[nH]5)CC4)CC3)c(F)c2)C(=O)N1] | LCMS (ESI, m/z): 680.25 [M + H]+, 1H NMR (300 MHz, DMSO-d6) δ 11.93 (br, 1H), 10.76 (s, 1H), 6.95-6.76 (m, 3H), 6.6-6.4 (m, 2H), 5.78 (d, J = 7.6 Hz, 1H), 4.40 (s, 2H), 4.30-4.20 (m, 1H), 3.98 (d, J = 7.2 Hz, 2H), 3.6-3.3 (m, 2H), 2.89-2.65 (m, 7H), 2.60-2.50 (m, 5H), 2.18-2.05 (m, 4H), 1.95- 1.78 (m, 3H), 1.59- 1.41 (m, 2H), 1.36- 1.10 (m, 2H), 0.66-0.52 (m, 2H), 0.41-0.30 (m, 2H). |
| 46 | 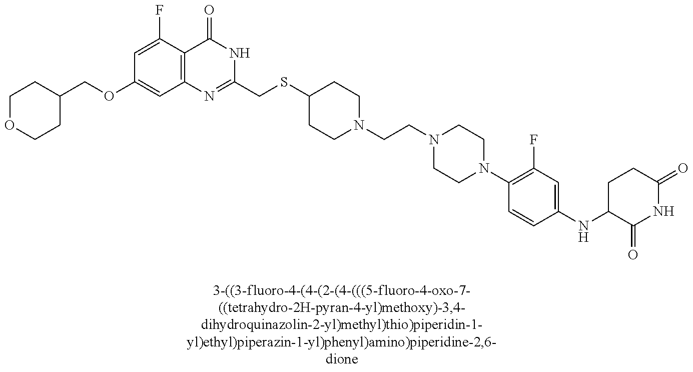 [SMILES: O=C1CCC(Nc2ccc(N3CCN(CCN4CCC(SCc5nc6cc(OCC7CCOCC7)cc(F)c6c(=O)[nH]5)CC4)CC3)c(F)c2)C(=O)N1] | LCMS (ESI, m/z): 740.25 [M + H]⁺. H- NMR: 1H NMR (400 MHz, DMSO-d6) δ 12.11 (br, 1H), 10.77 (s, 1H), 7.19-6.62 (m, 3H), 6.60-6.25 (m, 2H), 5.80 (d, J = 7.6 Hz, 1H), 4.30-4.17 (m, 1H), 3.99 (d, J = 6.4 Hz, 2H), 3.88 (dd, J = 10.3, 4.3 Hz, 2H), 3.60 (s, 2H), 3.39-3.34 (m, 2H), 3.31-3.29 (m, 2H), 2.97-2.66 (m, 8H), 2.64-2.52 (m, 3H), 2.48-2.20 (m, 4H), 2.17-1.96 (m, 4H), 1.94-1.79 (m, 3H), 1.72-1.62 (m, 2H), 1.61-1.16 (m, 4H). |
| 47 | 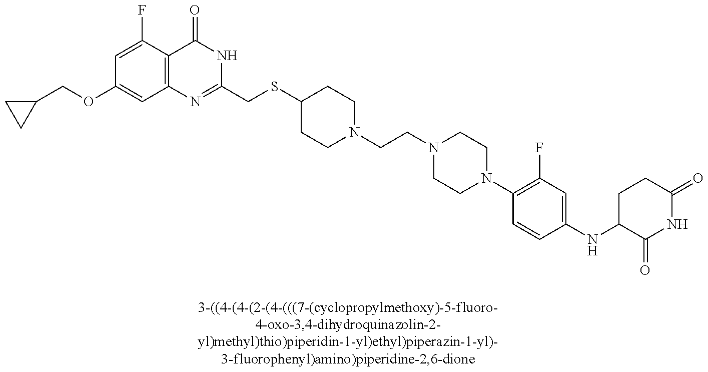 [SMILES: O=C1CCC(Nc2ccc(N3CCN(CCN4CCC(SCc5nc6cc(OCC7CC7)cc(F)c6c(=O)[nH]5)CC4)CC3)c(F)c2)C(=O)N1] | LCMS (ESI, m/z): 696.25 [M + H]⁺. 1H NMR (300 MHz, DMSO-d6) δ 12.10 (br, 1H), 10.75 (s, 1H), 6.91-6.79 (m, 3H), 6.50 (dd, J = 15.1, 2.5 Hz, 1H), 6.41 (dd, J = 8.7, 2.6 Hz, 1H), 5.77 (d, J = 7.6 Hz, 1H), 4.31-4.20 (m, 1H), 3.96 (d, J = 7.1 Hz, 2H), 3.59 (s, 2H), 2.90-2.72 (m, 8H), 2.70-2.60 (m, 2H), 2.50-2.40 (m, 5H), 2.20-2.03 (m, 3H), 1.95-1.74 (m, 3H), 1.53-1.34 (m, 2H), 1.33-1.14 (m, 2H), 0.90-0.80 (m, 1H), 0.65-0.51 (m, 2H), 0.40-0.29 (m, 2H). |
| 48 | 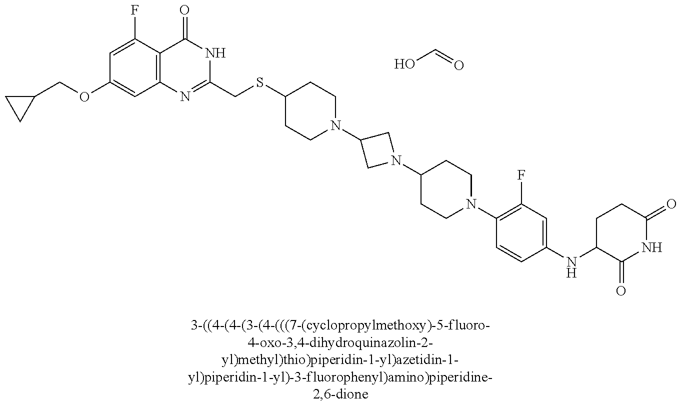 [SMILES: O=C1CCC(Nc2ccc(N3CCC(N4CC(N5CCC(SCc6nc7cc(OCC8CC8)cc(F)c7c(=O)[nH]6)CC5)C4)CC3)c(F)c2)C(=O)N1 ; O=CO] | LCMS (ESI, m/z): 722.80 [M + H]⁺. 1H NMR (300 MHz, DMSO-d6) δ 12.15 (s, 1H), 10.79 (s, 1H), 8.15 (s, 1H) HCOOH, 7.01-6.78 (m, 3H), 6.65-6.45 (m, 1H), 6.43-6.33 (m, 1H), 5.83 (d, J = 7.6 Hz, 1H), 4.35-4.21 (m, 1H), 3.97 (d, J = 6.8 Hz, 2H), 3.78-3.65 (m, 2H), 3.60 (s, 2H), 3.40-3.24 (m, 2H), 3.23-2.90 (m, 4H), 2.89-2.80 (m, 1H), 2.75-2.67 (m, 4H), 2.63-2.56 (m, 4H), 2.15-2.03 (m, 1H), 1.99-1.78 (m, 7H), 1.58-1.31 (m, 4H), 1.29-1.19 (m, 1H), 0.78-0.59 (m, 2H), 0.45-0.36 (m, 2H). |
| 49 | 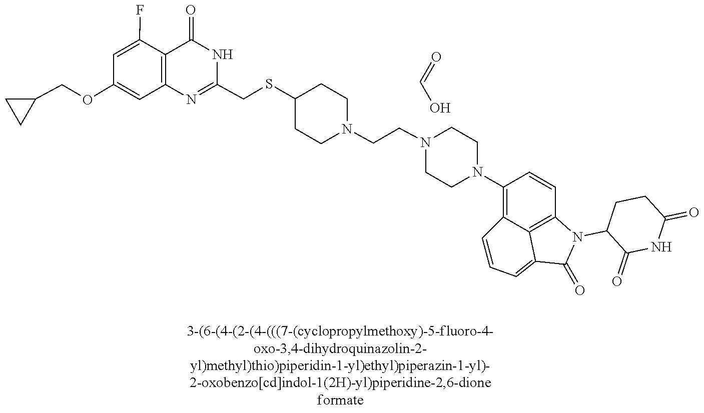 | LCMS (ESI, m/z): 754.25 [M + H]⁺. ¹H NMR (400 MHz, DMSO-d₆) δ 12.15 (s, 1H), 11.11 (s, 1H), 8.24 (d, J = 8.3 Hz, 1H), 8.19 (s, 1H), 8.08 (d, J = 7.0 Hz, 1H), 7.82 (dd, J = 8.2, 7.0 Hz, 1H), 7.05- 6.92 (m, 2H), 6.88 (d, J = 12.7 Hz, 2H), 5.42 (dd, J = 12.8, 5.4 Hz, 1H), 3.97 (d, J = 7.1 Hz, 2H), 3.61 (s, 2H), 3.14- 3.05 (m, 4H), 2.98- 2.89 (m, 1H), 2.89- 2.78 (m, 3H), 2.77- 2.59 (m, 6H), 2.55- 2.41 (m, 4H), 2.12- 2.04 (m, 3H), 1.97- 1.88 (m, 2H), 1.51- 1.39 (m, 2H), 1.30- 1.17 (m, 1H), 0.64- 0.53 (m, 2H), 0.40- 0.31 (m, 2H). |

### Example 50: Synthesis of 3-(5-(4-((4-(((7-(cyclopropylmethoxy)-5-fluoro-4-oxo-3,4-dihydroquinazolin-2-yl)methyl)thio)piperidin-1-yl)methyl)piperidin-1-yl)-1-oxoisoindolin-2-yl)piperidine-2,6-dione

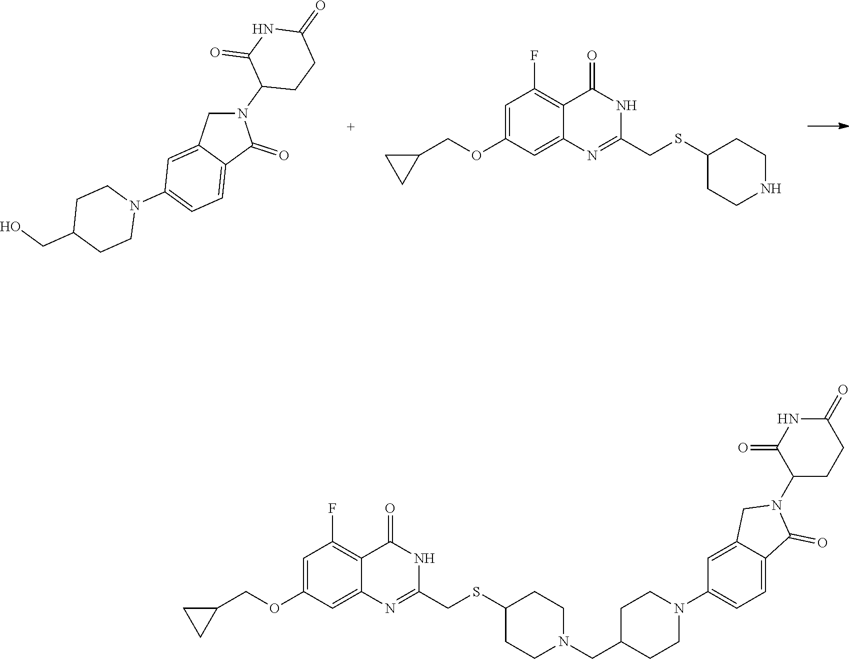 [SMILES: O=C1CCC(N2Cc3cc(N4CCC(CO)CC4)ccc3C2=O)C(=O)N1 ; O=c1[nH]c(CSC2CCNCC2)nc2cc(OCC3CC3)cc(F)c12 ; O=C1CCC(N2Cc3cc(N4CCC(CN5CCC(SCc6nc7cc(OCC8CC8)cc(F)c7c(=O)[nH]6)CC5)CC4)ccc3C2=O)C(=O)N1]

A solution of 3-(5-(4-(hydroxymethyl)piperidin-1-yl)-1-oxoisoindolin-2-yl)piperidine-2,6-dione (120 mg, 0.336 mmol, 1 equiv) and DMP (285 mg, 0.672 mmol, 2 equiv) in DCM (2 mL) was stirred for 1 hour at room temperature. To the above mixture was added 7-(cyclopropylmethoxy)-5-fluoro-2-((piperidin-4-ylthio)methyl)quinazolin-4(3H)-one (123 mg, 0.338 mmol, 1 equiv) and NaBH₃CN (42.4 mg, 0.68 mmol, 2 equiv) at 0° C. The resulting mixture was stirred for 1 hour at room temperature. The residue was purified by reverse-phase flash chromatography with the following conditions: column, C18 silica gel; mobile phase, MeCN in water (10 mmol/L NH₄HCO₃), 10% to 45% gradient in 10 min; detector, UV 254 nm. The crude product (60 mg) was purified by Prep-HPLC with the following conditions (Column: XSelect CSH Prep C18 OBD Column, 19*250 mm, 5 m; Mobile Phase A: Water (10 mmol/L NH₄HCO₃), Mobile Phase B: ACN; Flow rate: 20 mL/min; Gradient: 55% B to 60% B in 8 min, 60% B; Wavelength: 254 nm; RT (min): 8) to afford 3-(5-(4-((4-(((7-(cyclopropylmethoxy)-5-fluoro-4-oxo-3,4-dihydroquinazolin-2-yl)methyl)thio)piperidin-1-yl)methyl)piperidin-1-yl)-1-oxoisoindolin-2-yl)piperidine-2,6-dione (28.6 mg, 12%) as a white solid. LCMS (ESI, m/z): 703.30 [M+H]⁺. 1H NMR (400 MHz, DMSO-d6) δ 12.14 (s, 1H), 10.93 (s, 1H), 7.49 (d, J=8.4 Hz, 1H), 7.03 (d, J=8.1 Hz, 2H), 6.88 (d, J=15.7 Hz, 2H), 5.04 (dd, J=13.3, 5.1 Hz, 1H), 4.32-4.19 (m, 2H), 3.96 (d, J=7.1 Hz, 2H), 3.85 (d, J=12.5 Hz, 2H), 3.60 (s, 2H), 2.93-2.68 (m, 6H), 2.64-2.54 (m, 2H), 2.40-2.32 (m, 1H), 2.11 (d, J=6.6 Hz, 2H), 2.01-1.85 (m, 4H), 1.79-1.65 (m, 3H), 1.52-1.39 (m, 2H), 1.29-1.06 (m, 3H), 0.70-0.60 (d, J=7.8 Hz, 2H), 0.38-0.27 (m, 2H).

### Example 51: Synthesis of 3-((3-fluoro-4-(4-(3-(4-(((5-fluoro-4-oxo-7-((tetrahydro-2H-pyran-4-yl)methoxy)-3,4-dihydroquinazolin-2-yl)methyl)thio)piperidin-1-yl)azetidin-1-yl)piperidin-1-yl)phenyl)amino)piperidine-2,6-dione formate

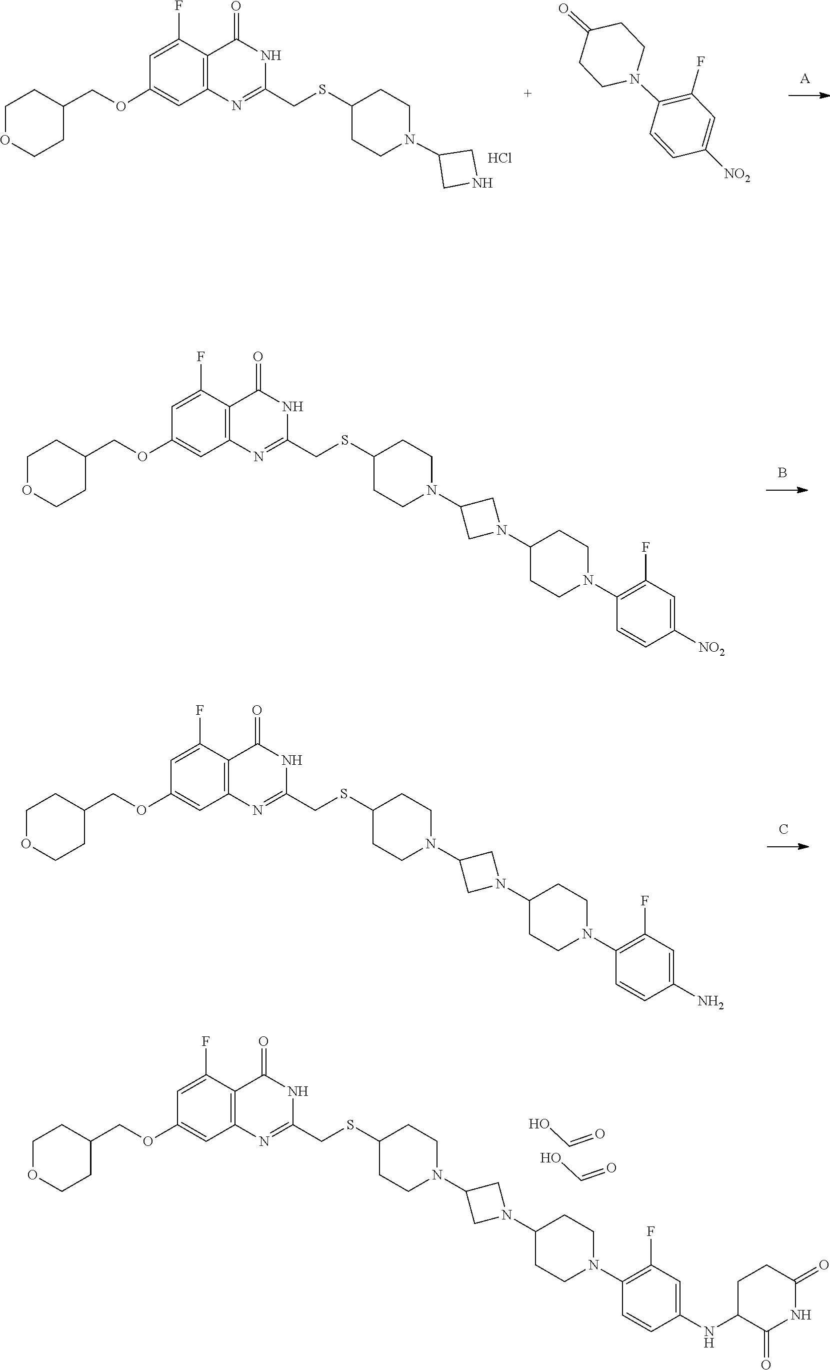

### Step A

A solution of 2-(((1-(azetidin-3-yl)piperidin-4-yl)thio)methyl)-5-fluoro-7-((tetrahydro-2H-pyran-4-yl)methoxy)quinazolin-4(3H)-one hydrochloride (1.04 g, 2.26 mmol, 1 equiv), 1-(2-fluoro-4-nitrophenyl)piperidin-4-one (0.75 g, 3.14 mmol, 1.5 equiv) and STAB (0.89 g, 4.18 mmol, 2 equiv) in DCM (8 mL) was stirred for 2 hours. The mixture was concentrated and the residue was purified by silica gel column chromatography, eluting with DCM/MeOH (91:9) to afford 5-fluoro-2-(((1-(1-(1-(2-fluoro-4-nitrophenyl)piperidin-4-yl)azetidin-3-yl)piperidin-4-yl)thio)methyl)-7-((tetrahydro-2H-pyran-4-yl)methoxy)quinazolin-4(3H)-one (733 mg, 51%) as a yellow solid. LCMS (ESI, m/z): 685.29 [M+H]⁺.

### Step B

A solution of Fe (295 mg, 5.28 mmol, 5 equiv) and NH₄Cl (113 mg, 2.11 mmol, 2 equiv) in EtOH (6 mL) and water (3 mL) was stirred for 10 min at 80° C. followed by the addition of 5-fluoro-2-(((1-(1-(1-(2-fluoro-4-nitrophenyl)piperidin-4-yl)azetidin-3-yl)piperidin-4-yl)thio)methyl)-7-((tetrahydro-2H-pyran-4-yl)methoxy)quinazolin-4(3H)-one (723 mg, 1.06 mmol, 1 equiv) portion-wise at 80° C. The final reaction mixture was irradiated with microwave radiation for 30 min at 80° C. The mixture was concentrated and the residue was purified by silica gel column chromatography, eluting with DCM/MeOH (91:9) to afford 2-(((1-(1-(1-(4-amino-2-fluorophenyl)piperidin-4-yl)azetidin-3-yl)piperidin-4-yl)thio)methyl)-5-fluoro-7-((tetrahydro-2H-pyran-4-yl)methoxy)quinazolin-4(3H)-one (669 mg, 97%) as a yellow solid. LCMS (ESI, m/z): 655.32 [M+H]⁺.

### Step C

A solution of 2-(((1-(1-(1-(4-amino-2-fluorophenyl)piperidin-4-yl)azetidin-3-yl)piperidin-4-yl)thio)methyl)-5-fluoro-7-((tetrahydro-2H-pyran-4-yl)methoxy)quinazolin-4(3H)-one (200 mg, 0.305 mmol, 1 equiv), 3-bromopiperidine-2,6-dione (176 mg, 0.915 mmol, 3 equiv) and NaHCO₃ (180 mg, 2.14 mmol, 7 equiv) in ACN (4 mL) was stirred overnight at 90° C. The mixture was concentrated and the residue was purified by reverse-phase flash chromatography with the following conditions: column, C18 silica gel; mobile phase, MeCN in water (10 mmol/L NH₄HCO₃), 10% to 50% gradient in 20 min; detector, UV 254 nm to afford the crude material as a white solid. The crude product was further purified by Prep-HPLC with the following conditions (Column: Xselect CSH OBD Column 30*150 mm, 5 μm; Mobile Phase A: water (0.1% FA), Mobile Phase B: ACN; Flow rate: 60 mL/min; Gradient: 5% B to 5% B in 2 min, 9% B to 19% B in 10 min; Wavelength: 254 nm/220 nm) to afford 3-((3-fluoro-4-(4-(3-(4-(((5-fluoro-4-oxo-7-((tetrahydro-2H-pyran-4-yl)methoxy)-3,4-dihydroquinazolin-2-yl)methyl)thio)piperidin-1-yl)azetidin-1-yl)piperidin-1-yl)phenyl)amino)piperidine-2,6-dione formate (27.8 mg, 12%) as a white solid. LCMS (ESI, m/z): 766.25 [M+H]⁺. 1H NMR (400 MHz, DMSO-d6) δ 10.78 (s, 1H), 8.22 (s, 2H), 6.93-6.79 (m, 3H), 6.50 (dd, J=15.0, 2.6 Hz, 1H), 6.41 (dd, J=8.7, 2.6 Hz, 1H), 5.79 (d, J=7.6 Hz, 1H), 4.30-4.20 (m, 1H), 3.993 (d, J=6.4 Hz, 2H), 3.92-3.83 (m, 2H), 3.66-3.50 (m, 4H), 3.40-3.28 (m, 2H), 3.11-3.01 (m, 3H), 2.95-2.80 (m, 2H), 2.79-2.70 (m, 1H), 2.68-2.58 (m, 3H), 2.57-2.54 (m, 1H), 2.51-2.50 (m, 3H), 2.40-2.30 (m, 1H), 2.11-1.63 (m, 10H), 1.50-1.30 (m, 6H).

Examples 52-53 were synthesized according to the procedure described for the synthesis 3-((3-fluoro-4-(4-(3-(4-(((5-fluoro-4-oxo-7-((tetrahydro-2H-pyran-4-yl)methoxy)-3,4-dihydroquinazolin-2-yl)methyl)thio)piperidin-1-yl)azetidin-1-yl)piperidin-1-yl)phenyl)amino)piperidine-2,6-dione formate Example 51 using appropriate building blocks and modified reaction conditions (such as reagents, reagent ratio, temperature, and reaction time) and purification conditions as needed.

| Ex- | | |
|---|---|---|
| am- | | |
| ple | Structure | Analytical Data |
| 52 | 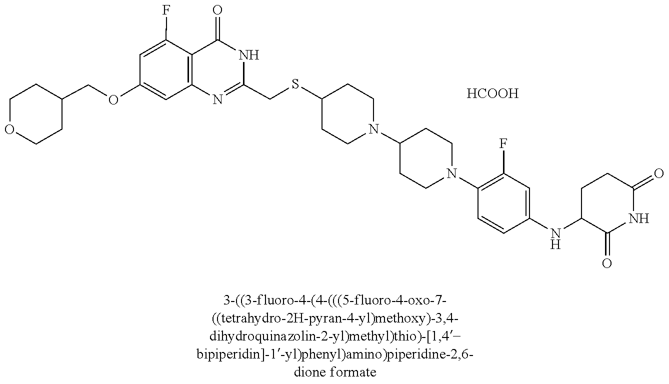 [SMILES: O=C1CCC(Nc2ccc(N3CCC(N4CCC(SCc5nc6cc(OCC7CCOCC7)cc(F)c6c(=O)[nH]5)CC4)CC3)c(F)c2)C(=O)N1 ; "HCOOH"] | LCMS (ESI, m/z): 711.50 [M + H]⁺. 1H NMR (400 MHz, DMSO-d6) δ 12.16 (s, 1H), 10.77 (s, 1H), 8.15 (s, 1H), 6.96-6.78 (m, 2H), 6.76-6.60 (m, 1H), 6.50 (dd, J = 14.9, 2.6 Hz, 1H), 6.41 (dd, J = 8.7, 2.6 Hz, 1H), 5.79 (d, J = 7.7 Hz, 1H), 4.35-4.20 (m, 1H), 4.00 (d, J = 6.4 Hz, 2H), 3.94-3.80 (m, 2H), 3.65 (s, 2H), 3.21-3.10 (m, 3H), 3.00-2.80 (m, 3H), 2.79-2.65 (m, 1H), 2.63-2.52 (m, 3H), 2.42- 2.25 (m, 3H), 2.20- 1.90 (m, 5H), 1.89-1.71 (m, 3H), 1.70-1.63 (m, 2H), 1.62-1.51 (m, 2H), 1.50-1.42 (m, 2H), 1.40- 1.25 (m, 2H). |
| 53 | 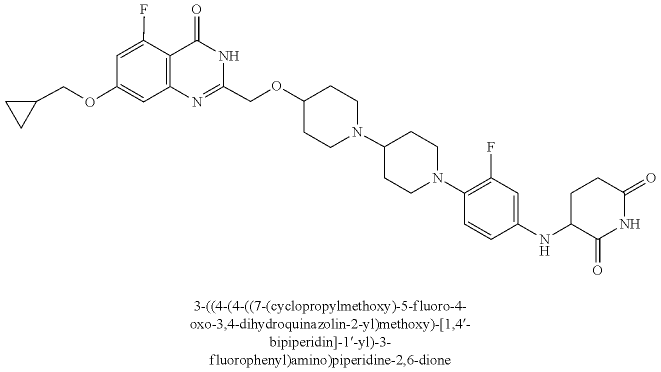 [SMILES: O=C1CCC(Nc2ccc(N3CCC(N4CCC(OCc5nc6cc(OCC7CC7)cc(F)c6c(=O)[nH]5)CC4)CC3)c(F)c2)C(=O)N1] | LCMS (ESI, m/z): 651.30 [M + H]+. 1H NMR (400 MHz, DMSO-d6) δ 10.77 (s, 1H), 6.98-6.86 (m, 2H), 6.82 (t, J = 9.3 Hz, 1H), 6.50 (dd, J = 15.0, 2.6 Hz, 1H), 6.41 (dd, J = 8.6, 2.6 Hz, 1H), 5.79 (d, J = 7.7 Hz, 1H), 4.35 (s, 2H), 4.30-4.18 (m, 1H), 3.97 (d, J = 7.1 Hz, 2H), 3.55-3.45 (m, 1H), 3.16 (d, J = 10.9 Hz, 2H), 2.90- 2.78 (m, 2H), 2.73- 2.62 (m, 1H), 2.60- 2.52 (m, 3H), 2.37-2.19 (m, 3H), 2.08 (m, 1H), 1.94-1.76 (m, 5H), 1.70- 1.46 (m, 4H), 1.30-1.15 (m, 1H), 0.66-0.57 (m, 2H), 0.41-0.30 (m, 2H). |

### Example 54: Synthesis of 3-((4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)amino)piperidine-2,6-dione formate

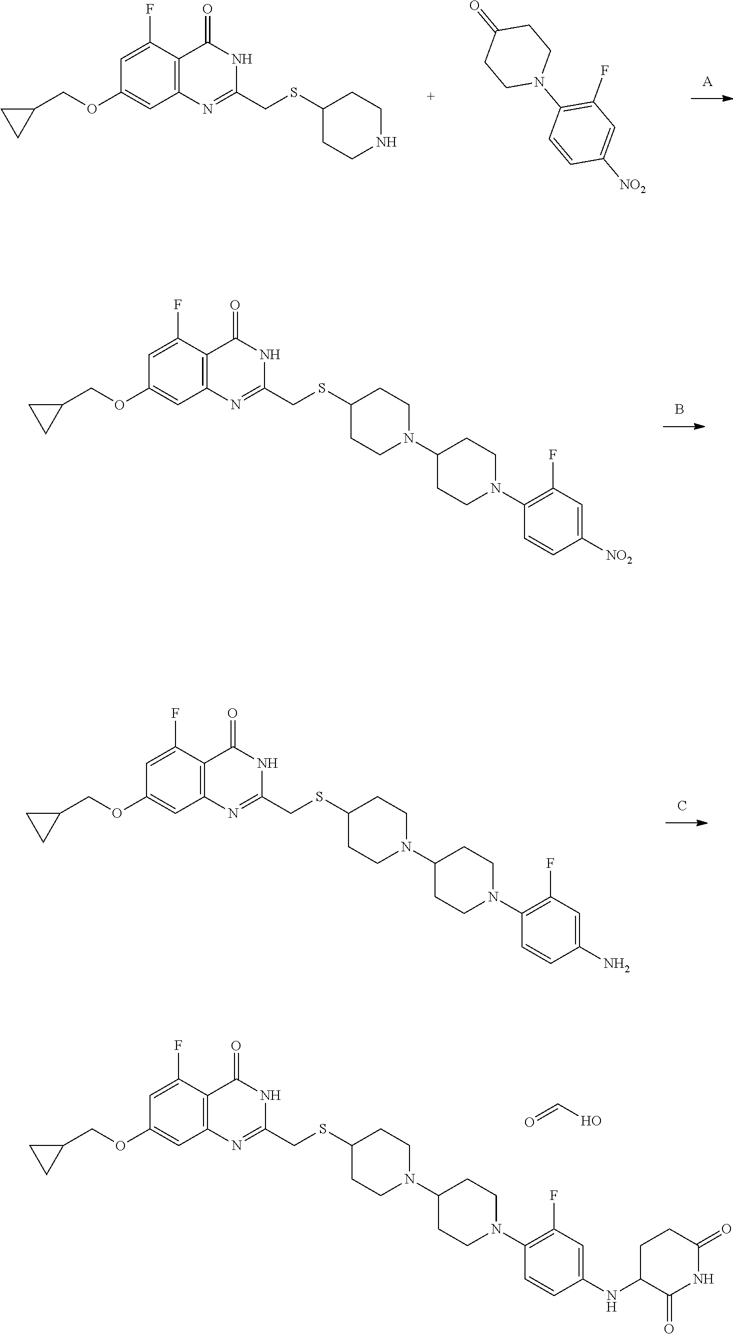

### Step A

A solution of 7-(cyclopropylmethoxy)-5-fluoro-2-((piperidin-4-ylthio)methyl)quinazolin-4(3H)-one (2 g, 5.50 mmol, 1 equiv) in DCE (100 mL) was treated with 1-(2-fluoro-4-nitrophenyl)piperidin-4-one (1.97 g, 8.26 mmol, 1.5 equiv) for 3 hours followed by the portion-wise addition of STAB (2.33 g, 11.0 mmol, 2 equiv). The resulting mixture was stirred for 1 day. The solution was concentrated and the residue was purified by silica gel column chromatography, eluting with DCM/MeOH (7:1) to afford 7-(cyclopropylmethoxy)-5-fluoro-2-(((1′-(2-fluoro-4-nitrophenyl)-[1,4′-bipiperidin]-4-yl)thio)methyl)quinazolin-4(3H)-one (1.4 g, 43%) as a yellow oil. LCMS (ESI, m/z): 586.35 [M+H]⁺.

### Step B

A solution of 7-(cyclopropylmethoxy)-5-fluoro-2-(((1′-(2-fluoro-4-nitrophenyl)-[1,4′-bipiperidin]-4-yl)thio)methyl)quinazolin-4(3H)-one (500 mg, 0.854 mmol, 1 equiv), 4,4′-bipyridine (13.3 mg, 0.085 mmol, 0.1 equiv) and B₂(OH)₄ (230 mg, 2.56 mmol, 3 equiv) in DMF (10 mL) was stirred for 30 min. The residue was purified by silica gel column chromatography, eluting with DCM/MeOH (5:1) to afford 2-(((1′-(4-amino-2-fluorophenyl)-[1,4′-bipiperidin]-4-yl)thio)methyl)-7-(cyclopropylmethoxy)-5-fluoroquinazolin-4(3H)-one (300 mg, 63%) as a yellow solid. LCMS (ESI, m/z): 556.45 [M+H]⁺.

### Step C

A solution of 2-(((1′-(4-amino-2-fluorophenyl)-[1,4′-bipiperidin]-4-yl)thio)methyl)-7-(cyclopropylmethoxy)-5-fluoroquinazolin-4(3H)-one (300 mg, 0.54 mmol, 1 equiv), 3-bromopiperidine-2,6-dione (1037 mg, 5.4 mmol, 10 equiv) and NaHCO₃ (453.5 mg, 5.4 mmol, 10 equiv) in ACN (30 mL) was stirred for 3 days at 90° C. The resulting mixture was concentrated and the residue purified by C18 reverse phase chromatography eluting with water/ACN (45:55) and further purified by Prep-HPLC with the following condition (Column: Xselect CSH OBD Column 30*150 mm, 5 μm; Mobile Phase A: water (0.1% FA), Mobile Phase B: ACN; Flow rate: 60 mL/min; Gradient: 5% B to 5% B in 2 min, 12% B to 22% B in 10 min; Wavelength: 254 nm/220 nm; RT (min): 9.6) to afford 3-((4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)amino) piperidine-2,6-dione formate (63.4 mg, 17%) as a grey solid. LCMS (ESI, m/z): 667.25 [M+H]⁺. ¹H NMR (400 MHz, DMSO-d₆) δ 12.13 (s, 1H), 10.76 (s, H), 8.18 (s, 1H), 6.93-6.76 (m, 3H), 6.50 (dd, J=15.0, 2.6 Hz, 1H), 6.40 (dd, J=8.8, 2.5 Hz, 1H), 5.77 (d, J=7.6 Hz, 1H), 4.30-4.19 (m, 1H), 3.99 (d, J=6.8 Hz, 2H), 3.65 (s, 2H), 3.15 (d, J=10.9 Hz, 2H), 2.89-2.76 (m, 4H), 2.76-2.67 (m, 2H), 2.35-2.27 (m, 1H), 2.27-2.23 (m, 2H), 2.13-2.04 (m, 1H), 2.03-1.74 (m, 5H), 1.57-1.39 (m, 4H), 1.31-1.13 (m, 2H), 0.64-0.54 (m, 2H), 0.40-0.32 (m, 2H).

### Example 55: Synthesis of 3-((4-(4-(2-(((1r,4r)-4-(((7-(cyclopropylmethoxy)-5-fluoro-4-oxo-3,4-dihydroquinazolin-2-yl)methyl)thio)cyclohexyl)oxy)ethyl)piperazin-1-yl)-3-fluorophenyl)amino)piperidine-2,6-dione

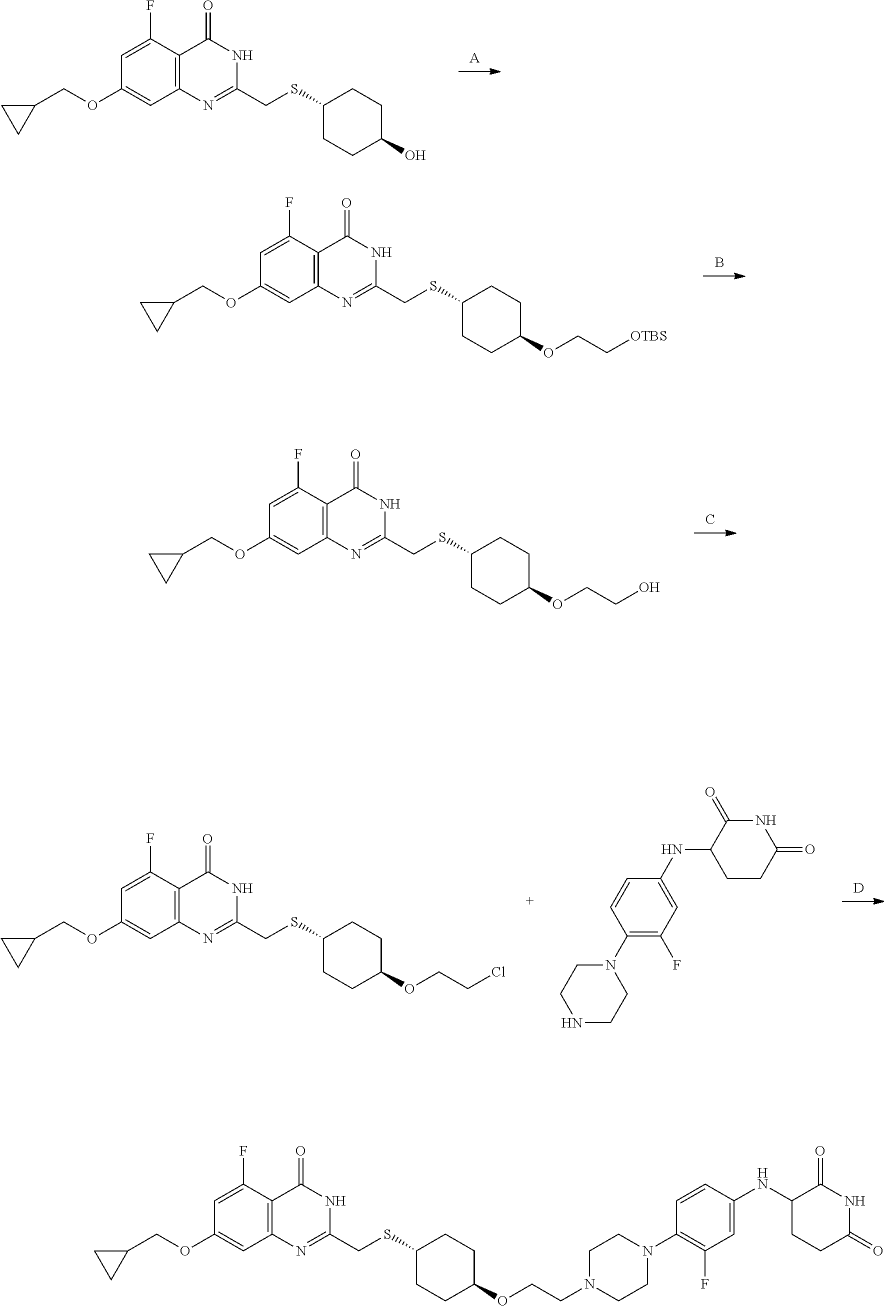

### Step A

A solution of 7-(cyclopropylmethoxy)-5-fluoro-2-((((1r,4r)-4-hydroxycyclohexyl)thio)methyl)quinazolin-4(3H)-one (1000 mg, 2.64 mmol, 1 equiv), (2-bromoethoxy)(tert-butyl)dimethylsilane (759 mg, 3.17 mmol, 1.2 equiv) and K₂CO₃ (1096 mg, 7.93 mmol, 3 equiv) in DMF (5 mL) was stirred for 3 hours at 80° C. The residue was purified by reversed-phase flash chromatography with the following conditions: column, C18 silica gel; mobile phase, MeCN in water (10 mmol/L NH₄HCO₃), 10% to 90% gradient in 30 min; detector, UV 254 nm. This afforded 2-((((1r,4r)-4-(2-((tert-butyldimethylsilyl)oxy)ethoxy)cyclohexyl)thio)methyl)-7-(cyclopropylmethoxy)-5-fluoroquinazolin-4(3H)-one (870 mg, 61%) as a white solid. LCMS (ESI, m/z): 537.20 [M+H]⁺.

### Step B

A solution of 2-((((1r,4r)-4-(2-((tert-butyldimethylsilyl)oxy)ethoxy)cyclohexyl)thio)methyl)-7-(cyclopropylmethoxy)-5-fluoroquinazolin-4(3H)-one (820 mg, 1.53 mmol, 1 equiv) in HCl in 1,4-dioxane (10 mL, 4 M) was stirred for 30 min. The resulting mixture was concentrated under vacuum to afford crude 7-(cyclopropylmethoxy)-5-fluoro-2-((((1r,4r)-4-(2-hydroxyethoxy)cyclohexyl)thio)methyl)quinazolin-4(3H)-one (990 mg) as a white solid. The crude product was used in the next step without further purification. LCMS (ESI, m/z): 423.50 [M+H]⁺.

### Step C

A solution 7-(cyclopropylmethoxy)-5-fluoro-2-((((1r,4r)-4-(2-hydroxyethoxy)cyclohexyl)thio)methyl)quinazolin-4(3H)-one (990 mg, 2.34 mmol, 1 equiv) and TEA (711 mg, 7.03 mmol, 3 equiv) in DCM (5 mL) was stirred for 1 hour at room temperature. To the above mixture was added 3-nitrobenzenesulfonyl chloride (779 mg, 3.51 mmol, 1.5 equiv) dropwise at 0° C. The resulting mixture was stirred for 3 hours at 40° C. The solution was concentrated and the residue was purified by silica gel column chromatography, eluting with DCM/MeOH (8:1) to afford 2-((((1r,4r)-4-(2-chloroethoxy)cyclohexyl)thio)methyl)-7-(cyclopropylmethoxy)-5-fluoroquinazolin-4(3H)-one (500 mg, 48%) as a white solid. LCMS (ESI, m/z): 441.90 [M+H]⁺.

### Step D

A solution 2-((((1r,4r)-4-(2-chloroethoxy)cyclohexyl)thio)methyl)-7-(cyclopropylmethoxy)-5-fluoroquinazolin-4(3H)-one (500 mg, 1.13 mmol, 1 equiv), 3-((3-fluoro-4-(piperazin-1-yl)phenyl)amino)piperidine-2,6-dione (521 mg, 1.70 mmol, 1.5 equiv), KI (37.7 mg, 0.227 mmol, 0.2 equiv), and K₂CO₃ (313 mg, 2.27 mmol, 2 equiv) in ACN (5 mL) was stirred for 3 hours at 80° C. The residue was purified by reversed-phase flash chromatography with the following conditions: column, C18 silica gel; mobile phase, MeCN in water (10 mmol/L NH₄HCO₃), 0% to 90% gradient in 30 min; detector, UV 254 nm. The crude product (200 mg) was purified by Prep-HPLC with the following conditions (Column: Xselect CSH OBD Column 30*150 mm, 5 μm; Mobile Phase A: Water (0.1% FA), Mobile Phase B: ACN; Flow rate: 60 mL/min; Gradient: 5% B to 5% B in 2 min, 16% B to 26% B in 10 min; Wavelength: 254 nm/220 nm, RT (min): 8.9) to afford 3-((4-(4-(2-(((1r,4r)-4-(((7-(cyclopropylmethoxy)-5-fluoro-4-oxo-3,4-dihydroquinazolin-2-yl)methyl)thio)cyclohexyl)oxy)ethyl)piperazin-1-yl)-3-fluorophenyl)amino)piperidine-2,6-dione (76 mg, 9%) as a white solid. LCMS (ESI, m/z): 711.50 [M+H]⁺. ¹H NMR (300 MHz, DMSO-d₆) δ 10.79 (s, 1H), 7.02-6.78 (m, 3H), 6.58-6.39 (m, 2H), 6.05-5.79 (m, 1H), 4.58-4.40 (m, 2H), 4.30-4.10 (m, 2H), 4.04-3.94 (m, 4H), 3.83-3.20 (m, 3H), 3.11-2.79 (m, 4H), 2.79-2.53 (m, 6H), 2.15-1.93 (m, 3H), 1.93-1.74 (m, 3H), 1.39-1.01 (m, 5H), 0.66-0.54 (m, 2H), 0.42-0.31 (m, 2H).

### Example 56: Synthesis of 3-((4-(4-((4-(((7-(cyclopropylmethoxy)-5-fluoro-4-oxo-3,4-dihydroquinazolin-2-yl)methyl)thio)piperidin-1-yl)methyl)piperidin-1-yl)-3-fluorophenyl)amino)piperidine-2,6-dione formate

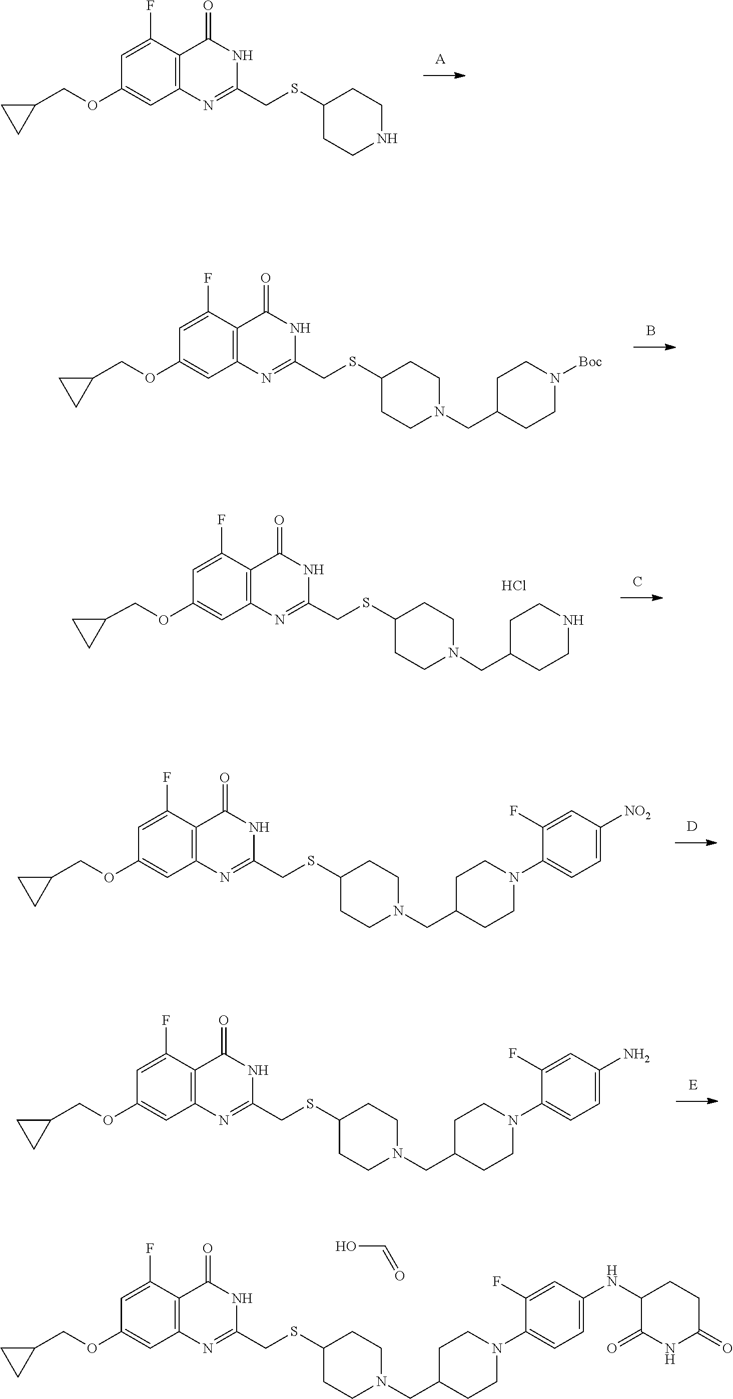

### Step A

A solution of 7-(cyclopropylmethoxy)-5-fluoro-2-[(piperidin-4-ylsulfanyl)methyl]-3H-quinazolin-4-one (1 g, 2.75 mmol, 1 equiv), tert-butyl 4-formylpiperidine-1-carboxylate (0.88 g, 4.13 mmol, 1.5 equiv) and STAB (1.75 g, 8.25 mmol, 3 equiv) in DCE (8 mL) was stirred for 1 hour. After concentration, the residue was purified by silica gel column chromatography, eluting with DCM/MeOH (9:1) to afford tert-butyl 4-((4-(((7-(cyclopropylmethoxy)-5-fluoro-4-oxo-3,4-dihydroquinazolin-2-yl)methyl)thio)piperidin-1-yl)methyl)piperidine-1-carboxylate (1.6 g, 99%) as an orange oil. LCMS (ESI, m/z): 561.30 [M+H]⁺.

### Step B

A solution of tert-butyl 4-((4-(((7-(cyclopropylmethoxy)-5-fluoro-4-oxo-3,4-dihydroquinazolin-2-yl)methyl)thio)piperidin-1-yl)methyl)piperidine-1-carboxylate (1.6 g, 2.85 mmol, 1 equiv) in HCl in 1,4-dioxane (8 mL, 4 M) was stirred for 1 hour. The mixture was concentrated to dryness to afford 7-(cyclopropylmethoxy)-5-fluoro-2-(((1-(piperidin-4-ylmethyl)piperidin-4-yl)thio)methyl)quinazolin-4(3H)-one hydrochloride (1 g, 65%) as a yellow solid. LCMS (ESI, m/z): 461.20 [M+H]⁺.

### Step C

A solution of 7-(cyclopropylmethoxy)-5-fluoro-2-(((1-(piperidin-4-ylmethyl)piperidin-4-yl)thio)methyl)quinazolin-4(3H)-one hydrochloride (1.00 g, 0.022 mmol, 1 equiv), 1,2-difluoro-4-nitrobenzene (415 mg, 0.026 mmol, 1.2 equiv), and NaHCO₃ (547 mg, 0.066 mmol, 3 equiv) in ACN (5 mL) was stirred for 5 hours at 90° C. The mixture was concentrated and the residue was purified by silica gel column chromatography, eluting with PE/EtOAc (3:97) to afford 7-(cyclopropylmethoxy)-5-fluoro-2-(((1-((1-(2-fluoro-4-nitrophenyl)piperidin-4-yl)methyl)piperidin-4-yl)thio)methyl)quinazolin-4(3H)-one (580 mg, 39%) as a yellow solid. LCMS (ESI, m/z): 600.30 [M+H]⁺.

### Step D

A solution of 7-(cyclopropylmethoxy)-5-fluoro-2-(((1-((1-(2-fluoro-4-nitrophenyl)piperidin-4-yl)methyl)piperidin-4-yl)thio)methyl)quinazolin-4(3H)-one (550 mg, 0.917 mmol, 1 equiv), Fe (256 mg, 0.085 mmol, 5 equiv), and NH₄Cl (98.1 mg, 1.83 mmol, 2 equiv) in EtOH (5 mL) and water (1 mL) was stirred for 1 hour at 80° C. The resulting mixture was filtered, the filter cake was washed with EtOH (5×10 mL). The filtrate was concentrated and the residue was purified by silica gel column chromatography, eluting with DCM/MeOH (9:1) to afford 2-(((1-((1-(4-amino-2-fluorophenyl)piperidin-4-yl)methyl)piperidin-4-yl)thio)methyl)-7-(cyclopropylmethoxy)-5-fluoroquinazolin-4(3H)-one (487 mg, 93%) as a yellow solid. LCMS (ESI, m/z): 570.15 [M+H]⁺.

### Step E

A solution of 2-(((1-((1-(4-amino-2-fluorophenyl)piperidin-4-yl)methyl)piperidin-4-yl)thio)methyl)-7-(cyclopropylmethoxy)-5-fluoroquinazolin-4(3H)-one (477 mg, 0.84 mmol, 1 equiv), 3-bromopiperidine-2,6-dione (482 mg, 2.51 mmol, 3 equiv) and NaHCO₃ (352 mg, 4.19 mmol, 5 equiv) in ACN (3 mL) was stirred overnight at 90° C. The mixture was concentrated and the residue was purified by reverse-phase flash chromatography with the following conditions: column, C18 silica gel; mobile phase, MeCN in water (0.1% FA), 10% to 50% gradient in 10 min; detector, UV 254 nm. This afforded 3-((4-(4-((4-(((7-(cyclopropylmethoxy)-5-fluoro-4-oxo-3,4-dihydroquinazolin-2-yl)methyl)thio)piperidin-1-yl)methyl)piperidin-1-yl)-3-fluorophenyl)amino)piperidine-2,6-dione formate (184 mg, 29%) as a dark-blue solid. LCMS (ESI, m/z): 681.25 [M+H]⁺. 1H NMR (400 MHz, DMSO-d6) δ 12.14 (s, 1H), 10.77 (s, 1H), 8.15 (s, 1H), 6.93-6.77 (m, 3H), 6.49 (dd, J=14.9, 2.6 Hz, 1H), 6.41 (dd, J=8.8, 2.6 Hz, 1H), 5.77 (d, J=7.6 Hz, 1H), 4.30-4.19 (m, 1H), 3.97 (d, J=7.1 Hz, 2H), 3.60 (s, 2H), 3.10 (d, J=11.1 Hz, 2H), 2.83-2.65 (m, 4H), 2.62-2.53 (m, 3H), 2.15 (d, J=7.1 Hz, 2H), 2.12-2.04 (m, 3H), 2.00-1.78 (m, 5H), 1.73 (d, J=12.1 Hz, 2H), 1.64-1.36 (m, 3H), 1.30-1.15 (m, 3H), 0.64-0.55 (m, 2H), 0.40-0.32 (m, 2H).

### Example 57: Synthesis of 3-((4-(4-(((7-(cyclopropylmethoxy)-5-fluoro-4-oxo-3,4-dihydroquinazolin-2-yl)methyl)thio)-[1,4′-bipiperidin]-1′-yl)-3-(trifluoromethyl)phenyl)amino)piperidine-2,6-dione

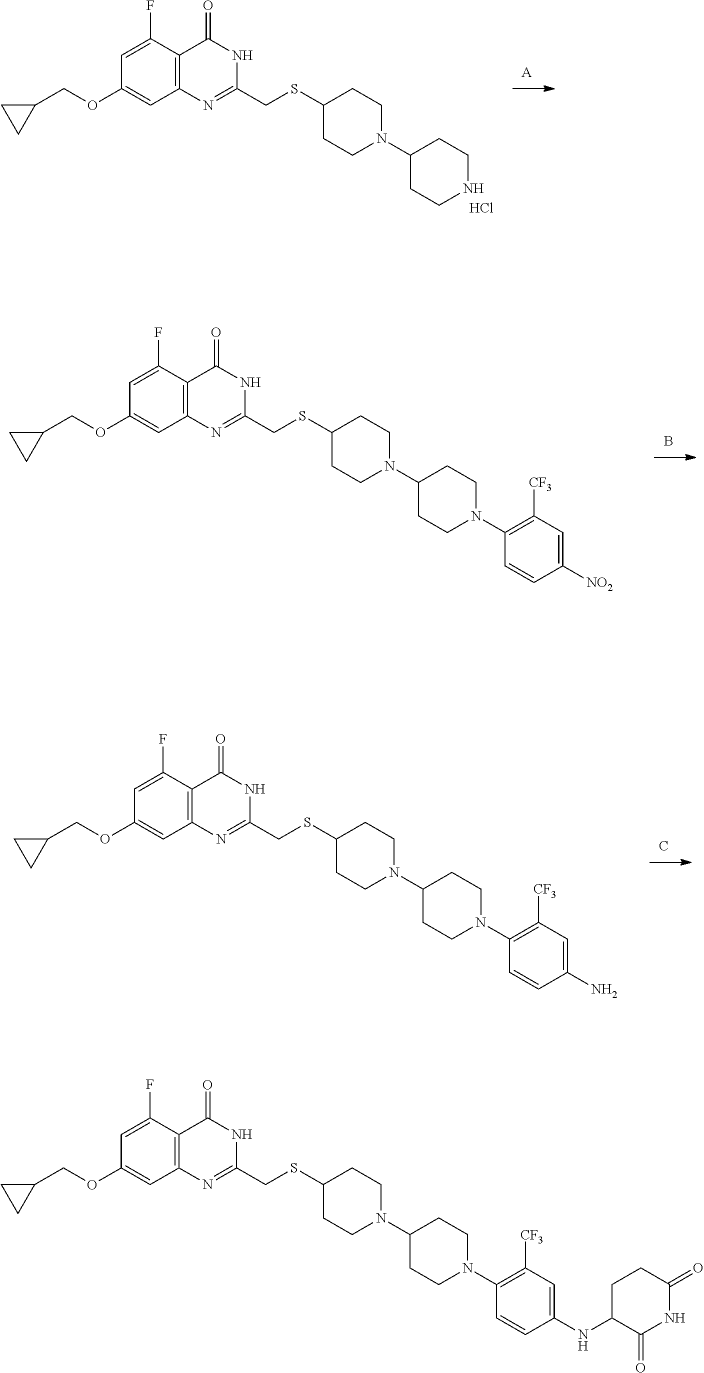

### Step A

A solution of 2-(([1,4′-bipiperidin]-4-ylthio)methyl)-7-(cyclopropylmethoxy)-5-fluoroquinazolin-4(3H)-one hydrochloride (600 mg, 1.24 mmol, 1 equiv), 1-fluoro-4-nitro-2-(trifluoromethyl)benzene (260 mg, 1.24 mmol, 1 equiv) and NaHCO₃ (313 mg, 3.73 mmol, 3 equiv) in ACN (10 mL) was stirred overnight at 80° C. The mixture was concentrated and the residue purified by silica gel column chromatography, eluting with DCM/MeOH (10:1) to afford 7-(cyclopropylmethoxy)-5-fluoro-2-(((1′-(4-nitro-2-(trifluoromethyl)phenyl)-[1,4′-bipiperidin]-4-yl)thio)methyl)quinazolin-4(3H)-one (420 mg, 53%) as a yellow solid. LCMS (ESI, m/z): 636.10 [M+H]⁺.

### Step B

A solution of 7-(cyclopropylmethoxy)-5-fluoro-2-(((1′-(4-nitro-2-(trifluoromethyl)phenyl)-[1,4′-bipiperidin]-4-yl)thio)methyl)quinazolin-4(3H)-one (420 mg, 0.661 mmol, 1 equiv), Fe (185 mg, 3.31 mmol, 5 equiv) and NH₄Cl (70.7 mg, 1.32 mmol, 2 equiv) in EtOH (5 mL) and water (1 mL) was stirred for 3 hours at 80° C. The mixture was concentrated and the residue was purified by silica gel column chromatography, eluting with DCM/MeOH (7:1) to afford 2-(((1′-(4-amino-2-(trifluoromethyl)phenyl)-[1,4′-bipiperidin]-4-yl)thio)methyl)-7-(cyclopropylmethoxy)-5-fluoroquinazolin-4(3H)-one (350 mg, 88%) as a yellow solid. LCMS (ESI, m/z): 606.10 [M+H]⁺.

### Step C

A solution of 2-(((1′-(4-amino-2-(trifluoromethyl)phenyl)-[1,4′-bipiperidin]-4-yl)thio)methyl)-7-(cyclopropylmethoxy)-5-fluoroquinazolin-4(3H)-one (320 mg, 0.528 mmol, 1 equiv), 3-bromopiperidine-2,6-dione (304 mg, 1.58 mmol, 3 equiv) and NaHCO₃ (222 mg, 2.64 mmol, 5 equiv) in ACN (5 mL) was stirred overnight at 90° C. The resulting mixture was concentrated and the residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. This afforded 3-((4-(4-(((7-(cyclopropylmethoxy)-5-fluoro-4-oxo-3,4-dihydroquinazolin-2-yl)methyl)thio)-[1,4′-bipiperidin]-1′-yl)-3-(trifluoromethyl)phenyl)amino)piperidine-2,6-dione (135 mg, 36%) as a white solid. LCMS (ESI, m/z): 717.25 [M+H]⁺. ¹H NMR (400 MHz, DMSO-d₆) δ 12.15 (s, 1H), 10.79 (s, 1H), 7.27 (d, J=8.7 Hz, 1H), 6.94-6.83 (m, 4H), 6.17 (d, J=7.8 Hz, 1H), 4.44-4.33 (m, 1H), 3.97 (d, J=7.1 Hz, 2H), 3.60 (s, 2H), 2.86-2.79 (m, 4H), 2.77-2.63 (m, 3H), 2.62-2.55 (m, 2H), 2.32-2.25 (m, 1H), 2.22-2.11 (m, 2H), 2.09-2.03 (m, 2H), 1.96-1.86 (m, 3H), 1.72 (d, J=8.0 Hz, 2H), 1.54-1.42 (m, 3H), 1.29-1.22 (m, 1H), 0.64-0.55 (m, 2H), 0.40-0.33 (m, 2H).

Examples 58-59 were synthesized according to the procedure described for the synthesis 3-((4-(4-(((7-(cyclopropylmethoxy)-5-fluoro-4-oxo-3,4-dihydroquinazolin-2-yl)methyl)thio)-[1,4′-bipiperidin]-1′-yl)-3-(trifluoromethyl)phenyl)amino)piperidine-2,6-dione

Example 57 using appropriate building blocks and modified reaction conditions (such as reagents, reagent ratio, temperature, and reaction time) and purification conditions as needed.

| Ex- | | |
|---|---|---|
| ample | Structure | Analytical Data |
| 58 | 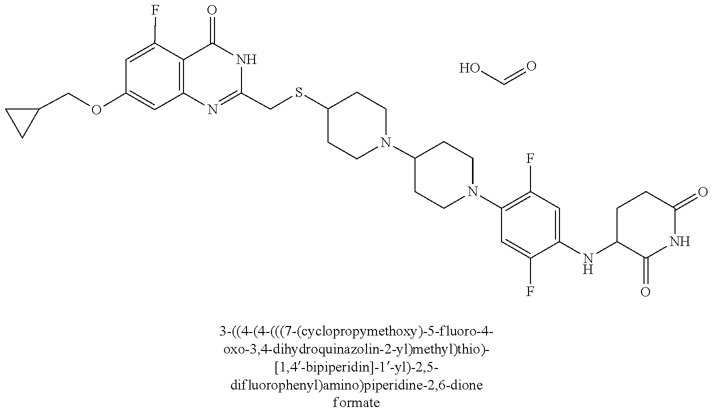 [SMILES: O=C1CCC(Nc2cc(F)c(N3CCC(N4CCC(SCc5nc6cc(OCC7CC7)cc(F)c6c(=O)[nH]5)CC4)CC3)cc2F)C(=O)N1 ; O=CO] | LCMS (ESI, m/z): 685.30 [M + H]⁺. 1H NMR (400 MHz, DMSO-d6) δ 12.14 (s, 1H), 10.79 (s, 1H), 8.18 (s, 1H), 6.94-6.77 (m, 3H), 6.72 (dd, J = 14.3, 8.3 Hz, 1H), 5.47 (d, J = 8.3 Hz, 1H), 4.40-4.29 (m, 1H), 3.97 (d, J = 7.1 Hz, 2H), 3.60 (s, 2H), 3.22-3.14 (m, 3H), 2.89-2.76 (m, 3H), 2.75-2.65 (m, 1H), 2.61- 2.52 (m, 2H), 2.36- 2.29 (m, 1H), 2.29- 2.15 (m, 2H), 2.11-1.96 (m, 2H), 1.93 (d, J = 12.6 Hz, 2H), 1.75 (d, J = 12.0 Hz, 2H), 1.61- 1.50 (m, 2H), 1.49- 1.35 (m, 2H), 1.31- 1.19 (m, 1H), 0.64- 0.55 (m, 2H), 0.40- 0.32 (m, 2H). |
| 59 | 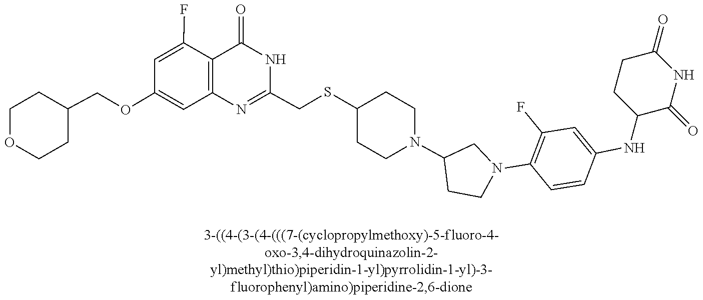 [SMILES: O=C1CCC(Nc2ccc(N3CCC(N4CCC(SCc5nc6cc(OCC7CCOCC7)cc(F)c6c(=O)[nH]5)CC4)C3)c(F)c2)C(=O)N1] | LCMS (ESI, m/z): 653.25 [M + H]⁺. 1H NMR (300 MHz, DMSO-d6) δ 12.10 (br, 1H), 10.75 (s, 1H), 6.95- 6.80 (m, 2H), 6.61 (t, J = 9.4 Hz, 1H), 6.50 (dd, J = 15.6, 2.5 Hz, 1H), 6.44-6.34 (m, 1H), 5.54 (d, J = 7.5 Hz, 1H), 4.27-4.13 (m, 1H), 3.96 (d, J = 7.1 Hz, 2H), 3.60 (s, 2H), 3.25-3.18 (m, 2H), 3.12-2.95 (m, 2H), 2.94-2.68 (m, 5H), 2.62-2.55 (m, 1H), 2.25-2.00 (m, 4H), 1.99-1.65 (m, |
| | | 4H), 1.55-1.35 (m, |
| | | 2H), 1.30-1.20 (m, |
| | | 1H), 0.70-0.60 (m, |
| | | 2H), 0.40-0.25 (m, 2H) |

### Example 60: Synthesis of 1-(4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)dihydropyrimidine-2,4(1H,3H)-dione

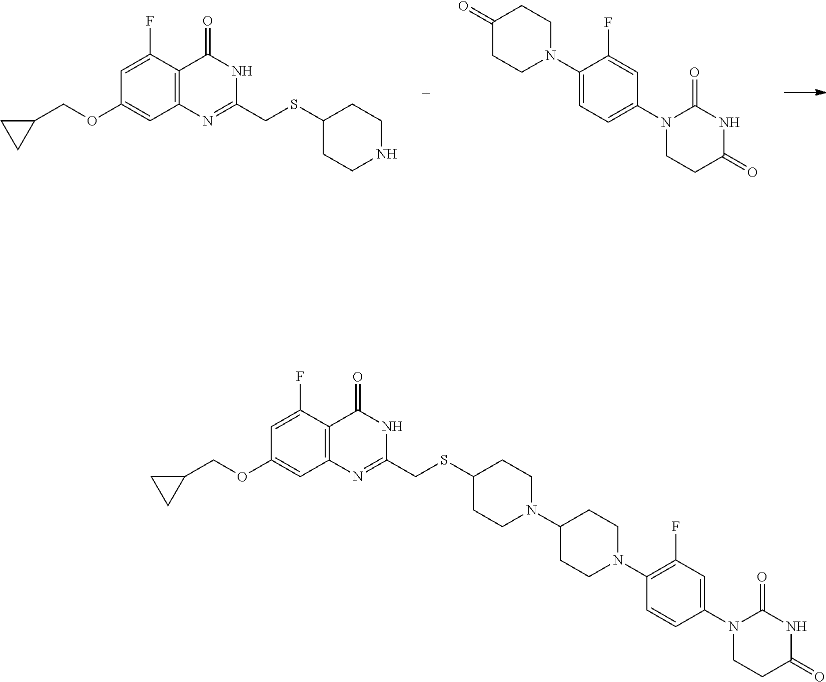 [SMILES: O=c1[nH]c(CSC2CCNCC2)nc2cc(OCC3CC3)cc(F)c12 ; O=C1CCN(c2ccc(N3CCC(=O)NC3=O)cc2F)CC1 ; O=C1CCN(c2ccc(N3CCC(N4CCC(SCc5nc6cc(OCC7CC7)cc(F)c6c(=O)[nH]5)CC4)CC3)c(F)c2)C(=O)N1]

A solution of 1-(3-fluoro-4-(4-oxopiperidin-1-yl)phenyl)dihydropyrimidine-2,4(1H,3H)-dione (140 mg, 0.46 mmol, 1 equiv), 7-(cyclopropylmethoxy)-5-fluoro-2-((piperidin-4-ylthio)methyl)quinazolin-4(3H)-one (167 mg, 0.46 mmol, 1 equiv) and STAB (194 mg, 0.92 mmol, 2 equiv) in DMF (8 mL) was stirred for 1 hour at 60° C. The mixture was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. This afforded crude product 100 mg as a white solid. The crude product was purified by Prep-HPLC with the following conditions (Column: Xselect CSH C18 OBD Column 30*150 mm 5 m; Mobile Phase A: water (0.1% FA), Mobile Phase B: ACN; Flow rate: 60 mL/min; Gradient: 5% B to 5% B in 2 min, 10% B to 20% B in 10 min; Wavelength: 254/220 nm; RT (min): 9.2) to afford 1-(4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)dihydropyrimidine-2,4(1H,3H)-dione (49.4 mg, 16%) as a white solid. LCMS (ESI, m/z): 653.30 [M+H]⁺. ¹H NMR (400 MHz, DMSO-d₆) δ 12.25 (br, 1H), 10.45-10.24 (m, 1H), 8.29 (d, J=16.3 Hz, 1H), 7.40-6.80 (m, 4H), 4.06-3.92 (m, 2H), 3.75 (dd, J=14.4, 7.6 Hz, 2H), 3.62 (d, J=11.5 Hz, 2H), 3.42-3.30 (m, 2H), 2.89-2.79 (m 3H), 2.78-2.54 (m, 4H), 2.40-2.32 (m, 1H), 2.23-2.17 (m, 2H), 2.00-1.90 (d, J=15.3 Hz, 2H), 1.89-1.70 (m, 2H), 1.68-1.34 (m, 4H), 1.32-1.14 (m, 1H), 0.70-0.50 (m, 2H), 0.46-0.26 (m, 2H).

Examples 61-62 were synthesized according to the procedure described for the synthesis 1-(4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)dihydropyrimidine-2,4(1H,3H)-dione Example 60 using appropriate building blocks and modified reaction conditions (such as reagents, reagent ratio, temperature, and reaction time) and purification conditions as needed.

| Example | Structure | Analytical Data |
|---|---|---|
| 61 | 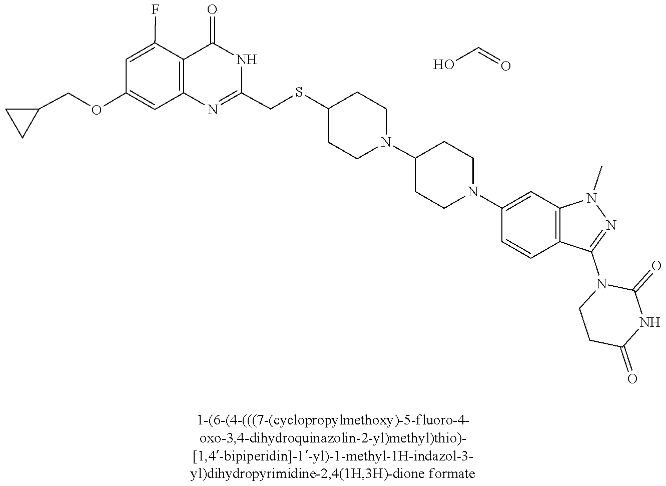 [SMILES: Cn1nc(N2CCC(=O)NC2=O)c2ccc(N3CCC(N4CCC(SCc5nc6cc(OCC7CC7)cc(F)c6c(=O)[nH]5)CC4)CC3)cc21 ; O=CO] | LCMS (ESI, m/z): 689.30 [M + H]⁺. 1H NMR (300 MHz, DMSO-d6) δ 12.12 (br, 1H), 10.48 (s, 1H), 8.20 (s, 1H), 7.44 (d, J = 9.0 Hz, 1H), 6.92-6.80 (m, 4H), 3.97 (d, J = 7.1 Hz, 2H), 3.90-3.82 (m, 7H), 3.60 (s, 2H), 2.90- 2.80 (m, 3H), 2.77-2.68 (m, 4H), 2.44-2.40 (m, 1H), 2.27-2.18 (m, 2H), 1.99-1.89 (m, 2H), 1.86-1.76 (m, 2H), 1.62-1.41 (m, 4H), 1.29-1.21 (m, 1H), 0.63-0.57 (m, 2H), 0.39-0.33 (m, 2H). |
| 62 | 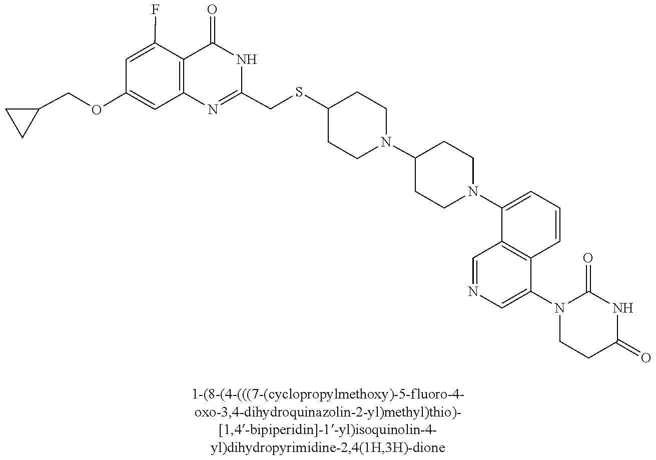 [SMILES: O=C1CCN(c2cncc3c(N4CCC(N5CCC(SCc6nc7cc(OCC8CC8)cc(F)c7c(=O)[nH]6)CC5)CC4)cccc23)C(=O)N1] | LCMS (ESI, m/z): 686.35 [M + H]⁺. 1H NMR (400 MHz, DMSO-d6) δ 12.12 (br, 1H), 10.53 (s, 1H), 9.39 (s, 1H), 8.52 (s, 1H), 7.70 (t, J = 7.9 Hz, 1H), 7.59 (d, J = 8.4 Hz, 1H), 7.24 (d, J = 7.5 Hz, 1H), 6.89-6.80 (m, 2H), 3.97 (d, J = 7.0 Hz, 2H), 3.95-3.87 (m, 1H), 3.73- 3.67 (m, 1H), 3.63- 3.59 (m, 2H), 3.51-3.46 (m, 2H), 3.01-3.70 (m, 7H), 2.49-2.40 (m, 1H), 2.38-2.22 (m, 2H), 2.01-1.79 (m, 6H), 1.55-1.40 (m, 2H), 1.29-1.21 (m, 1H), 0.66-0.56 (m, 2H), 0.40-0.30 (m, 2H) |

### Example 63: Synthesis of 3-((4-(4-(4-(2-(7-(cyclopropylmethoxy)-5-fluoro-4-oxo-3,4-dihydroquinazolin-2-yl)ethyl)piperazin-1-yl)piperidin-1-yl)-3-fluorophenyl)amino)piperidine-2,6-dione formic salt

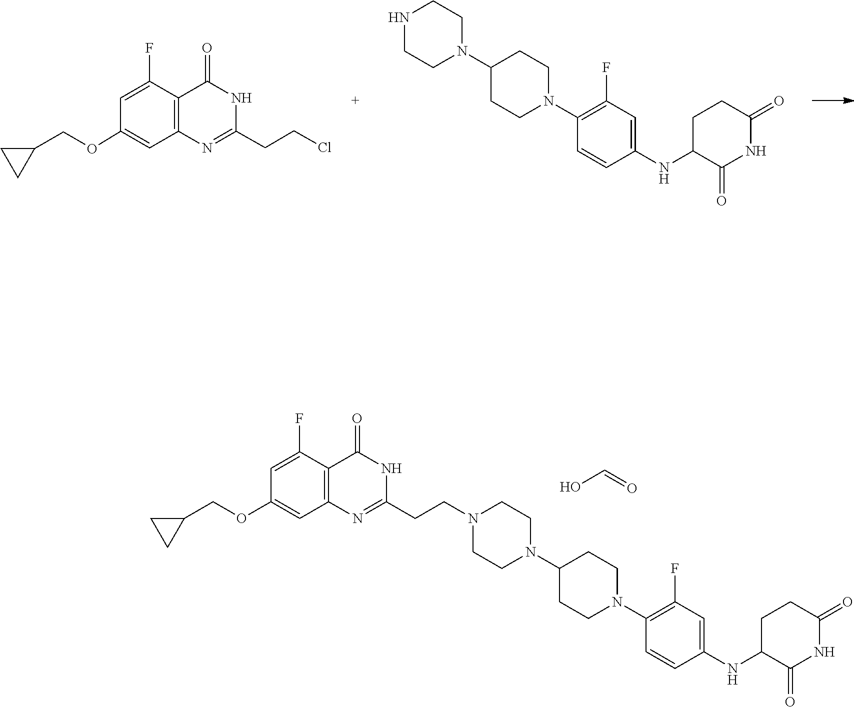 [SMILES: O=c1[nH]c(CCCl)nc2cc(OCC3CC3)cc(F)c12 ; O=C1CCC(Nc2ccc(N3CCC(N4CCNCC4)CC3)c(F)c2)C(=O)N1 ; O=C1CCC(Nc2ccc(N3CCC(N4CCN(CCc5nc6cc(OCC7CC7)cc(F)c6c(=O)[nH]5)CC4)CC3)c(F)c2)C(=O)N1 ; O=CO]

A solution of 2-(2-chloroethyl)-7-(cyclopropylmethoxy)-5-fluoroquinazolin-4(3H)-one (180 mg, 0.607 mmol, 1 equiv), 3-((3-fluoro-4-(4-(piperazin-1-yl)piperidin-1-yl)phenyl)amino)piperidine-2,6-dione (236 mg, 0.607 mmol, 1 equiv), K₂CO₃ (168 mg, 1.21 mmol, 2 equiv) and KI (10.1 mg, 0.061 mmol, 0.1 equiv) in ACN (20 mL) was stirred for 1 hour at 60° C. The resulting mixture was concentrated and purified by silica gel column chromatography, eluting with DCM/MeOH (8:1) then purified by Prep-HPLC with the following conditions (Column: XBridge Prep Phenyl OBD Column 19*250 mm; Mobile Phase A: water (0.1% FA), Mobile Phase B: ACN; Flow rate: 25 mL/min; Gradient: 16% B to 26% B in 10 min; Wavelength: 254/220 nm; RT (min): 12.8) to afford 3-((4-(4-(4-(2-(7-(cyclopropylmethoxy)-5-fluoro-4-oxo-3,4-dihydroquinazolin-2-yl)ethyl)piperazin-1-yl)piperidin-1-yl)-3-fluorophenyl)amino)piperidine-2,6-dione formic salt (17.6 mg, 4%) as a dark grey solid. LCMS (ESI, m/z): 650.25 [M+H]⁺. ¹H NMR (400 MHz, DMSO-d₆) δ 12.15 (br, 1H), 10.76 (s, 1H), 8.27 (s, 1H), 6.93-6.77 (m, 3H), 6.49 (dd, J=15.0, 2.6 Hz, 1H), 6.40 (dd, J=8.7, 2.5 Hz, 1H), 5.78 (d, J=7.6 Hz, 1H), 4.30-4.19 (m, 1H), 3.95 (d, J=7.0 Hz, 2H), 3.65-3.55 (m, 3H), 3.15 (d, J=11.0 Hz, 2H), 2.85-2.76 (m, 1H), 2.81-2.66 (m, 6H), 2.62-2.50 (m, 4H), 2.44-2.30 (m, 4H), 2.27-2.19 (m, 1H), 2.13-2.04 (m, 1H), 1.95-1.72 (m, 4H), 1.58-1.46 (m, 2H), 0.64-0.55 (m, 2H), 0.39-0.29 (m, 2H).

Example 64 was synthesized according to the procedure described for the synthesis 3-((4-(4-(4-(2-(7-(cyclopropylmethoxy)-5-fluoro-4-oxo-3,4-dihydroquinazolin-2-yl)ethyl)piperazin-1-yl)piperidin-1-yl)-3-fluorophenyl)amino)piperidine-2,6-dione formic salt Example 63 using appropriate building blocks and modified reaction conditions (such as reagents, reagent ratio, temperature, and reaction time) and purification conditions as needed.

| Example | Structure | Analytical Data |
|---|---|---|
| 64 | 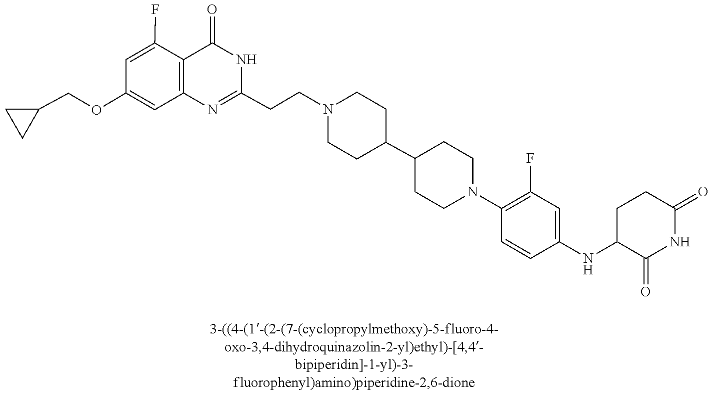 [SMILES: O=C1CCC(Nc2ccc(N3CCC(C4CCN(CCc5nc6cc(OCC7CC7)cc(F)c6c(=O)[nH]5)CC4)CC3)c(F)c2)C(=O)N1] | LCMS (ESI, m/z): 649.35 [M + H]⁺. 1H NMR (300 MHz, DMSO-d6) δ 10.75 (s, 1H), 6.89-6.76 (m, 3H), 6.54-6.38 (m, 2H), 5.76 (d, J = 7.6 Hz, 1H), 4.32-4.17 (m, 1H), 3.96 (d, J = 7.1 Hz, 2H), 3.18-3.12 (m, 3H), 2.97 (d, J = 10.9 Hz, 2H), 2.77-2.67 (m, 5H), 2.62-2.58 (m, 1H), 2.46-2.40 (m, 1H), 2.15-2.04 (m, 1H), 2.00-1.81 (m, 3H), 1.77-1.64 (m, 4H), 1.36-1.11 (m, 7H), 0.66-0.52 (m, 2H), 0.41-0.30 (m, 2H). |

### Example 65: Synthesis of 3-((4-(4-(2-(7-(cyclopropylmethoxy)-5-fluoro-4-oxo-3,4-dihydroquinazolin-2-yl)ethyl)-[1,4′-bipiperidin]-1′-yl)-3-fluorophenyl)amino)piperidine-2,6-dione

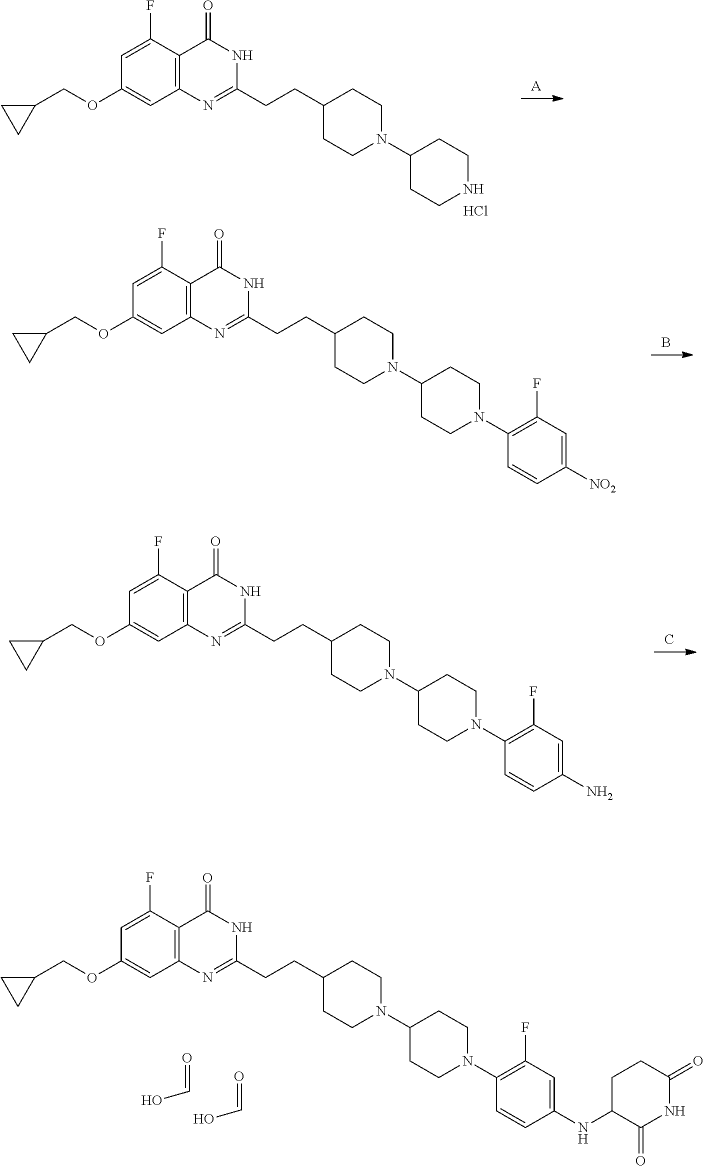

### Step A

A solution of 2-(2-([1,4′-bipiperidin]-4-yl)ethyl)-7-(cyclopropylmethoxy)-5-fluoroquinazolin-4(3H)-one hydrochloride (90 mg, 0.194 mmol, 1 equiv), 1,2-difluoro-4-nitrobenzene (30.8 mg, 0.194 mmol, 1 equiv) and DIEA (100 mg, 0.776 mmol, 4 equiv) in NMP (4 mL) was stirred for 1 hour at 80° C. The residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. This afforded 7-(cyclopropylmethoxy)-5-fluoro-2-(2-(1′-(2-fluoro-4-nitrophenyl)-[1,4′-bipiperidin]-4-yl)ethyl)quinazolin-4(3H)-one. (100 mg, 91%) as a yellow solid. LCMS (ESI, m/z): 568.16 [M+H]⁺.

### Step B

A solution of 7-(cyclopropylmethoxy)-5-fluoro-2-(2-(1′-(2-fluoro-4-nitrophenyl)-[1,4′-bipiperidin]-4-yl)ethyl)quinazolin-4(3H)-one (100 mg, 0.176 mmol, 1 equiv), Fe (49.2 mg, 0.880 mmol, 5 equiv) and NH₄Cl (18.9 mg, 0.352 mmol, 2 equiv) in EtOH (5 mL) and water (1 mL) was stirred for 1 hour at 80° C. The resulting mixture was concentrated and the residue was purified by silica gel column chromatography, eluting with DCM/MeOH (10:1) to afford 2-(2-(1′-(4-amino-2-fluorophenyl)-[1,4′-bipiperidin]-4-yl)ethyl)-7-(cyclopropylmethoxy)-5-fluoroquinazolin-4(3H)-one (80 mg, 85%) as a white solid. LCMS (ESI, m/z): 538.16 [M+H]⁺.

### Step C

A solution of 2-(2-(1′-(4-amino-2-fluorophenyl)-[1,4′-bipiperidin]-4-yl)ethyl)-7-(cyclopropylmethoxy)-5-fluoroquinazolin-4(3H)-one (70 mg, 0.13 mmol, 1 equiv), 3-bromopiperidine-2,6-dione (50.0 mg, 0.26 mmol, 2 equiv) and NaHCO₃ (43.8 mg, 0.52 mmol, 4 equiv) in ACN (10 mL) was stirred overnight at 90° C. The resulting mixture was concentrated and the residue purified by reverse-phase flash chromatography with the following conditions: column, C18 silica gel; mobile phase, MeCN in water (10 mmol/L NH₄HCO₃), 10% to 50% gradient in 10 min; detector, UV 254 nm. This afforded 3-((4-(4-(2-(7-(cyclopropylmethoxy)-5-fluoro-4-oxo-3,4-dihydroquinazolin-2-yl)ethyl)-[1,4′-bipiperidin]-1′-yl)-3-fluorophenyl)amino)piperidine-2,6-dione (22.2 mg, 24%) as a purple solid. LCMS (ESI, m/z): 649.30 [M+H]⁺. ¹H NMR (400 MHz, DMSO-d₆) δ 12.07 (br, 1H), 10.77 (s, 1H), 8.23 (s, 2H), 6.91-6.76 (m, 3H), 6.50 (dd, J=15.0, 2.6 Hz, 1H), 6.41 (dd, J=8.7, 2.6 Hz, 1H), 5.79 (d, J=7.6 Hz, 1H), 4.28-4.22 (m, 1H), 3.96 (d, J=7.1 Hz, 2H), 3.17 (d, J=11.0 Hz, 2H), 2.99-2.90 (m, 2H), 2.76-2.65 (m, 2H), 2.61-2.57 (m, 2H), 2.56-2.51 (m, 3H), 2.28-2.15 (m, 2H), 2.10-2.01 (m, 1H), 1.95-1.80 (m, 3H), 1.72-1.59 (m, 6H), 1.34-1.14 (m, 4H), 0.62-0.56 (m, 2H), 0.36 (t, J=5.1 Hz, 2H).

Example 66 was synthesized according to the procedure described for the 3-((4-(4-(2-(7-(cyclopropylmethoxy)-5-fluoro-4-oxo-3,4-dihydroquinazolin-2-yl)ethyl)-[1,4′-bipiperidin]-1′-yl)-3-fluorophenyl)amino)piperidine-2,6-dione Example 65 using appropriate building blocks and modified reaction conditions (such as reagents, reagent ratio, temperature, and reaction time) and purification conditions as needed.

| Example | Structure | Analytical Data |
|---|---|---|
| 66 | 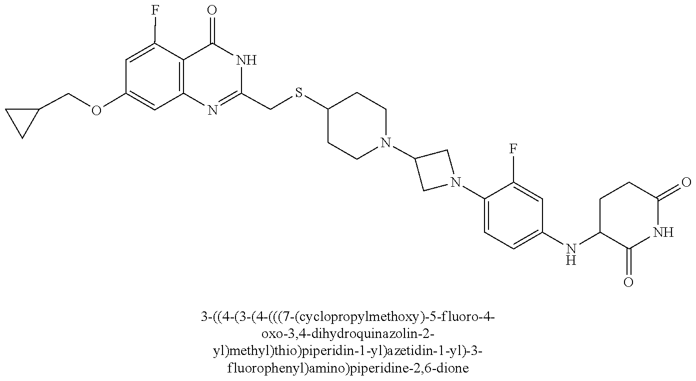 [SMILES: O=C1CCC(Nc2ccc(N3CC(N4CCC(SCc5nc6cc(OCC7CC7)cc(F)c6c(=O)[nH]5)CC4)C3)c(F)c2)C(=O)N1] | LCMS (ESI, m/z): 639.20 [M + H]⁺. 1H NMR (300 MHz, DMSO-d6) δ 12.10 (br, 1H), 10.74 (s, 1H), 6.93- 6.83 (m, 2H), 6.54- 6.40 (m, 3H), 5.57-5.49 (m, 1H), 4.22-4.10 (m, 1H), 4.01-3.93 (m, 2H), 3.84 (s, 2H), 3.60 (s, 2H), 3.19-3.05 (m, 1H), 2.98-2.58 (m, 8H), 2.18-2.07 (m, 1H), 2.18-1.62 (m, 4H), 1.49-1.39 (m, 2H), 1.28-1.15 (m, 1H), 0.70- 0.60 (m, 2H), 0.36- 0.25 (m, 2H). |

### Example 67: Synthesis of 3-((4-(4-(((7-(cyclopropylethynyl)-5-fluoro-4-oxo-3,4-dihydroquinazolin-2-yl)methyl)thio)-[1,4′-bipiperidin]-1′-yl)-3-fluorophenyl)amino)piperidine-2,6-dione formate

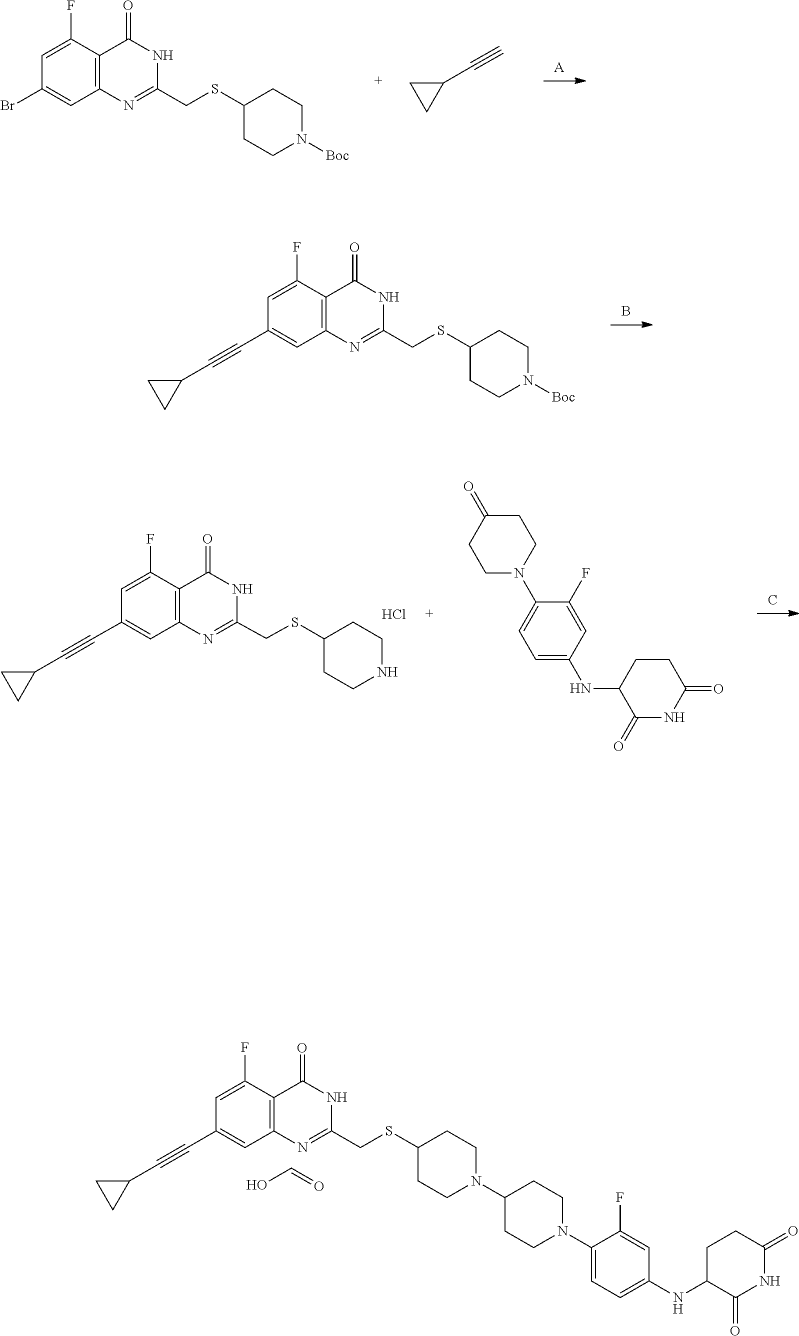

### Step A

A solution of tert-butyl 4-(((7-bromo-5-fluoro-4-oxo-3,4-dihydroquinazolin-2-yl)methyl)thio)piperidine-1-carboxylate (1000 mg, 2.12 mmol, 1 equiv), ethynylcyclopropane (420 mg, 6.35 mmol, 3 equiv), Pd(PPh₃)₂Cl₂ (149 mg, 0.21 mmol, 0.1 equiv), CuI (40.3 mg, 0.21 mmol, 0.1 equiv) and TEA (429 mg, 4.23 mmol, 2 equiv) in DMSO (10 mL) was stirred overnight at 100° C. under nitrogen atmosphere. The resulting mixture was concentrated and the residue was purified by silica gel column chromatography, eluting with PE/EtOAc (1:9) to afford tert-butyl 4-(((7-(cyclopropylethynyl)-5-fluoro-4-oxo-3,4-dihydroquinazolin-2-yl)methyl)thio)piperidine-1-carboxylate (1.3 g) as a brown oil. The crude product was used in the next step directly without further purification LCMS (ESI, m/z): 458.10 [M+H]⁺.

### Step B

A solution of tert-butyl 4-(((7-(cyclopropylethynyl)-5-fluoro-4-oxo-3,4-dihydroquinazolin-2-yl)methyl)thio)piperidine-1-carboxylate (260 mg, 0.57 mmol, 1 equiv) in HCl in 1,4-dioxane (5 mL, 4 M) was stirred for 1 hour. The mixture was concentrated to dryness to afford 7-(cyclopropylethynyl)-5-fluoro-2-((piperidin-4-ylthio)methyl)quinazolin-4(3H)-one hydrochloride (130 mg, 64%) as a white solid. LCMS (ESI, m/z): 358.10 [M+H]⁺.

### Step C

A solution of 7-(cyclopropylethynyl)-5-fluoro-2-((piperidin-4-ylthio)methyl)quinazolin-4(3H)-one hydrochloride (100 mg, 0.28 mmol, 1 equiv), 3-((3-fluoro-4-(4-oxopiperidin-1-yl)phenyl)amino)piperidine-2,6-dione (179 mg, 0.56 mmol, 2 equiv), STAB (119 mg, 0.56 mmol, 2 equiv) and TEA (2.83 mg, 0.028 mmol, 0.1 equiv) in DCE (1 mL) was stirred for 1 hour. The residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. The crude product (100 mg) was further purified by Prep-HPLC with the following conditions (Column: Xselect CSH C18 OBD Column 30*150 mm; Mobile Phase A: water (0.1% FA), Mobile Phase B: ACN; Flow rate: 25 mL/min; Gradient: 23% B to 33% B in 10 min; Wave Length: 254/220 nm; RT (min): 9) to afford 3-((4-(4-(((7-(cyclopropylethynyl)-5-fluoro-4-oxo-3,4-dihydroquinazolin-2-yl)methyl)thio)-[1,4′-bipiperidin]-1′-yl)-3-fluorophenyl)amino)piperidine-2,6-dione formate (14.3 mg, 8%) as a white solid. LCMS (ESI, m/z): 661.25 [M+H]⁺. 1H NMR (400 MHz, DMSO-d₆) δ 10.77 (s, 1H), 8.21 (s, 1H), 7.34 (s, 1H), 7.20 (d, J=11.2 Hz, 1H), 6.82 (t, J=9.3 Hz, 1H), 6.59-6.36 (m, 2H), 5.78 (d, J=7.6 Hz, 1H), 4.32-4.18 (m, 1H), 3.61-3.54 (m, 5H), 3.15 (d, J=10.7 Hz, 3H), 2.84-2.65 (m, 5H), 2.62-2.56 (m, 2H), 2.28-2.22 (m, 3H), 2.14-2.02 (m, 1H), 1.94-1.89 (m, 2H), 1.88-1.81 (m, 1H), 1.77-1.60 (m, 2H), 1.66-1.54 (m, 3H), 1.49-1.39 (m, 2H), 0.97-1.91 (m, 2H), 0.83-0.76 (m, 2H).

### Example A: NanoLuc Assay for the Degradation of PARP14

### NanoLuc Plasmid

The catalytic domain of human PARP14 (residues 1611 to 1801, GenBank Accession No. NM_017554) was inserted into the pcDNA3.1(−) vector. The insert also contained a NanoLuc tag on the N terminus of the PARP14 protein.

### Assay for Degradation of PARP14

Degradation of PARP14 protein was assessed using measurement of the NanoLuc tag as proxy for the PARP14 protein. PARP14 with NanoLuc tag was overexpressed in iEK-293T cells (ATCC) using the plasmid described in Table 1. Plasmid DNA was diluted in empty vector DNA then added to 1.163 mL of phenol red free OptiMEM (Thermo Fisher). Plasmid DNA concentrations used in each assay described in Table 1. The plasmid DNA was mixed with 78.5 μL of Fugene HD (Promega) and allowed to incubate 5 minutes.

TABLE 1

| | Concentration of | Concentration of Empty |
|---|---|---|
| Plasmid | Plasmid DNA | Vector |
| PARP14 | 0.0002 μg/μL | 0.0198 μg/μL |
| (pRTX240) | | |

Next, 1.125 mL were added to 10 million 293T cells in DMEM (Thermo Fisher) supplemented with 10% FBS (VWR) and 1× Glutamax (Gibco). The transfection was incubated for 24 hrs at 37 degrees in an incubator supplemented with 5% CO2. The cells were then trypsinized and brought up in phenol red free OptiMEM media. Transfected HEK-293T cells were diluted to 125,000 cells per mL and then added to assay plate (Corning 3574) using a Multidrop (Thermo Fisher) to add 40 μL per well of the 384 well plate, resulting in 5,000 cells per well. 40 nL of a dose response curve diluted in DMSO of each test compound was added to the cell plate using a Mosquito (TTP Labtech) and the plate was incubated at 37 degrees for 2 hours. Assay plate was brought to room temperature then 20 μL per well of NanoGlo (Promega) was added to the plate. Luminescence was measured on an Envision (Perkin Elmer).

The average DMSO was calculated from 32 wells containing 0.1% DMSO only in columns 12 and 24 of the assay plate. The % of DMSO values were calculated as described below:

$$\% of D M S O = \frac{Luminescence}{Avg D M S O} * 100$$

The % of DMSO values were plotted as a function of compound concentration and the following 4-parameter fit was applied to derive the DC₅₀ values:

$$Y = Bottom + \frac{( Top - Bottom )}{( 1 + {( \frac{X}{D {C}_{50}} )}^{Hill Coefficient}}$$

where top, bottom, and Hill Coefficient are allowed to float. Y is the % of DMSO and X is the compound concentration.

DC₅₀ data for the Example compounds is provided below in Table 2 (“+” is <0.1 μM; “++” is ≥0.1 μM and <1 μM; and “+++” is ≥1 μM).

### Example B: HiBiT Assay for the Degradation of PARP14

### HiBiT Tagged PARP14 in LgBiT Expressing Jurkat Cells

Jurkat cells stably transfected with LgBiT (Promega) were engineered to contain a HiBiT tag on both alleles of the PARP14 gene (Genbank Accession Number: NM_017554) via CRISPR/Cas9 editing. The HiBiT tag is an 11 amino acid tag created by Promega that associates with LgBiT protein to form the NanoLuc® tag on the C-terminus of PARP14. Clones were isolated and confirmed for the HiBiT tag via Sanger sequencing.

### Assay for Degradation of PARP14

Degradation of PARP14 protein was assessed by measuring the luminescence of the HiBiT tag associating with the LgBiT protein as a proxy for the PARP14 protein. Jurkat cells were diluted to 250,000 cells per mL and then added to assay plate (Corning 3574) using a Multidrop (Thermo Fisher) to add 20 μL per well of the 384 well plate, resulting in 5,000 cells per well. 20 nL of a dose response curve diluted in DMSO of each test compound was added to the cell plate using a Mosquito (TTP Labtech) and the plate was incubated at 37° C. for 2 or 24 hours. Assay plate was brought to room temperature then 5 μL per well of Live Cell Substrate (Promega) was added to the plate. Luminescence was measured on an Envision (Perkin Elmer).

The average DMSO was calculated from 32 wells containing 0.1% DMSO only in columns 12 and 24 of the assay plate. The % of DMSO values were calculated as described below:

$$\% of D M S O = \frac{Luminescence}{Avg D M S O} * 100$$

The % of DMSO values were plotted as a function of compound concentration and the following 4-parameter fit was applied to derive the DC₅₀ values:

$$Y = Bottom + \frac{( Top - Bottom )}{( 1 + {( \frac{X}{D {C}_{50}} )}^{Hill Coefficient}}$$

where top, bottom, and Hill Coefficient are allowed to float. Y is the % of DMSO and X is the compound concentration.

DC₅₀ data for the Example compounds is provided below in Table 2 (“+” is <0.03 μM; “++” is ≥0.03 μM).

TABLE 2
DC₅₀ Data for Example Compounds

| Example No. | DC₅₀ | Assay¹ |
|---|---|---|
| 1 | + | A |
| 2 | + | A |
| 3 | + | A |
| 4 | ++ | A |
| 5 | + | A |
| 6 | + | A |
| 7 | + | A |
| 8 | + | A |
| 9 | + | A |
| 10 | + | A |
| 11 | + | A |
| 12 | + | A |
| 13 | + | A |
| 14 | + | A |
| 15 | + | A |
| 16 | + | A |
| 17 | + | A |
| 18 | + | A |
| 19 | + | A |
| 20 | + | A |
| 21 | + | A |
| 22 | + | A |
| 23 | + | A |
| 24 | + | A |
| 25 | + | A |
| 26 | + | A |
| 27 | + | A |
| 28 | + | A |
| 29 | + | A |
| 30 | + | A |
| 31 | ++ | A |
| 32 | + | A |
| 33 | + | A |
| 34 | + | A |
| 35 | + | A |
| 36 | + | A |
| 37 | + | A |
| 38 | + | A |
| 39 | + | A |
| 40 | + | A |
| 41 | + | A |
| 42 | + | B |
| 43 | + | B |
| 44 | + | B |
| 45 | + | B |
| 46 | + | B |
| 47 | + | B |
| 48 | + | B |
| 49 | + | B |
| 50 | + | B |
| 51 | + | B |
| 52 | + | B |
| 53 | + | B |
| 54 | + | B |
| 55 | + | B |
| 56 | + | B |
| 57 | + | B |
| 58 | + | B |
| 59 | + | B |
| 60 | + | B |
| 61 | + | B |
| 62 | + | B |
| 63 | + | B |
| 64 | + | B |
| 65 | + | B |
| 66 | + | B |
| 67 | + | B |

¹“A” designates assay described in Example A; “B” designates assay described in Example B

### Example C: Decrease of Cell Counts and Cytokines in BALF and Lung Homogenate Following Alternaria-Sensitization and Treatment with a PARP14 Degrader

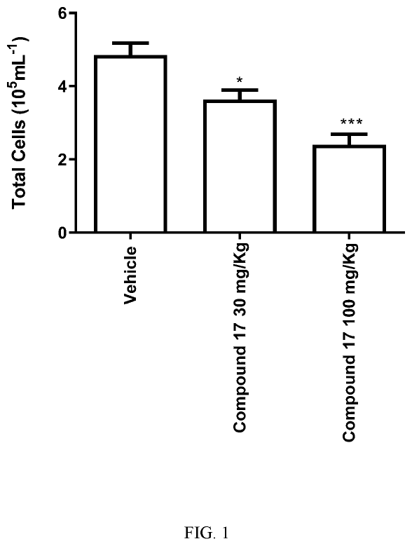

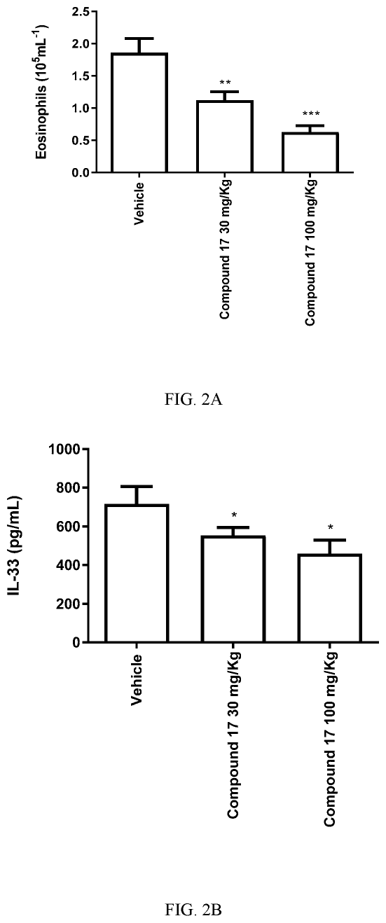

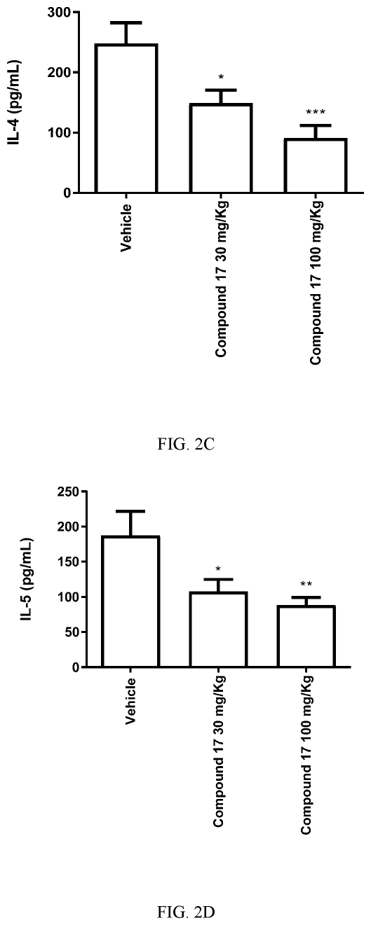

The effect of Compound 17 was studied in an Alternaria asthma mouse model. On days 1-5, male Balb/c mice under isoflurane anesthesia were challenged by instilling solution of 5 μg (protein weight) Alternaria in 40 μL of PBS into each nostril. Compound 17 was administered two days prior to Alternaria challenge (defined day as day −1) and animals were treated with vehicle (10% DMSO/45% PEG-400/45% ‘20% HP-3-CD’ in water) or Compound 17 (30 and 100 mg/kg) once daily for 7 days (defined as day −1 to day 5) by subcutaneous injection. Total and differential cell counts of the BALF fluid samples were measured using a XT-2000iV analyzer (Sysmex). Cytokine concentrations of BALF supernatant were measured in samples from all groups using ELISA kit (Biotechne, UK). Statistical significance is calculated using one-way ANOVA followed by Dunnett's post-tests in which the treatment groups were compared to vehicle control (P<0.05). FIG. 1 illustrates that Compound 17 significantly reduces total cell counts in a dose-dependent manner starting from 30 mg/kg dose. FIG. 2 illustrates that Compound 17 also significantly reduces eosinophils and cytokines IL-33, IL-4, and IL-5 in BALF in a dose-dependent manner starting from 30 mg/kg.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula (I): 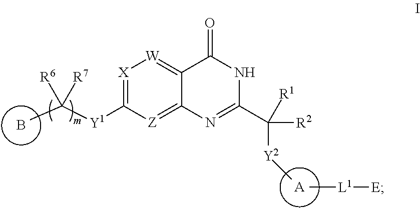
or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, —O—, and —(C₂₋₄ alkynyl)-;
Y² is selected from —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and (—NR³)CH₂—;
Ring A is selected from 6-10 membered aryl, 5-10 membered heteroaryl, C₃₋₁₄ cycloalkyl, and 3-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C_{2-M} dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{b2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR²C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2} NR^{c2}C(O)OR^{a2}, NR^{c2}C(O)NR^{c2}R^{d2}, NR^{c2}S(O)R^{b2}, NR^{c2}S(O)₂R^{b2} NR^{c2}S(O)₂NR^{c2}R², S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{o4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{o4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase;
L¹ is selected from the following:
(vii) bond, such that ring A is directly attached to moiety E;
(viii) —(C₁₋₄ alkyl)-;
(ix) —(C₂₋₄ alkenyl)-;
(x) —(C₂₋₄ alkynyl);
(xi) the following structure: 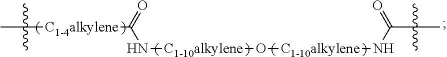
and
(xii) the following structure: 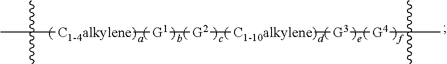
wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl;
G³ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G⁴ is 4-10 membered heterocycloalkyl;
each R^{G} is independently selected from H, methyl, and ethyl;
a is 0 or 1;
b is 0 or 1;
c is 0 or 1;
d is 0 or 1;
e is 0 or 1;
f is 0 or 1;
wherein at least one of b, c, e, and f is 1; and
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups;
with the proviso that the compound is other than:
2-(4-(((7-(Cyclopentylamino)-5-fluoro-4-oxo-3,4-dihydroquinazolin-2-yl)methyl)thio)piperidin-1-yl)-N-(6-((2-(2,6-dioxopiperidin-3-yl)-1,3-dioxoisoindolin-4-yl)amino)hexyl)acetamide;
(2S,4R)-1-((S)-2-(7-(2-(4-(((7-(cyclopentylamino)-5-fluoro-4-oxo-3,4-dihydroquinazolin-2-yl)methyl)thio)piperidin-1-yl)acetamido)heptanamido)-3,3-dimethylbutanoyl)-4-hydroxy-N-(4-(4-methylthiazol-5-yl)benzyl)pyrrolidine-2-carboxamide;
8-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)octanamide; and
3-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)propanamide.

2. A compound of Formula (I): 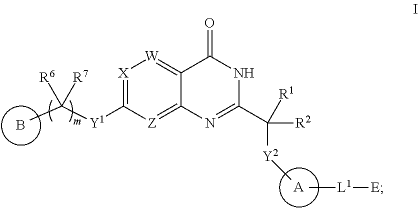
or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, and —O—;
Y² is selected from —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and (—NR³)CH₂—;
Ring A is selected from 6-10 membered aryl, 5-10 membered heteroaryl, C₃₋₁₄ cycloalkyl, and 3-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋₈ dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR²R², C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
R^{a4}, R^{b4}, R⁴, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{e2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R⁴, NR^{c4}S(O)₂R^{b4}, NR^{c4}S(O)₂NR^{c4}R^{d4}, and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase;
L¹ is selected from the following:
(xiii) bond, such that ring A is directly attached to moiety E;
(xiv) —(C₁₋₄ alkyl)-;
(xv) —(C₂₋₄ alkenyl)-;
(xvi) —(C₂₋₄ alkynyl)-;
(xvii) the following structure: 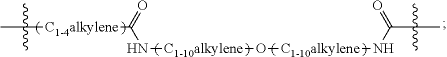
and
(xviii) the following structure: 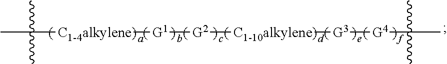
wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl;
G³ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G⁴ is 4-10 membered heterocycloalkyl;
each R^{G} is independently selected from H, methyl, and ethyl;
a is 0 or 1;
b is 0 or 1;
c is 0 or 1;
d is 0 or 1;
e is 0 or 1;
f is 0 or 1;
wherein at least one of b, c, e, and f is 1; and
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups;
with the proviso that the compound is other than:
2-(4-(((7-(Cyclopentylamino)-5-fluoro-4-oxo-3,4-dihydroquinazolin-2-yl)methyl)thio)piperidin-1-yl)-N-(6-((2-(2,6-dioxopiperidin-3-yl)-1,3-dioxoisoindolin-4-yl)amino)hexyl)acetamide;
(2S,4R)-1-((S)-2-(7-(2-(4-(((7-(cyclopentylamino)-5-fluoro-4-oxo-3,4-dihydroquinazolin-2-yl)methyl)thio)piperidin-1-yl)acetamido)heptanamido)-3,3-dimethylbutanoyl)-4-hydroxy-N-(4-(4-methylthiazol-5-yl)benzyl)pyrrolidine-2-carboxamide;
8-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)octanamide; and
3-((2-(2,6-dioxopiperidin-3-yl)-1,3-dioxoisoindolin-4-yl)amino)-N-((1r,4r)-4-(((5-fluoro-4-oxo-7-((tetrahydro-2H-pyran-4-yl)methoxy)-3,4-dihydroquinazolin-2-yl)methyl)thio)cyclohexyl)propanamide.

3. The compound of claim 1, or a pharmaceutically acceptable salt thereof, wherein W is CR^{W}.

4. The compound of claim 1, or a pharmaceutically acceptable salt thereof, wherein X is CR^{X}.

5. The compound of claim 1, or a pharmaceutically acceptable salt thereof, wherein Z is CR^{Z}.

6. The compound of claim 1, or a pharmaceutically acceptable salt thereof, wherein Y¹ is —O—.

7. The compound of claim 1, or a pharmaceutically acceptable salt thereof, wherein Y¹ is —NR³—.

8. The compound of claim 1, or a pharmaceutically acceptable salt thereof, wherein Y¹ is —(C₂₋₄ alkynyl)-.

9. The compound of claim 1, or a pharmaceutically acceptable salt thereof, wherein Y² is —S—.

10. The compound of claim 1, or a pharmaceutically acceptable salt thereof, wherein Y² is —CH₂—.

11. The compound of claim 1, or a pharmaceutically acceptable salt thereof, wherein Y² is —O—.

12. The compound of claim 1, or a pharmaceutically acceptable salt thereof, wherein Ring A is monocyclic or polycyclic 4-18 membered heterocycloalkyl, wherein Ring A is optionally substituted by 1, 2, 3, or 4 R^{A}.

13. The compound of claim 1, or a pharmaceutically acceptable salt thereof, wherein Ring A is monocyclic 4-7 membered heterocycloalkyl, wherein Ring A is optionally substituted by 1, 2, 3, or 4 R^{A}.

14. The compound of claim 1, or a pharmaceutically acceptable salt thereof, wherein Ring A is piperidinyl.

15. The compound of claim 1, or a pharmaceutically acceptable salt thereof, wherein Ring A is piperazinyl.

16. The compound of claim 1, or a pharmaceutically acceptable salt thereof, wherein Ring A is C₃₋₁₄ cycloalkyl, wherein Ring A is optionally substituted by 1, 2, 3, or 4 R^{A}.

17. The compound of claim 1, or a pharmaceutically acceptable salt thereof, wherein Ring A is C₃₋₇ cycloalkyl, wherein Ring A is optionally substituted by 1, 2, 3, or 4 R^{A}.

18. The compound of claim 1, or a pharmaceutically acceptable salt thereof, wherein Ring A is cyclohexyl.

19. The compound of claim 1, or a pharmaceutically acceptable salt thereof, wherein Ring B is 4-18 membered heterocycloalkyl, wherein Ring B is optionally substituted by 1, 2, 3, or 4 R^{B}.

20. The compound of claim 1, or a pharmaceutically acceptable salt thereof, wherein Ring B is piperidinyl or tetrahydro-2H-pyranyl optionally substituted by 1, 2, 3, or 4 R^{B}.

21. The compound of claim 1, or a pharmaceutically acceptable salt thereof, wherein Ring B is tetrahydro-2H-pyran-4-yl or 1-acetylpiperidin-4-yl.

22. The compound of claim 1, or a pharmaceutically acceptable salt thereof, wherein Ring B is C₃₋₁₄ cycloalkyl, wherein Ring B is optionally substituted by 1, 2, 3, or 4 R^{B}.

23. The compound of claim 1, or a pharmaceutically acceptable salt thereof, wherein Ring B is cyclopentyl or cyclopropyl.

24. The compound of claim 1, or a pharmaceutically acceptable salt thereof, wherein R¹ and R² are each H.

25. The compound of claim 1, or a pharmaceutically acceptable salt thereof, wherein R³ is H.

26. The compound of claim 1, or a pharmaceutically acceptable salt thereof, wherein R⁴ and R⁵ are each H.

27. The compound of claim 1, or a pharmaceutically acceptable salt thereof, wherein R⁶ and R⁷ are each H.

28. The compound of claim 1, or a pharmaceutically acceptable salt thereof, wherein each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

29. The compound of claim 1, or a pharmaceutically acceptable salt thereof, wherein each R^{A} is independently selected from halo, C₁₋₆ alkyl, C₁₋₆ haloalkyl, CN, NO₂, or OR^{a1}.

30. The compound of claim 1, or a pharmaceutically acceptable salt thereof, wherein each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

31. The compound of claim 1, or a pharmaceutically acceptable salt thereof, wherein each R^{B} is independently selected from halo, C₁₋₆ alkyl, C₁₋₆ haloalkyl, CN, NO₂, C(O)R^{b2}, or OR^{a2}.

32. The compound of claim 1, or a pharmaceutically acceptable salt thereof, wherein each R^{B} is C(O)CH₃.

33. The compound of claim 1, or a pharmaceutically acceptable salt thereof, wherein R^{W} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

34. The compound of claim 1, or a pharmaceutically acceptable salt thereof, wherein R^{W} is selected from H, halo, C₁₋₆ alkyl, C₂₋₆ alkenyl, C₂₋₆ alkynyl, C₁₋₆ haloalkyl, CN, NO₂, and OR^{a3}.

35. The compound of claim 1, or a pharmaceutically acceptable salt thereof, wherein R^{W} is selected from H, halo, and C₁₋₆ haloalkyl.

36. The compound of claim 1, or a pharmaceutically acceptable salt thereof, wherein R^{W} is F.

37. The compound of claim 1, or a pharmaceutically acceptable salt thereof, wherein R^{X} is selected from C₆₋₁₀ aryl and 5-10 membered heteroaryl, wherein said C₆₋₁₀ aryl and 5-10 membered heteroaryl are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, OR^{a3}, and SR^{a3}.

38. The compound of claim 1, or a pharmaceutically acceptable salt thereof, wherein R^{X} is selected from H, halo, C₁₋₆ alkyl, C₂₋₆ alkenyl, C₂₋₆ alkynyl, C₁₋₆ haloalkyl, CN, OR^{a3}, and C₆₋₁₀ aryl.

39. The compound of claim 1, or a pharmaceutically acceptable salt thereof, wherein R^{X} is H.

40. The compound of claim 1, or a pharmaceutically acceptable salt thereof, wherein R^{Z} is selected from H, halo, C₁₋₆ alkyl, C₂₋₆ alkenyl, C₂₋₆ alkynyl, C₁₋₆ haloalkyl, CN, NO₂, and OR^{a3}.

41. The compound of claim 1, or a pharmaceutically acceptable salt thereof, wherein R^{Z} is H.

42. The compound of claim 1, or a pharmaceutically acceptable salt thereof, wherein m is 0.

43. The compound of claim 1, or a pharmaceutically acceptable salt thereof, wherein m is 1.

44. The compound of claim 1, or a pharmaceutically acceptable salt thereof, wherein L¹ is a bond, such that ring A is directly attached to moiety E.

45. The compound of claim 1, or a pharmaceutically acceptable salt thereof, wherein L¹ is —(C₁₋₄ alkyl).

46. The compound of claim 1, or a pharmaceutically acceptable salt thereof, wherein L¹ is —(C₂₋₄ alkynyl).

47. The compound of claim 1, or a pharmaceutically acceptable salt thereof, wherein L¹ is the following structure:

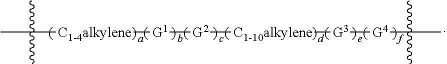

48. The compound of claim 1, or a pharmaceutically acceptable salt thereof, wherein L¹ is the following structure:

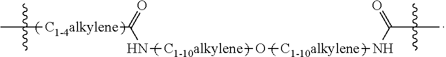

49. The compound of claim 1, or a pharmaceutically acceptable salt thereof, wherein G¹ is —NR^{G}C(O) or —C(O)—.

50. The compound of claim 1, or a pharmaceutically acceptable salt thereof, wherein G¹ is —NR^{G}C(O)—, —C(O)—, or —O—.

51. The compound of claim 1, or a pharmaceutically acceptable salt thereof, wherein G² is 4-10 membered heterocycloalkyl.

52. The compound of claim 1, or a pharmaceutically acceptable salt thereof, wherein G² is piperidinyl, piperazinyl, or azetidinyl.

53. The compound of claim 1, or a pharmaceutically acceptable salt thereof, wherein G² is pyrrolidinyl, piperidinyl, piperazinyl, or azetidinyl.

54. The compound of claim 1, or a pharmaceutically acceptable salt thereof, wherein G³ is —NR^{G}C(O)—, —NR^{G}— or —C(O)—.

55. The compound of claim 1, or a pharmaceutically acceptable salt thereof, wherein G⁴ is piperidinyl or piperazinyl.

56. The compound of claim 1, or a pharmaceutically acceptable salt thereof, wherein a is 0.

57. The compound of claim 1, or a pharmaceutically acceptable salt thereof, wherein a is 1.

58. The compound of claim 1, or a pharmaceutically acceptable salt thereof, wherein b is 0.

59. The compound of claim 1, or a pharmaceutically acceptable salt thereof, wherein b is 1.

60. The compound of claim 1, or a pharmaceutically acceptable salt thereof, wherein c is 0.

61. The compound of claim 1, or a pharmaceutically acceptable salt thereof, wherein c is 1.

62. The compound of claim 1, or a pharmaceutically acceptable salt thereof, wherein d is 0.

63. The compound of claim 1, or a pharmaceutically acceptable salt thereof, wherein d is 1.

64. The compound of claim 1, or a pharmaceutically acceptable salt thereof, wherein e is 0.

65. The compound of claim 1, or a pharmaceutically acceptable salt thereof, wherein e is 1.

66. The compound of claim 1, or a pharmaceutically acceptable salt thereof, wherein f is 0.

67. The compound of claim 1, or a pharmaceutically acceptable salt thereof, wherein f is 1.

68. The compound of claim 1, or a pharmaceutically acceptable salt thereof, wherein R^{G} is H.

69. The compound of claim 1, or a pharmaceutically acceptable salt thereof, wherein E is an E3 ubiquitin ligase binding moiety that binds to cereblon.

70. The compound of claim 1, or a pharmaceutically acceptable salt thereof, wherein E is selected from the following:

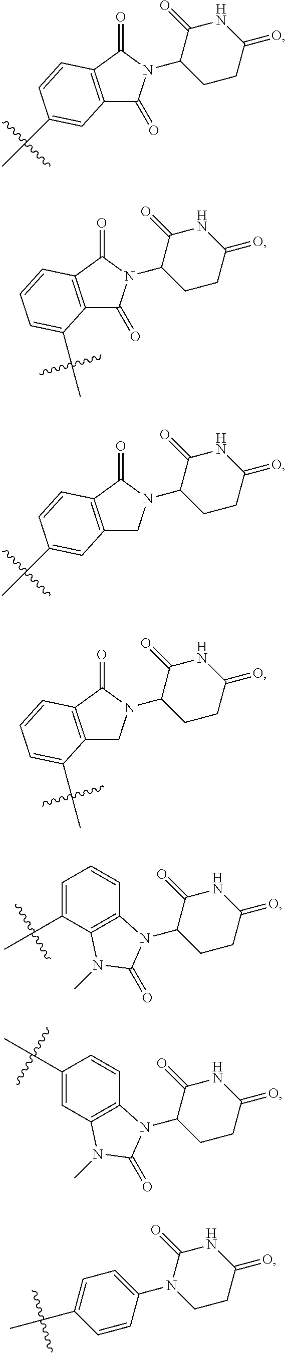

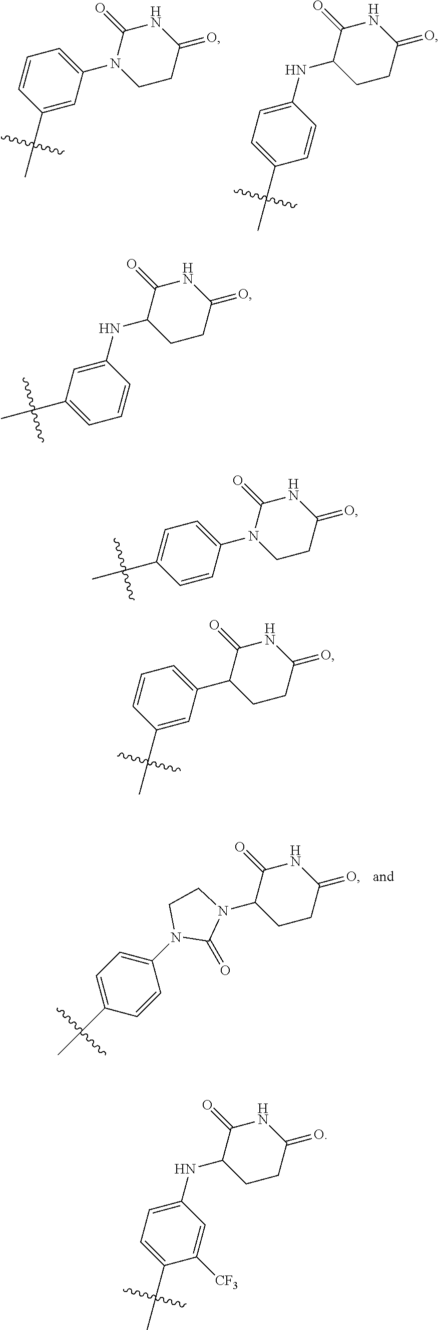

71. The compound of claim 1, or a pharmaceutically acceptable salt thereof, wherein E is selected from the following:

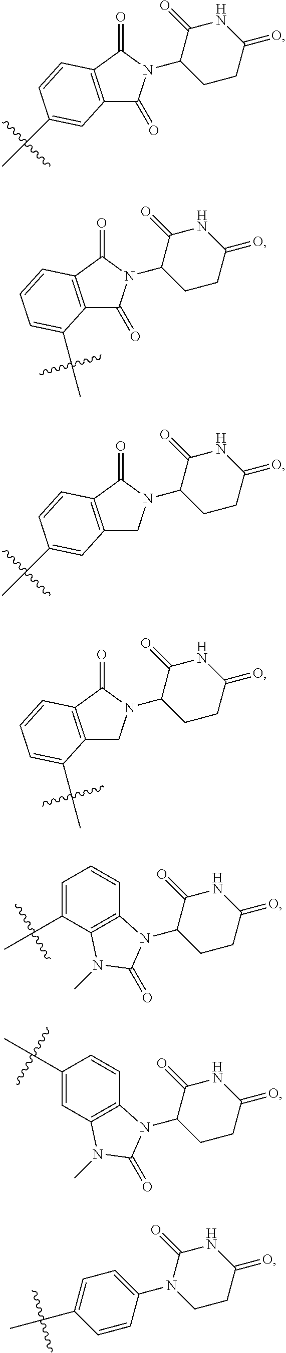

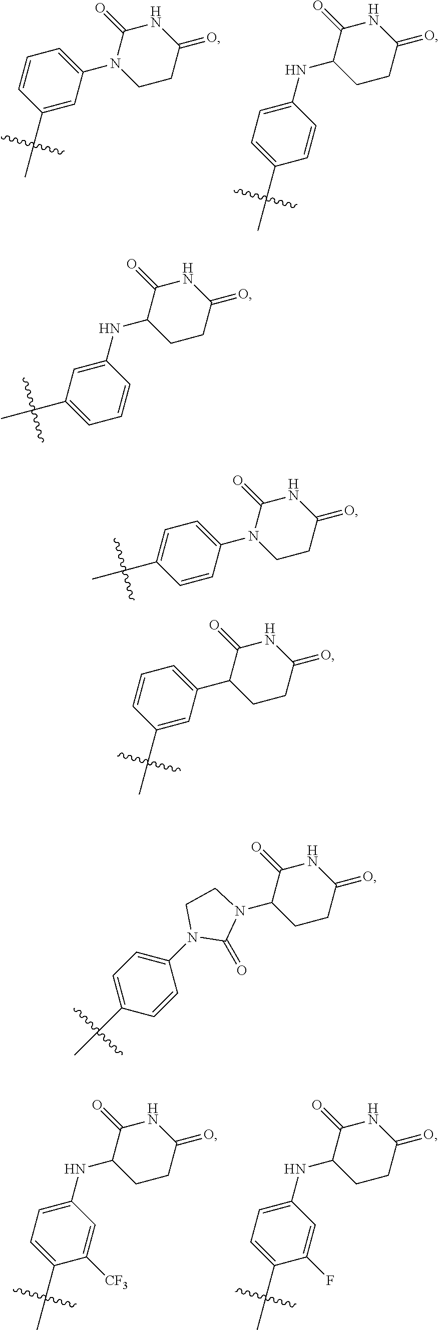

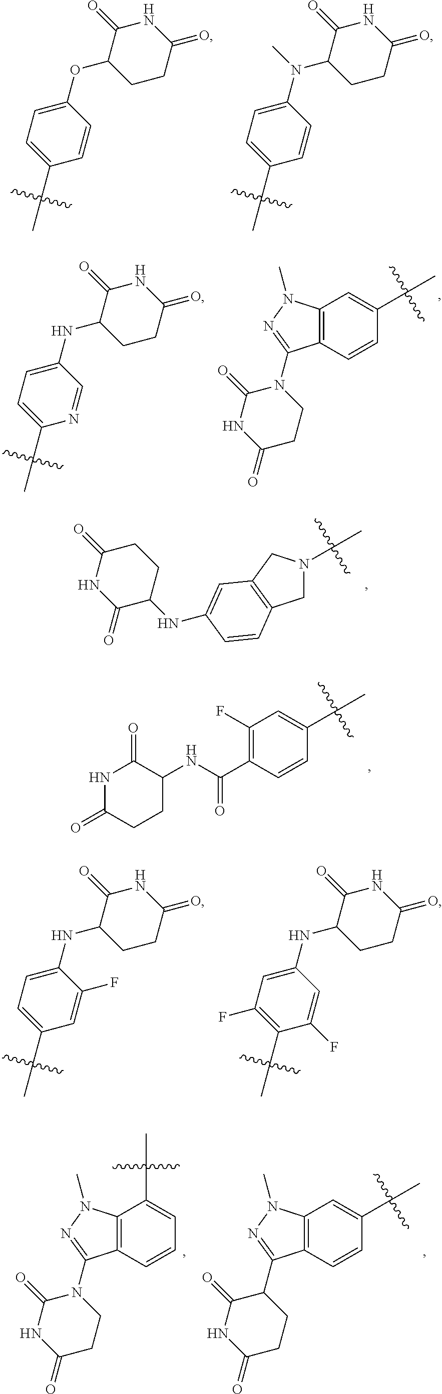

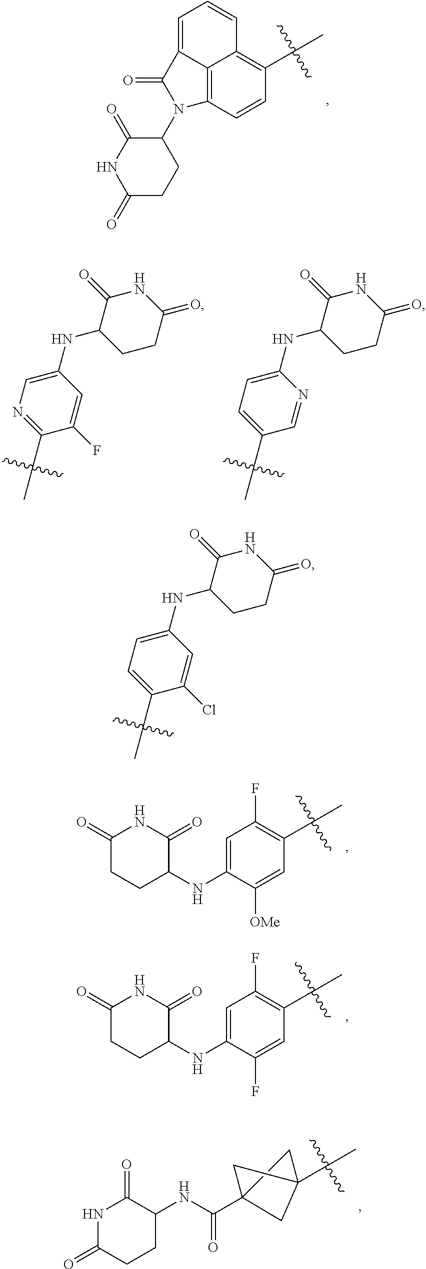

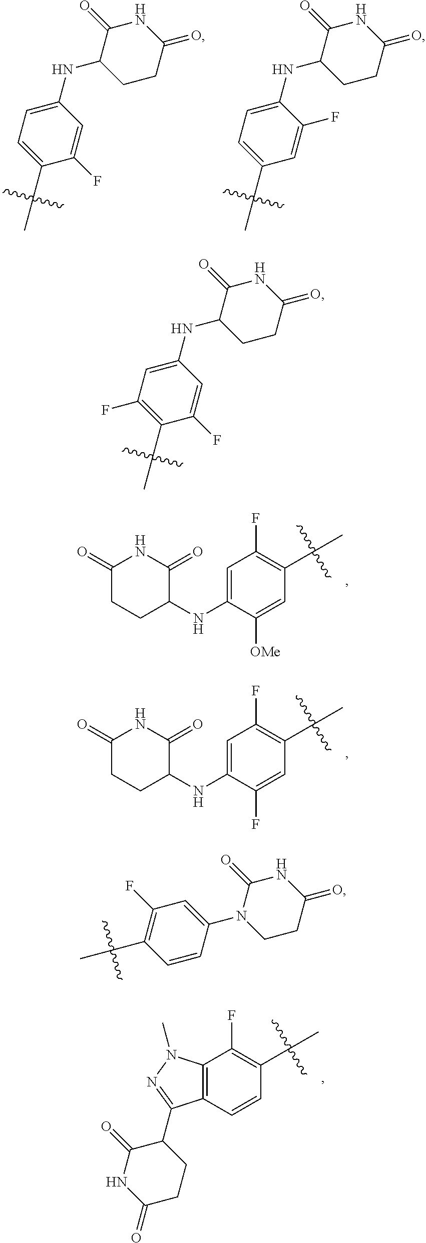

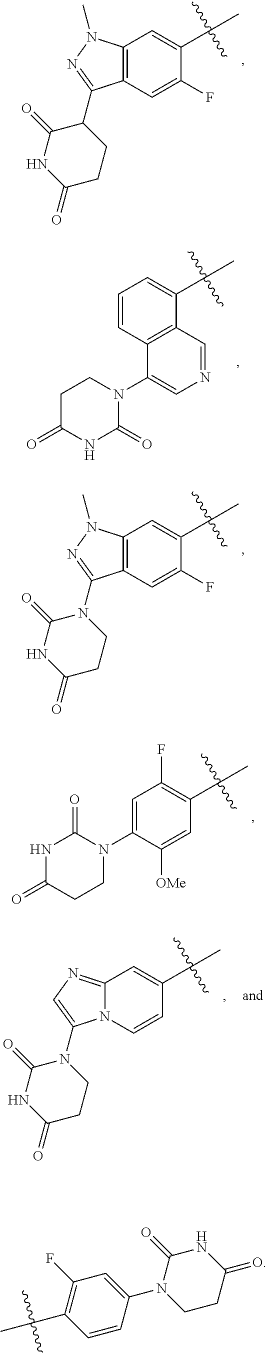 [SMILES: *c1cc2c(cc1F)c(C1CCC(=O)NC1=O)nn2C ; *c1cccc2c(N3CCC(=O)NC3=O)cncc12 ; *c1cc2c(cc1F)c(N1CCC(=O)NC1=O)nn2C ; *c1cc(OC)c(N2CCC(=O)NC2=O)cc1F ; *c1ccn2c(N3CCC(=O)NC3=O)cnc2c1 ; *c1ccc(N2CCC(=O)NC2=O)cc1F]

72. The compound of claim 1, or a pharmaceutically acceptable salt thereof, wherein E is selected from:

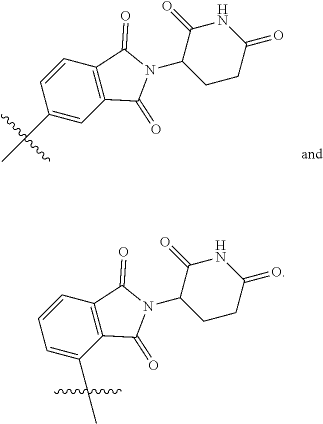 [SMILES: *c1ccc2c(c1)C(=O)N(C1CCC(=O)NC1=O)C2=O ; *c1cccc2c1C(=O)N(C1CCC(=O)NC1=O)C2=O]

73. The compound of claim 1, or a pharmaceutically acceptable salt thereof, wherein E is:

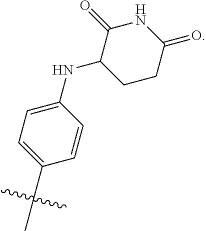 [SMILES: *c1ccc(NC2CCC(=O)NC2=O)cc1]

74. The compound of claim 1, having Formula II: 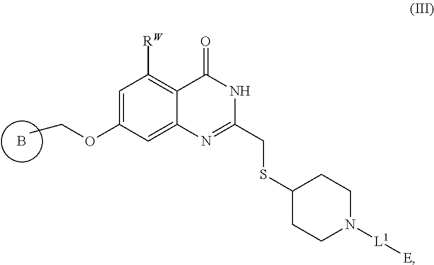
or a pharmaceutically acceptable salt thereof.

75. The compound of claim 1, having Formula IIIa: 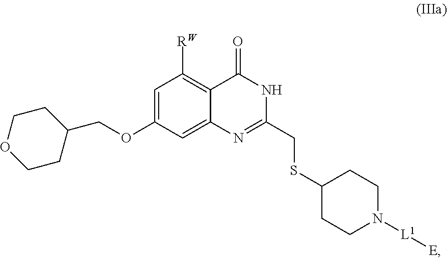
or a pharmaceutically acceptable salt thereof.

76. The compound of claim 1, having Formula IIIb: 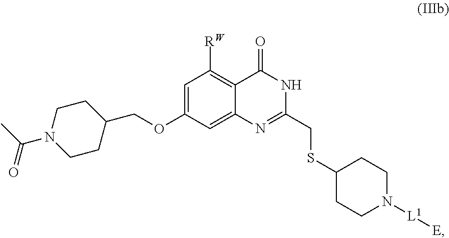
or a pharmaceutically acceptable salt thereof.

77. The compound of claim 1, having Formula IIIc: 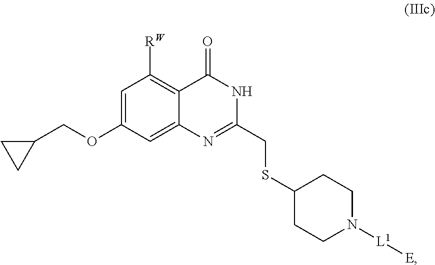
or a pharmaceutically acceptable salt thereof.

78. The compound of claim 1, wherein the compound is selected from:
4-(4-((1-(2-(4-(((7-(cyclopentylamino)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
2-(2,6-dioxopiperidin-3-yl)-4-(4-((1-(2-(4-(((5-fluoro-4-oxo-7-((tetrahydro-2H-pyran-4-yl)methoxy)-3,4-dihydroquinazolin-2-yl)methyl)thio)piperidin-1-yl)acetyl)piperidin-4-yl)methyl)piperazin-1-yl)isoindoline-1,3-dione;
3-(2,4-dioxotetrahydropyrimidin-1(2H)-yl)-N-(6-(2-(4-(((5-fluoro-4-oxo-7-((tetrahydro-2H-pyran-4-yl)methoxy)-3,4-dihydroquinazolin-2-yl)methyl)thio)piperidin-1-yl)acetamido)hexyl)benzamide;
3-(2,4-dioxotetrahydropyrimidin-1(2H)-yl)-N-(4-(2-(4-(((5-fluoro-4-oxo-7-((tetrahydro-2H-pyran-4-yl)methoxy)-3,4-dihydroquinazolin-2-yl)methyl)thio)piperidin-1-yl)acetamido)butyl)benzamide;
N-(6-(4-(4-((2,6-dioxopiperidin-3-yl)amino)phenyl)piperidin-1-yl)hexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(6-(4-(4-((2,6-dioxopiperidin-3-yl)amino)phenyl)piperidin-1-yl)-6-oxohexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(4-((2,6-dioxopiperidin-3-yl)amino)phenyl)piperidin-1-yl)butyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(4-((2,6-dioxopiperidin-3-yl)amino)phenyl)piperidin-1-yl)-4-oxobutyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(4-((2,6-dioxopiperidin-3-yl)amino)phenyl)piperidin-1-yl)ethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(4-((2,6-dioxopiperidin-3-yl)amino)phenyl)piperidin-1-yl)-2-oxoethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(3-((2,6-dioxopiperidin-3-yl)amino)phenyl)piperidin-1-yl)-2-oxoethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(6-(4-(3-((2,6-dioxopiperidin-3-yl)amino)phenyl)piperidin-1-yl)-6-oxohexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(3-((2,6-dioxopiperidin-3-yl)amino)phenyl)piperidin-1-yl)butyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(4-(4-(3-((2,6-dioxopiperidin-3-yl)amino)phenyl)piperidin-1-yl)-4-oxobutyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(2-(4-(3-((2,6-dioxopiperidin-3-yl)amino)phenyl)piperidin-1-yl)ethyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
N-(6-(4-(3-((2,6-dioxopiperidin-3-yl)amino)phenyl)piperidin-1-yl)hexyl)-2-(4-(((5-fluoro-4-oxo-7-((tetrahydro-2H-pyran-4-yl)methoxy)-3,4-dihydroquinazolin-2-yl)methyl)thio)piperidin-1-yl)acetamide;
3-((4-(1-((1-(2-(4-(((5-fluoro-4-oxo-7-((tetrahydro-2H-pyran-4-yl)methoxy)-3,4-dihydroquinazolin-2-yl)methyl)thio)piperidin-1-yl)acetyl)piperidin-4-yl)methyl)piperidin-4-yl)phenyl)amino)piperidine-2,6-dione;
3-(3-(4-(1-(2-(4-(((7-((1-acetylpiperidin-4-yl)methoxy)-5-fluoro-4-oxo-3,4-dihydroquinazolin-2-yl)methyl)thio)piperidin-1-yl)acetyl)piperidin-4-yl)phenyl)-2-oxoimidazolidin-1-yl)piperidine-2,6-dione;
4-(4-((1-(2-(4-(((7-((1-acetylpiperidin-4-yl)methoxy)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
1-(4-(1-((1-(2-(4-(((5-fluoro-4-oxo-7-((tetrahydro-2H-pyran-4-yl)methoxy)-3,4-dihydroquinazolin-2-yl)methyl)thio)piperidin-1-yl)acetyl)piperidin-4-yl)methyl)piperidin-4-yl)phenyl)dihydropyrimidine-2,4(1H,3H)-dione;
4-((2-(4-(((7-((1-acetylpiperidin-4-yl)methoxy)-5-fluoro-4-oxo-3,4-dihydroquinazolin-2-yl)methyl)thio)piperidin-1-yl)-2-oxoethyl)amino)-2-(2,6-dioxopiperidin-3-yl)isoindoline-1,3-dione;
4-(4-((4-(4-(((7-((1-acetylpiperidin-4-yl)methoxy)-5-fluoro-4-oxo-3,4-dihydroquinazolin-2-yl)methyl)thio)piperidine-1-carbonyl)piperidin-1-yl)methyl)piperidin-1-yl)-2-(2,6-dioxopiperidin-3-yl)isoindoline-1,3-dione;
4-(4-((4-(4-(((7-(cyclopentylamino)-5-fluoro-4-oxo-3,4-dihydroquinazolin-2-yl)methyl)thio)piperidine-1-carbonyl)piperidin-1-yl)methyl)piperidin-1-yl)-2-(2,6-dioxopiperidin-3-yl)isoindoline-1,3-dione;
5-((2-(4-(((7-((1-acetylpiperidin-4-yl)methoxy)-5-fluoro-4-oxo-3,4-dihydroquinazolin-2-yl)methyl)thio)piperidin-1-yl)-2-oxoethyl)amino)-2-(2,6-dioxopiperidin-3-yl)isoindoline-1,3-dione;
3-((4-(4-(((7-((1-acetylpiperidin-4-yl)methoxy)-5-fluoro-4-oxo-3,4-dihydroquinazolin-2-yl)methyl)thio)piperidine-1-carbonyl)phenyl)amino)piperidine-2,6-dione;
4-(4-((4-(4-(2-(7-((1-acetylpiperidin-4-yl)methoxy)-5-fluoro-4-oxo-3,4-dihydroquinazolin-2-yl)ethyl)piperidine-1-carbonyl)piperidin-1-yl)methyl)piperidin-1-yl)-2-(2,6-dioxopiperidin-3-yl)isoindoline-1,3-dione;
4-(4-((1-(2-(4-(2-(7-((1-acetylpiperidin-4-yl)methoxy)-5-fluoro-4-oxo-3,4-dihydroquinazolin-2-yl)ethyl)piperidin-1-yl)acetyl)piperidin-4-yl)methyl)piperazin-1-yl)-2-(2,6-dioxopiperidin-3-yl)isoindoline-1,3-dione;
5-(4-((1-(2-(4-(((7-((1-acetylpiperidin-4-yl)methoxy)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
5-(4-((1-(2-(4-(((7-(cyclopentylamino)-5-fluoro-4-oxo-3,4-dihydroquinazolin-2-yl)methyl)thio)piperidin-1-yl)acetyl)piperidin-4-yl)methyl)piperazin-1-yl)-2-(2,6-dioxopiperidin-3-yl)isoindoline-1,3-dione;
5-(4-(((7-((1-acetylpiperidin-4-yl)methoxy)-5-fluoro-4-oxo-3,4-dihydroquinazolin-2-yl)methyl)thio)piperidin-1-yl)-2-(2,6-dioxopiperidin-3-yl)isoindoline-1,3-dione;
2-(4-(((7-((1-acetylpiperidin-4-yl)methoxy)-5-fluoro-4-oxo-3,4-dihydroquinazolin-2-yl)methyl)thio)piperidin-1-yl)-N-(2-(2,6-dioxopiperidin-3-yl)-1,3-dioxoisoindolin-4-yl)acetamide;
2-(4-(((7-((1-acetylpiperidin-4-yl)methoxy)-5-fluoro-4-oxo-3,4-dihydroquinazolin-2-yl)methyl)thio)piperidin-1-yl)-N-(2-(2,6-dioxopiperidin-3-yl)-1,3-dioxoisoindolin-5-yl)acetamide;
5-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione;
5-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-2-(2,6-dioxopiperidin-3-yl)isoindoline-1,3-dione;
3-(4-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-3-methyl-2-oxo-2,3-dihydro-1H-benzo[d]imidazol-1-yl)piperidine-2,6-dione;
3-(5-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)-3-methyl-2-oxo-2,3-dihydro-1H-benzo[d]imidazol-1-yl)piperidine-2,6-dione;
1-(4-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)phenyl)dihydropyrimidine-2,4(1H,3H)-dione;
1-(3-(3-(4-(((7-((1-acetylpiperidin-4-yl)methoxy)-5-fluoro-4-oxo-3,4-dihydroquinazolin-2-yl)methyl)thio)piperidin-1-yl)prop-1-yn-1-yl)phenyl)dihydropyrimidine-2,4(1H,3H)-dione;
4-(4-((4-(((7-((1-acetylpiperidin-4-yl)methoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione;
3-(4-(3-((4-(((7-((1-acetylpiperidin-4-yl)methoxy)-5-fluoro-4-oxo-3,4-dihydroquinazolin-2-yl)methyl)thio)piperidin-1-yl)methyl)azetidin-1-yl)-1-oxoisoindolin-2-yl)piperidine-2,6-dione; and
5-(4-((4-(((7-(cyclopropylmethoxy)-5-fluoro-4-oxo-3,4-dihydroquinazolin-2-yl)methyl)thio)piperidin-1-yl)methyl)piperidin-1-yl)-2-(2,6-dioxopiperidin-3-yl)isoindoline-1,3-dione;
or a pharmaceutically acceptable salt of any of the aforementioned.

79. The compound of claim 1, wherein the compound is selected from:
3-((4-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)phenyl)amino)piperidine-2,6-dione;
3-((4-(4-(2-(4-(((5-fluoro-4-oxo-7-((tetrahydro-2H-pyran-4-yl)methoxy)-3,4-dihydroquinazolin-2-yl)methyl)thio)piperidin-1-yl)ethyl)piperazin-1-yl)phenyl)amino)piperidine-2,6-dione;
3-((3-fluoro-4-(4-(2-(4-((5-fluoro-4-oxo-7-((tetrahydro-2H-pyran-4-yl)methoxy)-3,4-dihydroquinazolin-2-yl)methoxy)piperidin-1-yl)ethyl)piperazin-1-yl)phenyl)amino)piperidine-2,6-dione;
3-((4-(4-(2-(4-((7-(cyclopropylmethoxy)-5-fluoro-4-oxo-3,4-dihydroquinazolin-2-yl)methoxy)piperidin-1-yl)ethyl)piperazin-1-yl)-3-fluorophenyl)amino)piperidine-2,6-dione;
3-((3-fluoro-4-(4-(2-(4-(((5-fluoro-4-oxo-7-((tetrahydro-2H-pyran-4-yl)methoxy)-3,4-dihydroquinazolin-2-yl)methyl)thio)piperidin-1-yl)ethyl)piperazin-1-yl)phenyl)amino)piperidine-2,6-dione;
3-((4-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)-3-fluorophenyl)amino)piperidine-2,6-dione;
3-((4-(4-(3-(4-(((7-(cyclopropylmethoxy)-5-fluoro-4-oxo-3,4-dihydroquinazolin-2-yl)methyl)thio)piperidin-1-yl)azetidin-1-yl)piperidin-1-yl)-3-fluorophenyl)amino)piperidine-2,6-dione;
3-(6-(4-(2-(4-(((7-(cyclopropylmethoxy)-5-fluoro-4-oxo-3,4-dihydroquinazolin-2-yl)methyl)thio)piperidin-1-yl)ethyl)piperazin-1-yl)-2-oxobenzo[cd]indol-1(2H)-yl)piperidine-2,6-dione;
3-(5-(4-((4-(((7-(cyclopropylmethoxy)-5-fluoro-4-oxo-3,4-dihydroquinazolin-2-yl)methyl)thio)piperidin-1-yl)methyl)piperidin-1-yl)-1-oxoisoindolin-2-yl)piperidine-2,6-dione;
3-((3-fluoro-4-(4-(3-(4-(((5-fluoro-4-oxo-7-((tetrahydro-2H-pyran-4-yl)methoxy)-3,4-dihydroquinazolin-2-yl)methyl)thio)piperidin-1-yl)azetidin-1-yl)piperidin-1-yl)phenyl)amino)piperidine-2,6-dione;
3-((3-fluoro-4-(4-(((5-fluoro-4-oxo-7-((tetrahydro-2H-pyran-4-yl)methoxy)-3,4-dihydroquinazolin-2-yl)methyl)thio)-[1,4′-bipiperidin]-1′-yl)phenyl)amino)piperidine-2,6-dione;
3-((4-(4-((7-(cyclopropylmethoxy)-5-fluoro-4-oxo-3,4-dihydroquinazolin-2-yl)methoxy)-[1,4′-bipiperidin]-1′-yl)-3-fluorophenyl)amino)piperidine-2,6-dione;
3-((4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)amino) piperidine-2,6-dione;
3-((4-(4-(2-(((1r,4r)-4-(((7-(cyclopropylmethoxy)-5-fluoro-4-oxo-3,4-dihydroquinazolin-2-yl)methyl)thio)cyclohexyl)oxy)ethyl)piperazin-1-yl)-3-fluorophenyl)amino)piperidine-2,6-dione;
3-((4-(4-((4-(((7-(cyclopropylmethoxy)-5-fluoro-4-oxo-3,4-dihydroquinazolin-2-yl)methyl)thio)piperidin-1-yl)methyl)piperidin-1-yl)-3-fluorophenyl)amino)piperidine-2,6-dione;
3-((4-(4-(((7-(cyclopropylmethoxy)-5-fluoro-4-oxo-3,4-dihydroquinazolin-2-yl)methyl)thio)-[1,4′-bipiperidin]-1′-yl)-3-(trifluoromethyl)phenyl)amino)piperidine-2,6-dione;
3-((4-(4-(((7-(cyclopropylmethoxy)-5-fluoro-4-oxo-3,4-dihydroquinazolin-2-yl)methyl)thio)-[1,4′-bipiperidin]-1′-yl)-2,5-difluorophenyl)amino)piperidine-2,6-dione;
3-((4-(3-(4-(((7-(cyclopropylmethoxy)-5-fluoro-4-oxo-3,4-dihydroquinazolin-2-yl)methyl)thio)piperidin-1-yl)pyrrolidin-1-yl)-3-fluorophenyl)amino)piperidine-2,6-dione;
1-(4-(4-(((7-(cyclopropylmethoxy)-5-fluoro-4-oxo-3,4-dihydroquinazolin-2-yl)methyl)thio)-[1,4′-bipiperidin]-1′-yl)-3-fluorophenyl)dihydropyrimidine-2,4(1H,3H)-dione;
1-(6-(4-(((7-(cyclopropylmethoxy)-5-fluoro-4-oxo-3,4-dihydroquinazolin-2-yl)methyl)thio)-[1,4′-bipiperidin]-1′-yl)-1-methyl-1H-indazol-3-yl)dihydropyrimidine-2,4(1H,3H)-dione;
1-(8-(4-(((7-(cyclopropylmethoxy)-5-fluoro-4-oxo-3,4-dihydroquinazolin-2-yl)methyl)thio)-[1,4′-bipiperidin]-1′-yl)isoquinolin-4-yl)dihydropyrimidine-2,4(1H,3H)-dione
3-((4-(4-(4-(2-(7-(cyclopropylmethoxy)-5-fluoro-4-oxo-3,4-dihydroquinazolin-2-yl)ethyl)piperazin-1-yl)piperidin-1-yl)-3-fluorophenyl)amino)piperidine-2,6-dione
3-((4-(1′-(2-(7-(cyclopropylmethoxy)-5-fluoro-4-oxo-3,4-dihydroquinazolin-2-yl)ethyl)-[4,4′-bipiperidin]-1-yl)-3-fluorophenyl)amino)piperidine-2,6-dione
3-((4-(4-(2-(7-(cyclopropylmethoxy)-5-fluoro-4-oxo-3,4-dihydroquinazolin-2-yl)ethyl)-[1,4′-bipiperidin]-1′-yl)-3-fluorophenyl)amino)piperidine-2,6-dione
3-((4-(3-(4-(((7-(cyclopropylmethoxy)-5-fluoro-4-oxo-3,4-dihydroquinazolin-2-yl)methyl)thio)piperidin-1-yl)azetidin-1-yl)-3-fluorophenyl)amino)piperidine-2,6-dione
3-((4-(4-(((7-(cyclopropylethynyl)-5-fluoro-4-oxo-3,4-dihydroquinazolin-2-yl)methyl)thio)-[1,4′-bipiperidin]-1′-yl)-3-fluorophenyl)amino)piperidine-2,6-dione
or a pharmaceutically acceptable salt of any of the aforementioned.

80. A pharmaceutical composition comprising a compound of claim 1, or a pharmaceutically acceptable salt thereof, and at least one pharmaceutically acceptable carrier.

81. A method of degrading PARP14, comprising contacting a compound of claim 1, or a pharmaceutically acceptable salt thereof, with said PARP14.

82. A method of treating cancer in a patient in need of treatment comprising administering to said patient a therapeutically effective amount of a compound of claim 1, or a pharmaceutically acceptable salt thereof.

83. A method of treating cancer in a patient in need of treatment comprising administering to said patient a therapeutically effective amount of a compound of claim 1, or a pharmaceutically acceptable salt thereof, in combination with an additional therapy or therapeutic agent.

84. The method of claim 82 wherein said cancer is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

85. A method of treating an inflammatory disease in a patient in need of treatment comprising administering to said patient a therapeutically effective amount of a compound of claim 1, or a pharmaceutically acceptable salt thereof.

86. The method of claim 85, wherein the inflammatory disease is selected from asthma, atopic dermatitis, psoriasis, rhinitis, systemic sclerosis, keloids, an eosinophilic disorder, pulmonary fibrosis, and a type 2 cytokine pathology.

87. The method of claim 85, wherein the inflammatory disease is atopic dermatitis.

88. The method of claim 85, wherein the inflammatory disease is systemic sclerosis.

89. The method of claim 86, wherein said asthma is steroid-insensitive asthma, steroid-refractory asthma, steroid-resistant asthma, atopic asthma, nonatopic asthma, persistent asthma, severe asthma, or steroid-refractory severe asthma.

90. The method of claim 89, wherein said severe asthma is T2 high endotype, T2 low endotype, or non-T2 endotype.

91. The method of claim 89, wherein said severe asthma is T2 low endotype or non-T2 endotype.
